(19) United States
(12) Patent Application Publication
(10) Pub. No.: US 2010/0204297 A1
(43) Pub. Date: Aug. 12, 2010

(54) INFLUENZA THERAPEUTIC

(75) Inventors: Jianzhu Chen, Brookline, MA (US); Qing Ge, Cambridge, MA (US); Herman N. Eisen, Waban, MA (US)

Correspondence Address: CHOATE, HALL & STEWART LLP, TWO INTERNATIONAL PLACE, BOSTON, MA, 02110, US

(73) Assignee: MASSACHUSETTS INSTITUTE OF TECHNOLOGY, Cambridge, MA (US)

(21) Appl. No.: 11/909,413

(22) PCT Filed: Mar. 22, 2006
(86) PCT No.: PCT/US2006/010491

§ 371 Date: Jan. 25, 2010

Related U.S. Application Data
(63) Continuation of application No. 11/102,097, filed on Apr. 8, 2005, now abandoned.
(60) Provisional application No. 60/664,580, filed on Mar. 22, 2005.

(51) Int. Cl. A61K 31/713; C07K 7/08; C07K 14/00; A61P 31/16; C12N 15/63

(52) U.S. Cl. 514/44 A; 530/326; 530/325; 530/324; 435/320.1

(57) ABSTRACT

The present invention provides compositions comprising an RNAi-inducing entity targeted to an influenza virus transcript and any of a variety of delivery agents. The invention further includes methods of use of the compositions for inhibiting a biological activity of an influenza virus and/or for treatment or prevention of influenza. The invention provides target portion sequences that are favorably conserved for RNAi across a plurality of influenza virus A strains isolated from human hosts and/or avian hosts and RNAi-inducing entities, e.g., siRNAs and shRNAs, targeted to such favorably conserved target portions. The invention provides a variety of nucleic acids comprising sequences identical or complementary to at least a portion of one or more of these favorably conserved target portion sequences. The invention further provides methods and compositions for delivering RNAi-inducing agents to an organ or tissue of a mammalian subject, e.g., to the lung. Methods of diagnosing influenza and determining the susceptibility of an influenza virus to inhibition by an RNAi-inducing agent are also provided. Transgenic animals that express an RNAi-inducing agent targeted to an influenza gene are another aspect of the invention.

### CROSS-REFERENCE TO RELATED APPLICATIONS

This application claims priority to U.S. Provisional Patent Application 60/664,580, filed Mar. 22, 2005 and U.S. patent application entitled Influenza Therapeutic, Ser. No. 11/102,097 filed Apr. 8, 2005, which is hereby incorporated by reference.

### GOVERNMENT SUPPORT

The United States Government has provided grant support utilized in the development of the present invention. In particular, National Institutes of Health grant numbers 5-RO1-AI44477, 5-RO1-AI44478, 5-ROI-CA60686, and 1-RO1-AI50631 have supported development of this invention. The Government may have certain rights in the invention.

### BACKGROUND OF THE INVENTION

Influenza is one of the most widely spread infections worldwide. In the United States between 20 and 40 thousand people die from influenza A virus infection or its complications each year. An estimated 20 to 40 million people died during the 1918 influenza A virus pandemic. During epidemics the number of influenza related hospitalizations may reach over 300,000 in a single winter season.

Several properties contribute to the epidemiological success of influenza virus. First, it is spread easily from person to person by aerosol (droplet infection). Second, small changes in influenza virus antigens are frequent (antigenic drift) so that the virus readily escapes protective immunity induced by a previous exposure to a different variant of the virus. Third, new strains of influenza virus can be easily generated by reassortment or mixing of genetic material between different strains (antigenic shift). In the case of influenza A virus, such mixing can occur between subtypes or strains that affect different species. The 1918 pandemic is thought to have been caused by a hybrid strain of virus derived from reassortment between a swine and a human influenza A virus.

Despite intensive efforts, there is still no satisfactory therapy for influenza virus infection and existing vaccines are limited in value in part because of the properties of antigenic shift and drift described above. For these reasons, global surveillance of influenza A virus has been underway for many years, and the National Institutes of Health designates it as one of the top priority pathogens for biodefense. Although current vaccines based upon inactivated virus are able to prevent illness in approximately 70-80% of healthy individuals under age 65, this percentage is far lower in the elderly or immunocompromised. In addition, the expense and potential side effects associated with vaccine administration make this approach less than optimal. Although the four antiviral drugs currently approved in the United States for treatment and/or prophylaxis of influenza are helpful, their use is limited due to concerns about side effects, compliance, and possible emergence of resistant strains. Therefore, there remains a need for the development of effective therapies for the treatment and prevention of influenza infection.

### SUMMARY OF THE INVENTION

The present invention provides novel compositions and methods for the treatment of respiratory virus infections, e.g., influenza infection due to influenza virus types A, B, and/or C. The compositions and methods are based on RNA interference (RNAi). RNAi is a conserved cellular process in which the presence of double-stranded RNA containing a portion that is complementary to a target RNA inhibits expression of the target RNA in a sequence-specific manner. Inhibition can be caused by cleavage of the target or inhibition of its translation. In contrast to currently available influenza therapeutics, the RNAi-inducing entities of the invention inhibit expression of influenza virus transcripts and thus prevent viral protein synthesis. This represents a fundamentally new approach to controlling influenza virus infection.

The invention provides RNAi-inducing agents such as short interfering RNA (siRNA) and short hairpin RNA (shRNA) molecules targeted to one or more target transcripts involved in virus production, replication, infection, and/or transcription of viral RNA, etc. In addition, the invention provides vectors whose presence within a cell results in transcription of one or more RNAs that hybridize to each other or self-hybridize to form an siRNA or shRNA that inhibits expression of at least one target transcript involved in virus production, virus infection, virus replication, and/or transcription of viral mRNA, etc. Preferably the virus is a respiratory virus. Preferred viruses are RNA viruses. RNA viruses include positive-stranded viruses and negative-stranded RNA viruses such as influenza virus. The viral genome can be segmented or non-segmented. According to certain embodiments of the invention the target transcript encodes a protein selected from the group consisting of: a polymerase, a nucleocapsid protein, a neuraminidase, a hemagglutinin, a matrix protein, and a nonstructural protein. In specific embodiments the target transcript encodes an influenza virus protein selected from the group consisting of hemagglutinin, neuraminidase, membrane protein 1, membrane protein 2, nonstructural protein 1, nonstructural protein 2, polymerase protein PB1, polymerase protein PB2, polymerase protein PA, nucleoprotein NP.

The invention also provides compositions comprising RNAi-inducing agents and/or vectors, e.g., the RNAi-inducing agents and/or vectors described herein, wherein the composition further comprises a delivery agent that facilitates delivery of the RNAi-inducing agent or vector. Preferred delivery agents include cationic polymers.

The invention further provides methods of treating or preventing viral diseases, particularly diseases caused by a respiratory virus such as influenza, by administering an inventive composition comprising an RNAi-inducing entity to a subject within an appropriate time window prior to exposure to the virus, while exposure is occurring, or following exposure, or at any point during which a subject exhibits symptoms of a disease caused by the virus. The compositions may be administered by a variety of routes. Preferred routes include intravenous, or directly into the respiratory system by inhalation, intranasally, as an aerosol, etc.

The invention provides nucleic acid sequences that represent portions of influenza virus transcripts that are preferred targets for RNAi. Certain of the preferred target portions are functionally preferred targets for RNAi. Certain of the preferred target portions are favorably conserved among multiple variants so that an RNAi-inducing agent designed based on the sequence of a specific variant will also inhibit variants whose corresponding target portion differs in sequence. Certain of the preferred target portions are highly conserved among multiple variants.

The invention provides nucleic acids comprising one or more preferred target portions, complements thereof, and fragments of either. The invention further provides nucleic acids that are RNAi-inducing entities e.g., RNAi-inducing agents and RNAi-inducing vectors that are targeted to one or more of these target portions. In preferred embodiments the RNAi-inducing entities are targeted to the NP, PA, PB1, or PB2 gene. The invention provides highly effective RNAi-inducing entities targeted to certain preferred target portions. Such highly effective RNAi-inducing entities may be particularly useful for treatment or prevention of influenza virus infection.

In particular, the invention provides an RNAi-inducing agent targeted to an influenza virus transcript, wherein the RNAi-inducing agent comprises a nucleic acid portion whose sequence comprises a sequence selected from the group consisting of: SEQ ID NOs: 272-380, its complement, or a fragment of either having a length of at least 15 nucleotides. The invention further provides an RNAi-inducing agent targeted to an influenza virus gene selected from the group consisting of the polymerase protein PB1 gene, the polymerase protein PB2 gene, the polymerase protein PA gene, and the nucleoprotein NP gene.

The invention also provides an isolated nucleic acid, or its complement, whose sequence comprises a sequence selected from the group consisting of SEQ ID NOs: 272-380, or comprises a fragment of a sequence selected from the group consisting of SEQ ID NOs: 272-380 having a length of at least 15 nucleotides, wherein the nucleic acid has a length of 100 nucleotides or less. In certain embodiments the length is at least 16 nucleotides.

The invention provides methods for diagnosis of virus infection and for determining whether a subject suspected of having a viral infection is infected with a virus of a particular type, strain, etc. In certain embodiments the method comprises determining whether a subject is infected with an influenza virus that is susceptible to inhibition by one or more of the RNAi-inducing entities of the invention. In a preferred embodiment a patient is diagnosed with influenza infection, and an RNAi-inducing entity targeted to the particular influenza strain infecting the subject is administered. The invention therefore provides integrated methods of influenza diagnosis and treatment. Certain of the diagnostic methods employ one or more nucleic acids of the invention.

The invention also provides diagnostic kits for detecting influenza virus infection and/or determining whether an influenza virus is susceptible to inhibition by an RNAi-inducing entity. The kits may comprise one or more nucleic acids of the invention and/or a probe or primer for detecting a preferred target portion of an influenza virus transcript.

The invention further provides methods of delivering RNAi-inducing entities to the respiratory tract of a mammalian subject. The inventors have discovered that RNAi-inducing entities can effectively silence gene expression in the lung when delivered directly to the respiratory tract of a mammalian subject. The inventors have further discovered that RNAi-inducing agents such as siRNA can be delivered directly to the vascular system of a mammalian subject using conventional volumes and administration methods and can effectively silence gene expression in the respiratory system, e.g., in the lung. For example, siRNA targeted to influenza virus transcripts inhibited influenza production in the lung when delivered to mice by either the intravenous or the inhalational route, indicating that therapeutically effective inhibition of gene expression in the respiratory system can be achieved by either method. In addition, siRNA targeted to a luciferase transcript silenced luciferase expression in the lung when administered by either the inhalational or intravenous route to mice that expressed luciferase, indicating that expression of essentially any gene can be inhibited using either of these methods. siRNA targeted to an endogenous gene was also effective in inhibiting expression in the lung when delivered by inhalation. The invention therefore provides methods that allow the use of RNAi for treating a wide range of diseases that affect the respiratory system including infections caused by respiratory viruses. The methods of intravascular delivery can also be employed to deliver effective amounts of an RNAi-inducing agent to organs or tissues other than the lung.

In one aspect, the invention provides a method of inhibiting expression of a transcript in an organ or tissue of a mammalian subject comprising introducing an RNAi-inducing entity such as an RNAi-inducing agent targeted to the transcript directly into the respiratory system of the subject. In a preferred embodiment the organ or tissue is part of the respiratory tract, e.g., the lung. Thus the invention provides a method of inhibiting expression of a transcript in the respiratory system of a mammalian subject comprising introducing an RNAi-inducing entity such as an RNAi-inducing agent targeted to the transcript directly into the respiratory system of the subject. In other embodiments the RNAi-inducing entity is delivered directly to the respiratory system, enters a vessel, and is transported via the vascular system to a site of activity other than the respiratory system, i.e., the respiratory route is used for systemic delivery.

The invention further provides a method of inhibiting expression of a transcript in the respiratory system of a mammalian subject comprising introducing an RNAi-inducing agent targeted to the transcript directly into the vascular system of a mammalian subject.

In another aspect, the invention provides a method of inhibiting expression of a transcript in an organ or tissue of a mammalian subject comprising introducing an RNAi-inducing agent targeted to the transcript directly into the vascular system of a mammalian subject. In another embodiment the invention provides a method of inhibiting expression of a transcript in a solid organ or tissue of a mammalian subject comprising introducing an RNAi-inducing entity such as an RNAi-inducing agent targeted to the transcript into the respiratory system of the subject, wherein the RNAi-inducing agent enters the vascular system and is transported to a location elsewhere in the body. In certain embodiments of any of the foregoing methods the RNAi-inducing agent is administered in an aqueous medium essentially free of lipids or delivery-enhancing polymers. In other embodiments of the invention the RNAi-inducing agent is administered in a composition that comprises a cationic polymer.

The invention provides compositions suitable for delivery to the respiratory system. In particular, the invention provides respirable aerosol formulations containing liquid or solid particles (e.g., dry powders) that comprise one or more of the inventive RNAi-inducing agents and/or RNAi-inducing vectors. The formulations may comprise a delivery agent and/or excipient. The invention also provides nasal sprays comprising the RNAi-inducing agents or RNAi-inducing vectors. The invention further provides a device for delivering a composition of the invention, e.g., a device such as an inhaler or nebulizer for delivery of a dry or liquid aerosol formulation to the respiratory system. The device may deliver single or multiple doses of the composition. An inventive composition may be provided inside the device and/or it may be provided separately (e.g. as a refill). The device may be disposable.

In another aspect, the invention provides non-human transgenic animals that express an RNAi-inducing agent targeted to an influenza gene.

This application refers to various patents, journal articles, and other publications, all of which are incorporated herein by reference. In addition, the following standard reference works are incorporated herein by reference: Ausubel, F., et al. (eds.) Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Goodman and Gilman's The Pharmacological Basis of Therapeutics, 10^{th} Ed. McGraw Hill, 2001.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In the case of conflict, the present specification, including definitions, will control. In addition, the materials, methods, and examples are illustrative only and are not intended to be limiting. Other features and advantages of the invention will be apparent from the following detailed description and claims. Where elements are listed in Markush group format, it is to be understood that each subgroup of these elements is also disclosed, and any element(s) can be removed from the group. Where ranges are given, endpoints are included unless otherwise stated or otherwise evident from the context.

### BRIEF DESCRIPTION OF THE DRAWING

FIG. 1A, adapted from Julkunen, I., et al., infra, presents a schematic of the influenza virus.

FIG. 1B, adapted from Fields' Virology, infra, shows the genome structure of the influenza virus and the transcripts derived from the influenza genome. Thin lines at the 5′ and 3′-termini of the mRNAs represent untranslated regions. Shaded or hatched areas represent coding regions in the 0 or +1 reading frames, respectively. Introns are depicted by V-shaped lines. Small rectangles at the 5′ ends of the mRNAs represent heterogenous cellular RNAs covalently linked to the viral nucleic acids. A₍ₙ₎ symbolizes the polyA tail.

FIG. 2, adapted from Julkunen, I., et al., infra, shows the influenza virus replication cycle.

FIG. 3 shows the structure of siRNAs observed in the Drosophila system.

FIG. 4 schematically represents of the steps involved in RNA interference in Drosophila.

FIG. 5 shows a variety of exemplary siRNA and shRNA structures useful in accordance with the present invention.

FIG. 6 represents an alternative inhibitory pathway, in which the DICER enzyme cleaves a substrate having a base mismatch in the stem to generate an inhibitory product that binds to the 3′ UTR of a target transcript and inhibits translation.

FIG. 7 presents one example of a construct that may be used to direct transcription of both strands of an inventive siRNA.

FIG. 8 depicts one example of a construct that may be used to direct transcript of a single RNA molecule that hybridizes to form an shRNA in accordance with the present invention.

FIG. 9 shows a sequence comparison between six strains of influenza virus A that have a human host of origin. Dark shaded areas were used to design siRNAs that were tested as described in Example 2. The base sequence is the sequence of strain A/Puerto Rico/8/34. Lightly shaded letters indicate nucleotides that differ from the base sequence.

FIG. 10 shows a sequence comparison between two strains of influenza virus that have a human host of origin and five strains of influenza virus A that have an animal host of origin. Darkly shaded areas were used to design siRNAs that were tested as described in Example 2. The base sequence is the sequence of strain A/Puerto Rico/8/34. Lightly shaded letters indicate nucleotides that differ from the base sequence.

FIGS. 11A-11F show the results of experiments indicating that siRNA inhibits influenza virus production in MDCK cells. Six different siRNAs that target various viral transcripts were introduced into MDCK cells by electroporation, and cells were infected with virus 8 hours later. FIG. 11A is a time course showing viral titer in culture supernatants as measured by hemagglutinin assay at various times following infection with viral strain A/PR/8/34 (H1N1) (PR8), at a multiplicity of infection (MOI) of 0.01 in the presence or absence of the various siRNAs or a control siRNA. FIG. 11B is a time course showing viral titer in culture supernatants as measured by hemagglutinin assay at various times following infection with influenza virus strain A/WSN/33 (H1N1) (WSN) at an MOI of 0.01 in the presence or absence of the various siRNAs or a control siRNA. FIG. 11C shows a plaque assay showing viral titer in culture supernatants from virus infected cells that were either mock transfected or transfected with siRNA NP-1496. FIG. 11D shows inhibition of influenza virus production at different doses of siRNA. MDCK cells were transfected with the indicated amount of NP-1496 siRNA followed by infection with PR8 virus at an MOI of 0.01. Virus titer was measured 48 hours after infection. Representative data from one of two experiments are shown. FIG. 11E shows inhibition of influenza virus production by siRNA administered after virus infection. MDCK cells were infected with PR8 virus at an MOI of 0.01 for 2 hrs and then transfected with NP-1496 (2.5 nmol). Virus titer was measured at the indicated times after infection. Representative data from one of two experiments are shown.

FIG. 12 shows a sequence comparison between a portion of the 3′ region of NP sequences among twelve influenza A virus subtypes or isolates that have either a human or animal host of origin. The shaded area was used to design siRNAs that were tested as described in Examples 2 and 3. The base sequence is the sequence of strain A/Puerto Rico/8/34. Shaded letters indicate nucleotides that differ from the base sequence.

FIG. 13 shows positions of various siRNAs relative to influenza virus gene segments, correlated with effectiveness in inhibiting influenza virus.

FIG. 14A is a schematic of a developing chicken embryo indicating the area for injection of siRNA and siRNA/delivery agent compositions.

FIG. 14B shows the ability of various siRNAs to inhibit influenza virus production in developing chicken embryos.

FIG. 15 is a schematic showing the interaction of nucleoprotein with viral RNA molecules.

FIGS. 16A and 16B show schematic diagrams illustrating the differences between influenza virus vRNA, mRNA, and cRNA (template RNA) and the relationships between them. The conserved 12 nucleotides at the 3′ end and 13 nucleotides at the 5′ end of each influenza A virus vRNA segment are indicated in FIG. 16B. The mRNAs contain an m⁷GpppN^{m} cap structure and, on average, 10 to 13 nucleotides derived from a subset of host cell RNAs. Polyadenylation of the mRNAs occurs at a site in the mRNA corresponding to a location 15 to 22 nucleotides before the 5′ end of the vRNA segment. Arrows indicate the positions of primers specific for each RNA species. (Adapted from ref. (1)).

FIG. 17 shows amounts of viral NP and NS RNA species at various times following infection with virus, in cells that were mock transfected or transfected with siRNA NP-1496 6-8 hours prior to infection.

FIG. 18A shows that inhibition of influenza virus production requires a wild type (wt) antisense strand in the duplex siRNA. MDCK cells were first transfected with siRNAs formed from wt and modified (m) strands and infected 8 hrs later with PR8 virus at MOI of 0.1. Virus titers in the culture supernatants were assayed 24 hrs after infection. Representative data from one of the two experiments are shown. FIG. 18B shows that M-specific siRNA inhibits the accumulation of specific mRNA. MDCK cells were transfected with M-37, infected with PR8 virus at MOI of 0.01, and harvested for RNA isolation 1, 2, and 3 hrs after infection. The levels of M-specific mRNA, cRNA, and vRNA were measured by reverse transcription using RNA-specific primers, followed by real time PCR. The level of each viral RNA species is normalized to the level of γ-actin mRNA (bottom panel) in the same sample. The relative levels of RNAs are shown as mean value±S.D. Representative data from one of the two experiments are shown.

FIGS. 19A-D show that NP-specific siRNA inhibits the accumulation of not only NP- but also M- and NS-specific mRNA, vRNA, and cRNA. MDCK (A-C) and Vero (D) cells were transfected with NP-1496, infected with PR8 virus at MOI of 0.1, and harvested for RNA isolation 1, 2, and 3 hrs after infection. The levels of mRNA, cRNA, and vRNA specific for NP, M, and NS were measured by reverse transcription using RNA-specific primers followed by real time PCR. The level of each viral RNA species is normalized to the level of γ-actin mRNA (not shown) in the same sample. The relative levels of RNAs are shown. Representative data from one of three experiments are shown.

FIGS. 19E-G, right side in each figure, show that PA-specific siRNA inhibits the accumulation of not only PA- but also M- and NS-specific mRNA, vRNA, and cRNA. MDCK cells were transfected with PA-1496, infected with PR8 virus at MOI of 0.1, and harvested for RNA isolation 1, 2, and 3 hrs after infection. The levels of mRNA, cRNA, and vRNA specific for PA, M, and NS were measured by reverse transcription using RNA-specific primers followed by real time PCR. The level of each viral RNA species is normalized to the level of γ-actin mRNA (not shown) in the same sample. The relative levels of RNAs are shown.

FIG. 19H shows that NP-specific siRNA inhibits the accumulation of PB1- (top panel), PB2- (middle panel) and PA- (lower panel) specific mRNA. MDCK cells were transfected with NP-1496, infected with PR8 virus at MOI of 0.1, and harvested for RNA isolation 1, 2, and 3 hrs after infection. The levels of mRNA specific for PB1, PB2, and PA mRNA were measured by reverse transcription using RNA-specific primers followed by real time PCR. The level of each viral RNA species is normalized to the level of γ-actin mRNA (not shown) in the same sample. The relative levels of RNAs are shown.

FIG. 20A shows sequences of siRNA CD8-61 and its hairpin derivative CD8-61F.

FIG. 20B shows inhibition of CD8α expression by CD8-61 and CD8-61F. A CD8⁺CD4⁺ T cell line was transfected with either CD8-61 or CD8-61F by electroporation. CD8α expression was assayed by flow cytometry 48 hrs later. Unlabeled line, mock transfection.

FIG. 20C shows a schematic diagram of the pSLOOP III vector, in which expression of CD8-61F hairpin RNA is driven by H1 RNA pol III promoter. Terminator, termination signal sequence.

FIG. 20D presents plots showing silencing of CD8α in HeLa cells using pSLOOP III. Untransfected cells did not express CD8α. Cells were transfected with the CD8α expression vector and either a promoterless pSLOOP III-CD8-61F construct, synthetic siRNA, or a pSLOOP III-CD8-61F containing a promoter.

FIG. 21A shows schematic diagrams of NP-1496 and GFP-949 siRNA and their hairpin derivatives/precursors.

FIG. 21B shows tandem arrays of NP-1496H and GFP-949H in two different orders.

FIG. 21C shows pSLOOP III expression vectors. Hairpin precursors of siRNA are cloned in the pSLOOP III vector alone (top), in tandem arrays (middle), or simultaneously with independent promoter and termination sequence (bottom).

FIG. 22A is a plot showing that siRNA inhibits influenza virus production in mice when administered together with the cationic polymer PEI prior to infection with influenza virus. Filled squares (no treatment); Open squares (GFP siRNA); Open circles (30 μg NP siRNA); Filled circles (60 μg NP siRNA). Each symbol represents an individual animal. p values between different groups are shown.

FIG. 22B is a plot showing that siRNA inhibits influenza virus production in mice when administered together with the cationic polymer PLL prior to infection with influenza virus. Filled squares (no treatment); Open squares (GFP siRNA); Filled circles (60 μg NP siRNA). Each symbol represents an individual animal. p values between different groups are shown.

FIG. 22C is a plot showing that siRNA inhibits influenza virus production in mice when administered together with the cationic polymer jetPEI prior to infection with influenza virus significantly more effectively than when administered in PBS. Open squares (no treatment); Open triangles (GFP siRNA in PBS); Filled triangles (NP siRNA in PBS); Open circles (GFP siRNA with jetPEI); Filled circles (NP siRNA with jetPEI). Each symbol represents an individual animal. p values between different groups are shown.

FIG. 22D is a plot showing that siRNA targeted to NP inhibits influenza virus production in mice when administered intravenously together with a cationic poly(beta amino ester) (J28). Open circles (no treatment); Filled squares (NP siRNA with J28).

FIG. 22E is a plot showing that siRNA targeted to NP inhibits influenza virus production in mice when administered intraperitoneally together with a cationic poly(beta amino ester) (J28 or C32) while a control RNA (GFP) has no significant effect. Open circles (no treatment); Open squares (GFP siRNA with J28) Filled squares (NP siRNA with J28); Open triangles (GFP siRNA with C32); Filled triangles (NP siRNA with C32). p values between control and treated groups are shown.

FIG. 23 is a plot showing that siRNAs targeted to influenza virus NP and PA transcripts exhibit an additive effect when administered together prior to infection with influenza virus. Filled squares (no treatment); Open circles (60 μg NP siRNA); Open triangles (60 μg PA siRNA); Filled circles (60 μg NP siRNA+60 μg PA siRNA). Each symbol represents an individual animal. p values between different groups are shown.

FIG. 24 is a plot showing that siRNA inhibits influenza virus production in mice when administered following infection with influenza virus. Filled squares (no treatment); Open squares (60 μg GFP siRNA); Open triangles (60 μg PA siRNA); Open circles (60 μg NP siRNA); Filled circles (60 μg NP+60 μg PA siRNA). Each symbol represents an individual animal. p values between different groups are shown.

FIG. 25A is a schematic diagram of a lentiviral vector expressing a shRNA. Transcription of shRNA is driven by the U6 promoter. EGFP expression is driven by the CMV promoter. SIN-LTR, Ψ, cPPT, and WRE are lentivirus components. The sequence of NP-1496 shRNA is shown.

FIG. 25B presents plots of flow cytometry results demonstrating that Vero cells infected with the lentivirus depicted in FIG. 25B express EGFP in a dose-dependent manner. Lentivirus was produced by co-transfecting DNA vector encoding NP-1496a shRNA and packaging vectors into 293T cells. Culture supernatants (0.25 ml or 1.0 ml) were used to infect Vero cells. The resulting Vero cell lines (Vero-NP-0.25 and Vero-NP-1.0) and control (uninfected) Vero cells were analyzed for GFP expression by flow cytometry. Mean fluorescence intensity of Vero-NP-0.25 (upper portion of figure) and Vero-NP-1.0 (lower portion of figure) cells are shown. The shaded curve represents mean fluorescence intensity of control (uninfected) Vero cells.

FIG. 25C is a plot showing inhibition of influenza virus production in Vero cells that express NP-1496 shRNA. Parental and NP-1496 shRNA expressing Vero cells were infected with PR8 virus at MOI of 0.04, 0.2 and 1. Virus titers in the supernatants were determined by hemagglutination (HA) assay 48 hrs after infection.

FIG. 26 is a plot showing that influenza virus production in mice is inhibited by administration of DNA vectors that express siRNA targeted to influenza virus transcripts. Sixty μg of DNA encoding RSV, NP-1496 (NP) or PB1-2257 (PB1) shRNA were mixed with 40 μl Infasurf and were administered into mice by instillation. For no treatment (NT) group, mice were instilled with 60 μl of 5% glucose. Thirteen hrs later, the mice were infected intranasally with PR8 virus, 12000 pfu per mouse. The virus titers in the lungs were measured 24 hrs after infection by MDCK/hemagglutinin assay. Each data point represents one mouse. p values between groups are indicated.

FIG. 27A shows results of an electrophoretic mobility shift assay for detecting complex formation between siRNA and poly-L-lysine (PLL). siRNA-polymer complexes were formed by mixing 150 ng of NP-1496 siRNA with increasing amounts of polymer (0-1200 ng) for 30 min at room temperature. The reactive mixtures were then run on a 4% agarose gel and siRNAs were visualized with ethidium-bromide staining.

FIG. 27B shows results of an electrophoretic mobility shift assay for detecting complex formation between siRNA and poly-L-arginine (PLA). SiRNA-polymer complexes were formed by mixing 150 ng of NP-1496 siRNA with increasing amounts of polymer (0-1200 ng) for 30 min at room temperature. The reactive mixtures were then run on a 4% agarose gel and siRNAs were visualized with ethidium-bromide staining.

FIG. 28A is a plot showing cytotoxicity of siRNA/PLL complexes. Vero cells in 96-well plates were treated with siRNA (400 pmol)/polymer complexes for 6 hrs. The polymer-containing medium was then replaced with DMEM-10% FCS. The metabolic activity of the cells was measured 24 h later by using the MTT assay. Squares=PLL (MW ˜8K); Circles=PLL (MW ˜42K) Filled squares=25%; Open triangles=50%; Filled triangles=75%; X=95%. The data are shown as the average of triplicates.

FIG. 28B is a plot showing cytotoxicity of siRNA/PLA complexes. Vero cells in 96-well plates were treated with siRNA (400 pmol)/polymer complexes for 6 hrs. The polymer-containing medium was then replaced with DMEM-10% FCS. The metabolic activity of the cells was measured 24 h later by using the MTT assay. The data are shown as the average of triplicates.

FIG. 29A is a plot showing that PLL stimulates cellular uptake of siRNA. Vero cells in 24-well plates were incubated with Lipofectamine+siRNA (400 pmol) or with siRNA (400 pmol)/polymer complexes for 6 hrs. The cells were then washed and infected with PR8 virus at a MOI of 0.04. Virus titers in the culture supernatants at different time points after infection were measured by HA assay. Polymer to siRNA ratios are indicated. Open circles=no treatment; Filled squares=Lipofectamine; Filled triangles=PLL (MW ˜42K); Open triangles=PLL (MW ˜8K).

FIG. 29B is a plot showing that poly-L-arginine stimulates cellular uptake of siRNA. Vero cells in 24-well plates were incubated with siRNA (400 pmol)/polymer complexes for 6 hrs. The cells were then washed and infected with PR8 virus at a MOI of 0.04. Virus titers in the culture supernatants at different time points after infection were measured by HA assay. Polymer to siRNA ratios are indicated. 0, 25, 50, 75, and 95% refer to percentage of c-amino groups on PLL substituted with imidazole acetyl groups. Closed circles=no transfection; Open circles=Lipofectamine; Open and filled squares=0% and 25% (Note that the data points for 0% and 25% are identical); Filled triangles=50%; Open triangles=75%; X=95%.

FIG. 30A is a bar graph showing that PEI mediates DNA transfection in the lungs after intravenous injection. Luc activities in relative light units in 0.5 mg of protein in different organs over 4 days are shown. Data are from one of two experiments.

FIG. 30B is a bar graph showing that PEI mediates DNA transfection in the lungs after intratracheal administration. The figure shows average Luc activities in relative light units in 0.5 mg of protein in the indicated organs of three mice per group 24 hr after DNA administration. Error bars indicate standard deviation.

FIG. 30C is a bar graph showing inhibition of luciferase activity in the lung by intravenous delivery of siRNA. The figure shows average Luc activities in relative light units in 0.5 mg of protein in the indicated organs for three mice per group 24 hr after administration of a luciferase construct. Error bars indicate standard deviation.

FIG. 30D is a bar graph showing inhibition of luciferase activity in the lung by inhalational delivery of siRNA. The figure shows average Luc activities in relative light units in 0.5 mg of protein in the lungs of mice 24 hr after administration of a luciferase construct. Error bars indicate standard deviation. Experiments were performed in duplicate using 3 mice per group, and similar results were obtained. Results of one of the experiments are presented here.

FIGS. 31A and 31B are plots showing that administration of siRNA without a delivery agent inhibits influenza virus production in mice.

FIGS. 32A-32J show sequences of influenza virus strain PR8 transcripts that were used for selection of target portions for RNAi.

FIG. 33 is Table 17, which shows the sequences of functional target portions for RNAi to inhibit influenza virus.

FIG. 34 is Table 18, which shows the sequences of influenza virus target portions that are favorably conserved for RNAi among influenza virus strains derived from humans.

FIG. 35 is Table 20, which shows the sequences of influenza virus target portions that are favorably conserved for RNAi among influenza virus strains derived from humans and avians.

### ABBREVIATIONS

DNA: deoxyribonucleic acid

RNA: ribonucleic acid

vRNA: virion RNA in the influenza virus genome, negative strand

cRNA: complementary RNA, a direct transcript of vRNA, positive strand

mRNA: messenger RNA transcribed from vRNA or cellular genes, positive strand, a template for protein synthesis

dsRNA: double-stranded RNA

siRNA: short interfering RNA

shRNA: short hairpin RNA

miRNA: microRNA

RNAi: RNA interference

bp: base pair(s)

nt: nucleotide(s)

### DEFINITIONS

As used herein, the terms “approximately” or “about” in reference to a number are generally taken to include numbers that fall within a range of 5% of the number in either direction (greater than or less than the number) unless otherwise stated or otherwise evident from the context (except where such number would exceed 100% of a possible value).

The term “avian” as used herein is intended to refer to any species, subspecies or race of organism of the taxonomic class ava, e.g., chicken, turkey, duck, goose, quail, pheasants, parrots, finches, hawks, and crows. The term includes the various known strains of Gallus gallus, or chickens, (for example, White Leghorn, Brown Leghorn, Barred-Rock, Sussex, New Hampshire, Rhode Island, Ausstralorp, Minorca, Amrox, Calif. Gray, Italian Partidge-colored), as well as strains of turkeys, pheasants, quails, duck, ostriches and other poultry commonly bred.

The term “complementary” is used herein in accordance with its art-accepted meaning to refer to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence when the strands are aligned in anti-parallel orientation, the nucleotides form a complementary base pair, and the nucleic acids are complementary at that position. The percent complementarity of a first nucleic acid to a second nucleic acid may be evaluated by aligning them in antiparallel orientation for maximum complementarity over a window of evaluation along the second nucleic acid, determining the total number of nt in both strands that form complementary base pairs within the window, dividing by the total number of nt within the window, and multiplying by 100. For example, AAAAAAAA and TTTGTTAT are 75% complementary since there are 12 nt in complementary base pairs out of a total of 16. Nucleic acids that are at least 70% complementary over a window of evaluation are considered substantially complementary over that window. When computing the number of complementary nt needed to achieve a particular % complementarity, fractions are rounded to the nearest whole number. A position occupied by non-complementary nucleotides constitutes a mismatch, i.e., the position is occupied by a non-complementary base pair. In order to achieve maximum complementarity gaps may be introduced into either or both of the nucleic acids within a window of evaluation. Nucleotide(s) opposite a gap is/are unpaired and constitute a bulge, i.e., there is no nucleotide located opposite in the other nucleic acid. Typically a percent complementarity is determined over a window of evaluation of at least 15 nt in length, e.g., 19 nt, where the length does not include gaps. For purposes of determining % complementarity, a 1 nt bulge is considered to be a single non-complementary nt; a bulge of between 2 and 5 nt is considered to be 2 non-complementary nt; a bulge of between 6 and 10 nt is considered to be 3 non-complementary nt. A bulge of length K nt, where K is greater than 10, is considered to be 3+(K−10) non-complementary nt.

“Directly into the respiratory system” refers to administration via the nose, mouth, or trachea, preferably the nose or mouth, such that a significant fraction of an active agent in the composition (e.g., more than 10%, preferably more than 25% of the active agent, by weight) enters the upper and/or lower respiratory tract.

“Directly into the vascular system” refers to administration into a vessel (e.g., an artery or vein) by injection or catheter or any other method in which the vascular system is entered from outside the body, typically involving penetrating the wall of a vessel. “Indirectly into the vascular system” refers to a mode of administration in which the vascular system is not penetrated. A preferred example of indirect delivery of a substance to the vascular system is direct delivery of the substance to the respiratory system, followed by passage of the substance across a vessel wall. The substance may then be transported to a target tissue or organ elsewhere in the body (and may return to the lung).

An “effective amount” of an active agent refers to the amount of the active agent sufficient to elicit a desired biological response. As will be appreciated by those of ordinary skill in this art, the absolute amount of a particular agent that is effective may vary depending on such factors as the desired biological endpoint, the agent to be delivered, the target tissue, etc. An “effective amount” may be administered in a single dose or multiple doses. For example, an effective amount of an RNAi-inducing entity may be an amount sufficient to achieve one or more of the following: (i) reduce expression of a target transcript by at least 20%, preferably at least 40%; (ii) reduce virus titer by at least 25%; (iii) reduce virus titer by at least 2-fold; (iv) delay or prevent the development of clinically significant virus infection; (v) reduce the duration or severity of at least one symptom of a virus infection, etc.

A composition is “essentially free” of a substance if the composition contains less than 1% of the substance by weight, preferably less than 0.5%, more preferably less than 0.1%. More preferably the substance is entirely absent from the composition. A composition is considered essentially free of delivery-enhancing polymers or lipids if no such polymer or lipid has been deliberately included in the composition.

The term “hybridize”, as used herein, refers to the interaction between two nucleic acid sequences comprising or consisting of complementary portions such that a duplex structure is formed that is stable under the particular conditions of interest, e.g., in a eukaryotic cell, in a Drosophila lysate, etc. Typically a first nucleic acid is considered to hybridize to a second nucleic acid if the Tm of a duplex formed by the first and second nucleic acids is less than 15° C. below, preferably less than 10° C. below the Tm of a duplex that would be formed by the second nucleic acid and a third nucleic acid that is the same length as, and 100% complementary to, the second nucleic acid and contains nucleosides and internucleosidic linkages of the same type. Hybridization conditions suitable for various applications are known in the art and/or found in standard reference works, e.g., Ausubel, supra, and Sambrook, supra. In an exemplary embodiment, stringent hybridization conditions comprise 6× sodium chloride/sodium citrate (SSC) and 0.1% SDS at a temperature 10-15° C. below the Tm of a perfectly complementary duplex, followed by washing 1-2 times for 30 minutes in 2×SSC and 0.1% SDS at a temperature 25° C. below the Tm of a perfectly complementary duplex.

“Identity” refers to the extent to which the sequence of two or more nucleic acids is the same. The percent identity between first and second nucleic acids over a window of evaluation may be computed by aligning the nucleic acids in parallel orientation, determining the number of nucleotides within the window of evaluation that are opposite an identical nucleotide, dividing by the total number of nucleotides in the window, and multiplying by 100. When computing the number of identical nucleotides needed to achieve a particular % identity, fractions are to be rounded to the nearest whole number. Nucleic acids that are at least 70% identical over a window of evaluation, e.g., at least 80%, at least 90%, of more, are considered substantially identical over that window. Typically the window of evaluation is at least 15 nt in length along the second nucleic acid, e.g., 19 nt, where the length does not include gaps. For purposes of determining % identity, a 1 nt gap is considered to be a single nt that is not opposite an identical nt; a gap of between 2 and 5 nt is considered to be 2 nt that are not opposite an identical nt; a gap of between 6 and 10 nt is considered to be 3 nt that are not opposite an identical nt. A gap of length K nt, where K is greater than 10, is considered to be 3+(K−10) nt that are not opposite an identical nt.

The term “influenza virus” is used here to refer to any strain of influenza virus that is capable of causing disease in an animal or human subject, or that is an interesting candidate for experimental analysis. Influenza viruses are described in Fields, B., et al., Fields' Virology, 4^{th}. ed., Philadelphia: Lippincott Williams and Wilkins; ISBN: 0781718325, 2001. In particular, the term encompasses any strain of influenza A virus that is capable of causing disease in an animal or human subject, or that is an interesting candidate for experimental analysis. A large number of influenza A isolates have been partially or completely sequenced. Appendix A presents merely a partial list of complete sequences for influenza A genome segments that have been deposited in a public database (The Influenza Sequence Database (ISD), see Macken, C., Lu, H., Goodman, J., & Boykin, L., “The value of a database in surveillance and vaccine selection.” in Options for the Control of Influenza IV. A. D. M. E. Osterhaus, N. Cox & A. W. Hampson (Eds.) Amsterdam: Elsevier Science, 2001, 103-106). This database also contains complete sequences for influenza B and C genome segments. The database is available on the World Wide Web at the Web site having URL http://www.flu.lanl.gov/ along with a convenient search engine that allows the user to search by genome segment, by species infected by the virus, and by year of isolation. Influenza sequences are also available on Genbank. Sequences of influenza genes are therefore readily available to, or determinable by, those of ordinary skill in the art.

The term “in vivo”, as used herein with respect to the synthesis, processing, or activity of an RNAi-inducing agent generally refers to events that occur within a cell as opposed to in a cell-free system. In general, the cell can be maintained in tissue culture or can be part of an intact organism.

“Isolated”, as used herein, means 1) separated from at least some of the components with which it is usually associated in nature; 2) prepared or purified by a process that involves the hand of man; and/or 3) not occurring in nature. Any of the nucleic acids and nucleic acid structures described herein may be in an isolated form.

“Ligand”, as used herein, means a molecule that specifically binds to a second molecule through a mechanism other than an antigen-antibody interaction. The term encompasses, for example, polypeptides, peptides, and small molecules, either naturally occurring or synthesized, including molecules whose structure has been invented by man.

“Nucleic acid based assay” refers to any assay or method in which the presence of a nucleic acid is detected and/or a nucleic acid is identified. The assay can be qualitative or quantitative.

“Nucleobase”, as used herein, means a nitrogen-containing heterocyclic moiety capable of forming hydrogen bonds, preferably Watson-Crick hydrogen bonds, in pairing with a complementary nucleobase or nucleobase analog, e.g., a purine or a pyrimidine. Typical nucleobases are the naturally occurring nucleobases adenine, guanine, cytosine, uracil, thymine, and analogs of the naturally occurring nucleobases (Fasman, Practical Handbook of Biochemistry and Molecular Biology, pp. 385-394, CRC Press, Boca Raton, Fla., 1989). The terms “nucleobase” and “base” are used interchangeably herein.

A “nucleotide” comprises a nitrogenous base, a sugar molecule, and a phosphate group. A nucleoside comprises a nitrogenous base (nucleobase) linked to a sugar molecule. In a naturally occurring nucleic acid, phosphate groups covalently link adjacent nucleosides to form a polymer. A nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose). A nucleoside can comprise a universal base, i.e., a base can that substitute for one, or preferably any, of the natural bases commonly found in nucleic acids when opposite them in a duplex (see, e.g., 142-149). In certain embodiments of the invention a nucleic acid comprises an abasic residue.

“Operably linked”, as used herein, refers to a relationship between two nucleic acids sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

The term “organ” is used as in the art, to refer to a tissue or group of tissues that constitute a morphologically and functionally distinct part of an organism. Examples include lung, heart, liver, pancreas, breast, kidney, intestine, bladder, bone, skin, etc. The term “tissue” is used as in the art, to refer to a group of cells, usually of similar structure, typically organized to perform one or more identical or related functions. Red blood cells, white blood cells, and platelets are considered to be circulating tissues comprising individual cells or cell fragments.

“Preventing” refers to causing a disease, disorder, condition, or symptom or manifestation of such, or worsening of the severity of such, not to occur. Preventing includes reducing the risk that a disease, disorder, condition, or symptom or manifestation of such, or worsening of the severity of such, will occur. Thus if a composition or method reduces the risk that a disease, disorder, condition, or symptom or manifestation of such, or worsening of the severity of such will occur on an individual or population basis, the composition or method is said to prevent the disease, disorder, condition, or symptom or manifestation of such, or worsening of the severity of such.

The term “primer” as used herein refers to an oligonucleotide, whether natural or synthetic, that is capable of acting as a point of initiation of nucleic acid synthesis when hybridized to a nucleic acid template under conditions in which primer extension, e.g., polymerase-catalyzed primer extension, is initiated. The appropriate length of a primer depends on the intended use of the primer, but typically ranges from 15 to 35 nt.

In some cases a primer may be longer, e.g., up to about 60 nt in length. Short primer molecules generally require cooler temperatures to form sufficiently stable hybrid complexes with a template. A primer need not reflect the exact sequence of the template but must be sufficiently complementary to hybridize with a template for primer elongation to occur.

The term “probe” as used herein, when referring to a nucleic acid, refers to a nucleic acid that can hybridize with and thereby detect the presence of a complementary nucleic acid The probe should be sufficiently complementary to the nucleic acid being detected so that specific hybridization can occur under the hybridization stringency conditions used. The probe may be modified with labels such as fluorescent moieites, biotin, etc.

“Purified”, as used herein, means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

The term “regulatory sequence” is used herein to describe a region of nucleic acid sequence that directs, enhances, or inhibits the expression (particularly transcription, but in some cases other events such as splicing or other processing) of sequence(s) with which it is operatively linked. The term includes expression signals such as promoters, enhancers and other transcriptional control elements. In some embodiments of the invention, regulatory sequences may direct constitutive expression of a nucleotide sequence; in other embodiments, regulatory sequences may direct tissue-specific and/or inducible expression. A regulatory sequence may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc. Alternately, the regulatory sequence may comprise a promoter and/or enhancer that is active in epithelial cells, e.g., respiratory epithelial cells. For example, a promoter for a gene that encodes a surfactant protein can be used.

“Respiratory system” refers to any component upper respiratory tract (e.g., e.g., nasal passages, nasopharynx, oropharynx) or lower respiratory tract (e.g., trachea, bronchi, bronchioles, and/or alveoli). The larynx can be considered a component of either the upper or lower respiratory tract. The terms “respiratory system” and “respiratory tract” are used interchangeably herein.

A “respiratory virus” is a virus that infects cells present in the upper and/or lower respiratory tract of a subject. Preferably the virus infects respiratory epithelial cells. Other cells that may be infected include, but are not limited to, bronchoalveolar macrophages, dendritic cells, etc. Examples of respiratory viruses include: influenza virus, parainfluenza virus (PIV), pneumovirus, metapneumovirus, coronavirus, adenovirus, rhinovirus, respiratory syncytial virus (RSV), reovirus, herpes virus, and hantavirus.

The term “RNAi-inducing agent” is used to refer to siRNAs, shRNAs, and other double-stranded structures (e.g., dsRNA) that can be processed to yield an siRNA or shRNA or other small RNA species that inhibits expression of a target transcript by RNA interference. In certain embodiments of the invention an RNAi-inducing agent inhibits expression of a target RNA via an RNA interference pathway that involves translational repression.

The term “RNAi-inducing entity”, encompasses RNA molecules and vectors whose presence within a cell results in RNAi and leads to reduced expression of a transcript to which the RNAi-inducing entity is targeted. The RNAi-inducing entity may be, for example, an RNAi-inducing agent such as an siRNA, shRNA, or an RNAi-inducing vector. Use of the terms “RNAi-inducing entity”, “RNAi-inducing agent”, or “RNAi-inducing vector” is not intended to imply that the entity, agent, or vector upregulates or activates RNAi in general, though it may do so, but simply to indicate that presence of the entity, agent, or vector within the cell results in RNAi-mediated reduction in expression of a target transcript. An “RNAi-inducing entity” as used herein is an entity that has been modified or generated by the hand of man and/or whose presence in a cell is a result of human intervention as distinct, e.g., from endogenous RNA species or RNA species that are produced in a cell during the natural course of viral infection.

An “RNAi-inducing vector” is a vector whose presence within a cell results in transcription of one or more RNAs that hybridize to each other or self-hybridize to form an RNAi-inducing agent such as an siRNA or shRNA. In various embodiments of the invention this term encompasses plasmids or viruses whose presence within a cell results in production of one or more RNAs that self-hybridize or hybridize to each other to form an RNAi-inducing agent. In general, the vector comprises a nucleic acid operably linked to expression signal(s) so that one or more RNA molecules that hybridize or self-hybridize to form an RNAi-inducing agent is transcribed when the vector is present in a cell. Thus the vector provides a template for intracellular synthesis of the RNAi-inducing agent. For purposes of inducing RNAi, presence of a viral genome in a cell constitutes presence of the virus within the cell. A vector is considered to be present within a cell if it is introduced into the cell, enters the cell, or is inherited from a parental cell, regardless of whether it is subsequently modified or processed within the cell. An RNAi-inducing vector is considered to be targeted to a transcript if the vector comprises a template for transcription of an RNAi-inducing agent that is targeted to the transcript. Such vectors have a number of other uses in addition to transcript inhibition in a cell. For example, they may be used for in vitro production of an RNAi-inducing agent and/or for production of the agent in a cell that may or may not contain a transcript to which the vector is targeted.

A “short, interfering RNA” comprises a double-stranded (duplex) RNA that is between 15 and approximately 29 nucleotides in length or any other subrange or specific value within the interval between 15 and 29, e.g., 16-18, 17-19, 21-23, 24-27, 27-29 nt long and optionally further comprises one or two single-stranded overhangs, e.g., a 3′ overhang on one or both strands. In certain embodiments the duplex is approximately 19 nt long. The overhang may be, e.g., 1-6 residues in length, e.g., 2 nt. An siRNA may be formed from two RNA molecules that hybridize together or may alternatively be generated from an shRNA. In certain embodiments of the invention one or both of the 5′ ends of an siRNA has a phosphate group while in other embodiments one or more of the 5′ ends lacks a phosphate group. In certain embodiments of the invention one or both of the 3′ ends has a hydroxyl group while in other embodiments they do not. One strand of an siRNA, which is referred to as the “antisense strand” or “guide strand” includes a portion that hybridizes with a target transcript. In certain preferred embodiments of the invention, the antisense strand of the siRNA is 100% complementary with a region of the target transcript, i.e., it hybridizes to the target transcript without a single mismatch or bulge over a target region between 15 and approximately 29 nt in length, preferably at least 16 nt in length, more preferably 18-20, e.g., 19 nt in length. The region of complementarity may be any subrange or specific value within the interval between 17 and 29, e.g., 17-18, 19-21, 21-23, 19-23, 24-27, 27-29. In other embodiments the antisense strand is substantially complementary to the target region, i.e., one or more mismatches and/or bulges exists in the duplex formed by the antisense strand and a target transcript. The two strands of an siRNA are substantially complementary, preferably 100% complementary to each other within the duplex portion.

The term “short hairpin RNA” refers to an RNA molecule comprising at least two complementary portions hybridized or capable of hybridizing to form a double-stranded (duplex) structure sufficiently long to mediate RNAi (as described for siRNA duplexes), and at least one single-stranded portion that forms a loop connecting the regions of the shRNA that form the duplex. The structure is also referred to as a stem/loop structure, with the stem being the duplex portion. The structure may further comprise an overhang (e.g., as described for siRNA) on the 5′ or 3′ end. Preferably, the loop is about 1-20, more preferably about 4 -10, and most preferably about 6-9 nt long and/or the overhang is about 1-20, and more preferably about 2-15 nt long. The loop may be located at either the 5′ or 3′ end of the region that is complementary to the target transcript whose inhibition is desired (i.e., the antisense portion of the shRNA). In certain embodiments the overhang comprises one or more U residues, e.g., between 1 and 5 Us. As described further below, shRNAs are processed into siRNAs by the conserved cellular RNAi machinery. Thus shRNAs are precursors of siRNAs and are, in general, similarly capable of inhibiting expression of a target transcript that is complementary to a portion of the shRNA (referred to as the antisense or guide strand of the shRNA). In general, the features of the duplex formed between the antisense strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group while in other embodiments it does not.

The term “subject”, as used herein, refers to an individual susceptible to infection with a virus, e.g., influenza virus. The term includes birds and animals, e.g., domesticated birds and animals (such as chickens, mammals, including swine, horse, dogs, cats, etc.), and wild animals, non-human primates, and humans.

An RNAi-inducing agent is considered to be “targeted” to a target transcript for the purposes described herein if (1) the RNAi-inducing agent comprises a strand that is at least 80%, preferably at least about 85%, more preferably at least about 90%, 91%, 92%, 93%, 94%, 95%, 96%, 97%, 98%, 99%, or 100% complementary with the target transcript for a stretch of at least about 15, more preferably at least about 17, yet more preferably at least about 18 or 19 to about 21-23, or 24-29 nucleotides in length; and/or (2) one strand of the RNAi-inducing agent hybridizes to the target transcript. Suitable hybridization conditions are those typically found within the cytoplasm or nucleus of mammalian cells and/or in a Drosophila lysate as described, e.g., in US Pubs. 20020086356 and 20040229266 and in refs 21 and 28. In certain embodiments of the invention a GU or UG base pair in a duplex formed by an antisense strand and a target transcript is not considered a mismatch for purposes of determining whether an RNAi-inducing agent is targeted to the transcript. An RNA-inducing vector whose presence within a cell results in production of an RNAi-inducing agent that is targeted to a transcript is also considered to be targeted to the transcript. An RNAi-inducing agent targeted to a transcript is also considered to target the gene that directs synthesis of the transcript. An RNAi-inducing agent that inhibits expression of a target transcript involved in the production of, replication of, pathogenicity of, and/or infection by a virus is said to inhibit the virus.

A “target portion” is a region of a target transcript that hybridizes with an antisense strand of an RNAi-inducing agent.

The term “target transcript” refers to any RNA that is a target for RNAi. Messenger RNA is a preferred target. The terms “target RNA” and “target transcript” are used interchangeably herein.

As used herein, “treating” includes reversing, alleviating, and/or inhibiting the progress of, the disease, disorder, or condition to which such term applies, and/or reversing, alleviating, and/or inhibiting one or more symptoms or manifestations of such disease, disorder or condition.

The term “vector” refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (typically DNA molecules although RNA plasmids are also known), cosmids, and viral vectors. As is well known in the art, the term viral vector may refer either to a nucleic acid molecule (e.g., a plasmid) that includes virus-derived nucleic acid elements that typically facilitate transfer or integration of the nucleic acid molecule (examples include retroviral or lentiviral vectors) or to a virus or viral particle that mediates nucleic acid transfer (examples include retroviruses or lentiviruses). As will be evident to one of ordinary skill in the art, viral vectors may include various viral components in addition to nucleic acid(s).

### DETAILED DESCRIPTION OF CERTAIN PREFERRED EMBODIMENTS OF THE INVENTION

### I. Influenza Viral Life Cycle and Characteristics

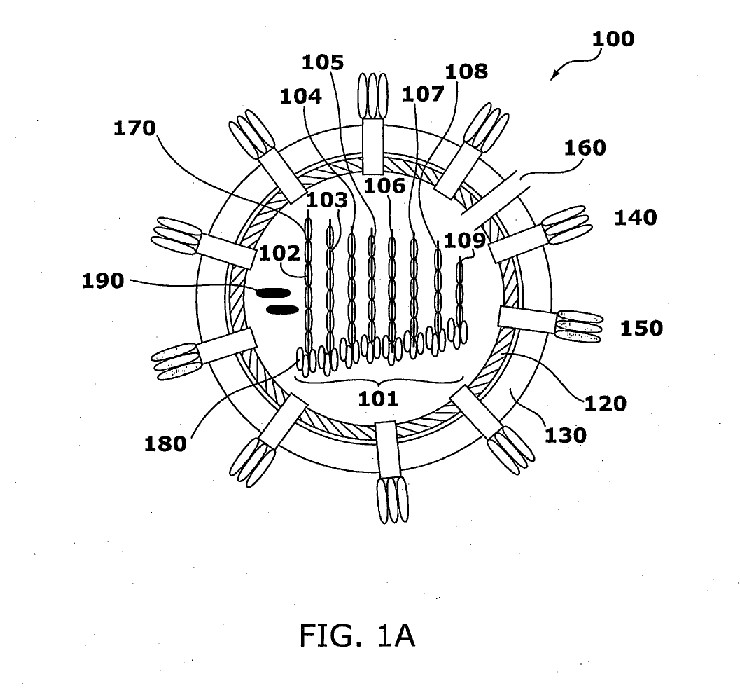

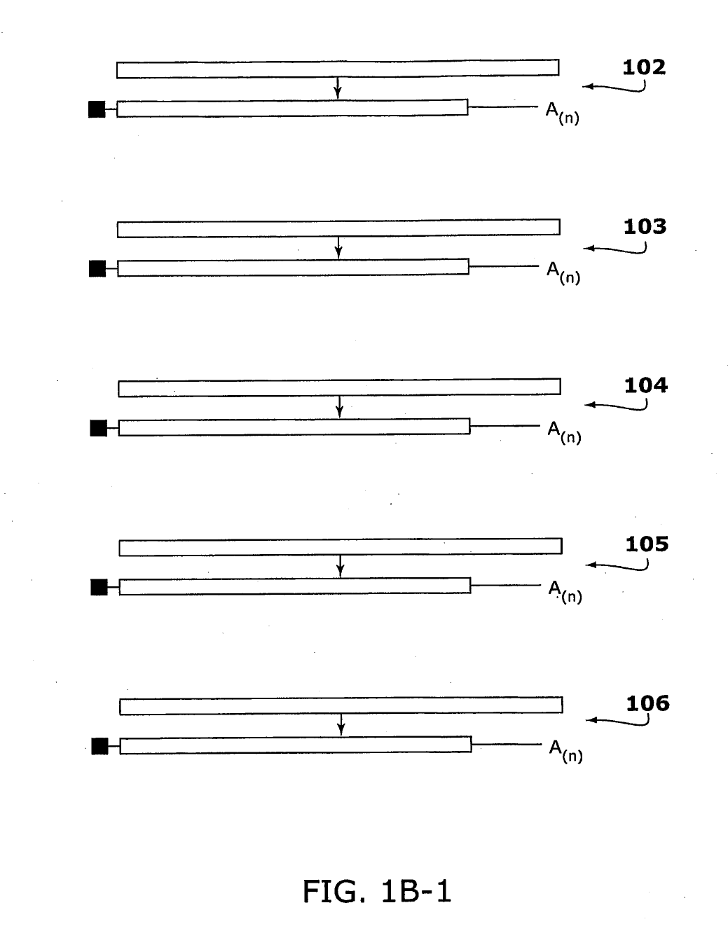

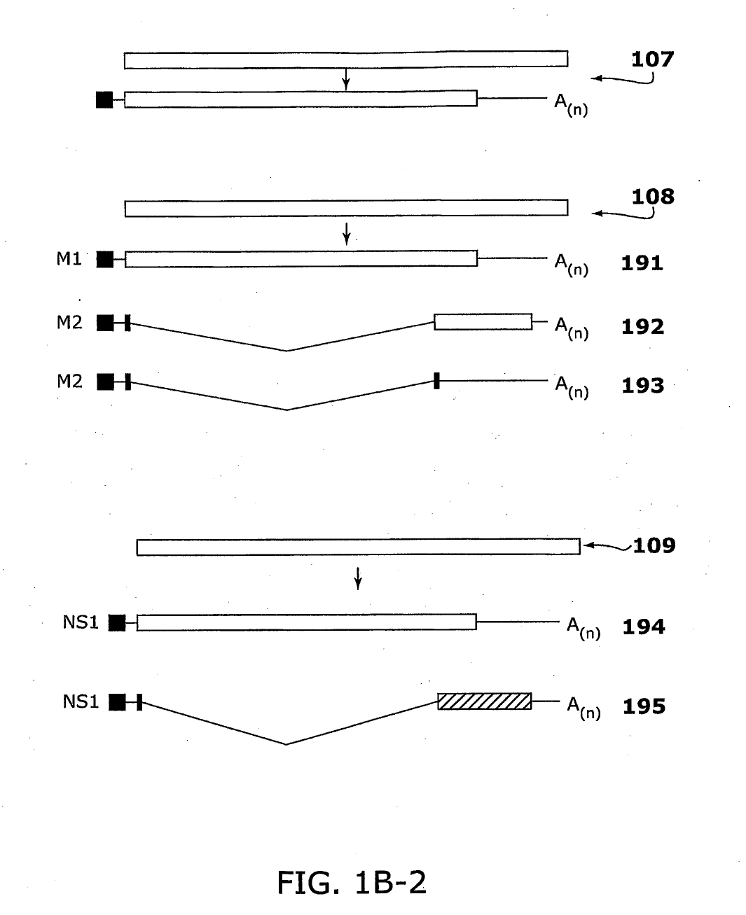

Influenza viruses are enveloped, negative-stranded RNA viruses of the Orthomyxoviridae family. They are classified as influenza types A, B, and C, of which influenza A is the most pathogenic and is believed to be the only type able to undergo reassortment with animal strains. Influenza types A, B, and C can be distinguished by differences in their nucleoprotein and matrix proteins (see FIG. 1). As discussed further below, influenza A subtypes are defined by variation in their hemagglutinin (HA) and neuraminidase (NA) genes and usually distinguished by antibodies that bind to the corresponding proteins.

The influenza A viral genome consists of ten genes distributed in eight RNA segments. The genes encode 10 proteins: the envelope glycoproteins hemagglutinin (HA) and neuraminidase (NA); matrix protein (M1); nucleoprotein (NP); three polymerases (PB1, PB2, and PA) which are components of an RNA-dependent RNA transcriptase also referred to as a polymerase or polymerase complex herein; ion channel protein (M2), and nonstructural proteins (NS1 and NS2). See Julkunen, I., et al., Cytokine and Growth Factor Reviews, 12: 171-180, 2001 for further details regarding the influenza A virus and its molecular pathogenesis. See also Fields, B., et al., Fields' Virology, 4^{th}. ed., Philadelphia: Lippincott Williams and Wilkins; ISBN: 0781718325, 2001. The organization of the influenza B viral genome is extremely similar to that of influenza A while the influenza C viral genome contains seven RNA segments and lacks NA.

Influenza A virus classification is based on the hemagglutinin (H1-H15) and neuraminidase (N1-N9) genes. World Health Organization (WHO) nomenclature defines each virus strain by its animal host of origin (specified unless human), geographical origin, strain number, year of isolation, and antigenic description of HA and NA. For example, A/Puerto Rico/8/34 (H1N1) designates strain A, isolate 8, that arose in humans in Puerto Rico in 1934 and has antigenic subtypes 1 of HA and NA. As another example, A/Chicken/Hong Kong/258/97 (H5N1) designates strain A, isolate 258, that arose in chickens in Hong Kong in 1997 and has antigenic subtype 5 of HA and 1 of NA. Human epidemics have been caused by viruses with HA types H1, H2, and H3 and NA types N1 and N2.

As mentioned above, genetic variation occurs by two primary mechanisms in influenza virus A. Genetic drift occurs via point mutations, which often occur at antigenically significant positions due to selective pressure from host immune responses, and genetic shift (also referred to as reassortment), involving substitution of a whole viral genome segment of one subtype by another. Many different types of animal species including humans, swine, birds, horses, aquatic mammals, and others, may become infected with influenza A viruses. Some influenza A viruses are restricted to a particular species and will not normally infect a different species. However, some influenza A viruses may infect several different animal species, principally birds, swine, and humans. This capacity is considered to be responsible for major antigenic shifts in influenza A virus. For example, suppose a swine becomes infected with an influenza A virus from a human and at the same time becomes infected with a different influenza A virus from a duck. When the two different viruses reproduce in the swine cells, the genes of the human strain and duck strain may “mix,” resulting in a new virus with a unique combination of RNA segments. This process is called genetic reassortment.

Like other viruses, influenza viruses replicate intracellularly. Influenza A viruses replicate in epithelial cells of the respiratory tract. However, monocytes/macrophages and other white blood cells can also be infected. Numerous other cell types with cell surface glycoproteins containing sialic acid, which acts as a viral receptor, are susceptible to infection in vitro.

The influenza A infection/replication cycle is depicted schematically in FIG. 1. As shown in FIG. 1A, the influenza A virion 100 comprises genome 101, consisting of eight negative stranded RNA segments: PB2 (102), PB1 (103), PA (104), HA (105), NP (106), NA (107), M (108), and NS (109). There are conventionally numbered from 1 to 8, with PB2=1, PB1=2, PA=3, HA=4, NP=5, NA=6, M=7, and NS=8. The genomic RNA segments are packaged inside a layer of membrane protein M1 120 which is surrounded by a lipid bilayer 130 from which the extracellular domains of the envelope glycoproteins HA 140 and NA 150 and the ion channel M2 160 protrude. RNA segments 102-108 are covered with nucleoprotein MP 170 and contain the viral polymerase complex 180 consisting of polymerases PB1, PB2, and PA. Nonstructural protein NS2 190 is also found within virions. Nonstructural protein NS1 (not shown) is found within infected cells.

FIG. 1B shows the genome structure of the influenza virus and the transcripts generated from the influenza genome (not drawn to scale). Six of the eight genomic RNA segments (PB1 (102), PB2 (103), PA (104), HA (105), NP (106), and NA (107)) each serve as template for a single, unspliced transcript that encodes the corresponding protein. Three mRNA transcripts have been identified as being derived from influenza virus A segment M (108): a colinear transcript 191 that encodes the M₁ protein, a spliced mRNA 192 that encodes the M₂ protein and contains a 689 nucleotide intron, and another alternatively spliced mRNA 193 that has the potential to encode a 9 amino acid peptide (M3) that has not been detected in virus-infected cells. Two mRNA transcripts are derived from influenza virus A segment NS: an unspliced mRNA 194 that encodes the NS₁ protein and a spliced mRNA 195 that encodes the NS₂ protein and includes a 473 nucleotide intron.

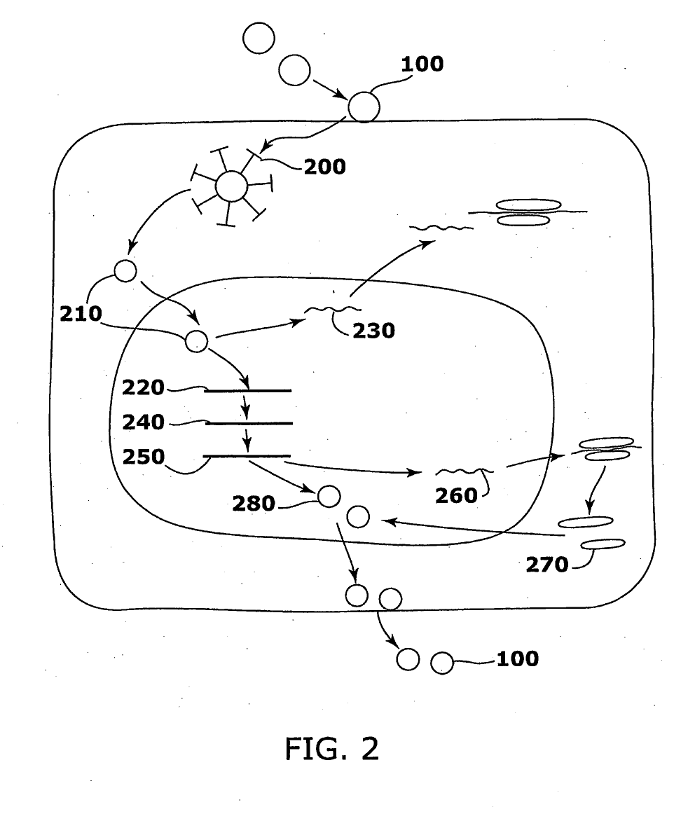

The infective cycle (FIG. 2) begins when the virion 100 attaches via its hemagglutinin to the surface of a susceptible cell. Attached virus is endocytosed into coated vesicles 200 via clathrin-dependent endocytosis. Low pH in endosomes triggers fusion of viral and endosomal membranes, resulting in liberation of viral ribonucleoprotein (vRNP) complexes (nucleocapsids) 210 into the cytoplasm. Viral nucleocapsids are imported into the cell nucleus, following which primary viral mRNA synthesis is initiated by a viral RNA polymerase complex that consists of the PB1, PB2, and PA polymerases. Primers produced by the endonuclease activity of the PB2 protein on host cell pre-mRNA is used to initiate viral mRNA synthesis using viral RNA (vRNA) 220 as a template. PB1 protein catalyzes the synthesis of virus specific mRNAs 230, which are transported into the cytoplasm and translated.

Newly synthesized polymerases are transported into the nucleus and regulate replication and secondary viral mRNA synthesis. Synthesis of complementary RNA (cRNA) 240 from viral RNA (vRNA) is initiated by PB1, PB2, PA, and NP, after which new vRNA molecules 250 are synthesized. The viral polymerase complex uses these vRNAs as templates for synthesis of secondary mRNA 260. Thus transcription of vRNA by the virus-encoded transcriptase produces mRNA that serves as a template for synthesis of viral proteins and also produces complementary RNA (cRNA), which serves as a template for synthesizing more vRNA for new virion production. Viral mRNAs are transported into the cytoplasm, where viral structural proteins 270 are produced. Proteins PB1, PB2, PA, and NP are transported into the nucleus, the site of assembly of vRNP complexes (nucleocapsids) 280. Budding and release of viral particles occur at the plasma membrane.

Influenza A virus replicates rapidly in cells, resulting in host cell death due to cytolytic effects or apoptosis. Infection causes changes in a wide variety of cellular activities and processes including inhibition of host cell gene expression. The viral polymerase complex binds to and cleaves newly synthesized cellular polymerase II transcripts in the nucleus. NS1 protein blocks cellular pre-mRNA splicing and inhibits nuclear export of host mRNA. Translation of cellular mRNA is greatly inhibited, whereas viral mRNA is efficiently translated. Maintenance of efficient translation of viral mRNAs is achieved in part through viral downregulation of the cellular interferon (IFN) response, a host response which typically acts to inhibit translation in virally infected cells. In particular, viral NS1 protein binds to IFN-induced PKR and inhibits its activity. Thus it is evident that infection with influenza virus results in profound changes in cellular biosynthesis, including changes in the processing and translation of cellular mRNA.

II. Selection, Design, and Synthesis of RNAi-Inducing Entities

A. Selection and Design of RNAi-Inducing Entities

The present invention provides RNAi-inducing entities targeted to one or more influenza virus transcripts. Various viral RNA transcripts (primary and secondary vRNA, primary and secondary viral mRNA, and viral cRNA) are present in cells infected with influenza virus and play important roles in the viral life cycle. Any of these transcripts are appropriate targets for RNAi mediated inhibition by either a direct or an indirect mechanism in accordance with the present invention. Preferred RNAi-inducing entities that target any viral mRNA transcript will specifically reduce the level of the transcript itself in a direct manner, e.g., by causing degradation of the transcript. In addition, RNAi-inducing agents that target certain influenza virus transcripts (e.g., NP, PA, PB1) will indirectly cause reduction in the levels of influenza virus transcripts to which they are not specifically targeted.

Viral transcripts that may serve as a target for RNAi based therapy according to the present invention include, for example, 1) any influenza virus genomic segment; 2) transcripts that encode any viral proteins including transcripts encoding the proteins PB1, PB2, PA, NP, NS1, NS2, M1, M2, HA, or NA. Transcripts may be targeted in their vRNA, cRNA, and/or mRNA form(s) by a single RNAi-inducing agent, although the inventors have obtained data suggesting that viral mRNA is the sole or primary target of RNAi. In particularly preferred embodiments the target transcript encodes influenza virus protein NP, PA, PB1, or PB2.

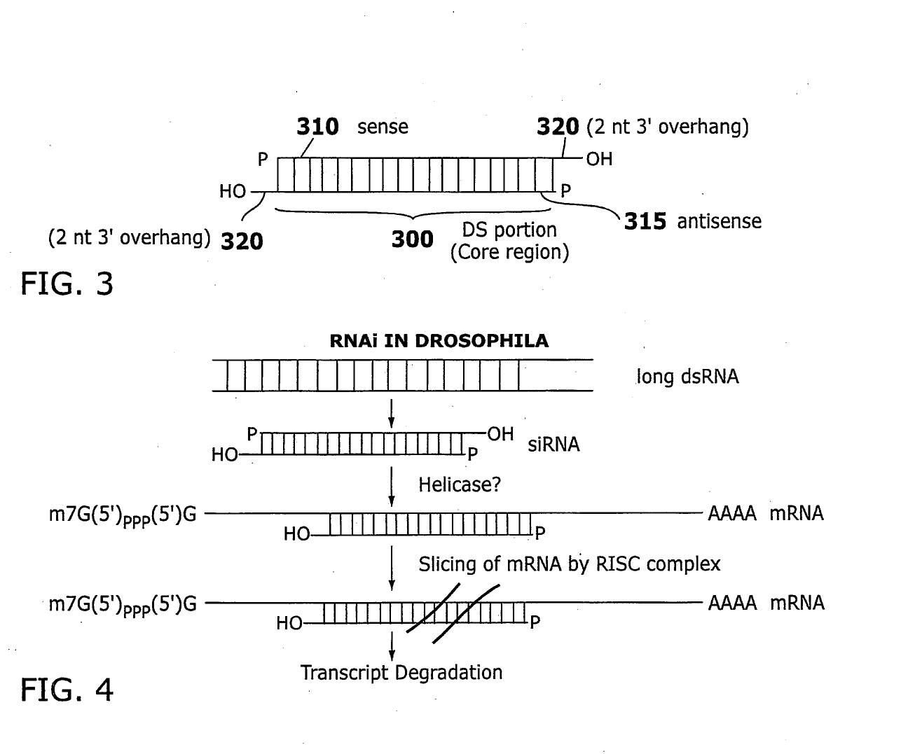

General features of RNAi-inducing agents are known in the art. RNA interference was initially recognized as a phenomenon in which the presence of long dsRNA (typically hundreds of nt) in a cell leads to sequence-specific degradation of mRNA containing a region complementary to one strand of the dsRNA (U.S. Pat. No. 6,506,559). siRNAs were first discovered in studies of RNAi in Drosophila, as described in WO 01/75164 and U.S. Pub. Nos. 20020086356 and 20030108923. In particular, it was found that, in Drosophila, long dsRNAs are processed by an RNase III-like enzyme called Dicer (Bernstein et al., Nature 409:363, 2001) into smaller dsRNAs comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. FIG. 3 shows a schematic diagram of siRNAs found in Drosophila. The structure includes a 19 nucleotide double-stranded (DS) portion 300, comprising a sense strand 310 and an antisense strand 315. Each strand has a 2 nt 3′ overhang 320.

These short dsRNAs (siRNAs) act to silence expression of any gene that includes a region complementary to one of the dsRNA strands, presumably because a helicase activity unwinds the 19 by duplex in the siRNA, allowing an alternative duplex to form between one strand of the siRNA (the “antisense” or “guide” strand) and the target transcript. The antisense strand is incorporated into an endonuclease complex, RISC, which is guided to the complementary target RNA. An enzymatic activity present in RISC cleaves (“slices”) at a single location, producing unprotected RNA ends that are promptly degraded by cellular machinery (FIG. 4). As mentioned below, additional mechanisms of silencing mediated by short RNA species (microRNAs) are also known (see, e.g., Ruvkun, G., Science, 294, 797-799, 2001; Zeng, Y., et al., Molecular Cell, 9, 1-20, 2002). It is noted that the discussion of mechanisms and the figures depicting them are not intended to suggest any limitations on the mechanism of RNA inteference employed in the present invention.

Homologs of the Dicer enzyme are found in diverse species ranging from C. elegans to humans (Sharp, Genes Dev. 15;485, 2001; Zamore, Nat. Struct. Biol. 8:746, 2001), raising the possibility that an RNAi-like mechanism might be able to silence gene expression in a variety of different cell types including mammalian, or even human, cells. However, long dsRNAs (e.g., dsRNAs having a double-stranded region longer than about 30-50 nucleotides) are known to activate the interferon response in mammalian cells. Thus, rather than achieving the specific gene silencing observed with the Drosophila RNAi mechanism, the presence of long dsRNAs in mammalian cells would be expected to lead to interferon-mediated non-specific suppression of translation, potentially resulting in cell death. Long dsRNAs are therefore not thought to be useful for inhibiting expression of particular genes in mammalian cells.

However, the inventors and others have found that siRNAs, when introduced into mammalian cells, can effectively reduce the expression of target genes, including viral genes. The inventors have found that a significant proportion of the sequences selected using a first set of design parameters described herein proved to be efficient suppressing sequences when included in an siRNA or shRNA and tested as described below and in co-pending patent application U.S. Ser. No. 10/674,159. Approximately 15% of siRNAs from an initially designed set (Example 1) showed a strong effect and potently inhibited virus production in cells infected with either PR8 or WSN strains of influenza virus; approximately 40% showed a significant effect (i.e., a statistically significant difference (p≦0.05) between virus production in the presence versus the absence of siRNA in cells infected with PR8 and/or in cells infected with WSN); approximately 45% showed no or minimal effect.

In particular, RNAs targeted to genes that encode the RNA-dependent RNA transcriptase and nucleoprotein NP, dramatically reduced the level of virus produced in infected mammalian cells (Examples 2, 4, 5, 6). The inventors have also shown that siRNAs targeted to influenza virus transcripts can inhibit influenza virus replication in vivo in intact organisms, namely chicken embryos infected with influenza virus (Example 3). In addition, the inventors have demonstrated that siRNAs targeted to influenza virus transcripts can inhibit virus production in mice when administered either before or after viral infection (Examples 12, 14, 16, 23-26, etc.). Furthermore, the inventors have shown that administration of a DNA vector from which siRNA precursors (shRNAs) can be expressed inhibits influenza virus production in mice. Additional effective RNAi-inducing agents, including a number of siRNAs that were highly effective when tested for their ability to inhibit influenza virus production in cells and/or mice, were designed using a second set of design criteria. Thus, the present invention demonstrates that treatment with RNAi agents such as siRNA, shRNA, or with vectors whose presence within a cell leads to expression of such agents are effective strategies for inhibiting infection and/or replication by a respiratory virus, e.g., influenza virus. Two of the highly effective siRNAs were subsequently tested against highly pathogenic avian influenza virus strains, and were shown to inhibit them, confirming their ability to inhibit a broad range of influenza virus strains (Tompkins, et al., Proc. Natl. Acad. Sci., 101(23):8682-6, 2004). Thus the invention provides RNAi-inducing agents that inhibit virus production in cells infected with any of multiple different influenza virus strains.

While not wishing to be bound by any theory, the inventors suggest that these findings are especially significant in view of the profound changes in cellular activities, e.g., metabolic and biosynthetic activities, that take place upon infection with influenza virus. Infection with influenza virus inhibits such fundamental cellular processes as cellular mRNA splicing, transport, and translation and results in inhibition of cellular protein synthesis. Despite these alterations, the finding that RNAi-inducing agents targeted to influenza viral transcripts inhibits viral replication suggests that the cellular mechanisms underlying the RNAi-mediated inhibition of gene expression continue to operate in cells infected with influenza virus at a level sufficient to inhibit influenza gene expression.

For any particular gene target that is selected, the design of RNAi-inducing agents for use in accordance with the present invention will preferably follow certain guidelines. In general, it is desirable to target sequences that are specific to the virus (as compared with the host), and that, preferably, are important or essential for viral function. Thus it is desirable to avoid sections of target transcript that may be shared with other transcripts whose degradation is not desired. A database search may be performed to determine whether either strand is substantially complementary to any sequence in the genome of an organism (e.g., a human) to which the agent is to be delivered, and such sequences may be avoided. As described herein, portions of the viral transcript that are conserved among multiple variants are preferred targets.

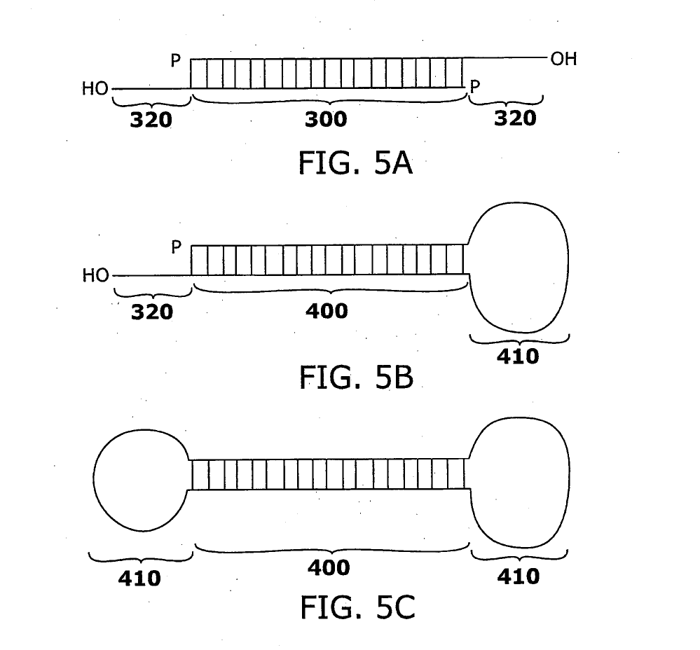

Preferred RNAi-inducing agents for use in accordance with the present invention include a base-paired region between 15 and approximately 29 nt long, e.g., approximately 19 nt in length, and may optionally have one or more free or looped ends. FIG. 5 presents various structures that could be utilized as an RNAi-inducing agent according to the present invention. FIG. 5A shows the structure found to be active in the Drosophila system described above and in mammalian cells. The present invention encompasses administration of an siRNA having the structure depicted in FIG. 5A to mammalian cells in order to treat or prevent influenza infection. However, it is not required that the administered agent have this structure. For example, the administered composition may include any structure capable of being processed in vivo to the structure of FIG. 5A, so long as the administered agent does not cause undesired or deleterious events such as induction of the interferon response. The invention may also comprise administration of agents that are not processed to precisely the structure depicted in FIG. 5A, so long as administration of such agents reduces viral transcript levels sufficiently as discussed herein. In some cases, the agent that is delivered to a cell according to the present invention may undergo one or more processing steps before becoming an active suppressing agent (see below for further discussion); in such cases, those of ordinary skill in the art will appreciate that the relevant agent will preferably be designed to include sequences that may be necessary for its processing.

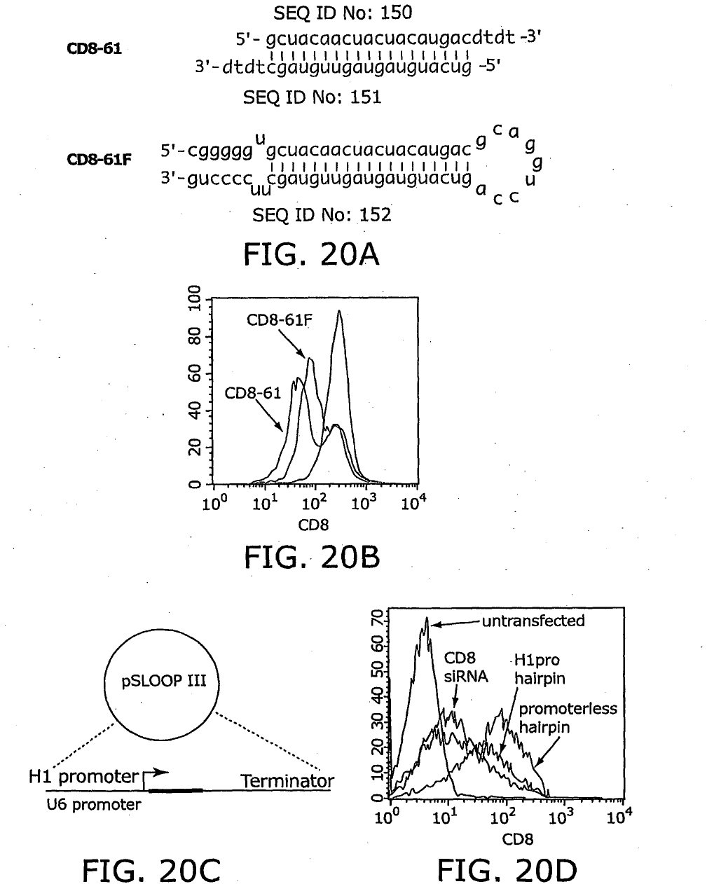

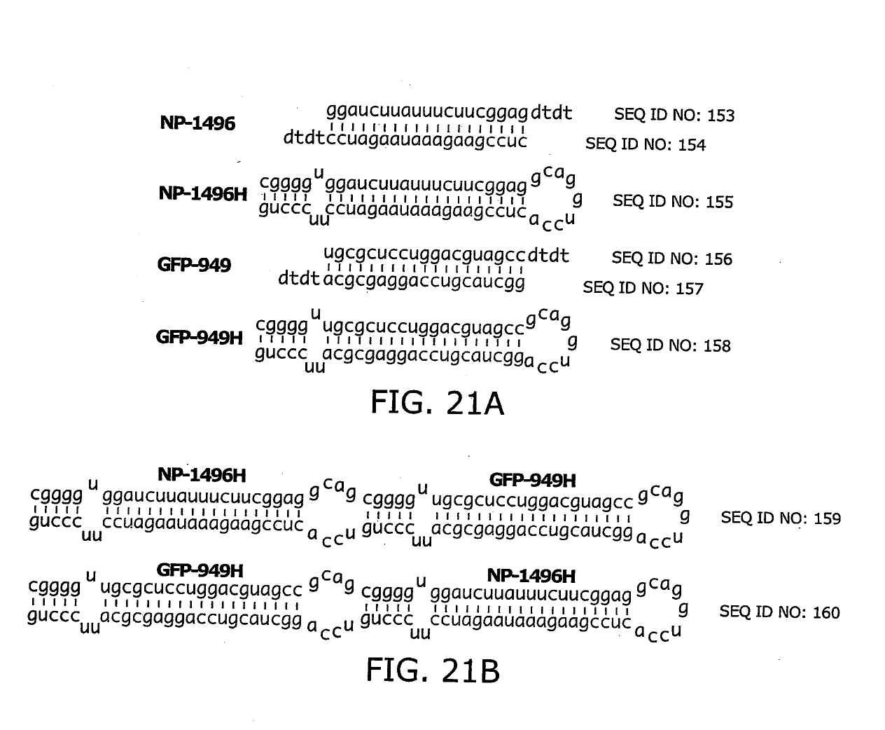

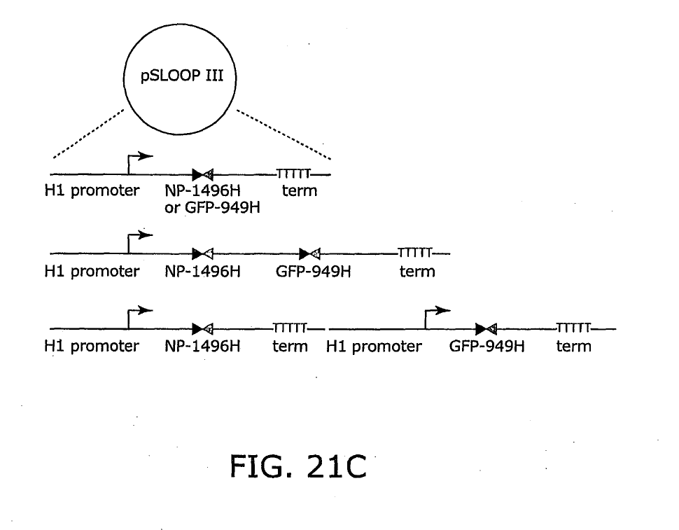

FIGS. 5B and 5C represent additional structures that may be used to mediate RNAi. These hairpin (stem-loop) structures may function directly as inhibitory RNAs or may be processed intracellularly (e.g., by Dicer) to yield an siRNA structure such as that depicted in FIG. 5A. FIG. 5B shows an agent comprising an RNA molecule containing two complementary regions that hybridize to one another to form a duplex region represented as stem 400, a loop 410, and an overhang 320. Such molecules are said to self-hybridize, and a structure of this sort is referred to as an shRNA. See also FIGS. 20 and 21 for examples of shRNA structures. FIG. 5C shows an agent comprising an RNA circle that includes complementary elements sufficient to form a stem 400 approximately 19 by long. Such an agent may show improved stability as compared with various other siRNAs described herein.

In describing RNAi-inducing agents and their activities it will frequently be convenient to refer to the agent as having two strands, as in the case of siRNAs. In general, the sequence of the duplex portion of one strand of the RNAi-inducing agent is substantially complementary to the target transcript in this region. The sequence of the duplex portion of the other strand of the RNAi-inducing agent is typically substantially identical to the targeted portion of the target transcript. The strand comprising the portion complementary to the target is referred to as the “antisense strand”, while the other strand is often referred to as the “sense strand”. The portion of the antisense strand that is complementary to the target may be referred to as the “inhibitory region”.

The duplex structure of an shRNA may be considered to comprise antisense and sense strands, where the antisense strand is a first portion of the molecule that forms or is capable of forming a duplex with a second portion of the molecule and is complementary to the targeted portion of the target transcript. The sense strand is the portion of the molecule which forms or is capable of forming a duplex with the first portion. One skilled in the art will appreciate that an “antisense strand” that targets a vRNA strand from a negative-strand RNA virus will be antisense to the vRNA, but be a “sense strand” relative to the viral cRNA. Likewise, an “antisense strand” that targets a cRNA strand from a negative-strand RNA virus will be antisense to the cRNA, but be a “sense strand” relative to the vRNA sequence.

For purposes of description, the discussion below will frequently refer to siRNA. However, as will be evident to one of ordinary skill in the art, teachings relevant to the two strands of an siRNA are generally applicable to the sense and antisense strands of the stem portion of any RNAi-inducing agent, e.g., a corresponding shRNA that can be processed intracellularly to yield an siRNA. Thus in general the considerations below apply also to the design, selection, and delivery of inventive RNAi-inducing agents such as shRNAs that are processed intracellularly to yield RNAs that mediate target cleavage or translational repression.

In general, preferred siRNA antisense strands hybridize with a target site that comprises or consists of exonic sequences in the target transcript. In certain embodiments of the invention the antisense strand hybridizes to a 5′ or 3′ untranslated region. In general, any site that is available for hybridization with a antisense strand, resulting in slicing and degradation and/or translational repression of the transcript may be utilized.

RNAi-inducing agents may be selected according to a variety of approaches. In general, as mentioned above, inventive RNAi-inducing agents preferably include a region (the “duplex region”), one strand of which contains an inhibitory region between 15-29 nt in length that is sufficiently complementary to a portion of the target transcript (the “target portion”), so that a hybrid can form in vivo between this strand and the target transcript. This duplex region, also referred to as the “core region” is understood not to include overhangs. Overhangs, if present, may, but need not be, complementary to the target transcript. Preferably, this duplex region includes most or all of the double-stranded structure depicted in FIGS. 3, 4, and 5.

Preferably the inhibitory region is 100% complementary to the target. However, one of ordinary skill in the art will recognize that mismatches and bulges may exist in a duplex formed by an antisense strand and the target. Thus the inhibitory region need only be sufficiently complementary to the target such that hybridization can occur, e.g., under physiological conditions in a cell and/or in an in vitro system that supports RNAi, such as the Drosophila extract system mentioned above. Preferably the inhibitory region and the target are at least 70%, more preferably at least 80%, more preferably at least 90%, and most preferably 100% complementary to each other. Preferably fewer than six residues or alternatively about 20%, e.g., 1, 2, 3, 4, of antisense strand residues in this region are mismatched with the target over a window of 15-29 nt, e.g., 19 nt. Preferably fewer than 4 residues or alternatively about 15% of antisense strand residues in this region are mismatched with the target. Preferably only 1 or 2 mismatches is present. One or more (or all) of the mismatches may be a U-G mismatch. For example, if the inhibitory region is 15-16 nt long, there may be 0-3 mismatches; if the inhibitory region is 17 nt long there may be 0-4 mismatches; if the inhibitory region is 18 nt long, there may be 0-5 mismatches; if the inhibitory region is 19 nt long, there may be 0-6 mismatches. The number of permissible mismatches increases by one nt for each additional nt present in the inhibitory region up to the upper limit of the inhibitory region of an RNAi-inducing agent, e.g., a length of approximately 30 nt. In certain embodiments the mismatches are not at continuous positions. In certain embodiments there is no stretch of mismatches longer than two nt in length. In preferred embodiments a window of evaluation of 15-19 nt contains 0-1 mismatch (preferably 0), and a window of evaluation of 20-29 nt contains 0-2 mismatches (preferably 0-1, more preferably 0). One of skill in the art will recognize that duplex structures interrupted by bulges will typically allow a greater number of unpaired nt. One of skill in the art will also recognize that it may be preferable to avoid mismatches in the central portion of the antisense strand/target RNA duplex (see, e.g., Elbashir et al., EMBO J. 20:6877, 2001). For example, the 3′ nucleotides of the antisense strand of the siRNA often do not contribute significantly to specificity of the target recognition and may be less critical for target cleavage. In certain embodiments the antisense strand and the target are complementary at position 10 of the inhibitory region of the antisense strand. In other embodiments they are not.

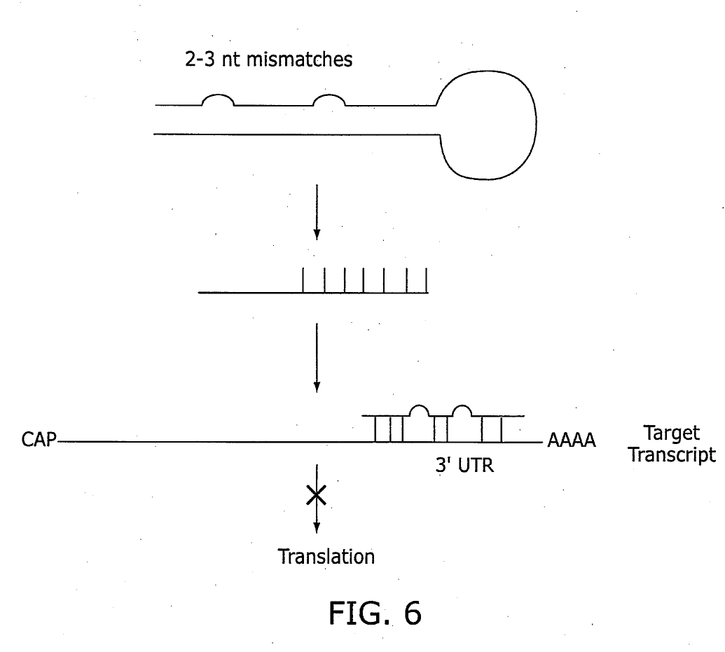

Certain RNAi-inducing agents contain a strand that hybridizes to a target site that includes or consists entirely of 3′ UTR sequences. One of ordinary skill in the art will appreciate that the resulting duplexes may tolerate a larger number of mismatches and/or bulges, particularly mismatches within the central region of the duplex while still leading to effective silencing. For example, one or both strands may include one or more “extra” nucleotides that form a bulge as shown in FIG. 6. One or more bulges of, e.g., 5-10 nt long, may be present. Typically the stretches of perfect complementarity are at least 5 nt in length, e.g., 6, 7, or more nt, while the regions of mismatch may be, for example, 1, 2, 3, or 4 nt in length. The duplexes frequently include two stretches of perfect complementarity separated by a region of mismatch. A variety of structures are possible. For example, there may be multiple areas of mismatch.

Some mismatches may be desirable, as duplex formation in the 3′ UTR or elsewhere may inhibit expression of a protein encoded by the transcript by a mechanism related to, but distinct from, the cleavage that is the hallmark of classic RNA inhibition. As shown in FIG. 6, the Dicer enzyme that generates siRNAs in the Drosophila system discussed above and also in a variety of organisms, is known to also process a small, temporal RNA (stRNA) substrate into an inhibitory agent that, when bound within the 3′ UTR of a target transcript, blocks translation of the transcript (see Grishok, A., et al., Cell 106, 23-24, 2001; Hutvagner, G., et al., Science, 293, 834-838, 2001; Ketting, R., et al., Genes Dev., 15, 2654-2659). Subsequent discoveries have shown that the genomes of organisms ranging from C. elegans to mammals encodes a class of short (˜19-25 nucleotide) RNAs known as microRNAs (miRNAs) that inhibit translation of endogenous mRNAs to which they are partially complementary. MicroRNAs are discussed in Bartel, D P., Cell, 116(2):281-97, 2004; Novina, C. and Sharp, P A, Nature, 430:161-164, 2004; and US Pub. No. 20050059005. An miRNA binds to target mRNA transcripts at partially complementary sites and prevents their translation. RNAi-inducing agents having the structure of siRNAs (two individual short strands hybridized to one another) can act in a manner similar to miRNAs, i.e., by reducing translation of the transcript rather than decreasing its stability (Doench, J G, et al. Genes & Development, 17:438-442, 2003). It is believed that such siRNAs are processed intracellularly to give rise to single-stranded RNAs that act via the miRNA translational repression pathway. For the purposes of the present invention, any partly or fully double-stranded short RNA as described herein, one strand of which binds to a target transcript and reduces its expression (i.e., reduces the level of the transcript and/or reduces synthesis of the polypeptide encoded by the transcript) is considered to be an RNAi-inducing agent, regardless of whether it acts by triggering degradation, inhibiting translation, or by other means. In addition any precursor RNA structure that may be processed in vivo (i.e., within a cell or organism) to generate such an RNAi-inducing agent is useful in the present invention.

In some cases the sequence of an RNAi-inducing agent is selected such that the entire antisense strand (including the 3′ overhang if present) is perfectly complementary to the target transcript. However, it is not necessary that overhang(s) are either complementary or identical to the target transcript. Any desired sequence (e.g., UU) may simply be appended to the 3′ ends of antisense and/or sense core regions to generate 3′ overhangs. In general, overhangs containing one or more pyrimidines, usually U, T, or dT, are employed. When synthesizing RNAi-inducing agents it may be more convenient to use T rather than U in the overhang(s). Use of dT rather than T may confer increased stability.

Preferably the strands of an RNAi-inducing agent are 100% complementary to each other within the core region. However, one of ordinary skill in the art will recognize that mismatches and bulges may exist in a duplex formed by an antisense and sense strand. The strands need only be sufficiently complementary to one another such that hybridization can occur, e.g., under physiological conditions in a cell and/or in an in vitro system that supports RNAi, such as the Drosophila extract system mentioned above. Preferably the two strands of an RNAi-inducing agent are substantially complementary within the core region, e.g., at least 70%, more preferably at least 80%, more preferably at least 90%, and most preferably 100% complementary to each other within the core region. For example, a core region 15-16 nt long may contain 0-3 mismatches; a core region 17 nt long may contain 0-4 mismatches; a core region 18 nt long may contain 0-5 mismatches; a core region 19 nt long may contain 0-6 mismatches. The number of permissible mismatches increases by one nt for each additional nt present in the inhibitory region up to the upper limit of the core region of an RNAi-inducing agent, e.g., a length of approximately 30 nt. In preferred embodiments a core region of 15-19 nt contains 0-1 mismatch (preferably 0), and a core region of 20-29 nt contains 0-2 mismatches (preferably 0-1, more preferably 0). One of skill in the art will recognize that duplex structures interrupted by bulges will typically allow a greater number of unpaired nt.

In summary, an RNAi-inducing agent may be designed by selecting a target portion and designing an RNAi-inducing agent comprising an antisense strand whose sequence is sufficiently complementary to hybridize to the target, e.g., substantially complementary or 100% complementary to the target transcript over 15-29 nucleotides, e.g., 19 nucleotides, and a sense strand whose sequence is sufficiently complementary to hybridize to the antisense strand, e.g., substantially or, preferably, 100% complementary to the antisense strand. 3′ overhangs such as those described above may then be added to these sequences to generate an siRNA structure.

One of ordinary skill in the art will appreciate that RNAi-inducing agents such as siRNAs may exhibit a range of melting temperatures (Tm) in accordance with the foregoing principles. The Tm is defined as the temperature at which 50% of a nucleic acid and its perfect complement are in duplex in solution. Representative examples of acceptable Tms may readily be determined using methods well known in the art, either experimentally or using appropriate empirically or theoretically derived equations, based on the siRNA sequences disclosed in the Examples herein. In certain embodiments of the invention the calculated Tm of a duplex formed by an antisense strand and a target transcript is up to 5° C. lower, up to 10° C. lower, or up to 15° C. lower than the calculated Tm of a duplex that would be formed between the target and an antisense strand having an inhibitory region that is perfectly complementary to the target. In certain embodiments of the invention the calculated Tm of a duplex formed by the antisense strand and the sense strand of an RNAi-inducing agent is up to 5° C. lower, or up to 10° C. lower, or up to 15° C. lower than the calculated Tm of a duplex that would be formed between antisense and sense strands that are perfectly complementary (optionally excluding overhangs).

One of ordinary skill in the art will be able to calculate Tm values. Several studies have derived accurate equations for Tm using thermodynamic basis sets for nearest neighbor interactions. Values for thermodynamic parameters are available in the literature. For RNA see Freier, S. M., et al., Proc. Natl. Acad. Sci. 83, 9373-9377, 1986. Rychlik, W., et al., Nucl. Acids Res. 18(21), 6409-6412, 1990. Preferably the more recent values and methods in Walter, A. E., Proc. Natl. Acad. Sci., 91, 9218-9222, 1994, or more preferably those in Mathews, D H, J. Mol. Biol., 288, 911-940, 1999, are used. Computer programs for calculating Tm are widely available. See, e.g., the Web site having URL www.basic.nwu.edu/biotools/oligocalc.html. Preferably a program for calculating relevant parameters such as ΔG and Tm, available on the mfold web server at the URL www.bioinfo.rpi.edu/applications/mfold, as described in Zuker, M., Nucl. Acids. Res., 31(13), 2003 is used.

One aspect of the present invention is the recognition that when multiple strains, subtypes, etc. (referred to collectively as variants), of an infectious agent exist, whose genomes vary in sequence, it will often be desirable to select and/or design RNAi-inducing agents that target regions that are highly conserved among different variants. By comparing a sufficient number of sequences and selecting highly conserved regions, it is possible to target multiple variants with a single RNAi-inducing agent targeted to such a highly conserved region, e.g., the antisense strand of the agent is substantially complementary to the highly conserved region over a sufficient length such that the RNAi-inducing agent mediates RNAi. According to certain embodiments of the invention a region is highly conserved among multiple variants if it is identical among the variants. According to certain embodiments of the invention a region targeted by the RNAi-inducing agent, e.g., a region of 15-29 nucleotides, preferably 19 nucleotides, is highly conserved if it differs by at most one nucleotide (i.e., at 0 or 1 nucleotide positions) among the variants. According to certain embodiments of the invention such a region is highly conserved among multiple variants if it differs by at most two nucleotides (i.e., at 0, 1, or 2 nucleotide positions) among the variants. According to certain embodiments of the invention a region is highly conserved among multiple variants if it differs by at most three nucleotides or (i.e., at 0, 1, 2, or 3 nucleotide positions) among the variants. According to certain embodiments of the invention an RNAi-inducing agent is targeted to a region that is highly conserved among at least 5, 10, 15, 20, 25, 30, 40, 50, or more variants.

In order to identify regions that are highly conserved among a set of multiple variants, the following procedure may be used. One member of the set of sequences is selected as the base sequence, i.e., the sequence to which other sequences are to be compared. According to different embodiments of the invention the base sequence may either be one of the sequences in the set being compared or may be a consensus sequence derived from sequences in the set, e.g., by determining for each position the most frequently found nucleotide at that position among the sequences in the set.

Having selected a base sequence, the sequence of each member of the set of multiple variants is compared with the base sequence. The number of differences between the base sequence and any member of the set of multiple variants over a region of the sequence (e.g., a region of 15-29 nucleotides, such as 19 nucleotides) is used to determine whether the particular region of interest is highly conserved between the base sequence and the member of the set to which it is being compared. As noted above, in various embodiments of the invention if the number of positions at which the sequence differs between two regions is either 0; 0 or 1; 0, 1, or 2; or 0, 1, 2, or 3, the regions are considered highly conserved. The antisense sequence of the RNAi-inducing agent may be selected to be complementary to the base sequence across a highly conserved region of 15-29 nucleotides, e.g., 19 nucleotides, or may be selected to be complementary to one of the other sequences across the highly conserved region. Typically, the sense strand sequence is selected to be identical to the base sequence or to one of the other sequences across the highly conserved region, such that the antisense strand and the sense strand are 100% complementary to each other within the duplex portion of the RNAi-inducing agent.

Generally the antisense strand sequence is selected with reference to the base sequence as described above. However in certain embodiments of the invention, particularly if a nucleotide present at a particular position in a second sequence in the set being compared is found in more of the sequences being compared than the correspondingly positioned nucleotide in the base sequence, then the antisense strand sequence may be selected with reference to the second sequence (e.g., 100% complementary to the highly conserved region present in the second sequence). In addition according to certain embodiments of the invention, if the consensus nucleotide (most commonly occurring nucleotide) at the position where the difference occurs is different to that found in the base sequence, the consensus nucleotide may be used. Note that this may result in a sequence that is not identical to any of the sequences being compared (as may the use of a consensus sequence as the base sequence).

Example 1 describes the selection of highly conserved target portions based on comparison of a set of sequences from six influenza A strains having a human host of origin and comparison of a set of sequences from seven influenza A strains having different animal hosts of origin (including human) and the design of siRNA sequences that target these portions. Different methods of selecting highly conserved regions may be used. The invention encompasses RNAi-inducing agents whose duplex portions (and, optionally, any overhangs) are selected based on highly conserved regions that meet the criteria provided herein, regardless of how the highly conserved regions are selected. It is also to be understood that the invention encompasses RNAi-inducing agents targeted to portions of influenza virus transcripts that do not meet the criteria for preferred target regions described herein. For example, RNAi-inducing agents that are not targeted to highly or favorably conserved target portions may still be effective inhibitors of influenza virus production for certain strains. Less effective RNAi-inducing entities may also be used for evaluating target specificity, identifying determinants of RNAi efficacy, improving RNAi design, etc.

Table 1A lists 21-nucleotide regions that are highly conserved among a set of influenza virus sequences for each of the viral gene segments. Each sequence listed in Table 1A includes a 19 nt region (nt 3-21) and an initial 2 nt sequence that is not present in the sense strand of the corresponding siRNA but is complementary to the 3′ overhang of the antisense strand of the siRNA. It will be appreciated that the 19 nt region may be used as the sense strand to design a variety of siRNA molecules having different 3′ overhangs in either or both the sense and antisense strands. Thus a variety of sense and antisense siRNA sequences may be obtained from each sequence listed in Table 1A. Twenty such siRNA sequences are listed in Table 2.

Table 1B lists additional siRNAs designed based on highly conserved regions of influenza virus. Both strands are shown in a 5′ to 3′ direction. A dTdT 3′ overhang is appended to each strand. Nucleotides 1 to 19 in each of the sense strand sequences listed in Table 1B has an identical sequence to a highly conserved region of an influenza virus transcript. The corresponding antisense sequence is complementary to the sense strand. In certain embodiments of the invention, a “highly conserved region” refers to nt 3-21 in any of the sequences listed in Table 1A or nt 1-19 of any of the sense strands listed in Table 1B. These regions are present in double-stranded form in certain of the inventive RNAi-inducing agents, so that the antisense strand of the agent is targeted to the highly conserved portion. The sequences of these regions are referred to as “highly conserved sequences” or “highly conserved target portions”.

Selection of highly conserved target portions represents one approach to the design of RNAi-inducing entities that will successfully inhibit multiple different influenza virus strains. However, the present invention also provides alternative methods for selecting preferred target portions that are favorably conserved so as to enhance the likelihood that an RNAi-inducing agent targeted to the target portion will inhibit expression of a target transcript in a plurality of different strains that differ in sequence within the target portion. According to the invention, if a target portion is favorably conserved, an RNAi-inducing agent whose antisense strand comprises an inhibitory region that is 100% complementary to the target portion as found in one or more strains (e.g., PR8), preferably inhibits expression of the corresponding transcript that is present in one or more other strains in which the corresponding target portion is less than 100% complementary to the inhibitory region of the antisense strand.

The invention provides a variety of RNAi-inducing agents that target a favorably conserved portion of an influenza A virus transcript, wherein the favorably conserved target portion is selected according to the inventive methods. In certain embodiments of the invention the target portions are favorably conserved across a plurality of influenza A virus strains isolated from humans. In certain embodiments of the invention the portions are favorably conserved across a plurality of influenza A virus strains isolated from animals other than humans, e.g., avians. In certain embodiments of the invention the portions are favorably conserved across a plurality of influenza A virus strains isolated from humans and also across a plurality of influenza A virus strains isolated from non-human animals, e.g., avians. For example, the portions may be favorably conserved among at least 5, 10, 15, 20, or more variants of human and/or animal origin. In certain embodiments of the invention a target portion is both favorably and highly conserved.

For any virus target, favorably conserved target portions may be identified by first aligning transcript sequences from a plurality of variants and comparing them with a selected base sequence to identify differences, i.e., positions at which the identity of a nucleotide in one or more of the sequences differs from that in the base sequence. The nature of the difference is evaluated to determine whether it is significant. When discussing a set of sequences that are aligned and compared in order to select conserved regions, a “difference” refers to a position at which one or more of the sequences differs from the base sequence, regardless of how many of the sequences differ from the base sequence. The base sequence can be selected in various ways. For example, it may be convenient to select a sequence from a strain that is highly prevalent or that is readily usable in a laboratory setting.

Favorably conserved target portions meet the following criteria when corresponding target portions present in the variants in the set are compared: (1) An A to G or C to U difference between the base sequence and a corresponding sequence is allowed at any position; (2) A G to A or C to A difference between the base sequence and a corresponding sequence is allowed only at one or more of positions 1, 18, and 19; (3) There are 0, 1, 2, or 3 differences between the base sequence and a corresponding sequence between positions 1 and 9; (4) There are no more than 2 consecutive differences between the base sequence and a corresponding sequence; and (5) There is at most 1 difference between the base sequence and a corresponding sequence between positions 11 and 17. Any strain may be selected as the base strain. Preferably at least 5 variants are compared to identify a favorably conserved target portion.

Influenza viruses circulating in avians and/or other animal hosts sometimes gain the capability of infecting humans. Such strains frequently result in a high mortality rate, possibly in part due to a lack of immunity among humans. Examples include the 1918 pandemic and deaths caused by infection of humans with avian influenza in Hong Kong (1997) and Vietnam (2004). Concern regarding the potential spread of strains with an avian or other animal host of origin into the human population is growing, and existing vaccines are not able to protect humans from avian or swine flu virus infection. The invention identifies favorably and/or highly conserved target portions across a plurality of strains originally isolated from humans (human derived strains) and strains originally isolated from non-human animals such as avians (avian derived strains) and provides RNAi-inducing agents targeted to such target portions. Such RNAi-inducing agents are capable of protecting against a wide variety of influenza virus strains, including both human-derived strains and strains derived from non-human animal hosts.

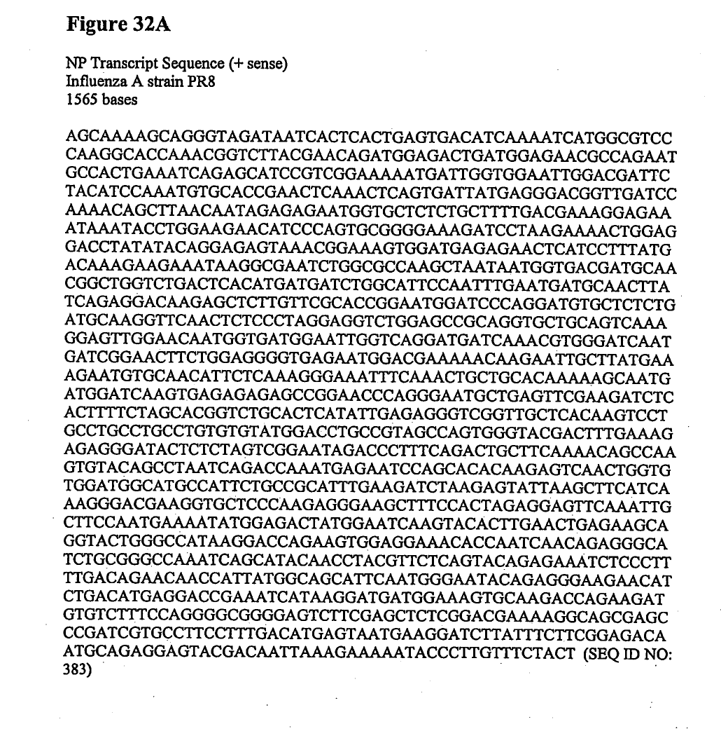

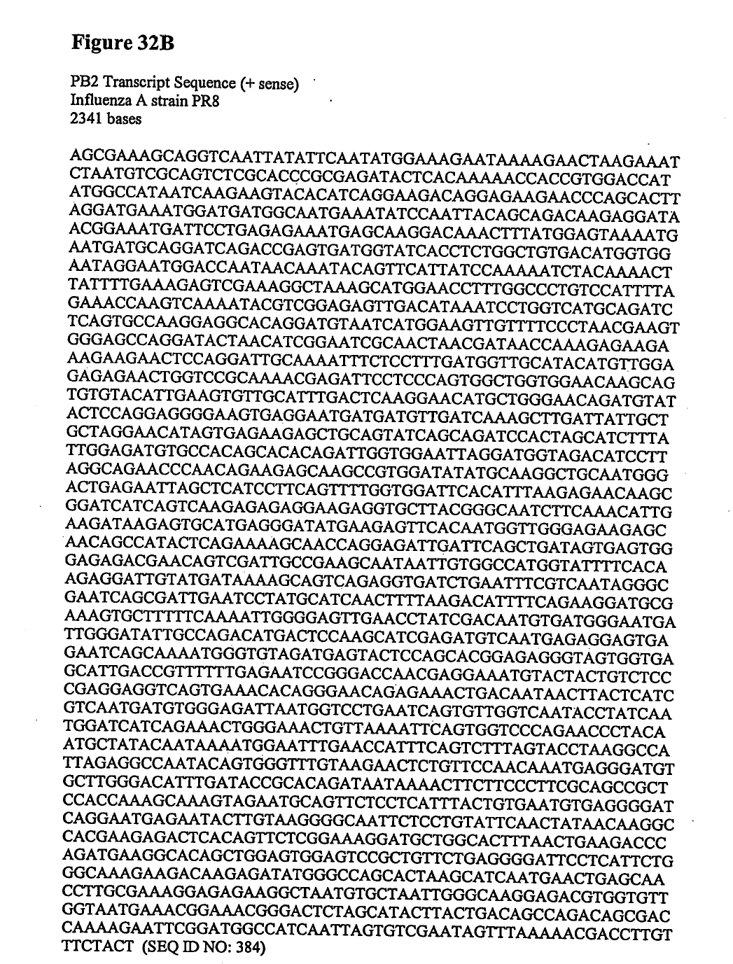

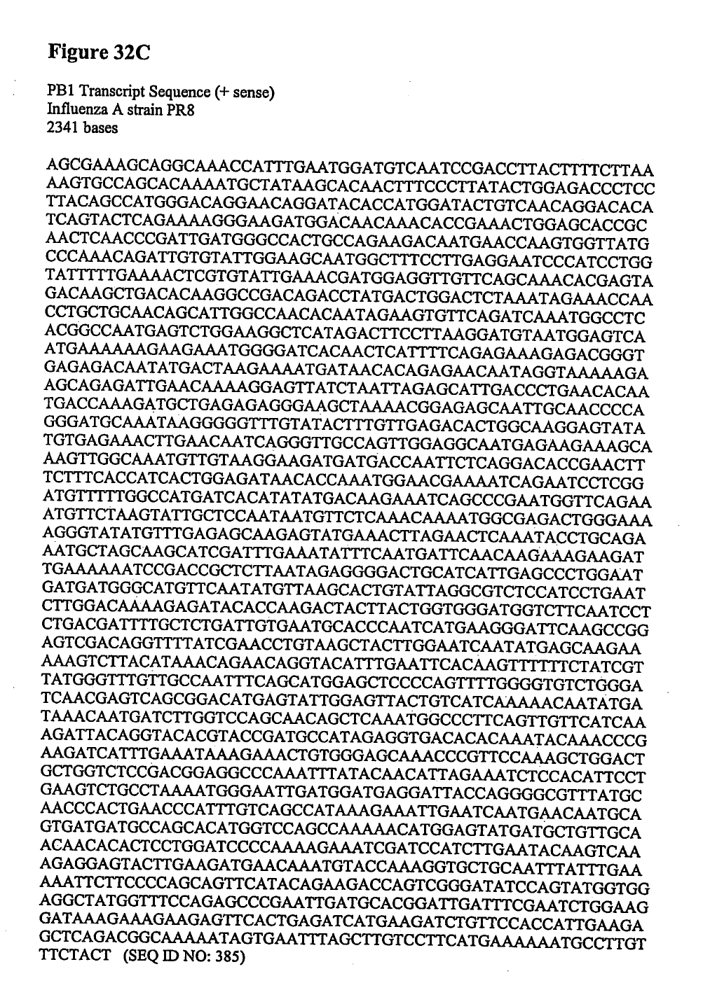

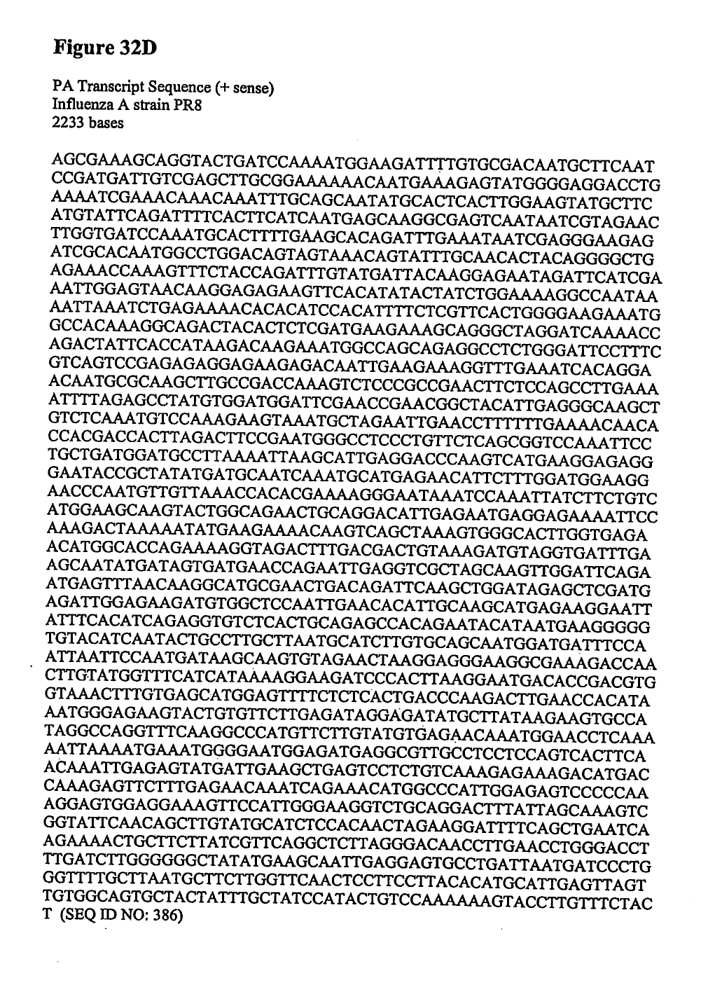

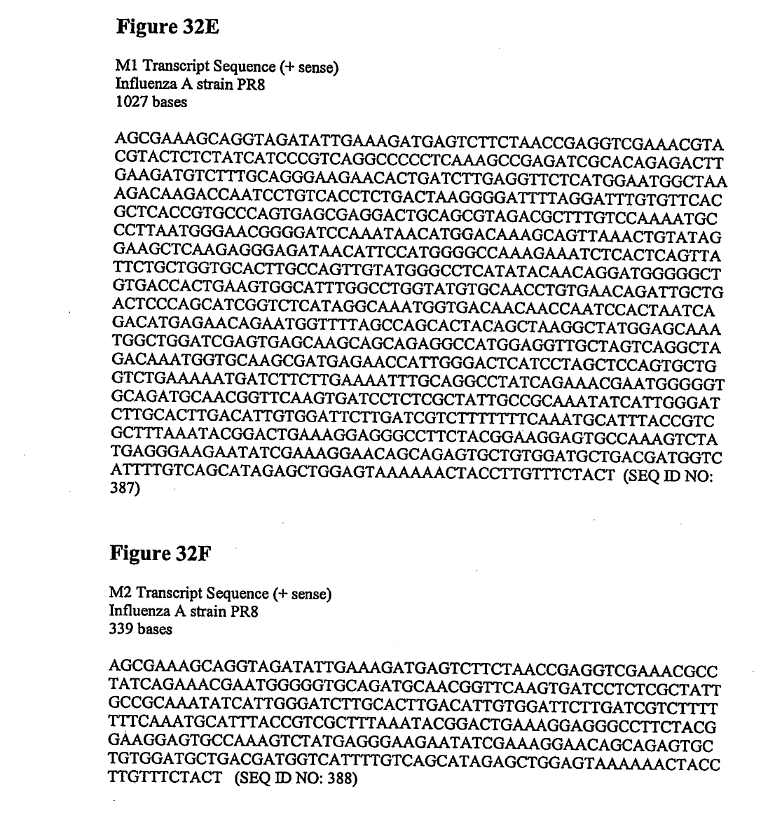

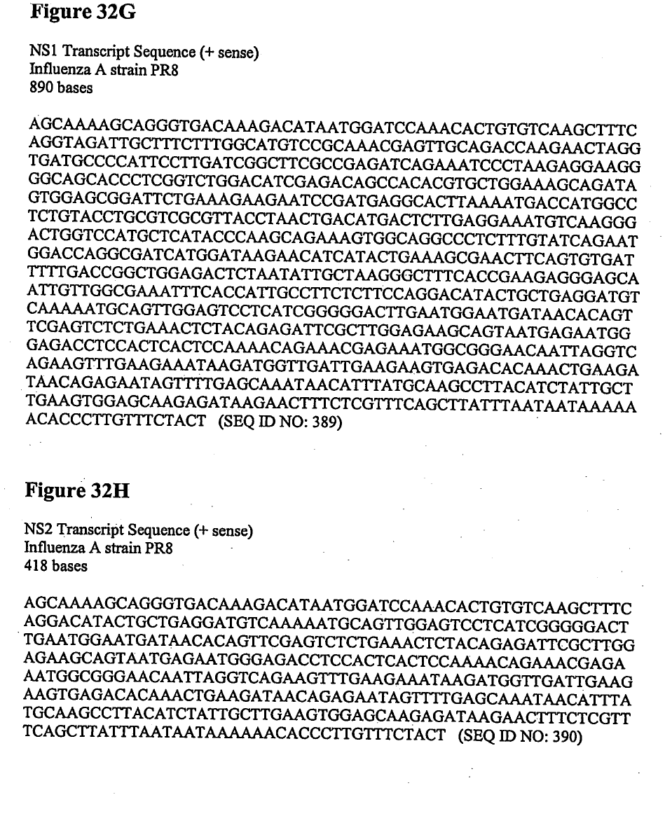

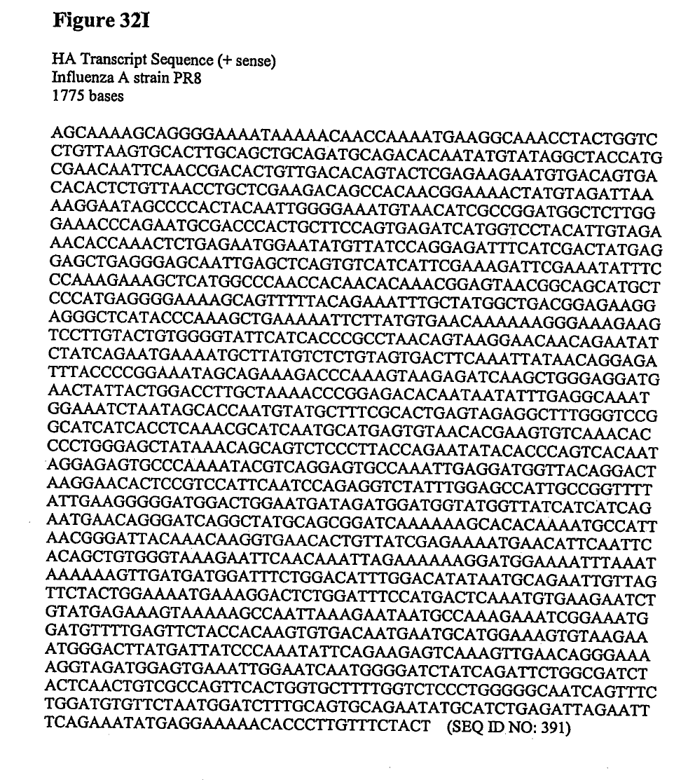

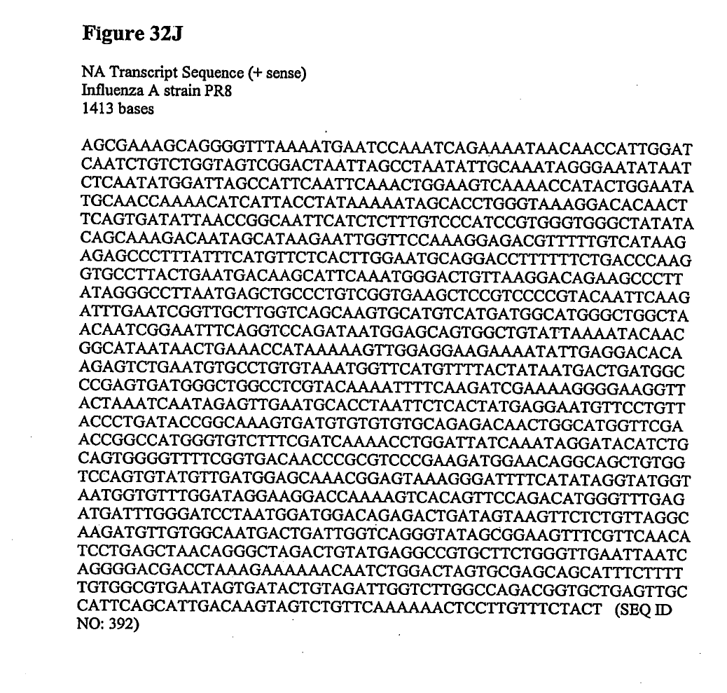

Example 17 describes identification of influenza transcript target portions that are favorably conserved targets for RNAi. The first step was to identify all potential 19 nucleotide influenza virus target portions. The sequence of each 19 nucleotide potential target portion as found in PR8 or in another influenza virus strain listed in Tables 15A-15H or Tables 19A-19F is considered to be listed herein although they are are not specifically set forth. They may readily be identified by reference to the sequences of influenza virus segments in FIGS. 32A-32J.

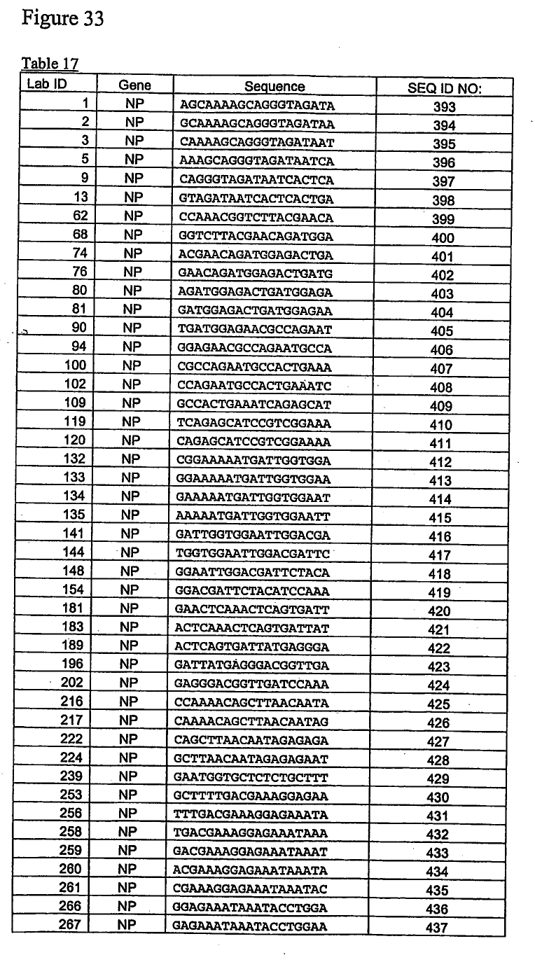

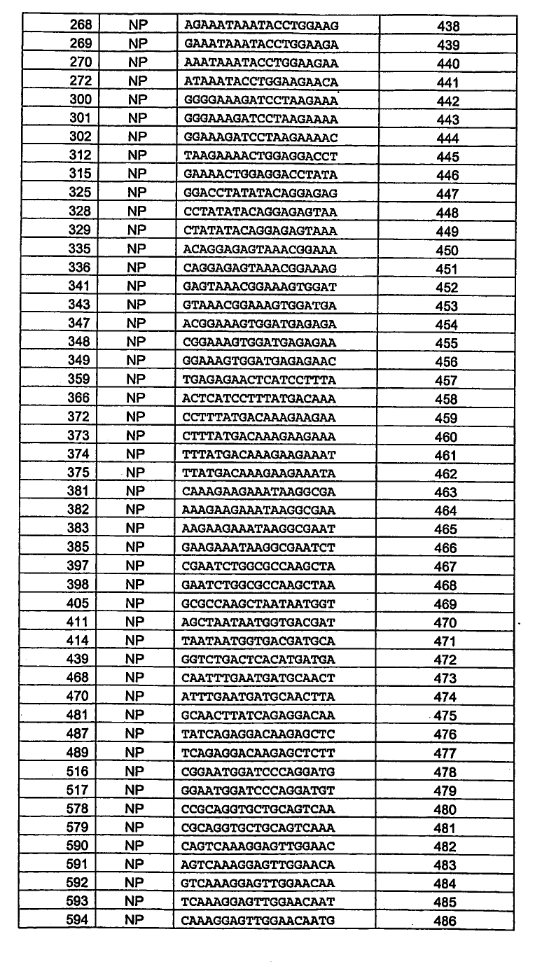

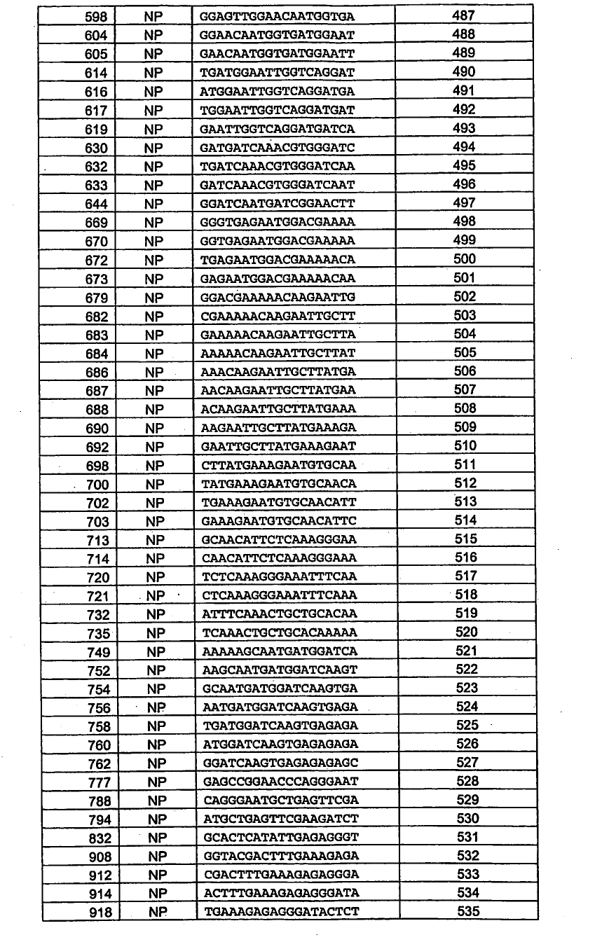

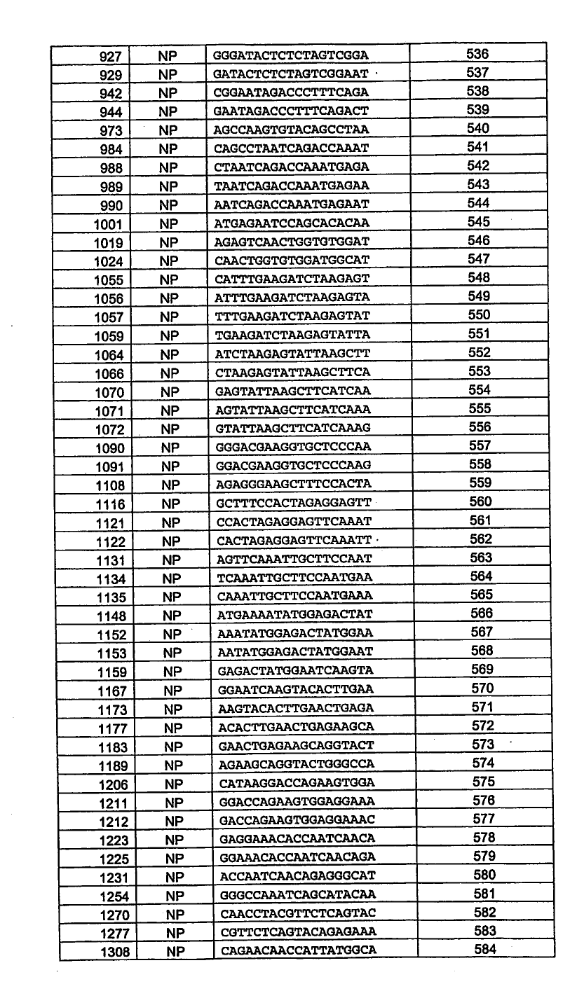

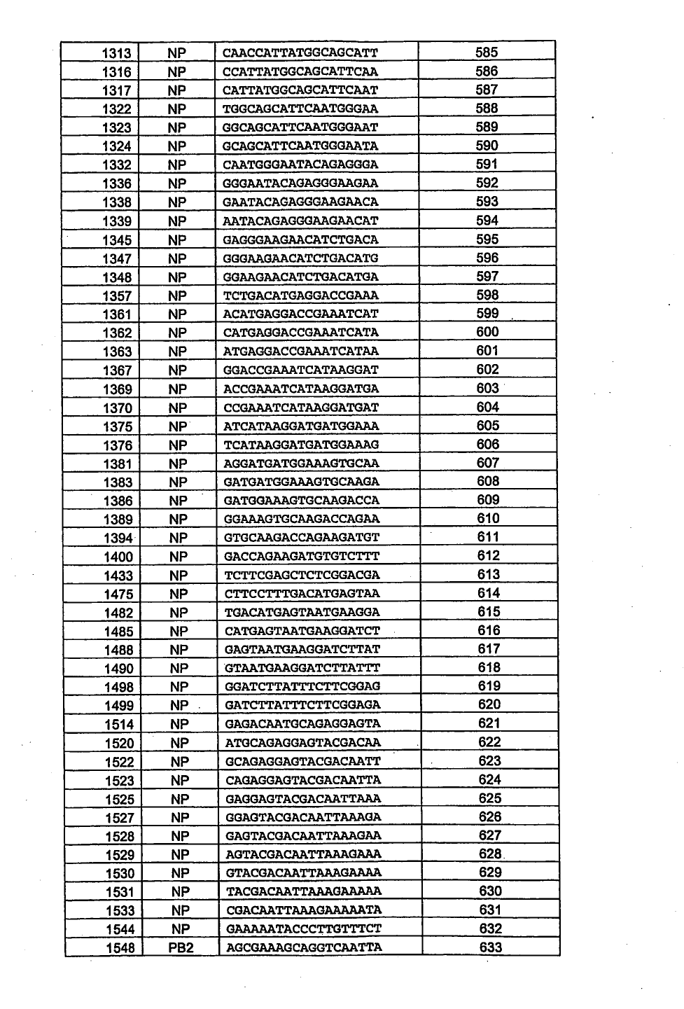

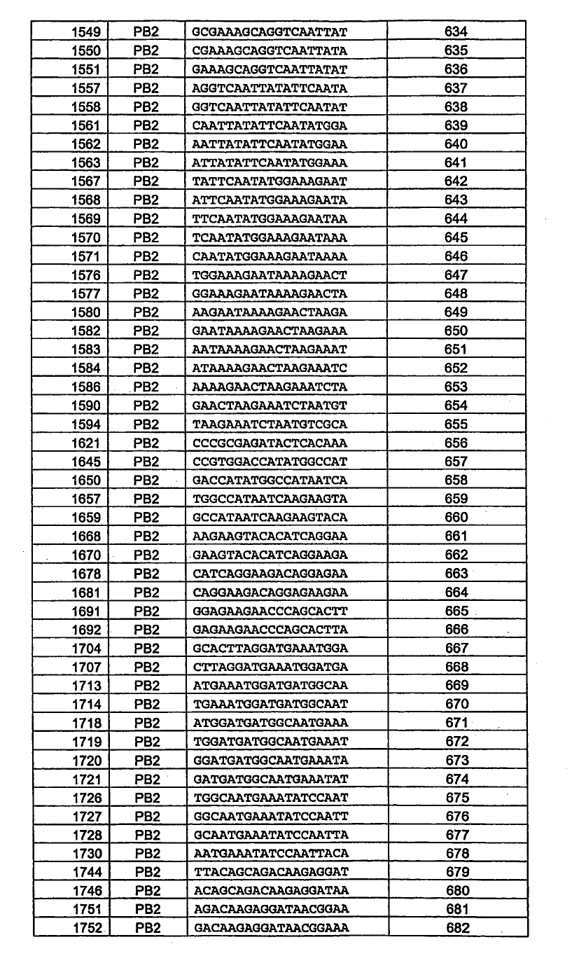

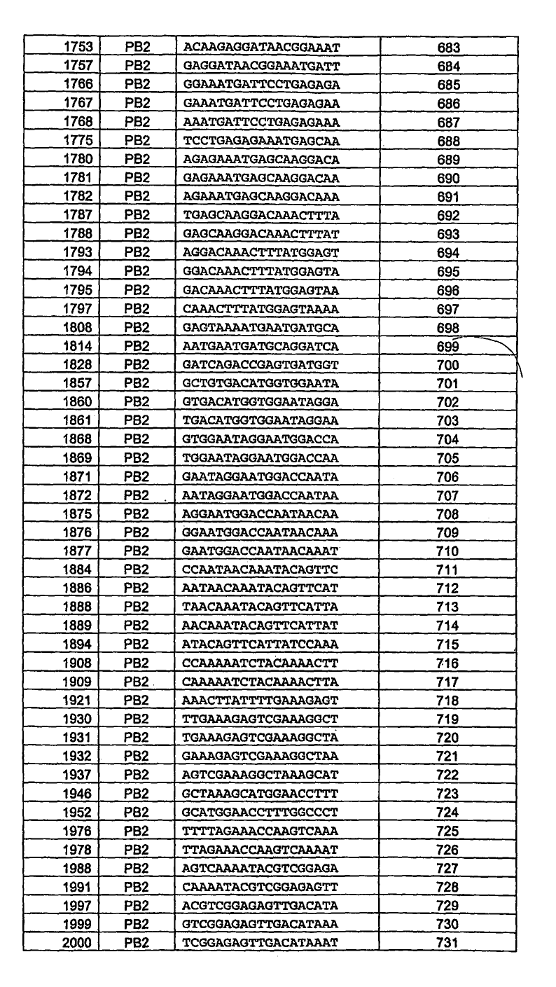

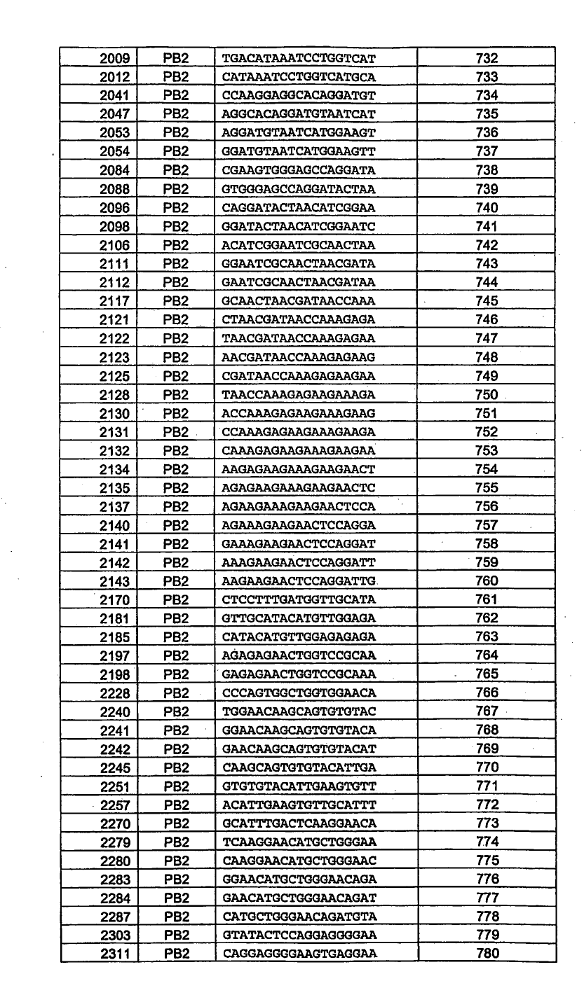

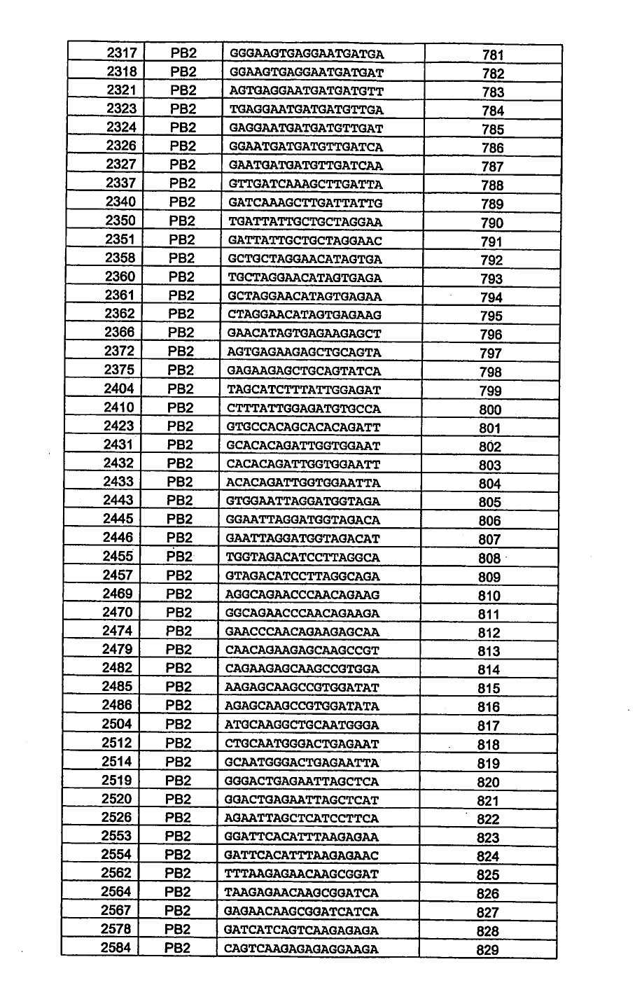

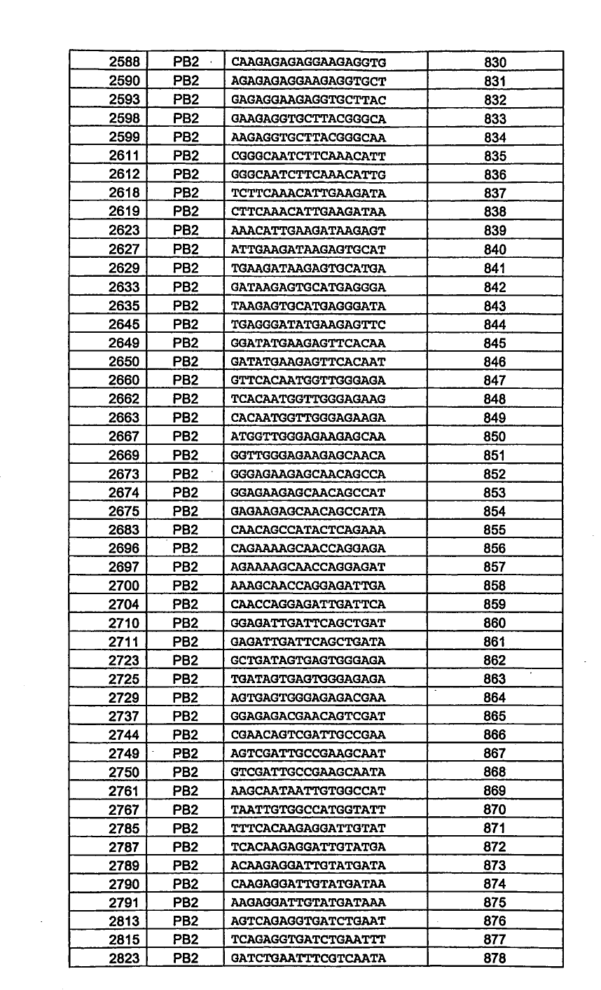

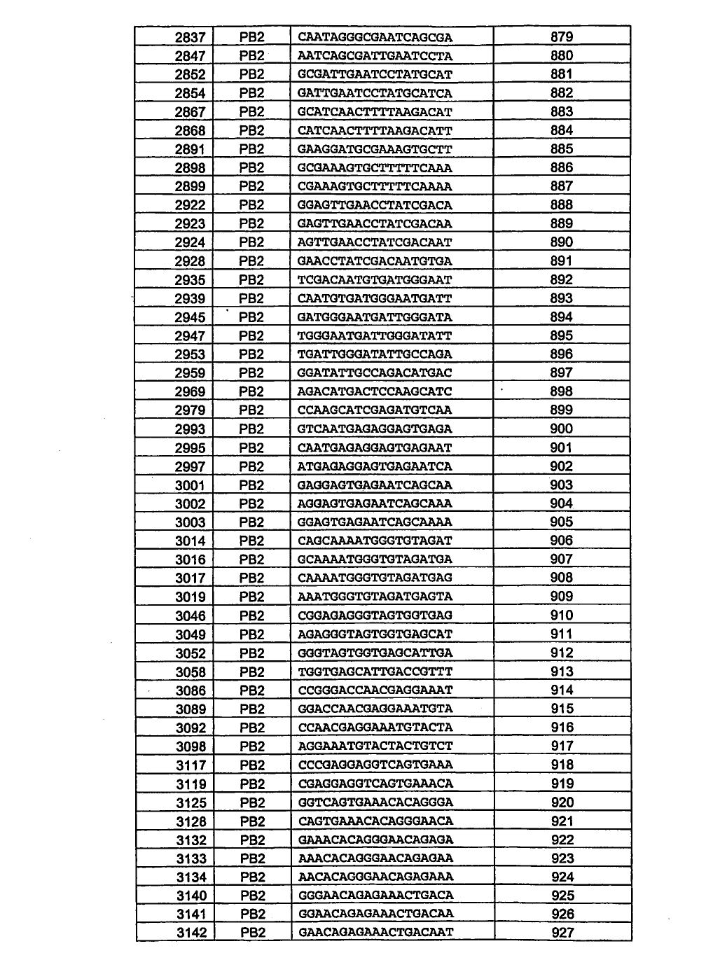

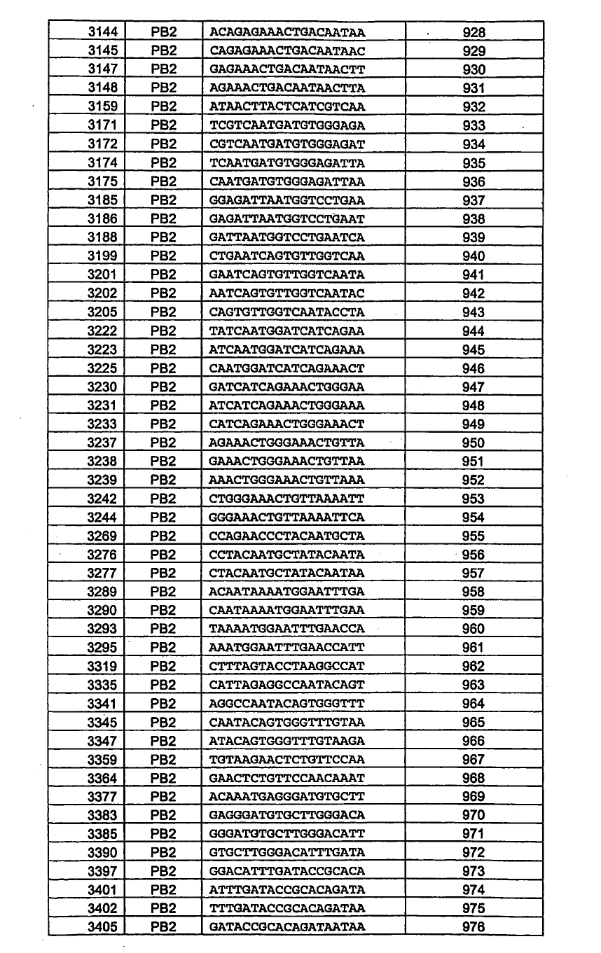

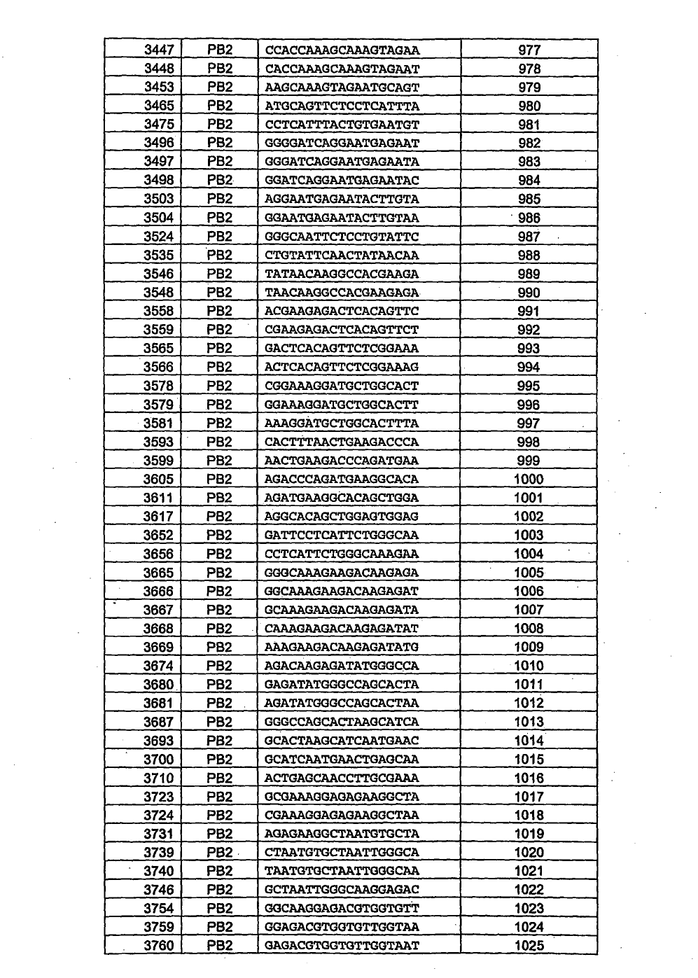

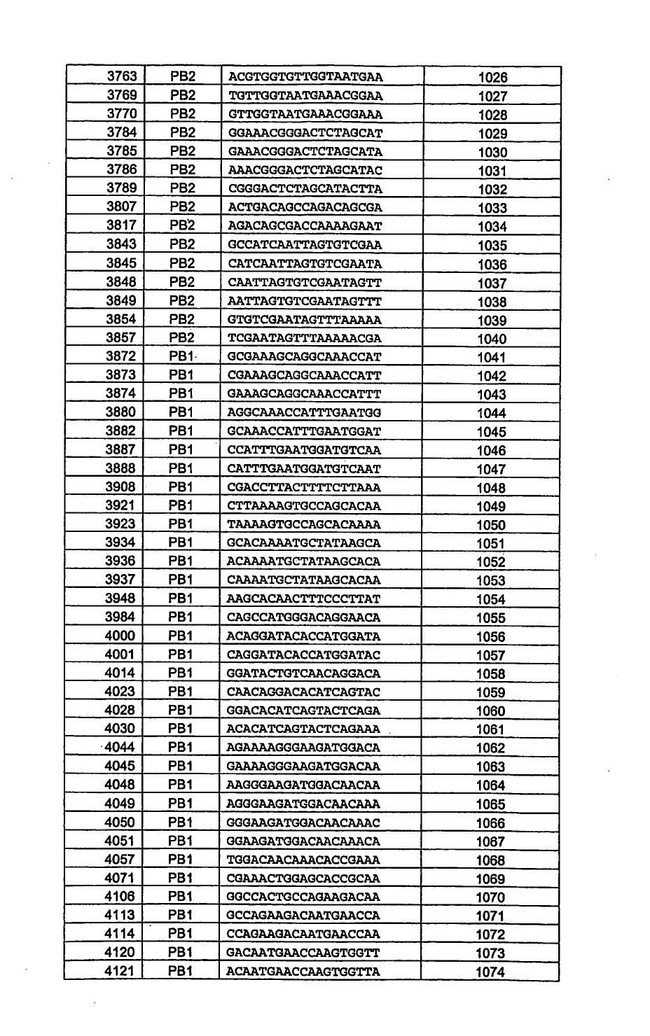

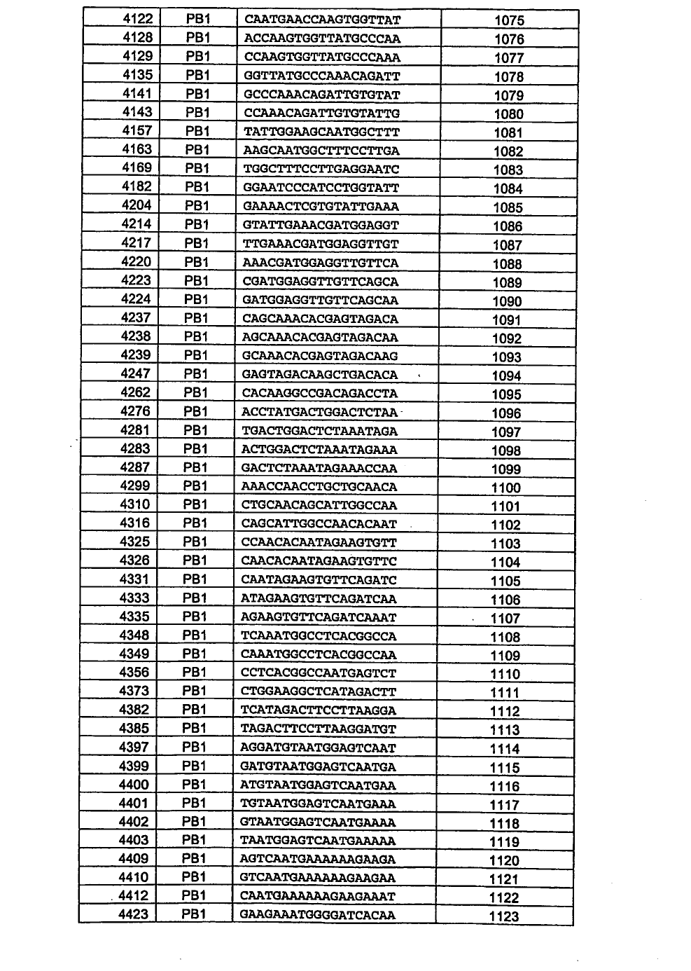

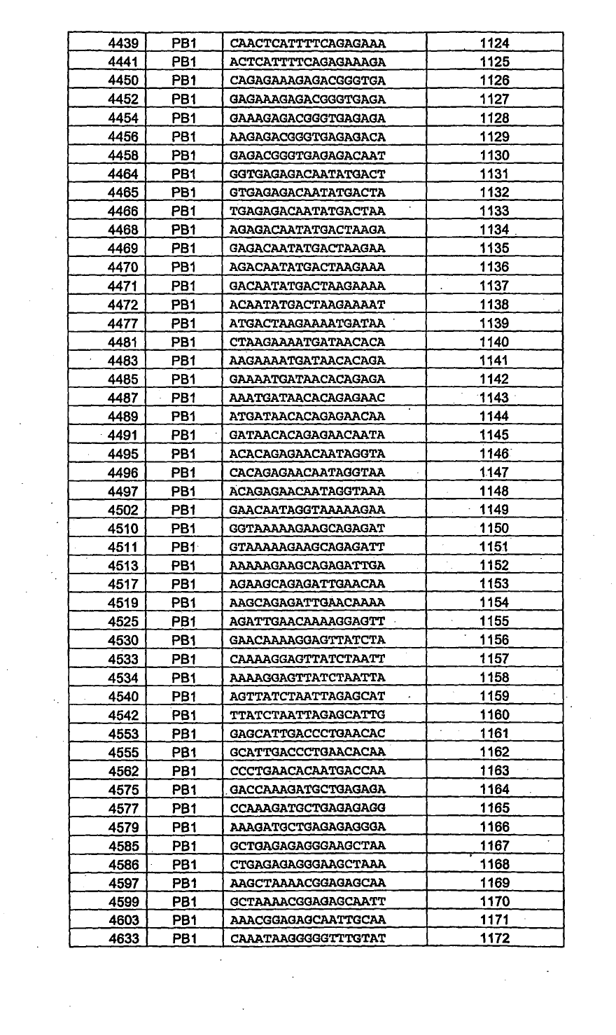

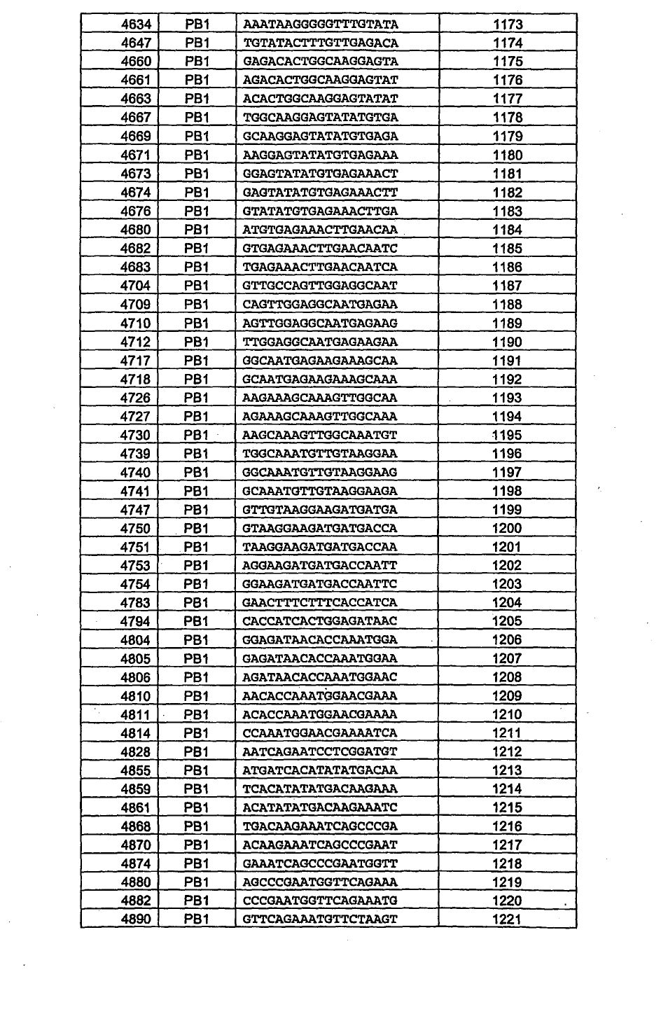

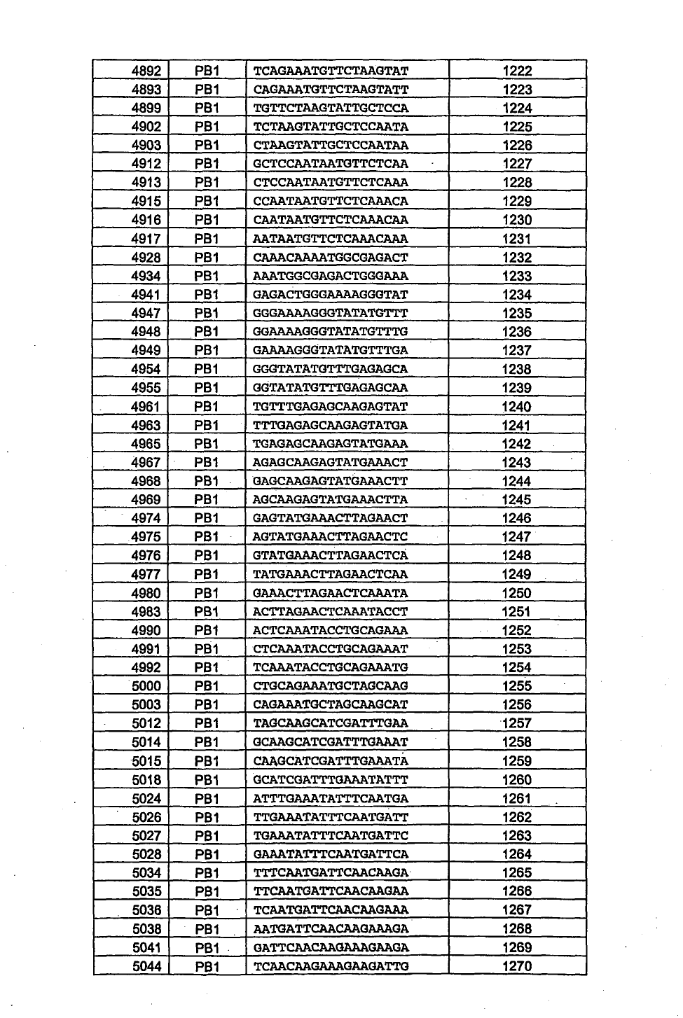

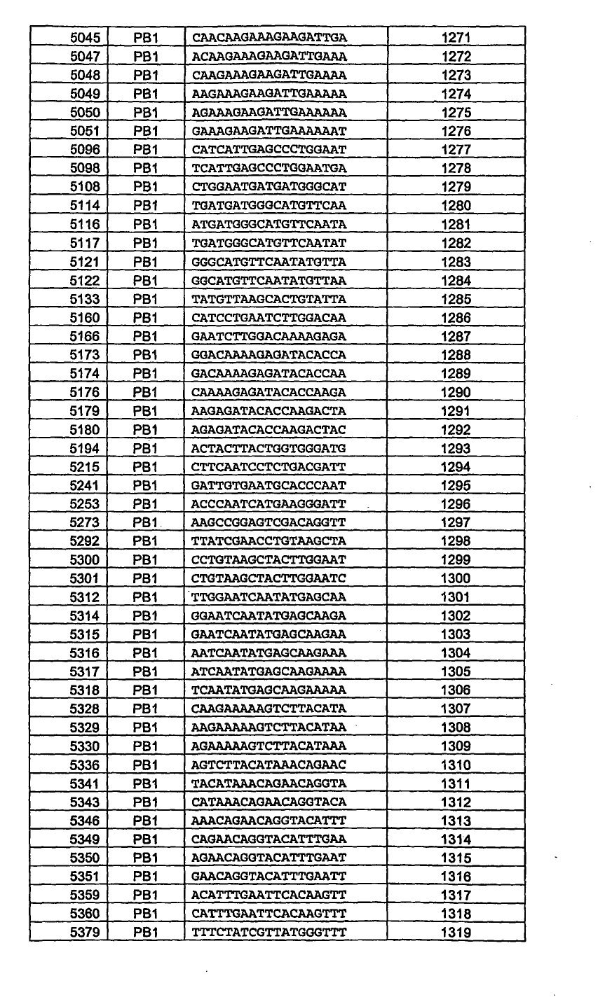

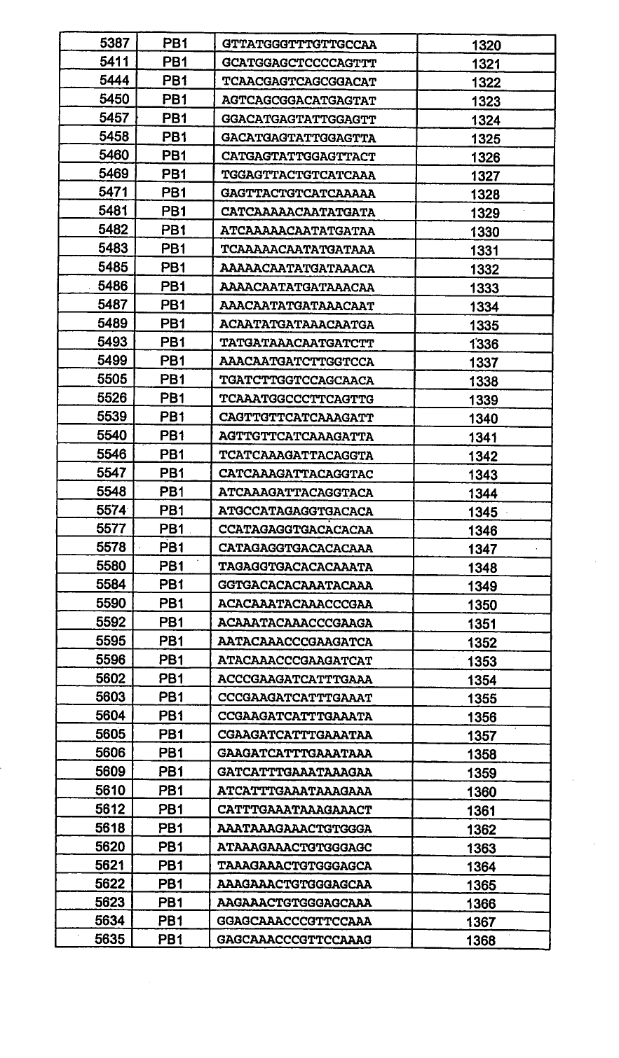

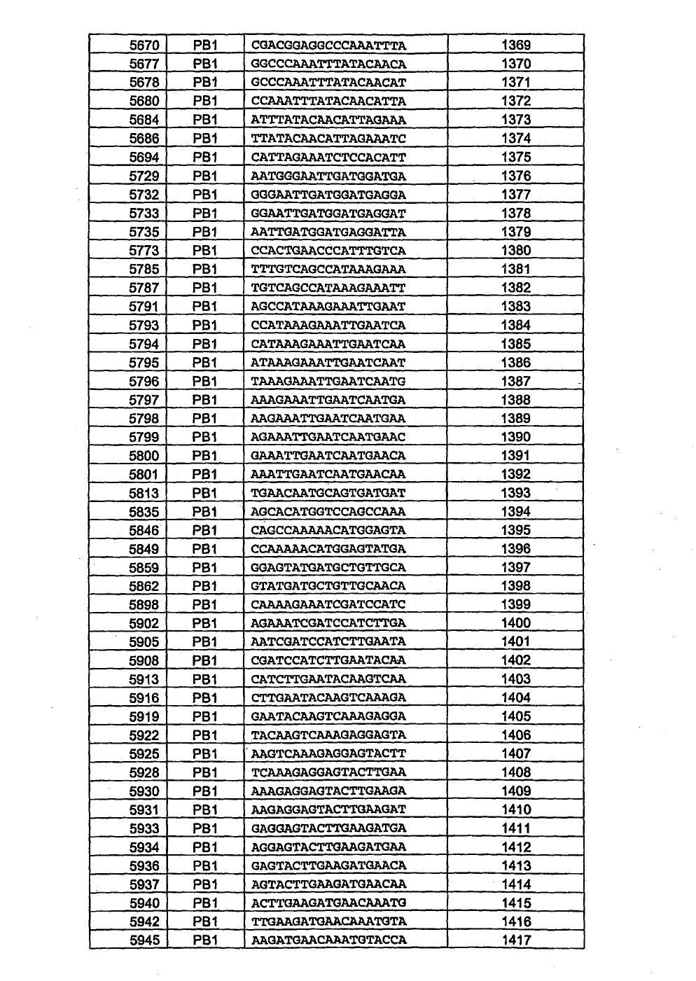

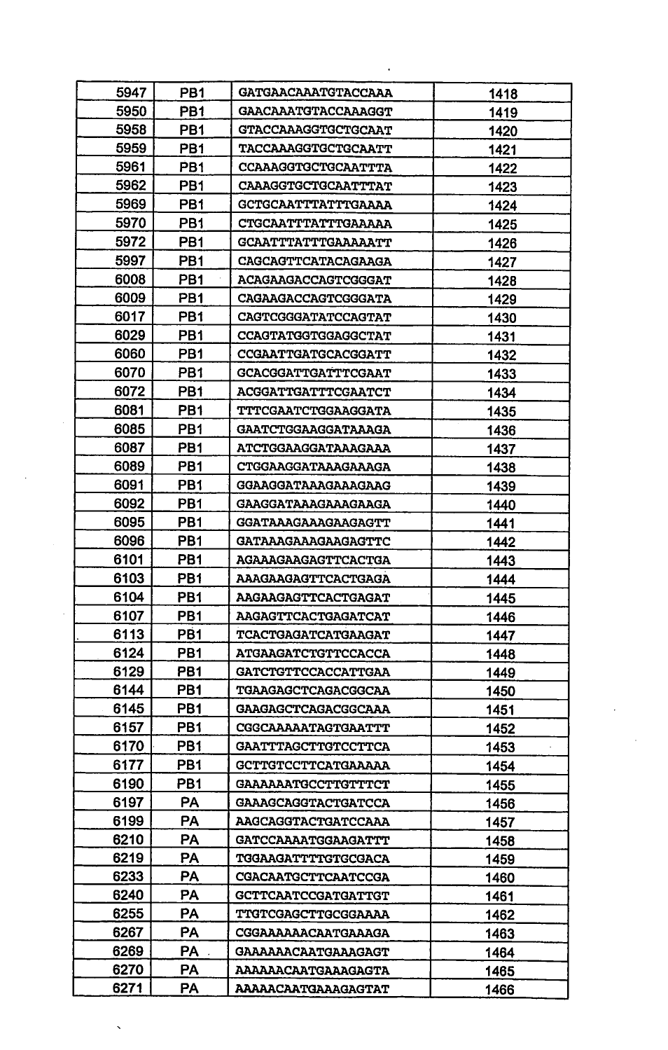

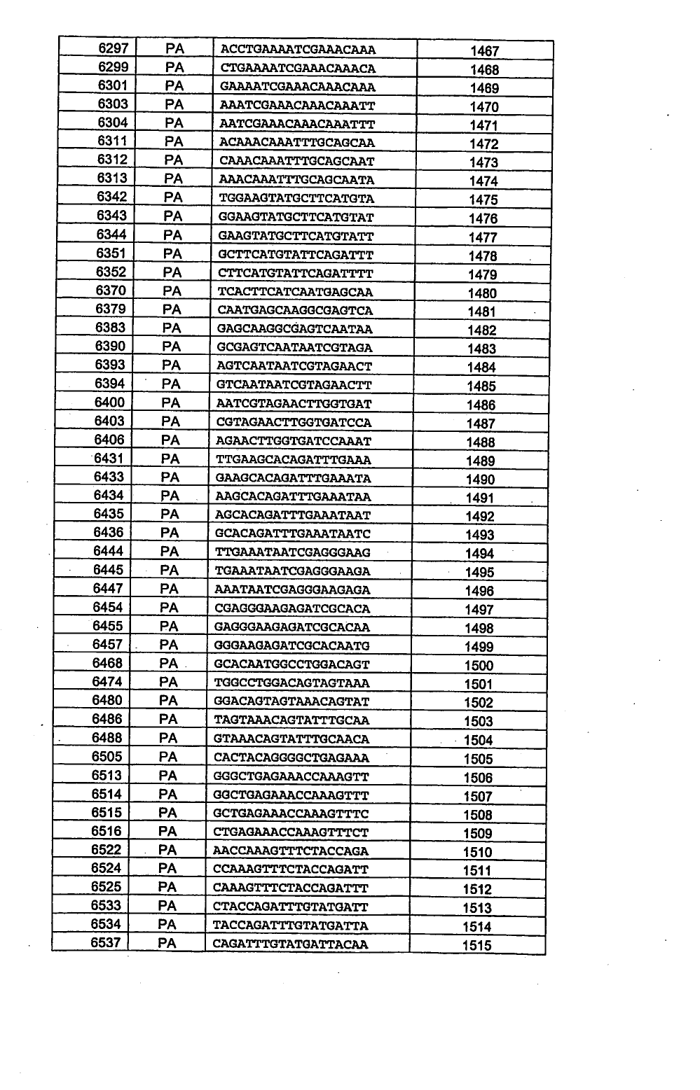

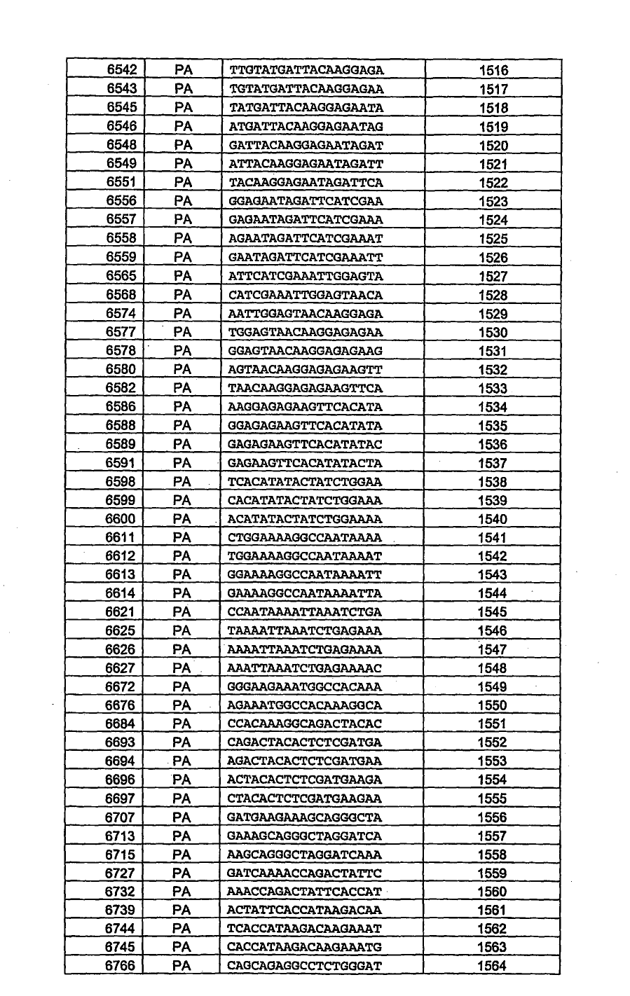

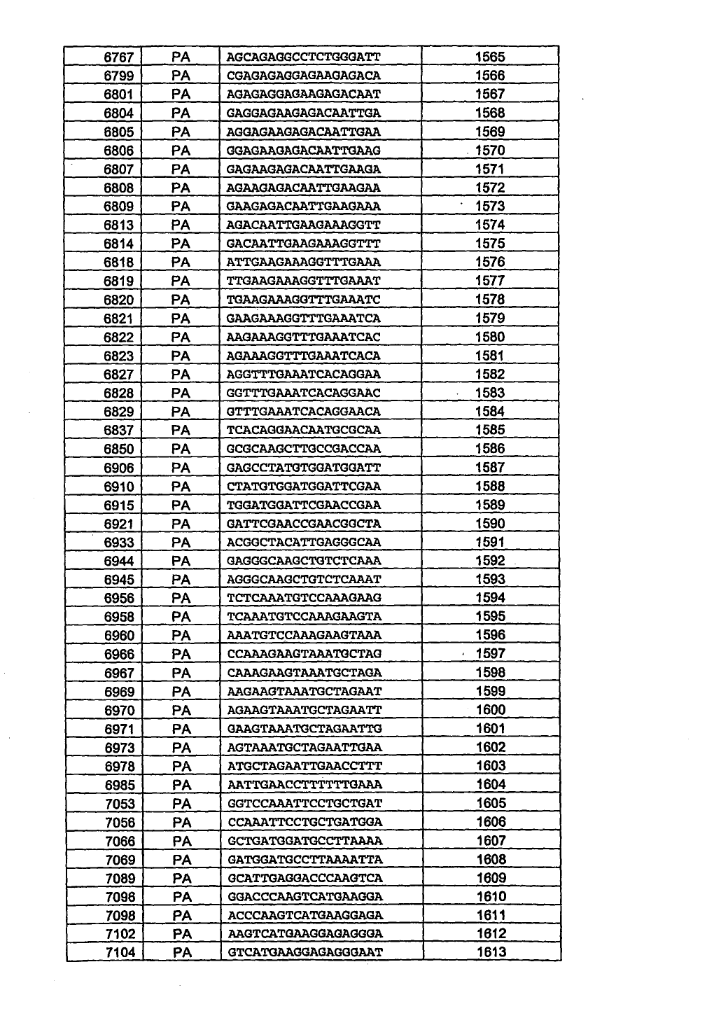

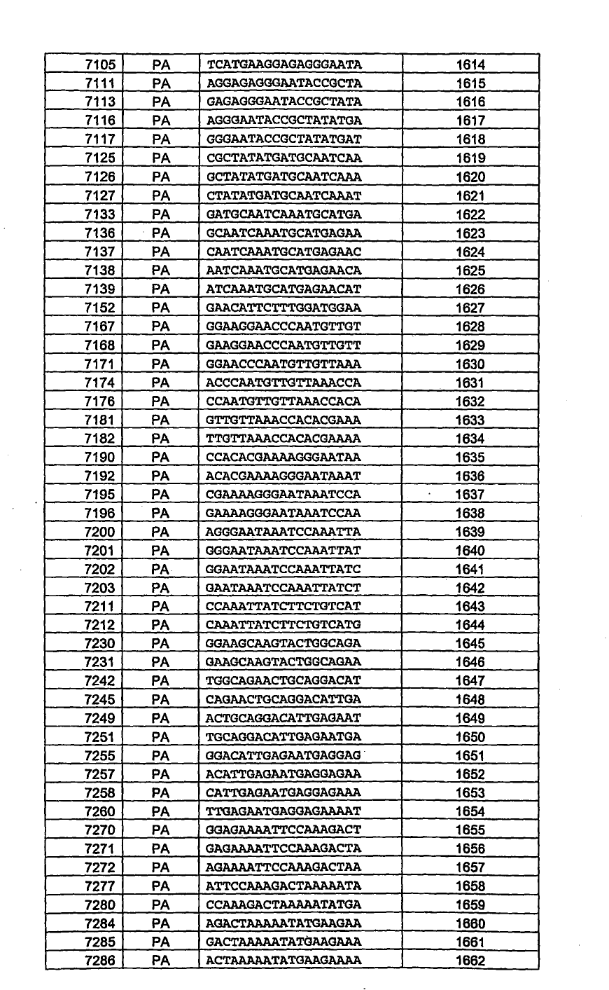

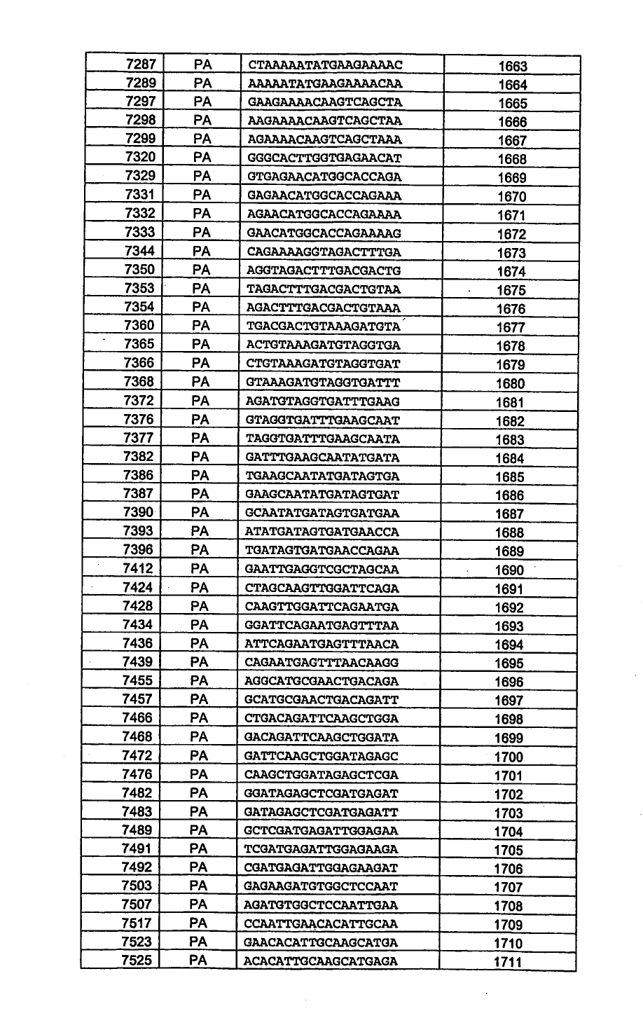

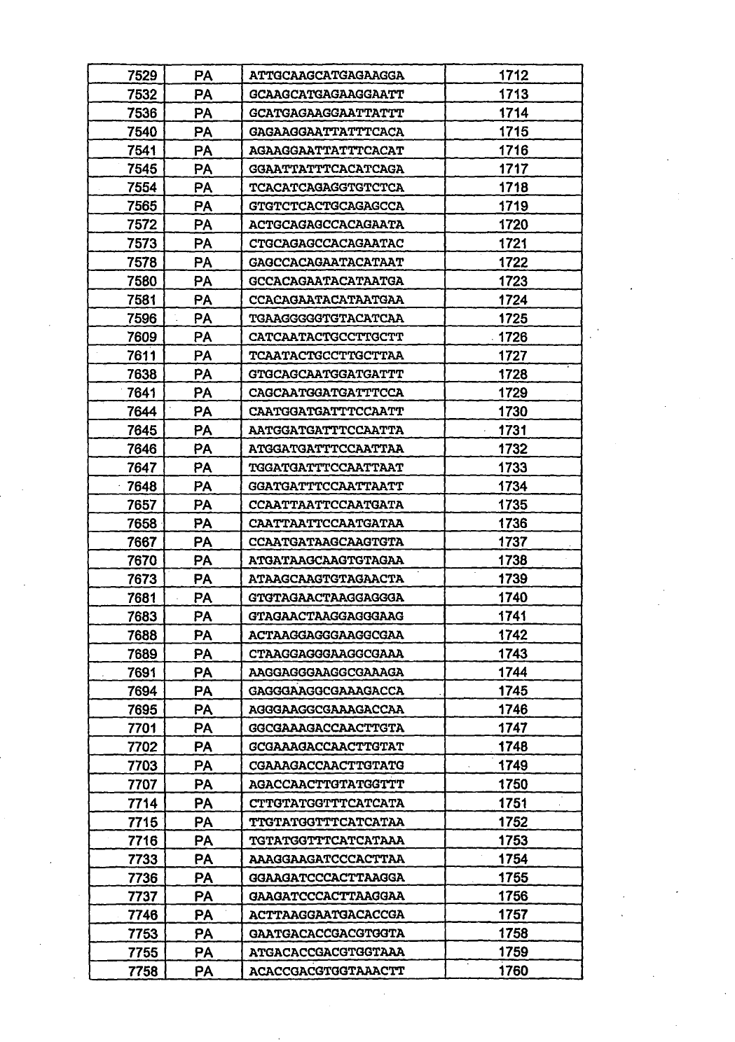

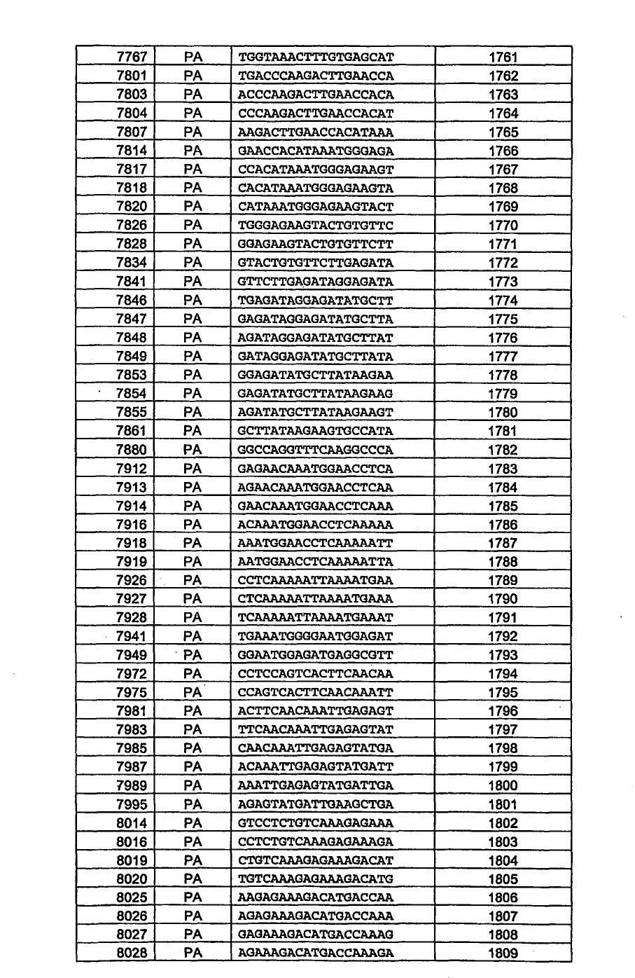

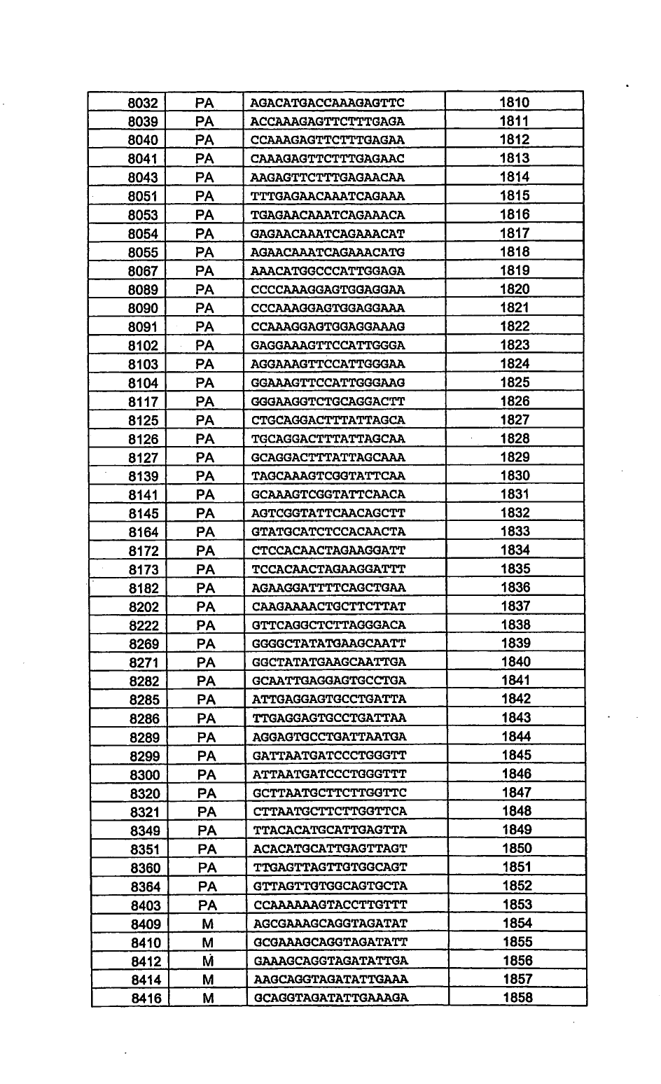

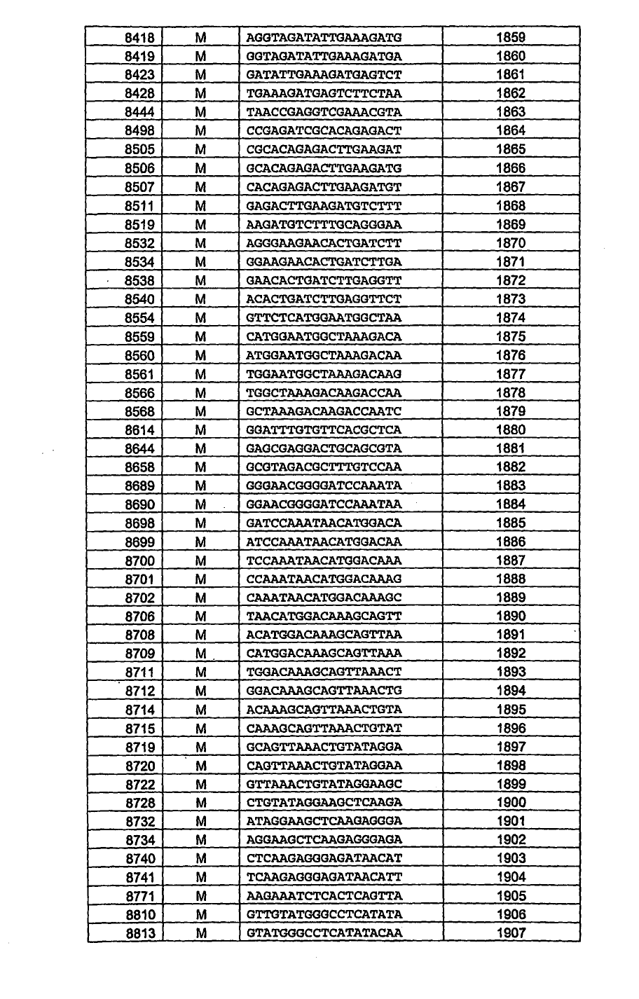

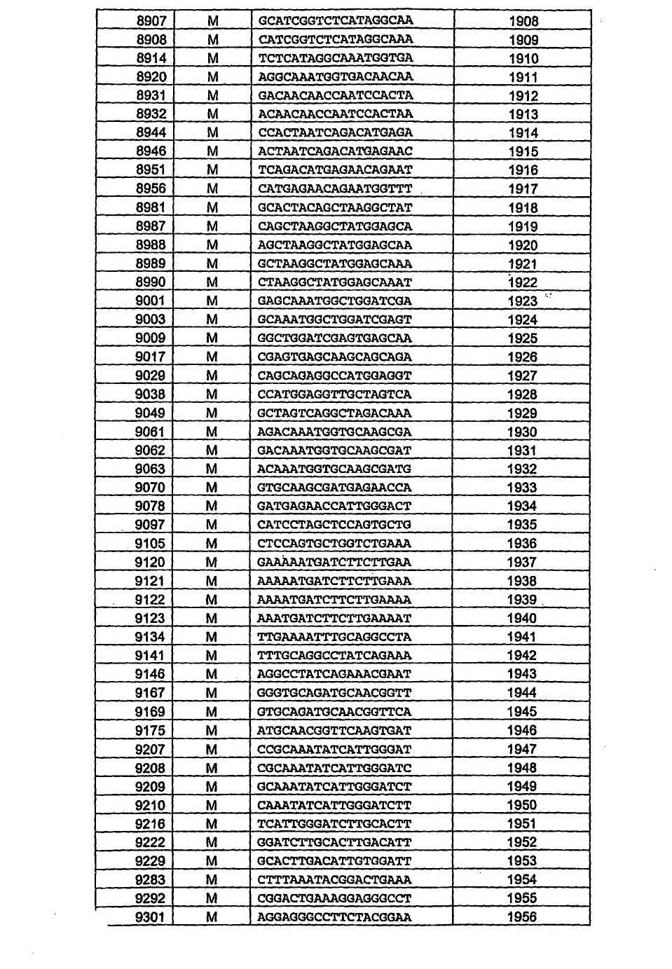

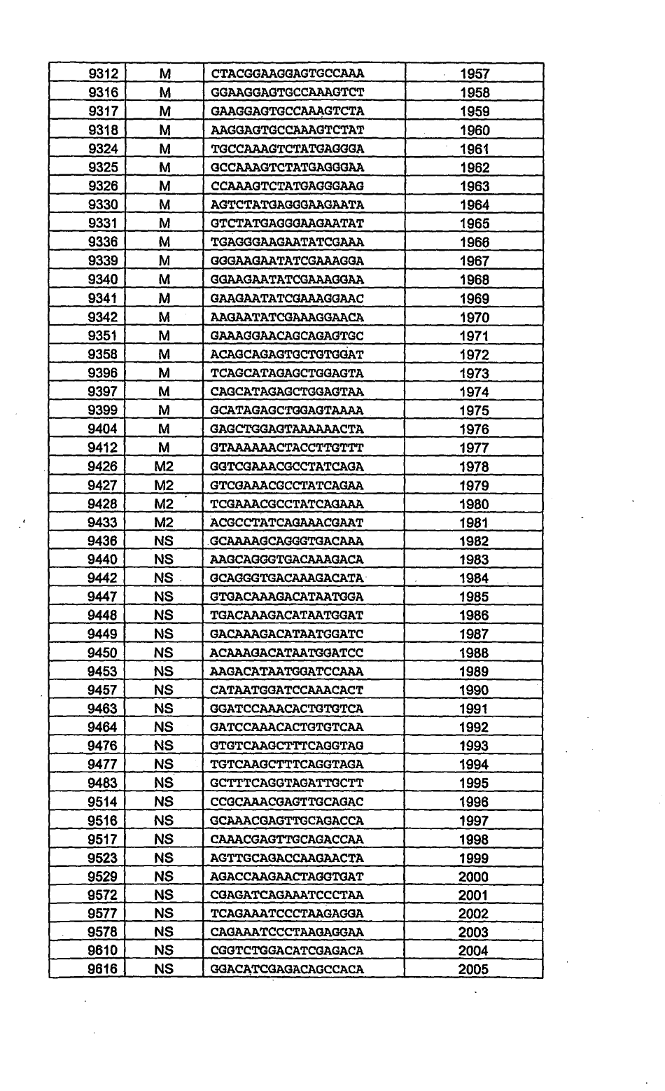

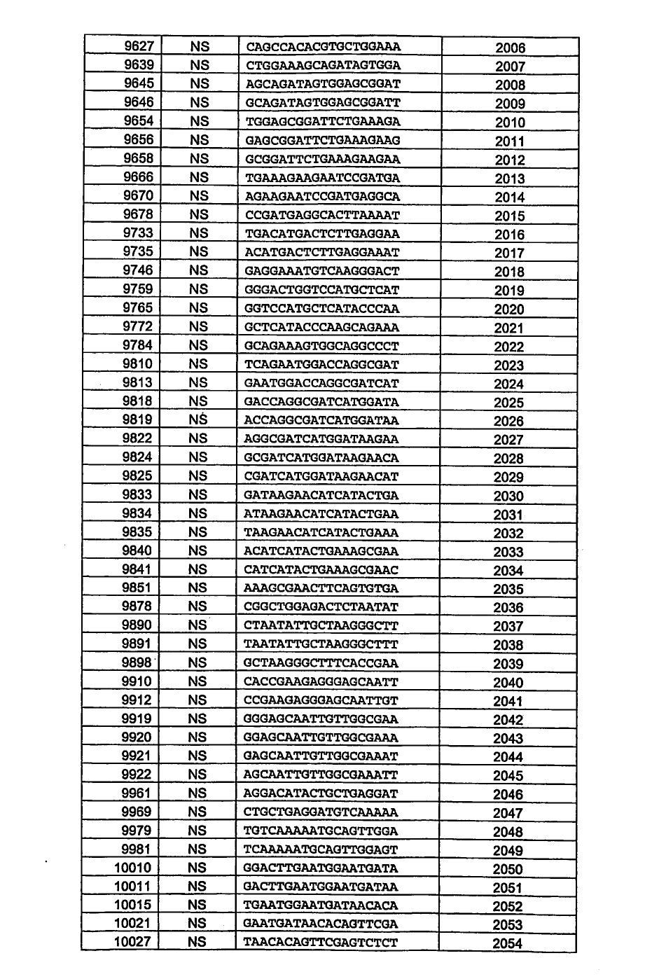

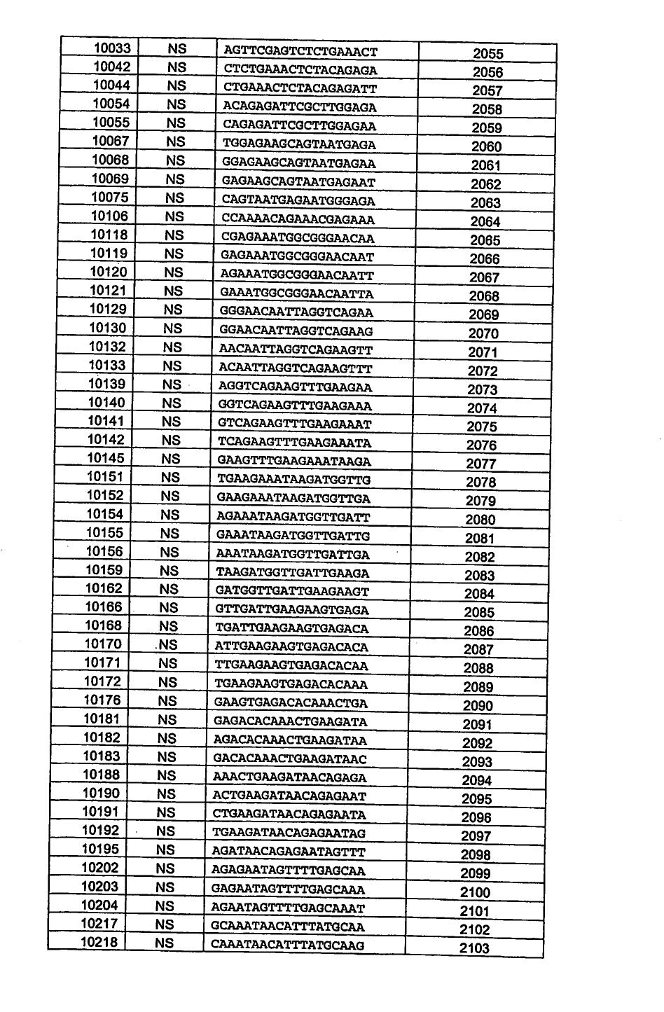

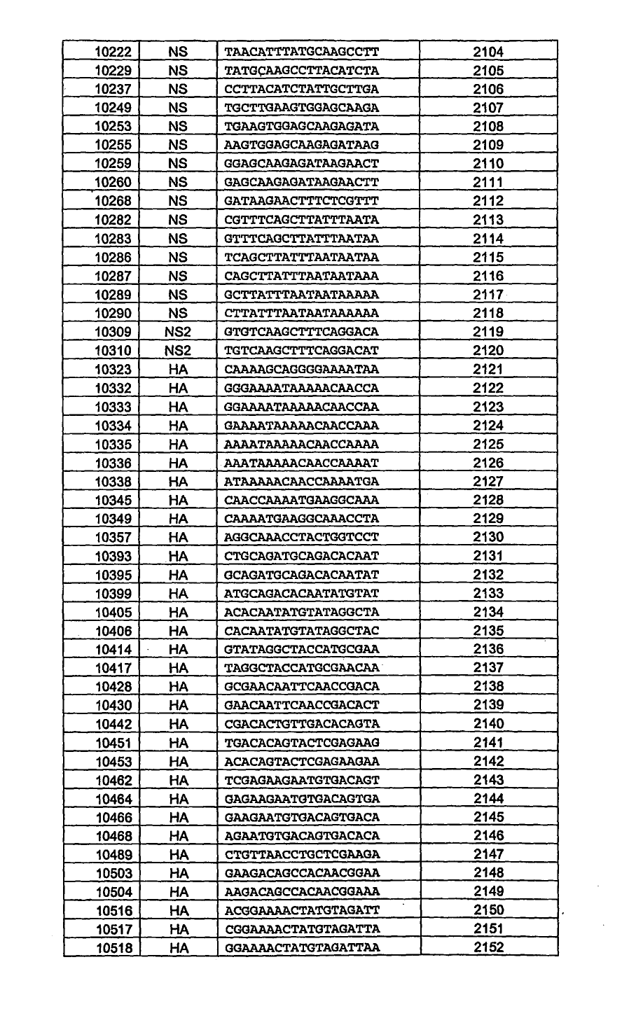

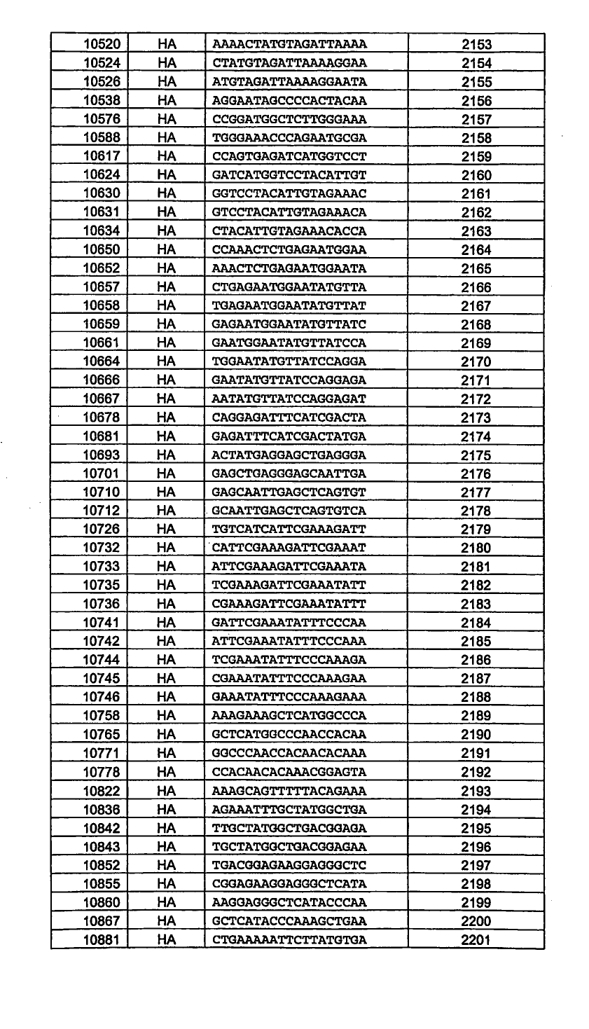

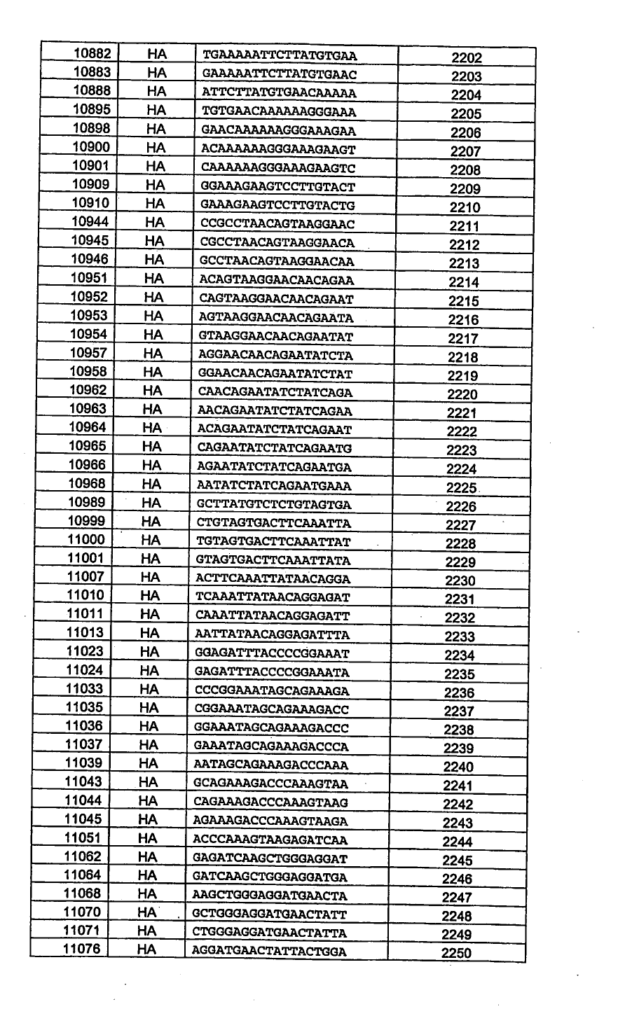

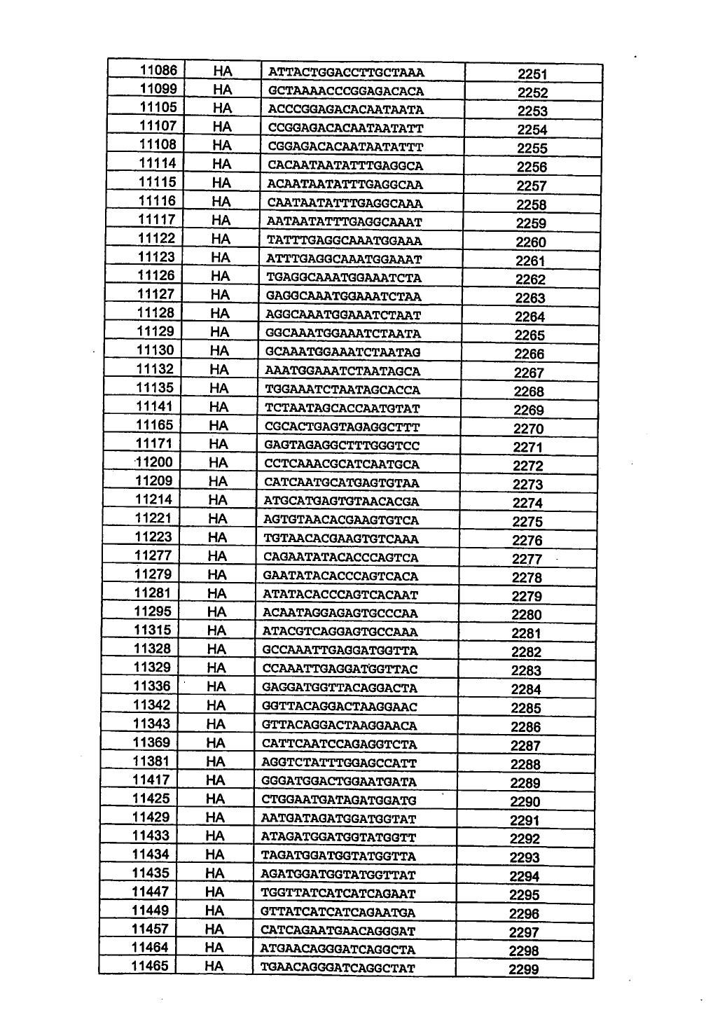

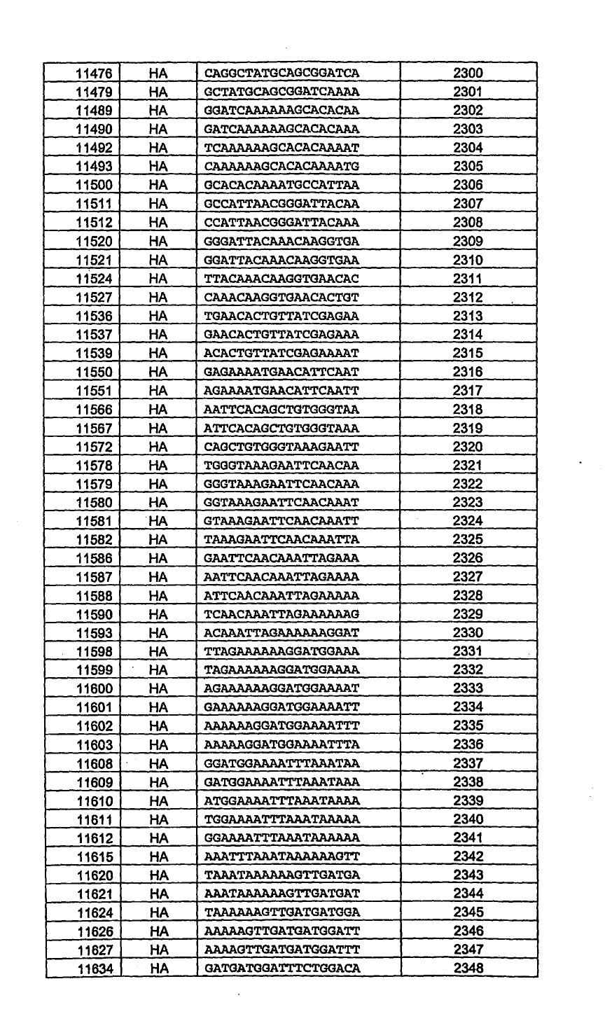

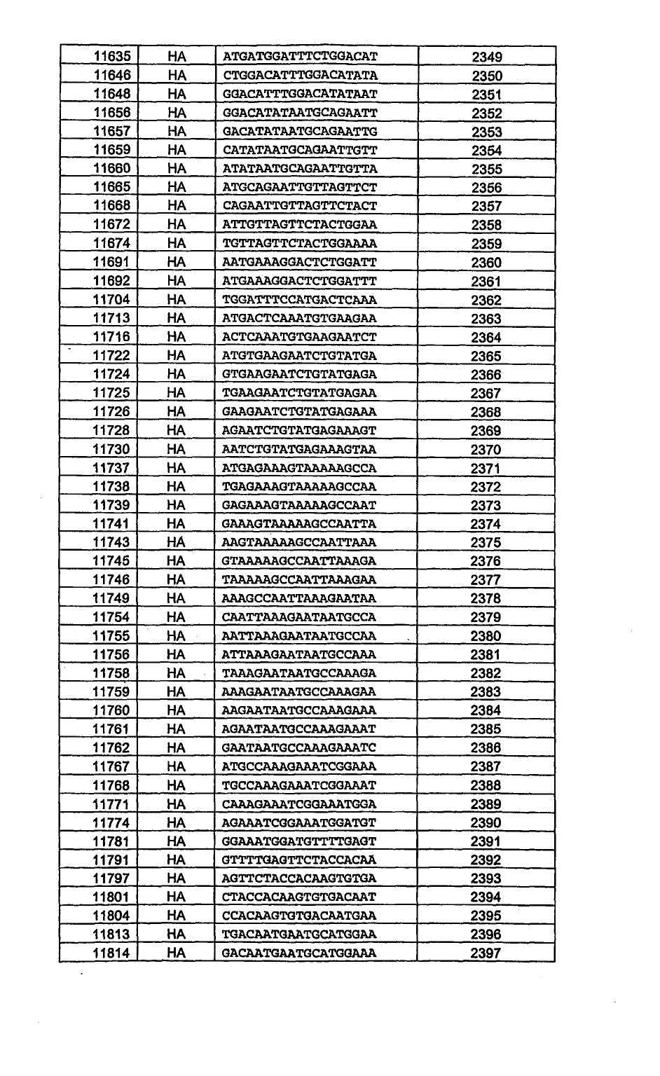

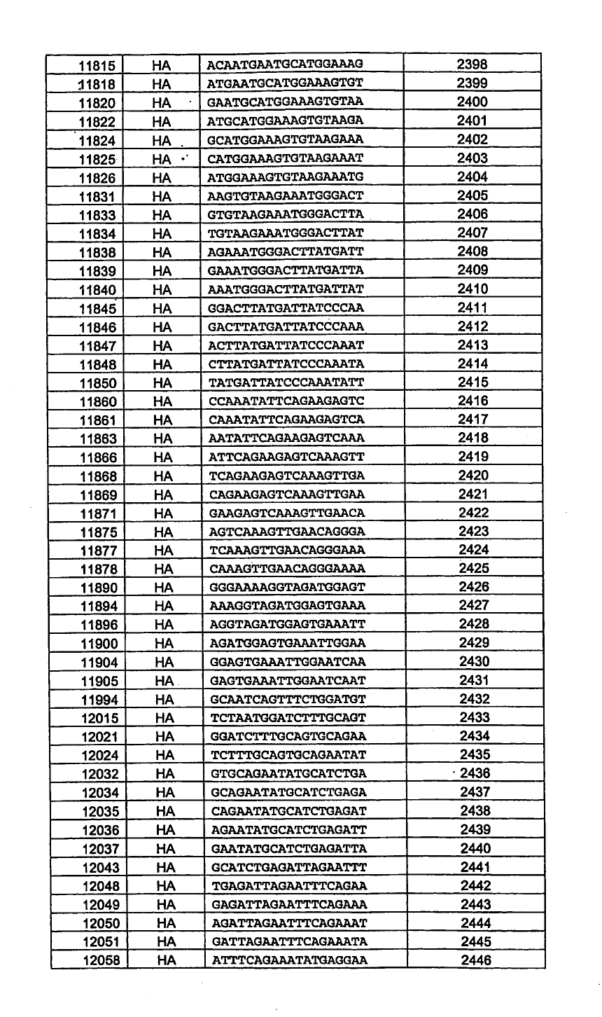

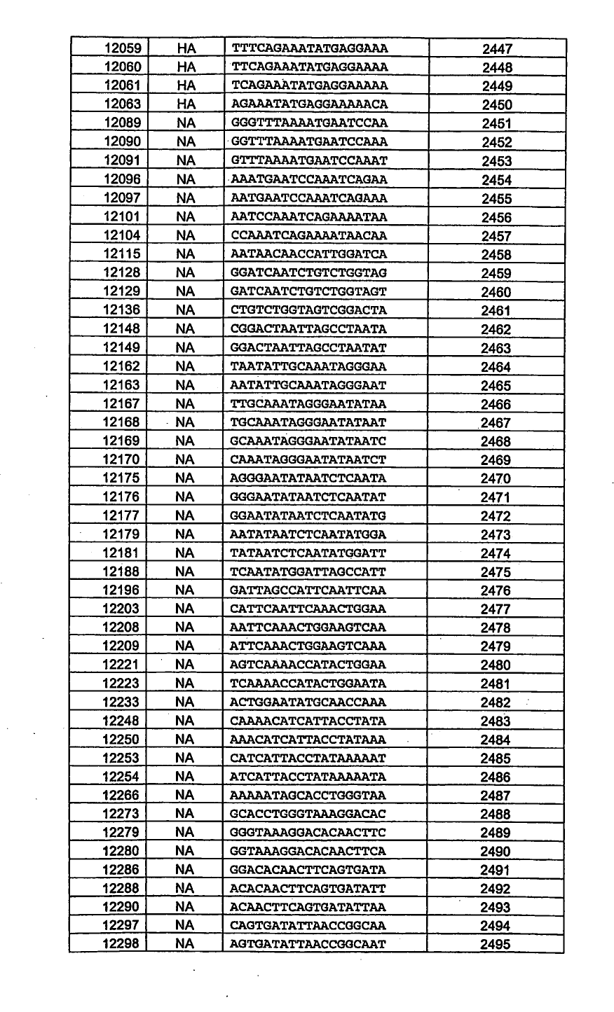

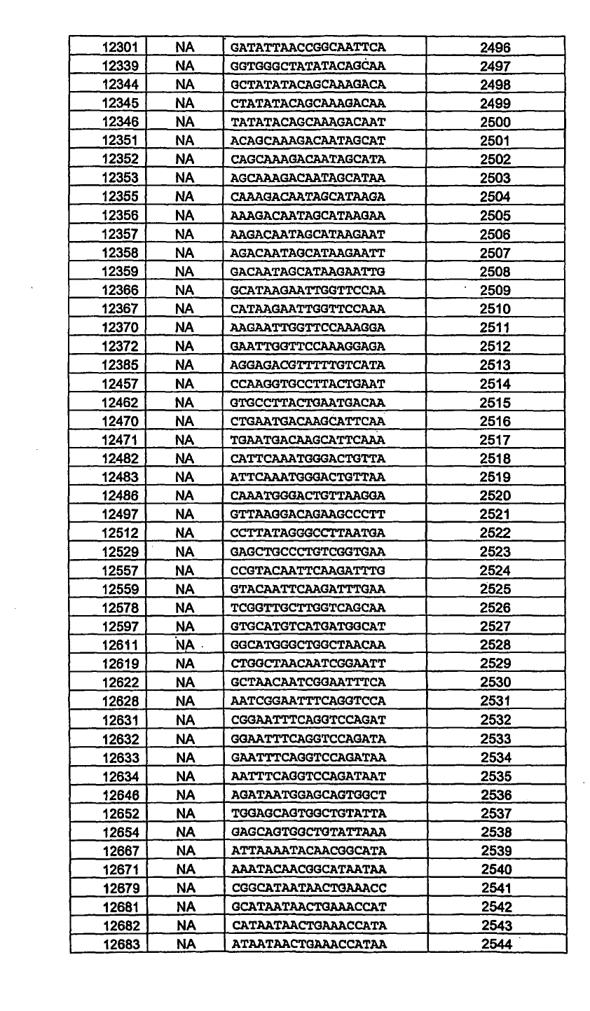

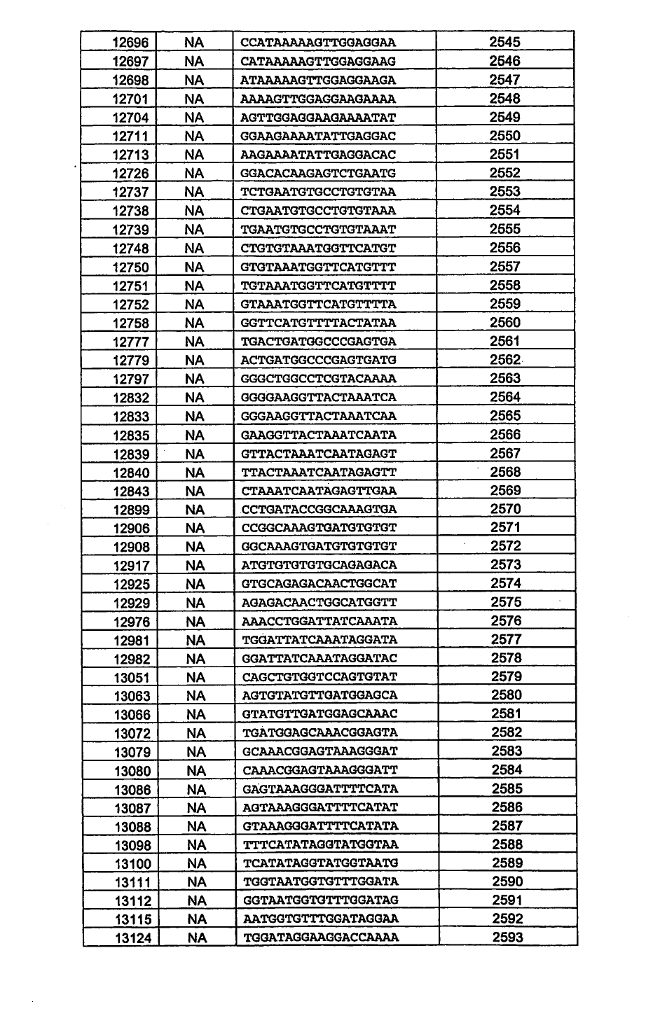

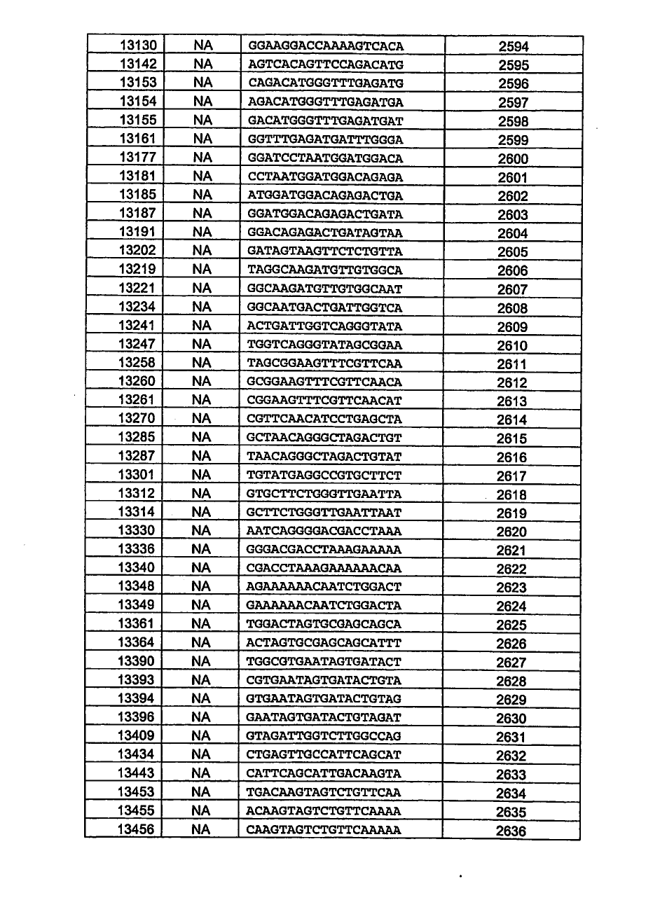

The next step was to identify preferred functional target portions for RNAi, i.e., regions of the gene whose sequence characteristics suggest that an RNAi-inducing agent having an antisense strand that hybridizes to the target will effectively inhibit its expression. The preferred functional target portions meet various criteria with respect to GC content and the absence of continuous stretches of G or C residues. Table 17 (FIG. 33) lists sequences of preferred functional target portions as present in base strain PR8.

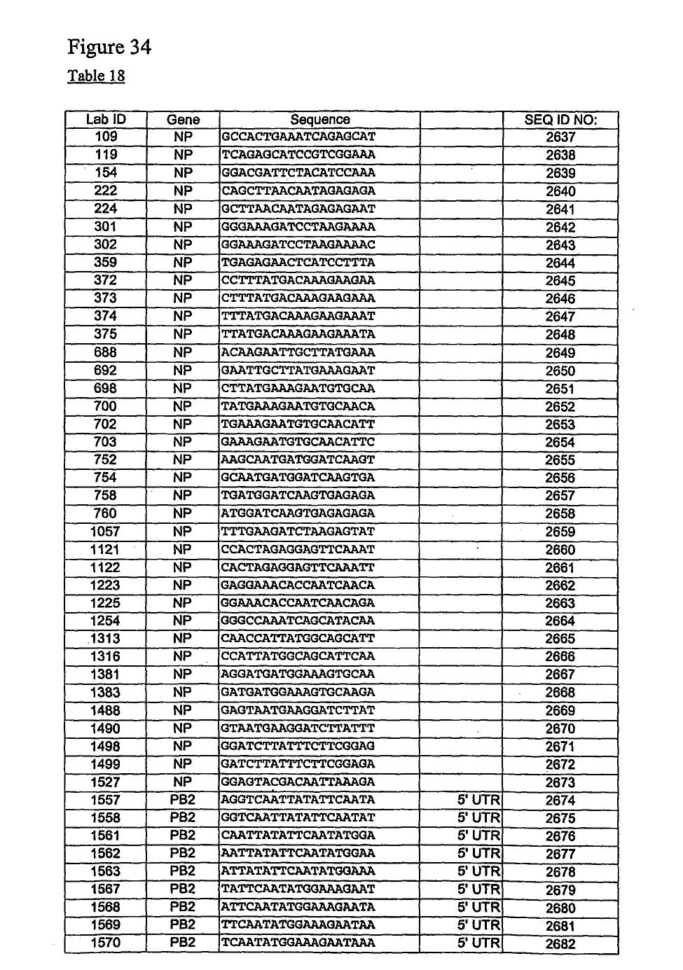

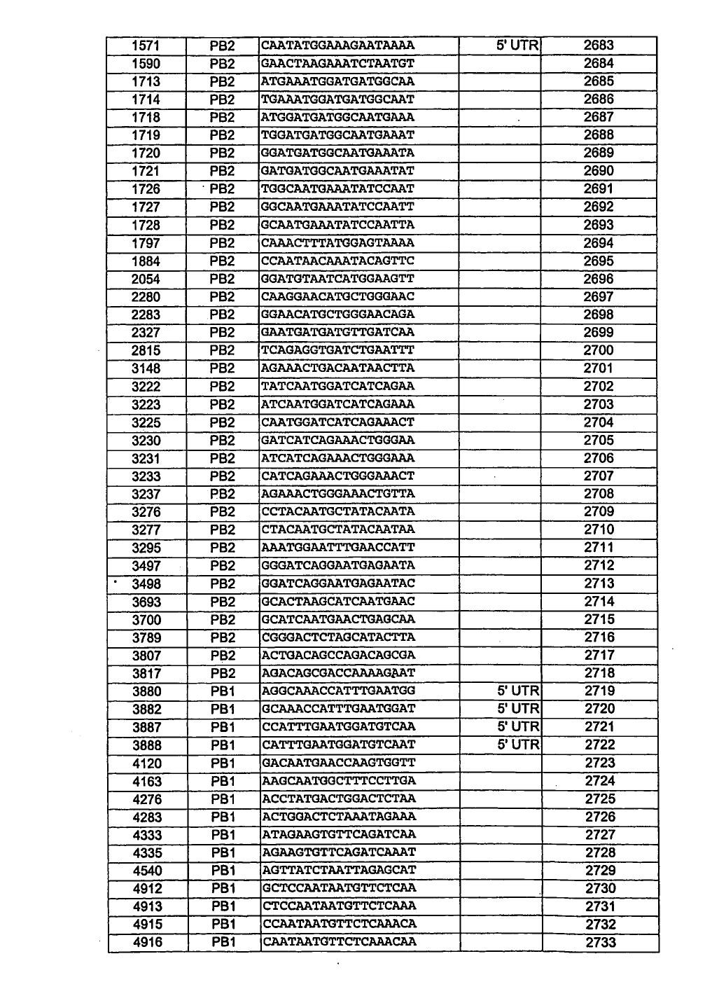

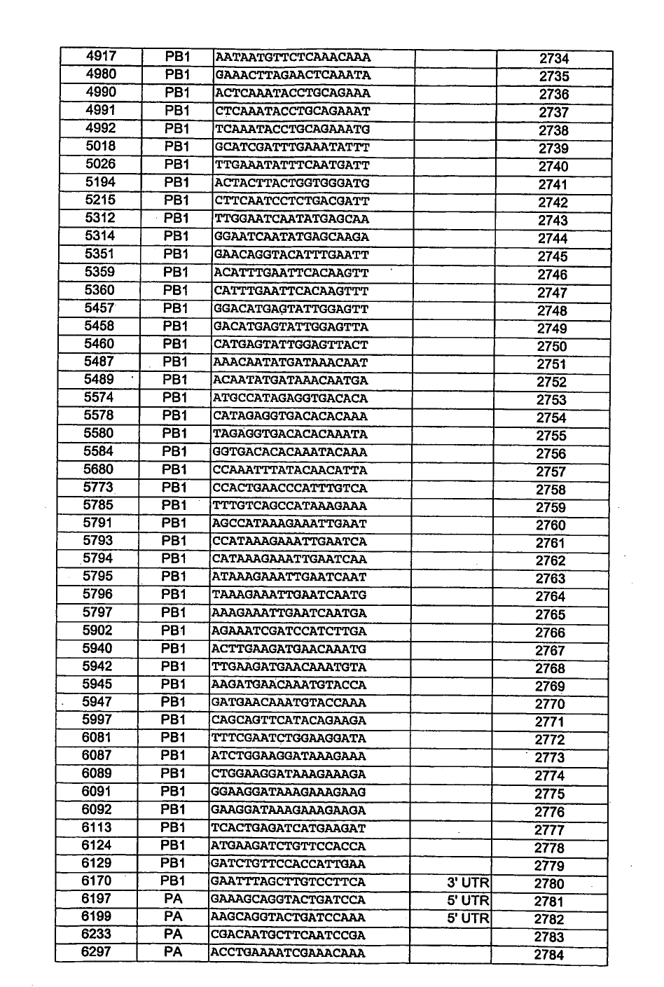

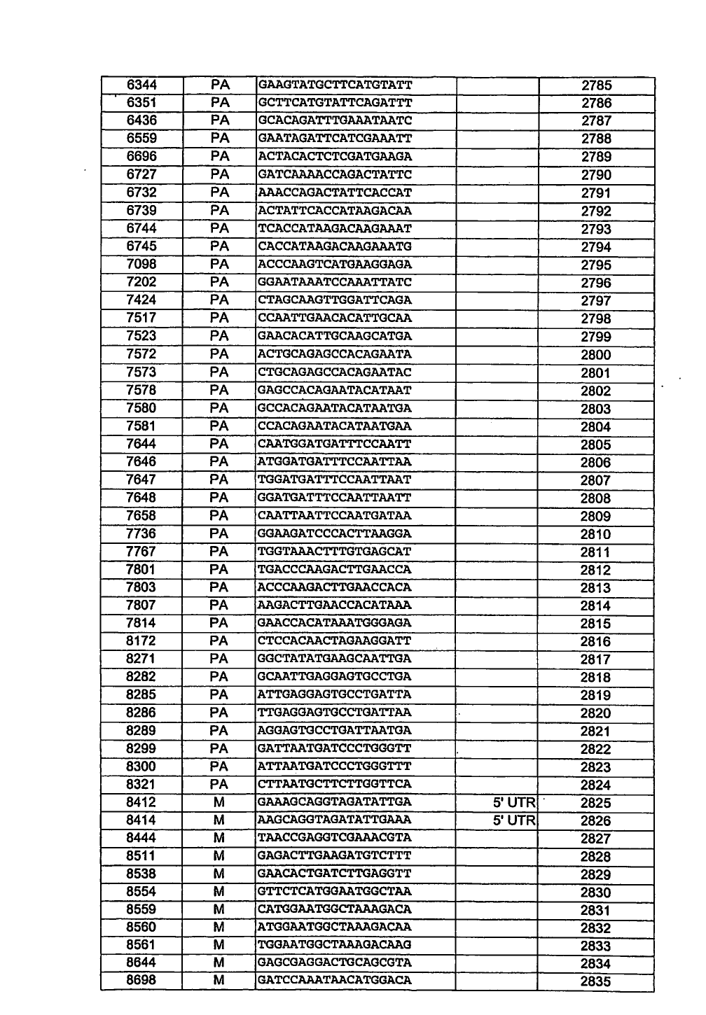

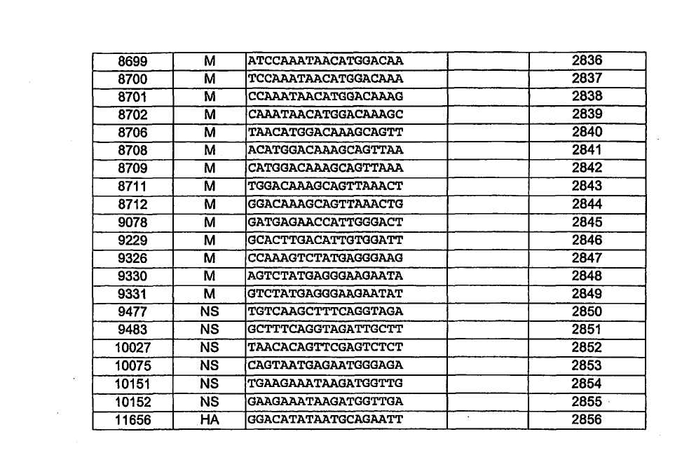

To identify favorably conserved target portions, corresponding functional target portions as found in a large number of human-derived influenza virus strains were then aligned. In general, “Corresponding target portions” in different strains are generally present at about the same position in the genome of the different strains when the genomic sequences are aligned to achieve maximum identity and/or are homologous, e.g., at least 50% identical in the different strains. Typically the degree of identity of corresponding target portions when two strains are compared is at least 60%, 70%, 80%, or more. For example, a corresponding target portion may differ from a target portion found in a base strain at 1, 2, 3, or 4 positions. The exact sequences of these preferred homologous target portions are readily identified by accessing the relevant influenza virus segment sequence in a database such as GenBank, aligning it with the base strain sequence, and locating a portion that is at about the same nucleotide position and/or at least 80% identical, preferably at least 90% identical to a target portion that is found in a base strain, e.g., PR8. Homologous target portions are an aspect of this invention. The criteria described above were used to select favorably conserved target portions. Table 18 (FIG. 34) lists sequences of target portions that are favorably conserved among influenza strains derived from humans, as present in base strain PR8.

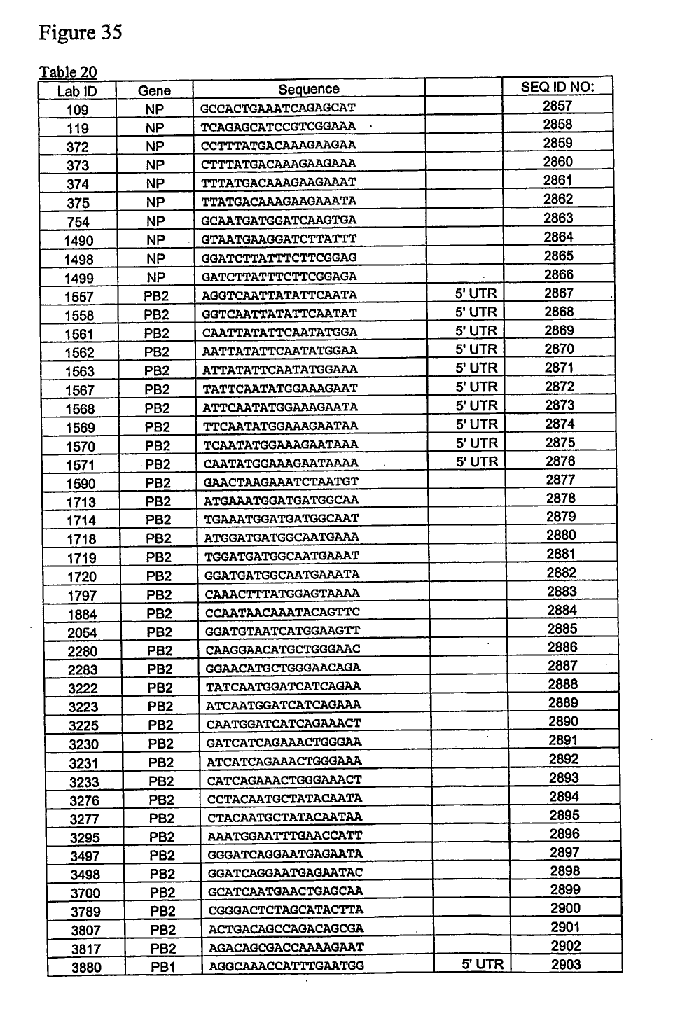

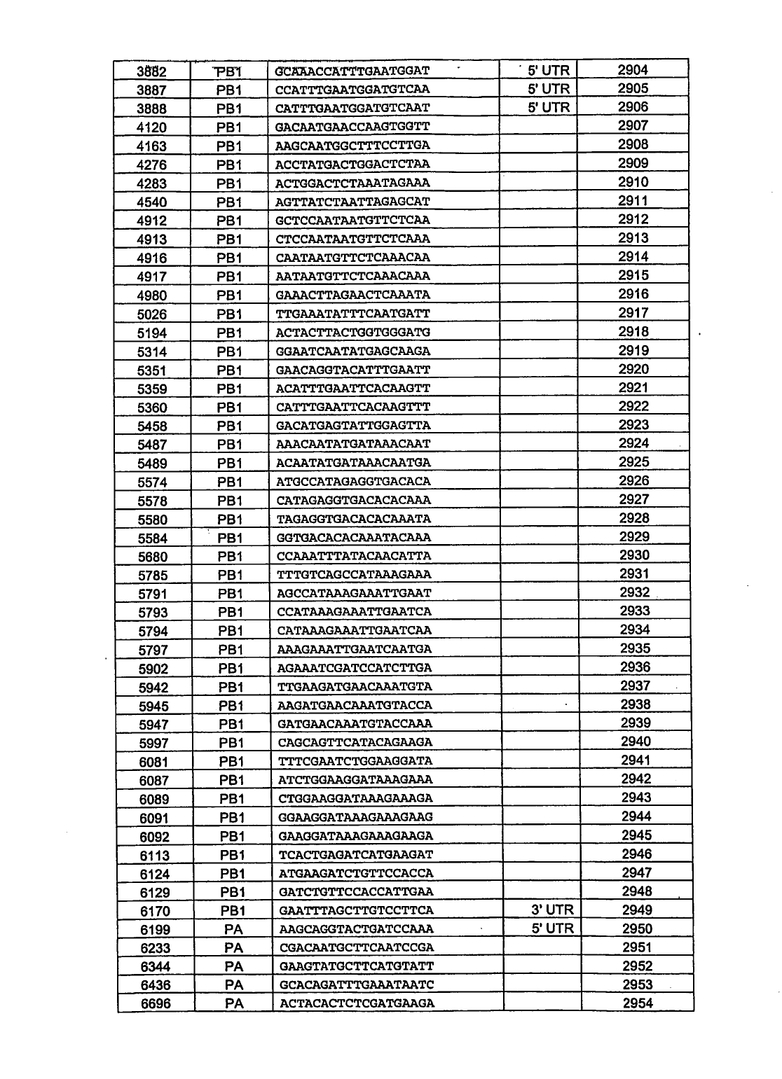

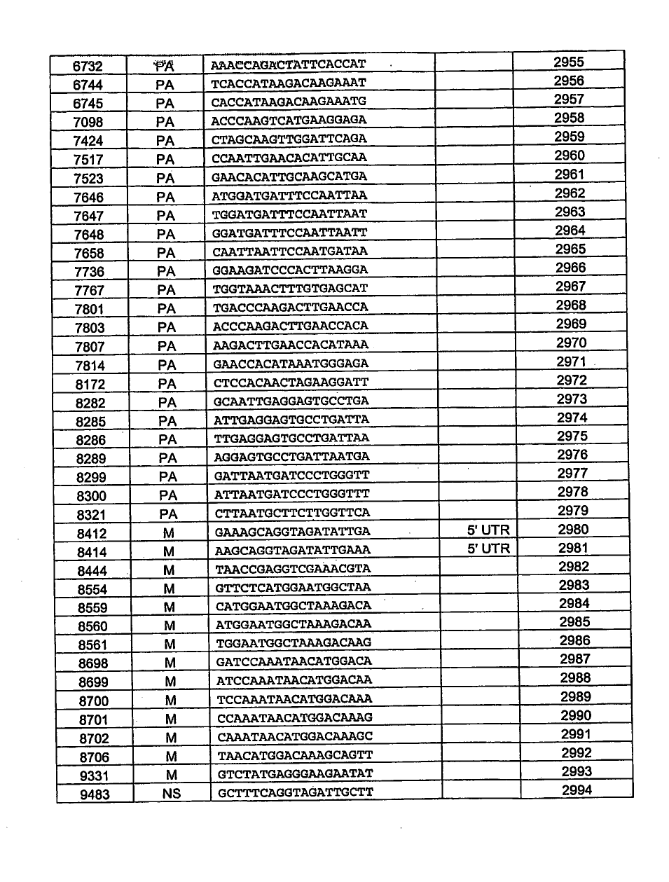

In addition to considering strains isolated from human hosts, strains isolated from avian hosts were aligned and compared in order to identify target portions that are favorably and/or highly conserved both among isolates from human hosts and isolates from avian hosts. Isolates from one or more other animal hosts could also be used for such comparisons. The favorably conserved target portions of the base sequence were compared with the aligned avian sequences, and favorably conserved target portions were selected using the same criteria that were used to identify favorably conserved target portions among the human isolates. Table 20 (FIG. 35) lists sequences of target portions that are favorably conserved among influenza strains derived from humans and avians, as present in base strain PR8.

Each 19 nucleotide potential target portion as found in PR8 or in another influenza virus strain listed in Tables 15A-15H or Tables 19A-19F is an aspect of this invention. Each preferred functional target portion as found in PR8 or in another influenza virus strain listed in Tables 15A-15H or Tables 19A-19F is an aspect of this invention. Each favorably and/or highly conserved target portion as found in PR8 or in another influenza virus strain listed in Tables 15A-15H or Tables 19A-19F is an aspect of this invention. The complement of each such sequence (i.e., potential, preferred functional, favorably conserved among human derived strains, favorably conserved among human derived strains and avian derived strains, and/or highly conserved among humans or avians or both) can serve as the sequence for the inhibitory region of the antisense strand of an RNAi-inducing agent targeted to the target portion. RNAi-inducing agents having antisense strands that comprise each of these sequences, or fragments of them at least 15 nucleotides in length, are an aspect of this invention. However, a variety of RNAi-inducing agents containing antisense strands that display less than perfect complementarity to the target portion or that are shorter or longer can also be used, as described herein. The sequence of a target portion can serve as the sense strand of an RNAi-inducing agent. Sense strands that are not perfectly complementary to the antisense strand can also be used as described herein.

As mentioned above, the target portions listed in Tables 17, 18, and 20 are identical to portions of influenza A virus PR8 transcripts. Corresponding target portions for each transcript, which are either identical to or highly homologous to the target portions listed in Tables 17, 18, and 20 are found in other influenza virus A strains. In certain embodiments of the invention the inhibitory region of an antisense strand strand of an RNAi-inducing agent targeted to a potential influenza virus target portion, e.g., a target portion listed in Table 17, 18, or 20 is not 100% complementary to a PR8 sequence but is 100% complementary to a corresponding target portion found in one or more of the other strains listed in Tables 15A-15H or Tables 19A-19F. The present invention provides RNAi-inducing entities, e.g., RNAi-inducing agents such as siRNA or shRNA targeted to each of the potential target portions, functional target portions, favorably conserved portions, and highly conserved target portions described herein. The invention also provides RNAi-inducing entities, e.g., RNAi-inducing agents such as siRNA or shRNA, targeted to target portions that comprise at least 15 continuous nt of a potential target portion, functional target portion, favorably conserved portion, or highly conserved target portion described herein. The invention also provides RNAi-inducing entities e.g., RNAi-inducing agents such as siRNA or shRNA, targeted to target portions up to approximately 29 nt in length that comprise a potential target portion, functional target portion, favorably conserved portion, or highly conserved target portion described herein. The additional nucleotides of the target portion are preferably located immediately 5′ and/or or 3′ from a 19 nt target portion listed herein. In other words, some of the additional up to approximately. 10 nt of these longer target portions may be upstream of the 19 nt target portion, and some may be downstream of the 19 nt target portion. The exact sequences may readily be identified by accessing the appropriate entry for a genome segment containing a listed target portion or a corresponding target portion from any strain and identifying the nucleotides that are immediately 5′ and/or 3′ of the listed or corresponding target portion. FIGS. 32A-32J present the genome segment sequences for PR8 (positive strand form).

The present invention provides RNAi-inducing agents that have been tested in cell culture and/or in animal models to verify their effectiveness and to identify portions of influenza virus transcripts that can be targeted in a highly effective manner. Example 18 describes a high throughput screen (HTS) that was used to identify siRNAs that effectively reduce levels of a targeted influenza virus transcript. Examples 19-22 describe high throughput screens that were used to identify siRNAs that effectively reduce influenza virus production in cells. Certain preferred effective siRNAs reduced influenza A virus titer by at least 4-fold when contacted with cells at a concentration of 100 nM. Certain preferred highly effective siRNAs reduced influenza virus titer by at least 8-fold, or to an even greater extent when contacted with cells at a concentration of 100 nM. Certain preferred effective siRNAs reduced influenza A virus titer by at least 4-fold when contacted with cells at a concentration of 1 nM. Certain preferred effective siRNAs reduced influenza A virus titer by at least 4-fold, at least 8-fold, or at least 16-fold when contacted with cells at a concentration of 100 nM. Certain highly effective siRNAs reduced influenza A virus titer by at least 2-fold when contacted with cells at concentrations of 5 nM or less. Certain even more highly effective siRNAs reduced influenza A virus titer by at least 2-fold when contacted with cells at concentrations of 1 nM or less. Yet more highly effective siRNAs reduce influenza A virus titer by at least 2-fold when contacted with cells at concentrations of 0.8 pM or less. Concentrations refer to concentrations at which the siRNA was present in a medium external to the cells at the time of introduction.

In certain embodiments an RNAi-inducing agent is targeted to a target portion within 200 nt from the 3′ end of a gene, e.g., the NP, PA, PB1, or PB2 gene. Seven highly effective siRNAs were found to target portions within this region. The inventors observed that the 5′ and 3′ ends of influenza gene segments contain ˜10 bases that are highly conserved both across different gene segments and across different influenza virus strains. Notably, 3 siRNAs that target the 5′ UTR of the PB1 transcript showed a 4-fold reduction in virus titer at 100 nM.

B. Synthesis of RNAi-Inducing Entities

Inventive RNAi-inducing agents may be prepared according to any available technique including, but not limited to chemical synthesis, enzymatic or chemical cleavage in vivo or in vitro, or template transcription in vivo or in vitro. For example, RNA may be produced enzymatically or by partial/total organic synthesis, and a modified nucleotide can be introduced by in vitro enzymatic or organic synthesis. In one embodiment, a siRNA is prepared chemically. Methods of synthesizing RNA molecules are known in the art, in particular, the chemical synthesis methods as de scribed in Verma and Eckstein, Annu. Rev. Biochem. 67:99-134 (1998). In another embodiment, an siRNA is prepared enzymatically. For example, an siRNA can be prepared by enzymatic processing of a long dsRNA having sufficient complementarity to the desired target RNA. Processing of long dsRNA can be accomplished in vitro, for example, using appropriate cellular lysates and siRNAs can be subsequently purified by gel electrophoresis or gel filtration. For example, RNA can be purified from a mixture by extraction with a solvent or resin, precipitation, electrophoresis, chromatography, or a combination thereof. Alternatively, the RNA may be used with no or minimum purification to avoid losses due to sample processing.

Inventive RNAi-inducing agents may be delivered as a single shRNA molecule or as two strands hybridized to one another. For instance, two separate 21 nt RNA strands may be generated, each of which contains a 19 nt region complementary to the other, and the individual strands may be hybridized together to generate a structure such as that depicted in FIG. 5A.

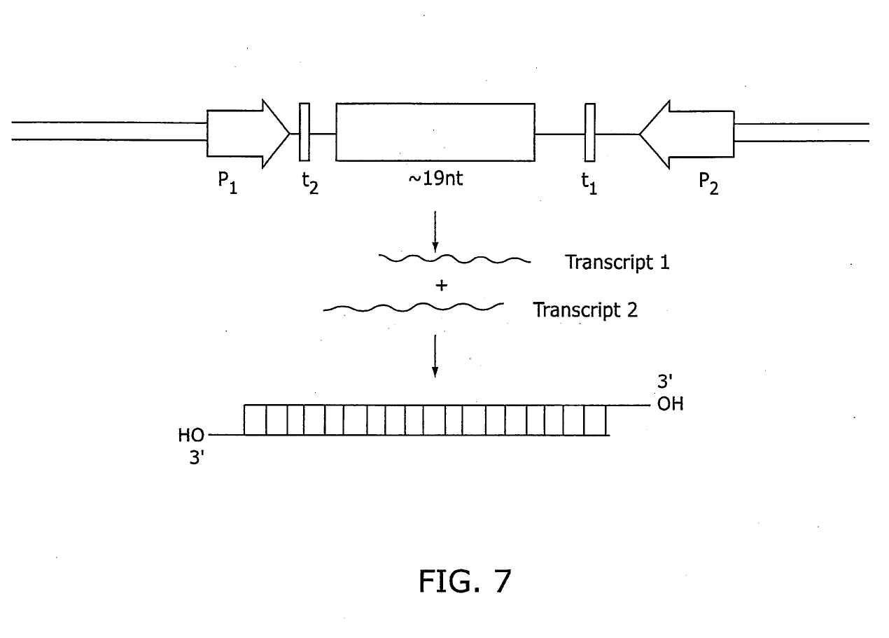

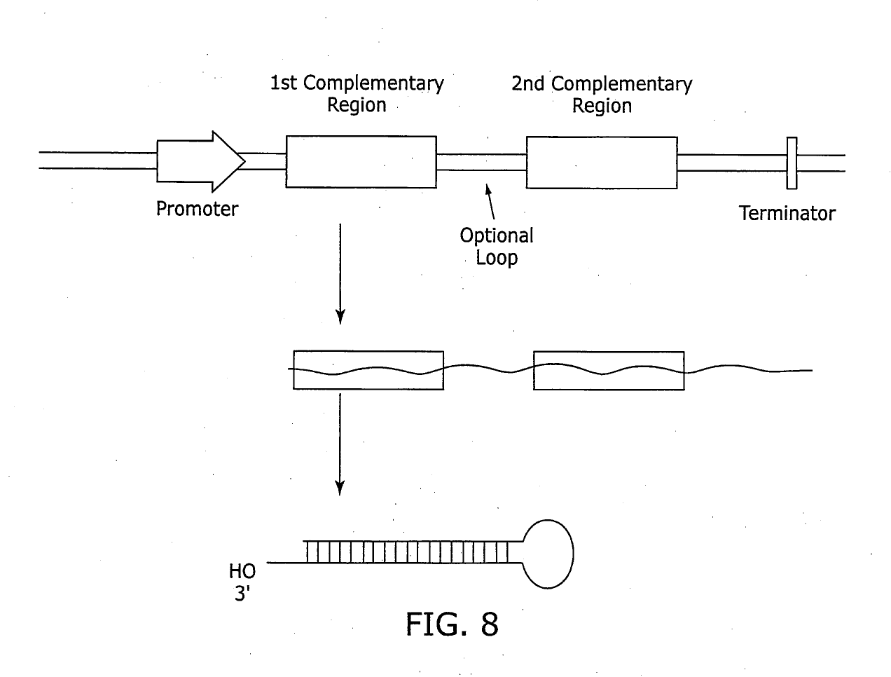

In some embodiments each strand of an siRNA is generated by transcription from a promoter, either in vitro or in vivo. For instance, a construct may contain two separate transcribable regions, each of which generates a 21 nt transcript containing a 19 nt region complementary with the other. Alternatively, a single construct may be utilized that contains opposing promoters P1 and P2 and terminators t1 and t2 positioned so that two different transcripts, each of which is at least partly complementary to the other, are generated (FIG. 7). In another embodiment, an shRNA is generated as a single transcript, e.g., by transcription of a single transcription unit encoding self-complementary regions. FIG. 8 depicts one such embodiment. As indicated, a template is employed that includes first and second complementary regions, and optionally includes a loop region. The present invention encompasses constructs encoding one or more siRNA and/or shRNA strands. In addition to a promoter, the construct optionally includes one or more other regulatory elements, e.g., terminator.

In vitro transcription may be performed using a variety of available systems including the T7, SP6, and T3 promoter/polymerase systems (e.g., those available from Promega, Clontech, New England Biolabs, etc.). When siRNAs are synthesized in vitro they may be allowed to hybridize before transfection or delivery to a subject. It will be appreciated that inventive siRNA compositions need not consist entirely of double-stranded (hybridized) molecules. For example, siRNA compositions may include a small proportion of single-stranded RNA. Generally, preferred compositions comprise at least approximately 80% dsRNA, at least approximately 90% dsRNA, at least approximately 95% dsRNA, or even at least approximately 99-100% dsRNA. However, the siRNA compositions may contain less than 80% hybridized RNA provided that they contain sufficient dsRNA to be effective.

Those of ordinary skill in the art will appreciate that if inventive siRNA or shRNA agents are generated in vivo, it is generally preferable that they be produced via transcription of one or more transcription units. The primary transcript may optionally be processed (e.g., by one or more cellular enzymes) in order to generate the final agent that accomplishes gene inhibition. It will further be appreciated that appropriate promoter and/or regulatory elements can readily be selected to allow expression of the relevant transcription units in mammalian cells. In some embodiments of the invention, it may be desirable to utilize a regulatable promoter; in other embodiments, constitutive expression may be desired. The term “expression” as used herein in reference to synthesis (transcription) of siRNA or siRNA precursors does not imply translation of the transcribed RNA.

In certain preferred embodiments of the invention, the promoter utilized to direct in vivo expression of one or more siRNA or shRNA transcription units is a promoter for RNA polymerase III (Pol III). Pol III directs synthesis of small transcripts that terminate upon encountering a stretch of 4-5 T residues in the template. Certain Pol III promoters such as the U6 or H1 promoters do not require cis-acting regulatory elements (other than the first transcribed nt) within the transcribed region and thus are preferred according to certain embodiments of the invention since they readily permit the selection of desired siRNA sequences. See, e.g., Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002). Promoters for Pol II may also be used as described, for example, in Xia, H., et al., Nat. Biotechnol., 20, pp. 1006-1010, 2002. As described therein, constructs in which a hairpin sequence is juxtaposed within close proximity to a transcription start site and followed by a polyA cassette, resulting in minimal to no overhangs in the transcribed hairpin, may be employed. In certain embodiments of the invention tissue-specific, cell-specific, or inducible Pol II promoters may be used, provided the foregoing requirements are met. Pol I promoters may also be used in various embodiments (McCown 2003).

It will be appreciated that in vivo expression of constructs that provide templates for synthesis of siRNA or shRNA, such as those depicted in FIGS. 7 and 8 can desirably be accomplished by introducing the constructs into a vector, such as, for example, a DNA plasmid or viral vector, and introducing the vector into mammalian cells. Any of a variety of vectors may be selected, though in certain embodiments it may be desirable to select a vector that can deliver the construct(s) to one or more cells that are susceptible to influenza virus infection. The present invention encompasses vectors containing siRNA and/or shRNA transcription units, as well as cells containing such vectors or otherwise engineered to contain transcription units encoding one or more siRNA or shRNA strands. In certain preferred embodiments of the invention, inventive vectors are gene therapy vectors appropriate for the delivery of an siRNA or shRNA expressing construct to mammalian cells, most preferably human cells. Such vectors may be administered to a subject before or after exposure to an influenza virus, to provide prophylaxis or treatment for diseases and conditions caused by infection with the virus.

The invention therefore provides a variety of viral and nonviral vectors whose presence within a cell results in transcription of one or more RNAs that self-hybridize or hybridize to each other to form an RNAi agent that inhibits expression of at least one influenza virus transcript in the cell. In certain embodiments of the invention two separate, complementary siRNA strands are transcribed using a single vector containing two promoters, each of which directs transcription of a single siRNA strand, i.e., is operably linked to a template for the siRNA so that transcription occurs. The two promoters may be in the same orientation, in which case each is operably linked to a template for one of the siRNA strands. Alternately, the promoters may be in opposite orientation flanking a single template so that transcription from the promoters results in synthesis of two complementary RNA strands.

In other embodiments of the invention a vector containing a promoter that drives transcription of a single RNA molecule comprising two complementary regions (e.g., an shRNA) is employed. In certain embodiments of the invention a vector containing multiple promoters, each of which drives transcription of a single RNA molecule comprising two complementary regions is used. Alternately, multiple different shRNAs may be transcribed, either from a single promoter or from multiple promoters. A variety of configurations are possible. For example, a single promoter may direct synthesis of a single RNA transcript containing multiple self-complementary regions, each of which may hybridize to generate a plurality of stem-loop structures. These structures may be cleaved in vivo, e.g., by Dicer, to generate multiple different shRNAs. It will be appreciated that such transcripts preferably contain a termination signal at the 3′ end of the transcript but not between the individual shRNA units. In another embodiment of the invention, the vector includes multiple promoters, each of which directs synthesis of a self-complementary RNA molecule that hybridizes to form an shRNA. The multiple shRNAs may all target the same transcript, or they may target different transcripts. Any combination of viral transcripts may be targeted. See Example 11 and FIG. 21.

Those of ordinary skill in the art will further appreciate that in vivo expression of RNAi-inducing agents according to the present invention may allow the production of cells that produce the agent over long periods of time (e.g., greater than a few days, preferably at least several weeks to months, more preferably at least a year or longer, possibly a lifetime). Such cells may be protected from influenza virus indefinitely.

Preferred viral vectors for use in the compositions to provide intracellular expression of RNAi-inducing agents include, for example, retroviral vectors, lentiviral vectors, adenoviral vectors, adeno-associated virus vectors, herpes virus vectors, etc. For example, see Kobinger, G. P., et al., Nat Biotechnol 19(3):225-30, 2001, describing a vector based on a Filovirus envelope protein-pseudotyped HIV vector, which efficiently transduces intact airway epithelium from the apical surface. See also Lois, C., et al., Science, 295: 868-872, Feb. 1, 2002, describing the FUGW lentiviral vector; Somia, N., et al. J. Virol. 74(9): 4420-4424, 2000; Miyoshi, H., et al., Science 283: 682-686, 1999; and U.S. Pat. No. 6,013,516.

It will be appreciated by those of ordinary skill in the art that agents such as the nucleic acids described herein, including but not limited to, nucleic acids having any of the structures depicted in FIG. 5, or any other effective structure described herein, may be comprised entirely of nucleotides such as those found in naturally occurring nucleic acids, or may instead include one or more analogs of such nucleotides or may otherwise differ from a naturally occurring nucleic acid. Nucleic acids containing modified backbones or non-naturally occurring internucleoside linkages can be used in the present invention.

Modified nucleic acids need not be uniformly modified along the entire length of the molecule. For example, different nucleotide modifications and/or backbone structures may exist at various positions in the nucleic acid. In certain embodiments of the invention it may be desirable to stabilize the siRNA structure, e.g., by including nucleotide analogs at one or more free strand ends in order to reduce digestion, e.g., by exonucleases. Including deoxynucleotides, e.g., pyrimidines such as deoxythymidines at one or more free ends may serve this purpose. Alternatively or additionally, it may be desirable to include one or more nucleotide analogs in order to increase or reduce stability of the 19 by stem, in particular as compared with any hybrid that will be formed by interaction of one strand of an RNAi agent with a target transcript. One of ordinary skill in the art will appreciate that the nucleotide analogs may be located at any position(s) where the target-specific activity, e.g., the RNAi mediating activity is not substantially affected, e.g., in a region at the 5′-end and/or the 3′-end of the RNA molecule. For example, in certain embodiments between 1-5 residues at the 5′ and/or 3′ end of an siRNA or shRNA strand is a nucleotide analog. In certain embodiments of the invention one or more of the nucleic acids in an inventive RNAi-inducing agent comprises at least 50% unmodified RNA, at least 80% modified RNA, at least 90% unmodified RNA, or 100% unmodified RNA. In certain embodiments of the invention one or more of the nucleic acids in an inventive RNAi-inducing agent comprises 100% unmodified RNA within the portion that participates in duplex formation in the RNAi-inducing agent.

According to certain embodiments of the invention various nucleotide modifications are used selectively in either the sense or antisense strand of an siRNA, shRNA, or microRNA precursor. For example, it may be preferable to utilize unmodified ribonucleotides in the antisense strand while employing modified ribonucleotides and/or modified or unmodified deoxyribonucleotides at some or all positions in the sense strand. According to certain embodiments of the invention only unmodified ribonucleotides are used in the duplex portion of the antisense and/or the sense strand while the overhang(s) of the antisense and/or sense strand may include modified ribonucleotides and/or deoxyribonucleotides. In certain embodiments of the invention one or both siRNA strands comprises one or more O-methylated ribonucleotides.

Numerous nucleotide analogs and nucleotide modifications are known in the art, and their effect on properties such as hybridization and nuclease resistance has been explored. A number of modifications have been shown to alter one or more aspects of the oligonucleotide such as its ability to hybridize to a complementary nucleic acid, its stability, bioavailability, nuclease resistance, etc. For example, 2′-modifications include halo, alkoxy and allyloxy groups. In some embodiments the 2′-OH group is replaced by a group selected from H, OR, R, halo, SH, SR₁, NH₂, NH_{R}, NR₂ or CN, wherein R is C₁-C₆ alkyl, alkenyl or alkynyl and halo is F, Cl, Br or I. Examples of modified linkages include phosphorothioate and 5′-N-phosphoramidite linkages. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.) “Antisense Drug Technology: Principles, Strategies, and Applications” (1^{st} ed), Marcel Dekker; ISBN: 0824705661; 1st edition (2001) and references therein. For purposes of the present invention, the chemical elements are identified in accordance with the Periodic Table of the Elements, CAS version, Handbook of Chemistry and Physics, 75th Ed., inside cover, and specific functional groups are generally defined as described therein. Analogs and modifications may be tested using, e.g., the assays described herein or other appropriate assays, in order to select those that effectively reduce expression of viral genes. In certain embodiments the RNAi-inducing agent comprises one or more modifications to a sugar, nucleoside, or internucleoside linkage such as any of those described in U.S. Pub. Nos. 20030175950, 20040192626, 20040092470, 20050020525, 20050032733 and/or references 137-139.

In certain embodiments of the invention the modification results in a nucleic acid with increased absorbability (e.g., increased absorbability across a mucus layer, increased oral absorption, etc.), increased stability in the blood stream or within cells, decreased clearance via the renal system, increased ability to cross cell membranes, increased ability to escape from an intracellular compartment such as an endosome, etc. As will be appreciated by one of ordinary skill in the art, analogs or modifications may result in altered Tm, which may result in increased tolerance of mismatches between the guide sequence and the target while still achieving effective suppression or may result in increased or decreased specificity for desired target transcripts.

It will further be appreciated by those of ordinary skill in the art that effective RNAi-inducing agents for use in accordance with the present invention may comprise one or more moieties that is/are not nucleotides or nucleotide analogs. In certain embodiments the nucleic acid comprises primarily nucleotide residues but comprises one or more residues that are not nucleotides. For example, in certain embodiments 1, 2, 3, 4, 5, or more of the residues in either strand of an effective silencing agent is not a nucleoside. In certain embodiments the portion of the RNAi-inducing agent that participates in duplex formation and/or is complementary to a target transcript consists of nucleosides while the overhang(s) consist of non-nucleoside residues. In certain embodiments of the invention sense and antisense strands of an RNAi-inducing agent are attached to one another by a non-nucleoside containing linker.

III. RNAi-Inducing Agents and Other Nucleic Acids Based on Identification of Preferred Influenza Virus Target Portions

The invention provides a variety of nucleic acids based on identification of potential target portions, functional target portions, favorably conserved target portions, and highly conserved target portions. In particular, the invention provides nucleic acids whose sequences comprise or consist of any of the potential target portions described above, including influenza sequences listed in Tables 1A, 1B, 17, 18, 20, and/or 34, or subsequences thereof that are at least 15 nucleotides in length (fragments). SiRNAs targeted to certain target portions showed unexpectedly high potency. To evaluate potency, siRNAs were administered to cells 6 hours prior to infection with influenza virus, and influenza virus production was tested 24 hours post-infection. In certain embodiments the sequence is selected from SEQ ID NOs: 272-380. SiRNAs targeted to these target portions (comprising an antisense strand 100% complementary to the target portion) showed a 4-fold reduction (75% decrease) in virus production in cells at 100 nM. In certain embodiments the sequence is selected from SEQ ID NOs: 274, 286, 287, 292, 297, 298, 304, 305, 309, 310, 311, 319, 324, 327, 334, 346, 347, 360, 361, 364, and 366. SiRNAs targeted to these target portions (comprising an antisense strand 100% complementary to the target portion) showed a 2-fold reduction (50% decrease) in virus production in cells at 5 nM. In certain embodiments the sequence is selected from SEQ ID NOs: 297, 309, 310, 311, 346, 347, 364, and 366. SiRNAs targeted to these target portions showed a 2-fold reduction (50% decrease) in virus production in cells at 5 nM, even when the target portion differed from the corresponding target portion in PR8 at up to two positions, i.e., there were up to two mismatches between the antisense siRNA strand and the target portion. Complements of these nucleic acids and fragments are also provided. In some embodiments the fragment is 16, 17, or 18 nt in length. The nucleic acids may be single stranded or double stranded and may be unmodified RNA or DNA, or modified versions thereof. The sequences may further include a 3′ overhang, e.g., a dTdT overhang. The invention also provides nucleic acids that are substantially identical to (e.g., at least 70%, at least 80% identical, at least 90% identical, 100% identical), 100% complementary to, or substantially complementary (e.g., at least 70%, at least 80% identical, at least 90% identical, 100% identical) to any of the sequences listed in Tables 1A, 1B, 17, 18, 20, and/or 34 or subsequences thereof that are at least 15 nt in length. The invention further provides vectors comprising one or more of the foregoing nucleic acids.

The nucleic acids include RNAi-inducing agents such as (i) siRNA; (ii) shRNA; (iii) single-stranded RNAs that hybridize with complementary single-stranded RNAs to form siRNAs; and (iv) vectors that comprise templates for transcription of any of the aforesaid nucleic acids. Where a sequence is presented as RNA, the corresponding DNA sequence is also provided by the invention (and vice versa). Where a sequence is presented herein, the invention encompasses a double-stranded nucleic acid comprising the sequence and its complement. Any of the nucleic acids of the invention may be limited in size. For example, the length of a nucleic acid may be 19 nt or less, 29 or 30 nt or less, 35 nt or less, 50 nt or less, or 100 nt or less.

The invention encompasses any nucleobase-containing structure in which residues, e.g., nucleotides, are linked together in an ordered manner, typically in a linear fashion, so that a nucleobase sequence can be assigned to the structure, wherein the sequence is any of the sequences disclosed herein. Various nucleobases and modified nucleotides and backbones are described above, any of which can be used. In various embodiments of the invention the structure is a nucleic acid, peptide nucleic acid (PNA), locked nucleic acid (LNA), or chimeric molecules, etc. See, e.g., WO92/20702, U.S. Pat. No. 6,316,230, and references therein. The invention also encompasses a structure comprising alternate nucleobases that have the same base pairing specificity or can otherwise substitute for a nucleobase present in the sequence.

The single-stranded nucleic acids may be used as antisense or sense strands of an RNAi-inducing agent such as an siRNA or shRNA (optionally with the addition of one or more nucleotides at the 3′ end to form an overhang). Nucleic acids of the invention may also be used, for example, as conventional antisense reagents, as probes (e.g., to detect influenza virus infection), etc. “Conventional antisense” refers to methods of inhibiting expression of a transcript by administering single-stranded oligonucleotides in vitro or to a subject. Such inhibition is believed to operate by mechanisms distinct from those of RNAi and does not require a double-stranded RNA molecule (other than the duplex formed between the antisense oligonucleotide and a target transcript). See, e.g., Crooke, S., infra.

The invention therefore provides a nucleic acid comprising a target portion of an influenza A virus transcript wherein the sequence of the nucleic acid comprises at least 15, 16, 17, 18, or 19 contiguous nt of a sequence listed in one or more of Tables 1A, 1B, 17, 18, 20, and 34. In certain embodiments the sequence consists of or is contained within a sequence listed in one or more of Tables 1A, 1B, 17, 18, 20, and 34. The invention further provides a nucleic acid comprising a target portion of an influenza A virus mRNA transcript wherein the sequence of the nucleic acid comprises at least 15, 16, 17, 18, or 19 contiguous nucleotides of any potential influenza virus target portion. The nucleotides at the 5′ and 3′ ends are considered to be contained within a sequence. In certain embodiments the length of the sequence is 30 nt or less, 35 nt or less, 50 nt or less, or 100 nt or less.

The invention further provides nucleic acids comprising a portion whose sequence is substantially identical (at least 70%, at least 80%, at least 90%, at least 95%, or at least 99% identical) to any potential influenza virus target portion, e.g., any of the target portions listed in Tables 1A, 1B, 17, 18, 20 and/or 34. Certain of these portions contain differences at 1, 2, 3, 4, or 5 positions with respect to a potential influenza virus target portion, e.g., a target portion listed in any of Tables 1A, 1B, 17, 18, 20, and/or 34. In certain embodiments of the invention the difference at 1, 2, 3, 4, or 5 of the positions is a replacement of C in a target portion by U in the substantially identical sequence, or a replacement of A in the target portion by G in the substantially identical sequence. The sequence of certain nucleic acids of the invention comprises a sequence that is found in a target transcript adjacent to the sequence of a potential influenza virus target portion, e.g., a target portion listed in Table 1A, 1B, 17, 18, 20, and/or 34, i.e., is located 5′ or 3′ of the target portion.

The invention provides RNAi-inducing agents having antisense strands that are complementary to a potential influenza virus target portion, e.g., a target portion listed in Table 1A, 1B, 17, 18, 20 and/or 34 over a window of at least 15 nt, at least 16, at least 17 nt, at least 18 nt, preferably over 19 nt. For example the antisense strand may be 100% complementary to the listed highly conserved target portion over 15, 16, 17, 18, or 19 nt or may be substantially complementary, e.g., having between 1 and 5 mismatches with respect to the target portion. In certain embodiments one or more, e.g., all, mismatches between the inhibitory region of an antisense strand and the target is a U-G mismatch. The sense strand of an RNAi-inducing agent may be 100% complementary to or substantially complementary to the antisense strand. The invention also provides RNAi-inducing agents having sense strands with sequences that are 100% identical to or substantially identical to a highly and/or favorably conserved sequence listed in one or more of Tables 1A, 1B, 17, 18, 20, and/or 34 over 15, 16, 17, 18, or 19 nt.

For example, the invention provides an RNAi-inducing agent targeted to an influenza virus transcript, wherein the RNAi-inducing agent comprises: a nucleic acid portion whose sequence comprises a sequence selected from the group consisting of: SEQ ID NOs: 272-380, its complement, or a fragment of either having a length of at least 15 nucleotides. The RNAi-inducing agent preferably comprises a second nucleic acid portion that forms a duplex structure with the first nucleic acid portion. In certain embodiments the first and second nucleic acid portions are each 50 nt or less in length, e.g., 35 nt or less in length, e.g., 21-23 nt in length, etc. In certain embodiments the sequence is selected from the group consisting of: SEQ ID NOs: 274, 286, 287, 292, 297, 298, 304, 305, 309, 310, 311, 319, 324, 327, 334, 346, 347, 360, 361, 364, and 366, its complement, or a fragment of either having a length of at least 15 nucleotides. In certain embodiments the sequence is selected from the group consisting of: SEQ ID NOs: 297, 309, 310, 311, 346, 347, 364, and 366, its complement, or a fragment of either having a length of at least 15 nucleotides. In certain embodiments of the invention, the sequence of the antisense strand of an RNAi-inducing agent designed based on a potential influenza virus target portion, e.g., a target portion whose sequence is listed in Table 1A, 1B, 17, 18, 20, and/or 34 includes at least 10, at least 12, at least 15, at least 17, or at least 19 consecutive nt that are 100% complementary to the listed sequence. The sense strand of such RNAi-inducing agents may be 100% complementary to or substantially complementary to the antisense strand. In certain embodiments of the invention, the sequence of the sense strand of an RNAi-inducing agent designed based on a potential influenza virus target portion, e.g., a sequence presented in Table 1A, 1B, 17, 18, 20 and/or 34 includes least 10, at least 12, at least 15, at least 17, and/or at least 19 consecutive nt of a listed sequence.

In certain embodiments of the invention, the sequence of the antisense strand of an RNAi-inducing agent designed based on a potential influenza virus target portion, e.g., a target portion presented in Table 1A, 1B, 17, 20, and/or 34 includes at least 10, at least 12, at least 15, at least 17, and/or at least 19 consecutive nt that are 100% complementary to the listed sequence, except that 1 or 2 mismatches may exist. The sense strand of such RNAi-inducing agents may be 100% complementary to or substantially complementary to the antisense strand. In certain embodiments of the invention, the sequence of the sense strand of an RNAi-inducing agent designed based on a potential influenza virus target portion, e.g., a sequence listed in Table 1A, 1B, 17, 18, 20 and/or 34 includes at least 10 consecutive nucleotides, more preferably at least 12 consecutive nt, more preferably at least 15 consecutive nt, more preferably at least 17 consecutive nt, and yet more preferably 19 consecutive nt of a listed sequence except that 1 or 2 nt may differ from the listed sequence.

In those embodiments of the invention in which the antisense strand is substantially or 100% complementary to the sequence over less than 19 nt, the remaining portion of the antisense strand may be, and preferably is, substantially complementary to or 100% complementary to influenza sequences that lie outside of and adjacent to the listed target portion. Thus the invention encompasses RNAi-inducing agents with antisense strands whose sequences are complementary to influenza sequences that are “shifted” by 1 or more nt, e.g, up to 9 nt, from the sequences in Tables 1A, 1B, 17, 18, 20 and/or 34. Adjacent sequences are found in FIG. 32.

According to certain embodiments of the invention the RNAi-inducing agent is targeted to a region that is favorably and/or highly conserved among influenza variants that naturally infect organisms of at least 2, 3, 4, 5, or more different species. The species may include human, equine (horse), avian, swine and others. In certain preferred embodiments of the invention the species include humans.

The invention also provides vectors from which the inventive RNAi-inducing agents can be transcribed, cells containing the vectors, and methods of use for the treatment and/or prevention of influenza A virus infection.

The invention provides variants of a potential influenza virus target portion, e.g., variants of any of the nucleic acids listed in Tables 1A, 1B, 17, 18, 20, and/or 34, and nucleic acids comprising such variants (e.g., RNAi-inducing agents comprising such variants), wherein the sequence of a variant differs from the listed sequence at 1, 2, 3, 4, 5, or 6 positions. In certain preferred embodiments of the invention a variant of a listed sequence is identical or substantially identical to a portion of the sequence of a transcript from an influenza virus strain other than PR8, such as any of the influenza A virus strains listed in Tables 15A-15H and/or Tables 19A-19F. In addition, subsequences of the various sequences disclosed herein are also encompassed. Preferred subsequences are between 15-18 nt in length, e.g., 15, 16, 17, or 18 nt in length. Sequences related to the specific sequences listed herein by deletion and/or addition of nucleotide(s) are also encompassed. For example, nucleic acid sequences in which 1, 2, 3, 4, 5, or 6 nt are deleted from or added to any of the sequences listed herein are encompassed. In addition, sequences in which 1, 2, 3, 4, 5, or 6 nt are deleted from or added to a variant (as described above) of any of the sequences listed herein are encompassed. The nucleotides that are deleted or added may be located contiguously or noncontiguously with respect to the original sequence. They may be located internally or at one or both ends. The nucleotides that are added may be positioned anywhere within the sequence or may be appended at one or both ends.

Inventive nucleic acids, e.g., RNAi-inducing agents or vectors may be introduced into cells by any available method. For instance, nucleic acids or vectors encoding them can be introduced into cells via conventional transformation or transfection techniques. As used herein, the terms “transformation” and “transfection” refer to a variety of art-recognized techniques for introducing foreign nucleic acids (e.g., DNA or RNA) into a cell, including calcium phosphate or calcium chloride co-precipitation, DEAE-dextran-mediated transfection, lipofection, injection, or electroporation. Delivery agents such as those described below can be used.

The present invention encompasses any cell manipulated to contain an inventive nucleic acid, e.g., an RNAi-inducing agent such as an siRNA, shRNA, or vector that provides a template for synthesis of an inventive RNAi-inducing agent. The cell can be a mammalian cell, e.g., a human cell or a non-human mammalian cell, or a non-mammalian cell. Preferably the cell is one found in the nasal and/or respiratory passages or lungs of a mammalian subject and is susceptible to infection by an influenza virus. Most preferably the cell is a respiratory epithelial cell. Optionally, such cells also contain influenza virus RNA.

IV. Diagnostic Methods and Kits

The invention encompasses the recognition that RNAi-based therapy of infectious diseases, e.g., infections caused by a respiratory virus, can desirably incorporate a diagnostic step that determines whether a subject in need of treatment is infected with an infectious agent that is susceptible to inhibition by one or more RNAi-inducing entities. By “susceptible to inhibition” is meant that one or more biological activities of the infectious agent can be effectively inhibited by administration of the RNAi-inducing entity to a subject. Preferably replication, pathogenicity, spread, and/or production of the infectious agent is inhibited. For example, preferably replication, pathogenicity, spread, and/or production of the agent is inhibited by at least 25% when the RNAi-inducing entity is administered to a subject at a tolerated dose. Preferably the inhibition is sufficient to produce a therapeutically useful effect.

Influenza virus is used as an example to illustrate the diagnostic methods of the invention, which are tailored to allow the selection of an RNAi-inducing entity that is suitable for a subject suffering from an infection. The selected RNAi-inducing entity may, of course, also be administered for prophylaxis, e.g., to individuals who have come in contact with the infected individual, regardless of whether those individuals have developed symptoms of infection.

The invention therefore provides methods for diagnosing influenza virus infection and for determining whether a subject is infected with an influenza virus. In certain embodiments the method comprises determining whether a subject is infected with an influenza virus that is inhibited by one or more of the RNAi-inducing entities of the invention. For example, a sample (e.g., sputum, saliva, nasal washings, nasal swab, throat swab, bronchial washings, broncheal alveolar lavage (BAL) fluid, biopsy specimens, etc.) is obtained from a subject who may be suspected of having a viral infection, e.g., influenza. The sample can be subjected to one or more processing steps. Any such processed sample is considered to be obtained from the subject. The sample is analyzed to determine whether it contains an influenza virus-specific nucleic acid. An “influenza virus-specific nucleic acid” is any nucleic acid, or its complement, that originates from or is derived from an influenza virus and can serve as an indication of the presence of an influenza virus in a sample and, optionally, be used to identify the influenza strain and/or the sequence of an influenza gene. The nucleic acid may have been subjected to processing steps following its isolation. For example, it may be reverse transcribed, amplified, cleaved, etc. Preferably the sequence is at least 15 nt in length, e.g., 20-25 nt, 25-30 nt, or longer. In certain embodiments the sequence is distinct from sequences found in other viruses, so that its presence is specifically indicative of the presence of an influenza virus.

In certain embodiments the sequence of an influenza virus-specific nucleic acid present in the sample, or its complement, is compared with the sequence of the antisense or sense strand of an RNAi-inducing agent such as an siRNA or shRNA. The word “comparison” is used in a broad sense to refer to any method by which a sequence can be evaluated, e.g., which it can be determined whether the sequence is the same as or different to a reference sequence at one or more positions, or by which the extent of difference can be assessed.

Any of a wide variety of nucleic acid-based assays can be used. In certain embodiments the diagnostic assay utilizes a nucleic acid comprising a favorably and/or highly conserved target portion or its complement, or a fragment of the favorably and/or highly conserved portion or its complement. In certain embodiments the nucleic acid serves as an amplification primer or a hybridization probe, e.g., in an assay such as those described below.

In certain embodiments an influenza-specific nucleic acid in the sample is amplified. Any suitable amplification method can be used, including exponential amplification, linked linear amplification, ligation-based amplification, and transcription-based amplification. An example of an exponential nucleic acid amplification method is the polymerase chain reaction (PCR) which is described, for example, in Mullis et al. Cold Spring Harbor Symp. Quant. Biol. 51:263-273 (1986); PCR Cloning Protocols: From Molecular Cloning to Genetic Engineering, Methods in Molecular Biology, White, B. A., ed., vol. 67 (1998); Mullis EP 201,184; Mullis et al., U.S. Pat. Nos. 4,582,788 and 4,683,195; Erlich et al., EP 50,424, EP 84,796, EP 258,017, EP 237,362; and Saiki R. et al., U.S. Pat. No. 4,683,194. Linked linear amplification is disclosed by Wallace et al. in U.S. Pat. No. 6,027,923. Examples of ligation-based amplification are the ligation amplification reaction (LAR), taught by Wu et al. (Genomics 4:560 (1989)) and the ligase chain reaction (EP Application No. 0320308 B1). Hampson et al. (Nucl. Acids Res. 24(23):4832-4835, 1996) describe a directional random oligonucleotide primed (DROP) method.

Isothermal target amplification methods include transcription mediated amplification (TMA), self-sustained sequence replication (3SR), Nucleic Acid Sequence Based Amplification (NASBA), and variations thereof. (See Guatelli et al. Proc. NatL Acad. Sci. U.S.A. 87:1874-1878 (1990); U.S. Pat. No. 5,766,849 (TMA); and U.S. Pat. No. 5,654,142 (NASBA)) and others (e.g., as described in Malek et al., U.S. Pat. No. 5,130,238; Kacian and Fultz, U.S. Pat. No. 5,399,491; Burg et al., U.S. Pat. No. 5,437,990).

Detection or comparison can be performed using any of a variety of methods known in the art, e.g., amplification-based assays, hybridization assays, primer extension assays (e.g., allele-specific primer extension in which the corresponding target portions of different influenza virus strains are analogous to different alleles of a gene), oligonucleotide ligation assays (U.S. Pat. Nos. 5,185,243, 5,679,524 and 5,573,907), cleavage assays, heteroduplex tracking analysis (HTA) assays, etc. Examples include the Taqman® assay, Applied Biosystems (U.S. Pat. No. 5,723,591). In this assay, two PCR primers flank a central probe oligonucleotide. The probe oligonucleotide contains two fluorescent moieties. During the polymerization step of the PCR process, the polymerase cleaves the probe oligonucleotide. The cleavage causes the two fluorescent moieties to become physically separated, which causes a change in the wavelength of the fluorescent emission. As more PCR product is created, the intensity of the novel wavelength increases. Cycling probe technology (CPT), which is a nucleic acid detection system based on signal or probe amplification rather than target amplification (U.S. Pat. Nos. 5,011,769, 5,403,711, 5,660,988, and 4,876,187), could also be employed. Invasive cleavage assays, e.g., Invader® assays (Third Wave Technologies), described in Eis, P. S. et al., Nat. Biotechnol. 19:673, 2001, can also be used to detect influenza-specific nucleic acids. Assays based on molecular beacons (U.S. Pat. Nos. 6,277,607; 6,150,097; 6,037,130) or fluorescence energy transfer (FRET) may be used. Molecular beacons are oligonucleotide hairpins which undergo a conformational change upon binding to a perfectly matched template. The conformational change of the oligonucleotide increases the physical distance between a fluorophore moiety and a quencher moiety present on the oligonucleotide. This increase in physical distance causes the effect of the quencher to be diminished, thus increasing the signal derived from the fluorophore. U.S. Pub. No. 20050069908 and references therein describe a variety of other methods that can be used for the detection of nucleic acids. Probes of the invention may thus comprise one or more portions that hybridize to an influenza-specific sequence and one or more portions designed according to the specific assay. U.S. Pat. Nos. 5,854,033, 6,143,495, and 6,239,150 describe compositions and a method for amplification of and multiplex detection of molecules of interest involving rolling circle replication. The method is useful for simultaneously detecting multiple specific nucleic acids in a sample. For example, it may be used for determining the presence of one or more influenza-specific nucleic acids in the sample. Optionally the nucleic acids are sequenced. U.S. Pub. No. 20050026180 describes methods for multiplexing nucleic acid reactions, including amplification, detection and genotyping, which can be adapted for detection of influenza-specific sequences and for determining the sequence at specific locations of interest for purposes of determining susceptibility to an RNAi-inducing entity.

In certain embodiments the assay determines whether an influenza-specific nucleic acid in the sample comprises a portion that is identical to or different from a sense or antisense strand of an RNAi-inducing entity. Optionally the exact differences, if any, are identified. This information is used to determine whether the influenza virus is susceptible to inhibition by the RNAi-inducing entity. In addition to those discussed above, suitable assays for detection and/or genotyping of infectious agents are described in Molecular Microbiology: Diagnostic Principles and Practice, Persing, D. H., et al., (eds.) Washington, D.C.: ASM Press, 2004. Any of the assays can be performed using an automated system. A number of systems for performing nucleic acid-based diagnostic assays are known in the art and can readily be adapted for purposes of the present invention. In one embodiment nucleic acids from a sample are applied to a microarray (also referred to as a “chip”) to which a multiplicity of nucleic acids complementary to various different influenza virus transcripts or portions thereof are attached. The hybridization pattern is detected and provides sufficient information to determine whether the influenza virus is susceptible to inhibition by an RNAi-inducing entity. In certain embodiments an influenza-specific nucleic acid present in the sample is sequenced (typically following amplification). Multiple different assays can be used.

The diagnostic assays may employ any of the nucleic acids described in section III. In certain embodiments of the invention the nucleic acid comprises a nucleic acid portion that is not substantially complementary or substantially identical to an influenza virus transcript. For example, the nucleic acid may comprise a primer binding site (e.g., a binding site for a universal sequencing primer or amplification primer), a hybridization tag (which may, for example, be used to isolate the nucleic acid from a sample comprising other nucleic acids), etc. In certain embodiments of the invention the nucleic acid comprises a non-nucleotide moiety. The non-nucleotide moiety may be attached to a terminal nucleotide of the nucleic acid, e.g., at the 3′ end. The moiety may protect the nucleic acid from degradation. In certain embodiments the non-nucleotide moiety is a detectable moiety such as a fluorescent dye, radioactive atom, member of a fluorescence energy transfer (FRET) pair, quencher, etc. In certain embodiments the non-nucleotide moiety is a binding moiety, e.g. biotin or avidin. In certain embodiments the non-nucleotide moiety is a hapten such as digoxygenin, 2,4-Dinitrophenyl (TEG), etc. In certain embodiments the non-nucleotide moiety is a tag usable for isolation of the nucleic acid.

In certain embodiments of the invention a nucleic acid is attached to a support, e.g., a microparticle such as a bead, which is optionally magnetic. The invention further provides an array comprising a multiplicity of nucleic acids of the invention, e.g., at least 10, 20, 50, etc. The nucleic acids are covalently or noncovalently attached to a support, e.g., a substantially planar support such as a glass slide. See, e.g., U.S. Pat. Nos. 5,744,305; 5,800,992; 6,646,243.

Information regarding whether an influenza virus of a particular strain, or having a particular sequence within a target portion, is susceptible to inhibition by a particular RNAi-inducing entity, e.g., siRNA or shRNA having an antisense strand with a particular sequence, is referred to as “susceptibility information”. Susceptibility information can include quantitative information regarding the degree of susceptibility. For purposes of the present invention, an influenza virus is considered susceptible to inhibition by an RNAi-inducing entity such as an siRNA or shRNA if the RNAi-inducing entity reduces virus production in infected cells by at least 25% when contacted with the cells or administered to a subject at a tolerated dose.

In a preferred embodiment, if an influenza virus transcript comprises a target portion that is 100% identical to any of SEQ ID NOs: 272-380, preferably 100% identical to any of SEQ ID NOs: 274, 286, 287, 292, 297, 298, 304, 305, 309, 310, 311, 319, 324, 327, 334, 346, 347, 360, 361, 364, and 366, yet more preferably 100% identical to any of SEQ ID NOs: 297, 309, 310, 311, 346, 347, 364, and 366, the influenza virus is considered susceptible to an RNAi-inducing entity that comprises an antisense strand that is 100% complementary to the target portion. In other embodiments, if an influenza virus transcript comprises a target portion that differs at 1, 2, or 3 positions, preferably 1 or 2 positions, more preferably only 1 position from any of SEQ ID NOs: 272-380, the influenza virus is considered susceptible to an RNAi-inducing entity that comprises an antisense strand that is 100% complementary to the target portion. In other embodiments, if an influenza virus transcript comprises a target portion that differs at 1, 2, or 3 positions, preferably 1 or 2 positions, more preferably only 1 position from any of SEQ ID NOs: 274, 286, 287, 292, 297, 298, 304, 305, 309, 310, 311, 319, 324, 327, 334, 346, 347, 360, 361, 364, and 366, the influenza virus is considered susceptible to an RNAi-inducing entity that comprises an antisense strand that is 100% complementary to the target portion. In other embodiments, if an influenza virus transcript comprises a target portion that differs at 1, 2, or 3 positions, preferably 1 or 2 positions, more preferably only 1 position from any of SEQ ID NOs: 297, 309, 310, 311, 346, 347, 364, and 366, the influenza virus is considered susceptible to an RNAi-inducing entity that comprises an antisense strand that is 100% complementary to the target portion.

Information obtained from experiments or from previous experience in treating an influenza virus having a particular sequence within the target portion can also be used to decide whether the virus is susceptible to inhibition by a given RNAi-inducing entity or combination thereof. Susceptibility information can also include theoretical predictions based, for example, on the expected effect of any mismatches that exist between the influenza virus sequence and the antisense strand of an inhibitory agent.

Susceptibility information can be stored in a computer-readable form on a computer-readable medium, e.g., in an organized manner in a database. The results of a diagnostic test performed on a sample obtained from a subject are provided to a computerized system that accesses the information and determines the susceptibility profile of an influenza virus that infects the subject. In certain embodiments the system recommends a particular RNAi-inducing agent or combination thereof and/or a dose. The invention therefore provides a computerized system for determining susceptibility of a virus, e.g., an influenza virus, to an RNAi-inducing entity. The invention further provides a database containing susceptibility information. The computerized system and an automated system for performing the assay may be part of a single integrated automatic system or may be provided separately.

The invention provides diagnostic kits for detecting influenza virus infection. Certain of the kits comprise one or more nucleic acids of the invention. Certain of the kits comprise one or more nucleic acids that can be used to detect a portion of an influenza virus transcript that comprises a preferred target portion for RNAi. The kits may comprise one or more items selected from the group consisting of: a probe, a primer, a sequence-specific oligonucleotide, an enzyme, a substrate, an antibody, a population of nucleotides, a buffer, a positive control, and a negative control. The nucleotides may be labeled. For example, one or more populations of fluorescently labeled nucleotides such as dNTPs, ddNTPs, etc. may be provided.

The probe can be a nucleic acid that includes all or part of a target portion, e.g., a highly or favorably conserved target portion, or its complement, or is at least 80% identical or complementary to a target portion, e.g., 100% identical or complementary. In certain embodiments a plurality of probes are provided. The probes differ at one or more positions and can be used for determining the exact sequence of an influenza virus transcript at such positions. For example, the probes may differentially hybridize to the transcript (e.g., hybridization occurs only if the probe is 100% complementary to a target portion of the transcript).

The primers can be complementary to sites located upstream and downstream of a target portion and can be used to amplify a region of influenza virus nucleic acid comprising the target portion, which can then be sequenced or subjected to additional processes. The length of the amplified region may be, e.g., 100-200 nt, 200-300 nt, or more. Primers that bind to sites a sufficient distance away from the target portion to amplify a region of a desired length are selected. Methods for selecting amplification primers are well known in the art. The kits can comprise sequence-specific oligonucleotides. The oligonucleotides are sequence-specific in that they will only support polymerase-mediated extension or ligation when hybridized to a substantially complementary nucleic acid (e.g., an influenza virus-specific nucleic acid) if the 3′ terminal nucleotide of the oligonucleotide is perfectly complementary to the nucleic acid. Preferably a plurality of sequence-specific oligonucleotides are provided. The oligonucleotides differ at the 3′ terminal position and can therefore be used to establish the identity of a nucleotide that is located opposite that position when the oligonucleotide is hybridized to a nucleic acid of interest (e.g., an influenza virus-specific nucleic acid).

Kits of the invention can comprise specimen collection materials, e.g., a swab, a tube, etc. The components of the kit may be packaged in individual vessels or tubes which will generally be provided in a container, e.g., a plastic or styrofoam container suitable for commercial sale, together with instructions for use of the kit.

V. Transgenic Animals

The present invention encompasses transgenic animals engineered to contain or express an inventive RNAi-inducing agent. Such animals are useful for studying the function and/or activity of inventive RNAi agents, and/or for studying the influenza virus infection/replication system. As used herein, a “transgenic animal” is a non-human animal in which one or more of the cells of the animal, preferably most or all of the cells, includes a transgene. A transgene is exogenous DNA or a rearrangement, e.g., a deletion of endogenous chromosomal DNA, which preferably is integrated into or occurs in the genome of the cells of a transgenic animal. Preferably the transgene comprises a promoter operably linked to a nucleic acid such that expression of the nucleic acid occurs in the cell.

A transgene can direct the expression of an RNAi-inducing agent in one or more cell types or tissues of the transgenic animal. Certain preferred transgenic animals are non-human mammals, e.g., rodents such as rats or mice. Other examples of transgenic animals include non-human primates, sheep, dogs, cows, goats, birds such as chickens, amphibians, and the like. According to certain embodiments of the invention the transgenic animal is of a variety used as an animal model (e.g., murine, ferret, or primate) for testing potential influenza therapeutics. Other non-human animals contemplated within the invention include domesticated animals, including but not limited to livestock and pets, or any animal used or kept for profit. Such animals are partly or fully resistant to influenza virus infection. The RNAi-inducing agent may be, for example, an siRNA or shRNA. The RNAi-inducing agent can be targeted to any potential influenza virus target portion, e.g., a target portion listed in any of Tables 1A, 1B, 17, 18, 20, and/or 34. In certain embodiments the RNAi-inducing agent is targeted to a target portion whose sequence is selected from SEQ ID NOs: 274, 286, 287, 292, 297, 298, 304, 305, 309, 310, 311, 319, 324, 327, 334, 346, 347, 360, 361, 364, and 366, e.g., any of SEQ ID NOs: 297, 309, 310, 311, 346, 347, 364, and 366. For example, in preferred embodiments the RNAi-inducing agent has an antisense strand that is complementary to any of the foregoing target portions and a sense strand that forms a duplex with the antisense strand.

Methods for making transgenic non-human animals are known in the art. Briefly, these methods include (i) introducing an appropriate vector comprising the transgene into nuclei of fertilized eggs by microinjection, followed by transfer of the egg into the genital tract of a pseudopregnant female; and (ii) introducing an appropriate vector comprising a transgene into a cultured somatic cell (e.g., using any convenient technique such as transection, electroporation, etc.), selecting cells in which the transgene has integrated into genomic DNA, transferring the nucleus from a selected cell into an oocyte or zygote, optionally culturing the oocyte or zygote in vitro to the morula or blastula stage, and transferring the embryo into a recipient female. According to other methods, a retroviral vector comprising the transgene is used. The retroviral vector is introduced into cells either as DNA plasmid or as a viral particle, by infection. Cytoplasmic microinjection of an appropriate vector into an oocyte or embryonic cell can also be used. Sperm-mediated transgenesis is also encompassed. Heterozygous or chimeric animals obtained using these methods are identified and bred to produce homozygotes.

The vector is preferably an RNAi-inducing vector targeted to an influenza virus transcript. In a preferred embodiment the vector comprises a template for transcription of an RNAi-inducing agent such as an siRNA or shRNA targeted to a target portion that is favorably and/or highly conserved among influenza viruses that are derived from organisms of the species of the transgenic animal and, optionally also among influenza viruses derived from another species such as humans. The vector comprises a promoter operably linked to a template for transcription of the RNAi-inducing agent. The RNAi-inducing agent may be produced as a single RNA molecule comprising complementary portions or as two RNA molecules that hybridize within the cell, as described above. The promoter may, but need not be, derived from the species of the transgenic animal. RNA Pol I, II, or III promoters can be used. The promoter can be constitutive or inducible.

In a preferred embodiment the transgenic animals are avians, e.g., chickens. Methods for making transgenic avians are known in the art and include those described above and variations thereof. Vectors and methods suitable for production of transgenic avians and other transgenic animals are described, for example, in U.S. Pat. No. 6,730,822, U.S. Pub. Nos. 20020108132 and 20030126629, and references in these. In certain embodiments the transgenic avian is produced using a retroviral vector, e.g., an avian leukosis virus vector. In other embodiments the transgenic avian is produced using a eukaryotic vector other than a retroviral vector, although the vector may comprise one or more sequences derived from a retrovirus. In certain embodiments the transgenic avian expresses a plurality of RNAi-inducing agents each having an antisense strand with a different inhibitory region sequence. The RNAi-inducing agents may each be targeted to a different influenza virus strain.

The invention provides flocks of transgenic avians in which different members of the flock express one or more different RNAi-inducing agents each having an antisense strand with a different inhibitory region sequence. For example, a first fraction of the flock expresses a first RNAi-inducing agent comprising an antisense strand that is 100% complementary to a target portion of a first avian influenza strain, a second fraction of the flock expresses a first RNAi-inducing agent comprising an antisense strand that is 100% complementary to a target portion of a second avian influenza strain, and a third fraction of the flock expresses a third RNAi-inducing agent comprising an antisense strand that is 100% complementary to a target portion of a third avian influenza strain, etc. Flocks whose members express different RNAi-inducing agents may be less susceptible to the emergence of a resistant influenza virus strain than would be the case if all members express identical RNAi-inducing agents.

In another preferred embodiment the transgenic animals are mammals, e.g., pigs (swine), bovines, etc. Methods suitable for making transgenic mammals include those discussed above, aspects of which are further described in Gordon et al., Proc. Natl. Acad. Sci U.S.A., 77:7380-7384, 1980 (germ line transfer by micro injection of DNA into one-cell embryos), Hooper et al., Nature, 326:292-295, 1987; Kuehn et al., Nature, 326:295-298, 1987 (transfer of genetically engineered embryonic stem-cells into blastocysts), and Campbell et al., Nature, 380:64-66, 1996 (transfer of nuclei from engineered cells into enucleated oocytes). Additional references describe applications of transgenic technology to swine (U.S. Pat. No. 6,558,663; Machaty, Z, et al., Cloning Stem Cells, 4(1):21-7, 2002; Wall et al., Proc. Natl. Acad. Sci. U.S.A., 88:1696-1700, 1991), sheep (Wright et al., Biotechnology, 9:830-834, 1991), goats (Wang, B., et al., Mol Reprod Dev., 63(4):437-43, 2002), and bovines (Krimpenfort et al., Biotechnology, 9:844-847, 1991; Galli, et al., Theriogenology, 59(2):599-616, 2003).

In another preferred embodiment the transgenic animals are rodents, e.g., mice. Mice and rats that express RNAi-inducing agents have been produced using a variety of different approaches (see, e.g., Hasuwa, et al, FEBS Lett. 2002 Dec. 4; 532(1-2):227-30. Xia, et al., Nat. Biotechnol., 20(10):1006-10, 2002; Rubinson, et al, Nat Genet., 33(3):401-6, 2003).

VI. Compositions and Methods for Delivery of RNAi-Inducing Entities

RNAi-inducing entities may be administered according to a variety of approaches. In one embodiment of the invention, a single species of RNAi-inducing agent is administered to a subject. A nonlimiting example is a single siRNA species comprising an antisense strand complementary to a favorably and/or highly conserved target portion from a variety of influenza virus strains. In related embodiments, a population of two or more different RNAi-inducing agents are administered to a subject. In one embodiment, the population of two or more RNAi-inducing agents include agents that contain antisense strands whose sequences are substantially complementary (preferably 100% complementary) to the same favorably and/or highly conserved region from a variety of strains of a particular virus, e.g., an influenza virus. In another embodiment, the population of two or more RNAi-inducing agents includes agents that contain antisense strands whose sequences are substantially complementary (preferably 100% complementary) to different conserved regions from the same virus strain. In yet another embodiment, the population of two or more RNAi-inducing agents include agents that contain antisense strands whose sequences are substantially complementary (preferably 100% complementary) to the same favorably and/or highly conserved region from a variety of strains of a particular virus, e.g., an influenza virus and RNAi-inducing agents includes agents that contain antisense strands whose sequences are substantially complementary (preferably 100% complementary) to different highly conserved regions from the same virus strain.

The inventors have recognized that effective RNAi therapy in general, including prevention and therapy of influenza virus infection, will be enhanced by efficient delivery of RNAi-inducing agents and/or RNAi-inducing vectors into cells in intact organisms. In the case of influenza virus, such agents must be introduced into cells in the respiratory tract, where influenza infection normally occurs. For use in humans, it may be preferable to employ non-viral methods that facilitate intracellular uptake of RNAi-inducing agents. The invention therefore provides compositions comprising any of a variety of non-viral delivery agents for enhanced delivery of RNAi-inducing agents and/or vectors to cells in intact organisms, e.g., mammals and avians. As used herein, the concept of “delivery” includes transport of an RNAi-inducing agent or RNAi-inducing vector from its site of entry into the body to the location of the cells in which it is to function, cellular uptake, and/or any subsequent steps involved in making the agent or available to the intracellular RNAi machinery (e.g., release of siRNA or shRNA from endosomes). Components that stabilize the RNAi-inducing agent either once it is in the body or during the process of formulating the agent for delivery, inhibit its degradation (e.g., RNase inhibiting agents such as RNasin), can also be included in the inventive compositions. In general, any agent that inhibits the activity of an RNase either fully or partially can be used. Examples include RNase inhibitors purified from human placenta or recombinant versions thereof. While the delivery agents are primarily of use for enhancing delivery of RNAi-inducing agents, they may also be used to enhance delivery of RNAi-inducing vectors.

In certain embodiments of the invention the delivery agent enhances stability, inhibits clearance, promotes cellular uptake of the composition, promotes release of the RNAi-inducing entity within the cell, reduces cytotoxicity, or directs the composition to a particular cell type, tissue, or organ. To “inhibit clearance” means to reduce the rate of removal of the composition from the body by the renal system. The delivery agent may inhibit uptake by cells of the reticulo-endothelial system such as macrophages. The RNAi-inducing entity itself may be modified (e.g., covalently modified) to enhance stability, inhibit clearance, promote cellular uptake, promote release of the RNAi agent and/or vector from an intracellular compartment such as an endosome, reduce cytotoxicity, or direct the composition to a particular cell type, tissue, or organ. For example, an RNAi-inducing agent may be pegylated, and/or an arginine-rich peptide may be conjugated to the RNAi-inducing agent.

The invention therefore encompasses compositions comprising (i) an RNAi-inducing agent targeted to a transcript, and/or an RNAi-inducing vector whose presence within a cell results in production of an RNAi-inducing agent targeted to a transcript; and (ii) any of a variety of delivery agents including, but not limited to, cationic polymers, modified cationic polymers, peptide molecular transporters (including arginine or histidine-rich peptides), carbohydrates, lipids (including cationic lipids, neutral lipids, and combinations thereof), liposomes, lipopolyplexes, non-cationic polymers, surfactants suitable for introduction into the lung, or mixtures of any of the foregoing, etc. Certain of the delivery agents incorporate a moiety that increases delivery or increases the selective delivery of the RNAi-inducing agent or vector to cells in which it is desired to inhibit the transcript. In certain embodiments the transcript is a respiratory virus transcript, e.g., an influenza virus transcript.

While use of specific delivery agents is preferred in certain embodiments of the invention, in other preferred embodiments an RNAi-inducing entity such as an RNAi-inducing agent is administered in “naked” form, i.e., in the absence of any delivery agent that enhances transfection, cellular entry, etc. For example, an RNAi-inducing agent can be administered in an aqueous medium that is essentially free of lipids and is essentially free of delivery-enhancing polymers, e.g., cationic or noncationic polymers such as those described below. RNAi-inducing agents can be administered in naked form intravenously or directly to the respiratory system (e.g., by inhalation through the nose or mouth and into the lungs). In certain embodiments the RNAi-inducing agent is administered in an amount effective to treat or prevent a respiratory virus infection while resulting in minimal absorption into the blood and thus minimal systemic delivery of the RNAi-inducing agent.

A. Delivery Methods

The invention provides a variety of methods for delivering a composition comprising an RNAi-inducing entity to a mammalian subject. In certain embodiments the composition is delivered directly to the vascular system and achieves inhibition of a target transcript in an organ or tissue of the subject, e.g., the lung. In other embodiments the composition is delivered directly to the respiratory system. Certain of the methods are employed in Examples 16, 22, 23, and 24, in which influenza virus production and luciferase or cyclophilin B expression are inhibited in a target organ of a mammalian subject using the methods. These results indicate that the methods are widely applicable to the inhibition of virtually any desired target transcript.

In particular, the invention provides a method of inhibiting expression of a gene in a tissue or organ of a mammalian subject comprising the step of: introducing a composition comprising an effective amount of an RNAi-inducing agent targeted to the gene directly into the vascular system of the subject without using a hydrodynamic transfection technique. Preferably the RNAi-inducing agent inhibits expression of a target transcript in the lung. The tissue may be a non-circulating tissue, i.e., a tissue other than blood. In a related embodiment, the invention further provides a method of method of inhibiting production of a virus in the respiratory system of a mammalian subject, wherein the virus infects respiratory epithelial cells, the method comprising the step of: introducing a composition comprising an effective amount of an RNAi-inducing agent targeted to a gene of the virus into the vascular system of the subject by injection without using a hydrodynamic transfection technique.

The invention further provides a method of inhibiting expression of a gene in the lung of a mammalian subject comprising the step of: introducing a composition comprising an effective amount of an RNAi-inducing agent targeted to the gene and a delivery agent directly into the respiratory system of the subject. In a preferred embodiment the gene is a respiratory virus gene, e.g., an influenza virus gene. Preferably the effective amount inhibits production of influenza virus in the respiratory system of the subject. In certain embodiments of the method or any other aspect of the invention, the virus is a respiratory virus other than RSV.

The composition may, for example, be administered via the nose or mouth, typically followed by inhalation. The composition may comprise particles that remain primarily in the upper respiratory tract, e.g., nose, pharynx, etc., as in a typical nasal or oral spray. In other embodiments the particles are inhaled into the lower respiratory tract. Respirable formulations that may be used to directly deliver a composition to the respiratory system are discussed below. In certain embodiments delivery directly to the respiratory system results in systemic delivery, e.g.,, the RNAi-inducing agent enters the vascular system from the lung and is transported to a target organ or tissue elsewhere in the body.

Methods for delivering an effective amount of an RNAi-inducing agent to the respiratory system of a mammalian subject have a wide variety of uses including, but not limited to, preventing or treating respiratory virus infection. RNAi-inducing agents targeted to appropriate transcripts can also be administered using the inventive methods for prevention and/or treatment of a variety of other diseases and conditions that affect the respiratory system. Examples include cancer, e.g., lung cancer, cystic fibrosis (See, e.g., U.S. Ser. No. 10/200,607), asthma (See,. e.g., U.S. Ser. No. 11/069,611), pulmonary hypertension, pulmonary fibrosis, emphysema, etc. Suitable target genes include, for example, oncogenes, genes that encode pro-angiogenic molecules and/or growth factors such as vascular endothelial growth factor, pro-inflammatory molecules, etc. Of course transcripts that play a role in diseases affecting any part of the body can be targeted when the RNAi-inducing agent is delivered systemically.

In certain embodiments of the invention the effective amount is between 0.1 mg/kg and 5 mg/kg of the subject's body weight. In other embodiments the effective amount is between 0.1 mg/kg and 10 mg/kg of the subject's body weight, or between 0.5 mg/kg and 20 mg/kg of the subject's body weight.

Compositions comprising RNAi-inducing entities may or may not include a delivery agent. Delivery agents suitable for use in the present invention include those described below and in co-pending U.S. Ser. No. 10/674,087. The delivery agents may be used in combination.

B. Cationic Polymers and Modified Cationic Polymers

The inventors have determined that delivery of RNAi-inducing agents by a number of different routes is enhanced by any of a variety of cationic polymers and modified cationic polymers. The invention therefore provides compositions comprising (i) an RNAi-inducing entity targeted to a target transcript and (ii) a cationic polymer. The invention further provides methods of inhibiting target gene expression comprising administering a composition comprising an RNAi-inducing agent targeted to a target transcript to a mammalian subject. In particular, the invention provides methods of treating and/or preventing influenza virus infection comprising administering a composition comprising an RNAi-inducing agent that targets an influenza virus transcript and a cationic polymer to a mammalian subject.

In general, a cationic polymer is a polymer that is positively charged at approximately physiological pH, e.g., a pH ranging from approximately 7.0 to 7.6, preferably approximately 7.2 to 7.6, more preferably approximately 7.4. Such cationic polymers include, but are not limited to, polylysine (PLL), polyarginine (PLA), polyhistidine, polyethyleneimine (PEI) (37), including linear or branched PEI and low molecular weight PEI as described, for example, in (76), polyvinylpyrrolidone (PVP) (38), chitosan (39, 40), protamine, polyphosphates, polyphosphoesters (such as those described in US Publication No. 20020045263), poly(N-isopropylacrylamide), etc.

Certain of these polymers comprise primary amine groups, imine groups, guanidine groups, and/or imidazole groups. Preferred cationic polymers have relatively low toxicity. References 85-87; U.S. Pat. No. 6,013,240; WO9602655; and U.S. Pub. Nos. 20040167087 and 20030157030 provide further information on PEI and other polymers useful in the practice of the invention. The commercially available PEI reagent known as jetPEI™ (Qbiogene, Carlsbad, Calif.), a linear form of PEI (U.S. Pat. No. 6,013,240) can be used.

Suitable cationic polymers also include blends of polymers of different molecular weight, copolymers comprising subunits of any of the foregoing polymers (or others), e.g., lysine-histidine copolymers, etc. The percentage of the various subunits need not be equal in the copolymers but may be selected, e.g., to optimize such properties as ability to form complexes with nucleic acids while minimizing cytotoxicity. Furthermore, the subunits need not alternate in a regular fashion. Appropriate assays to evaluate various polymers with respect to desirable properties are described in the Examples. Preferred cationic polymers also include polymers such as the foregoing, further incorporating any of various modifications. Appropriate modifications are discussed below and include, but are not limited to, modification with acetyl, succinyl, acyl, or imidazole groups (32).

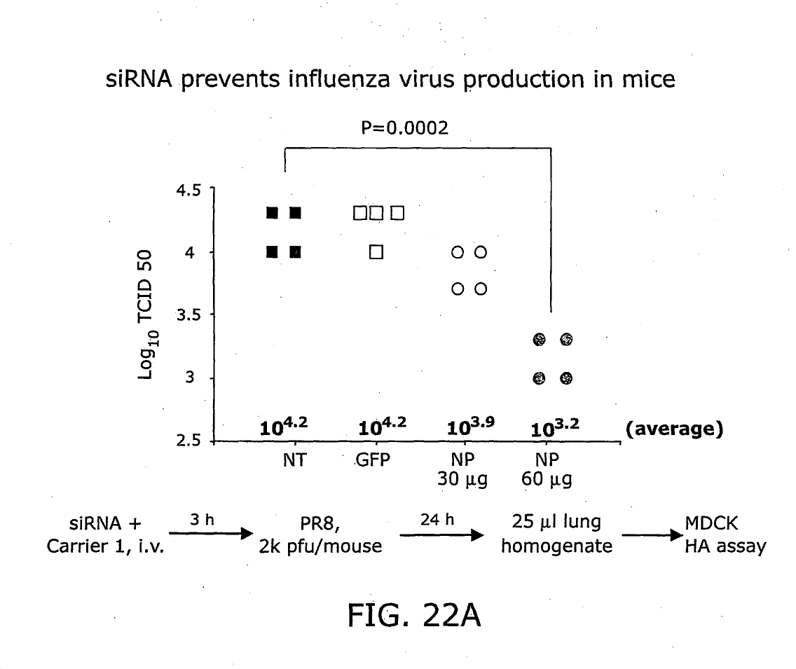

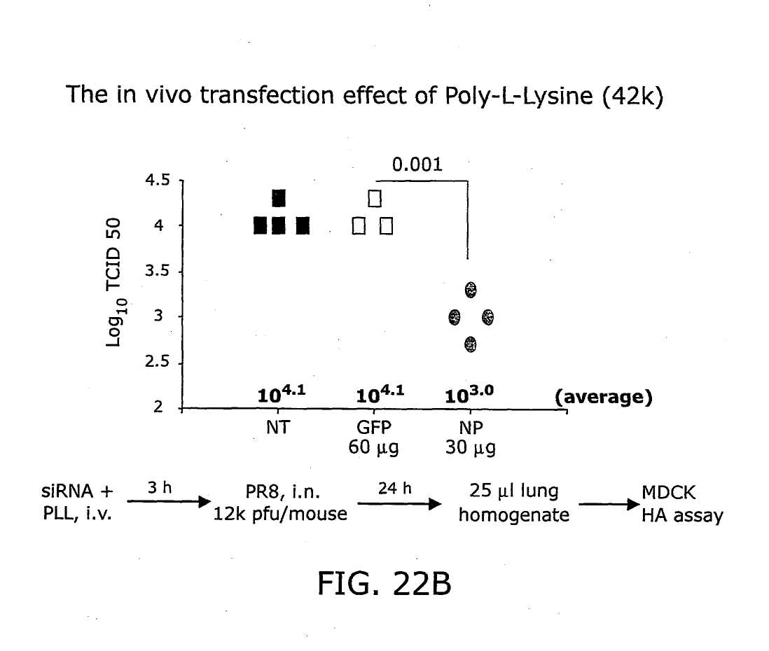

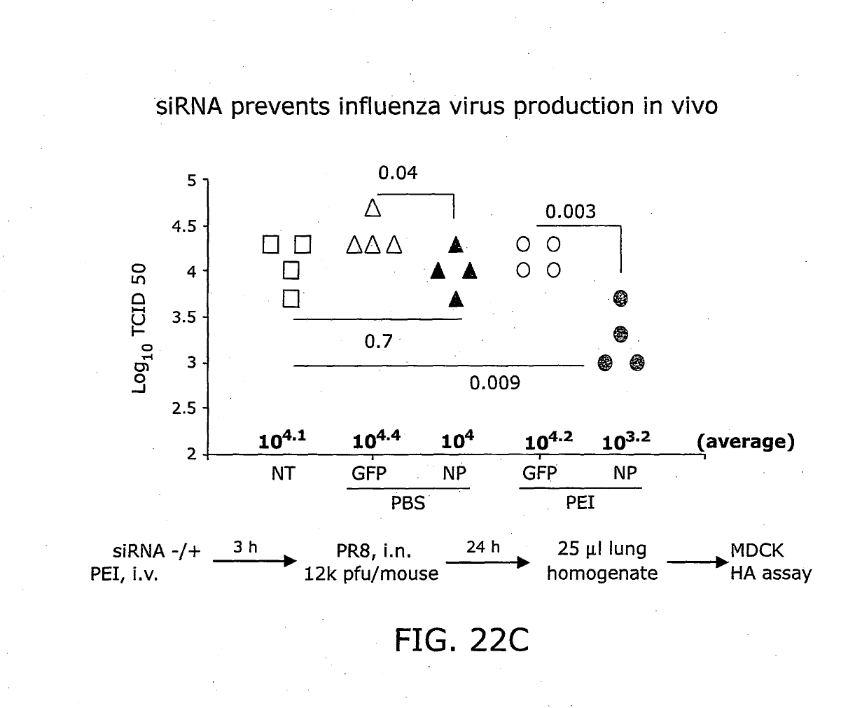

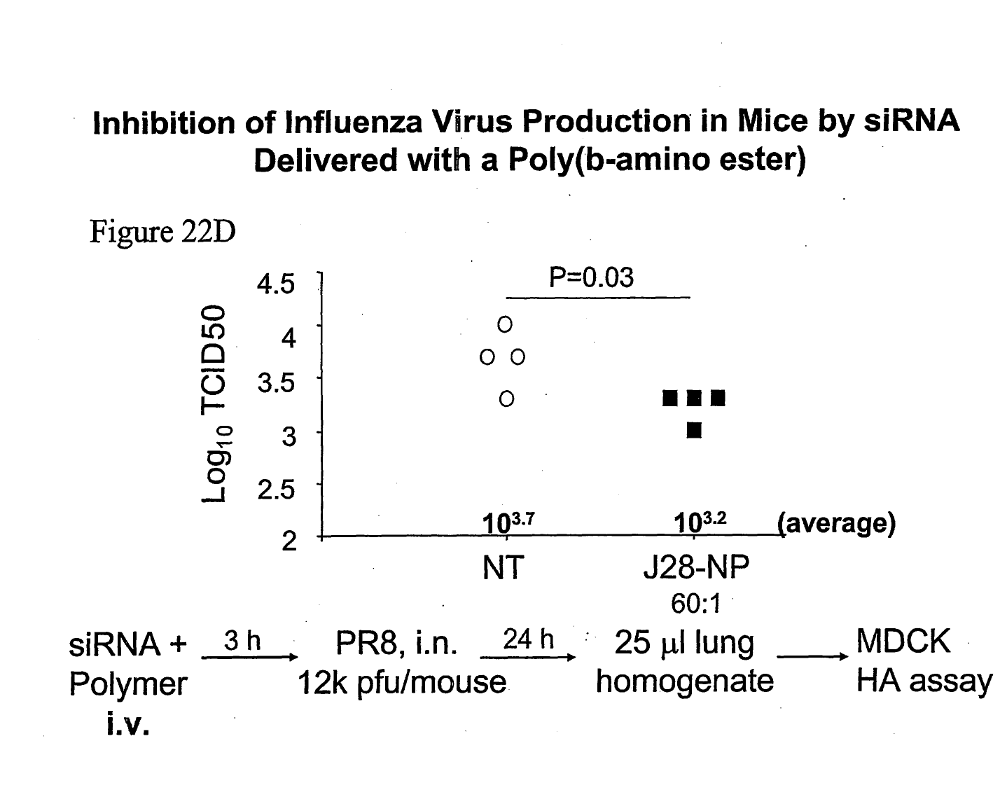

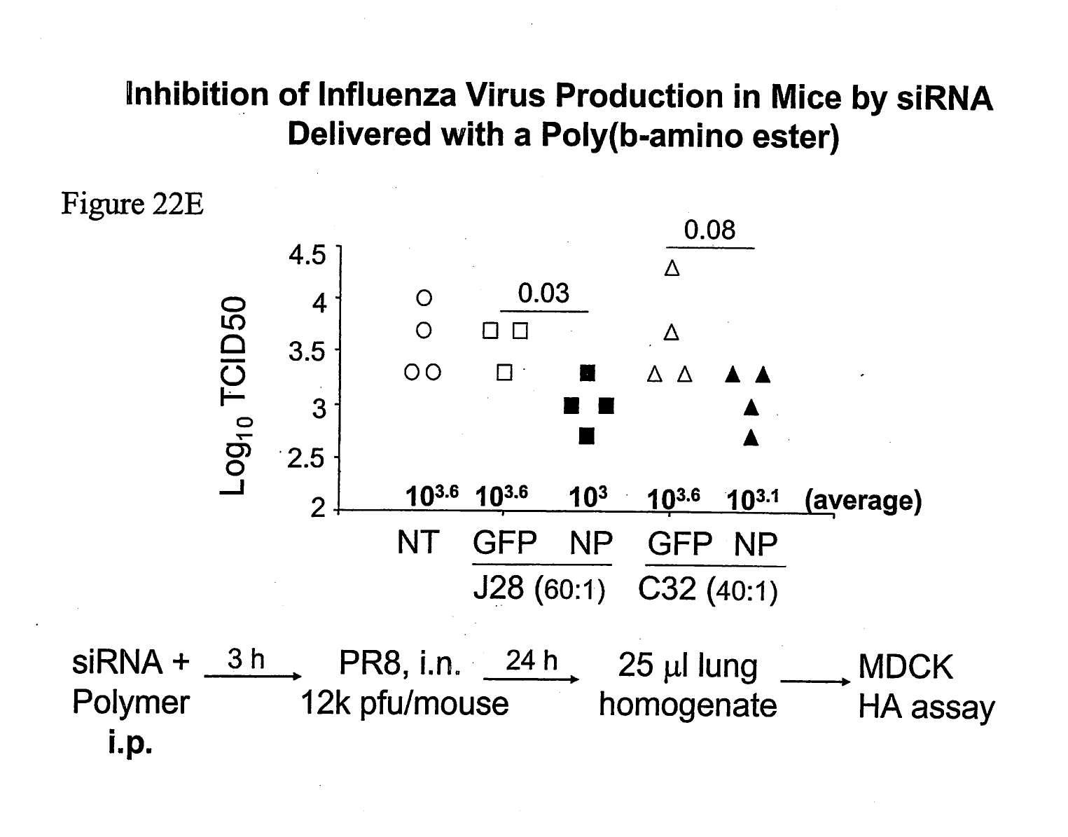

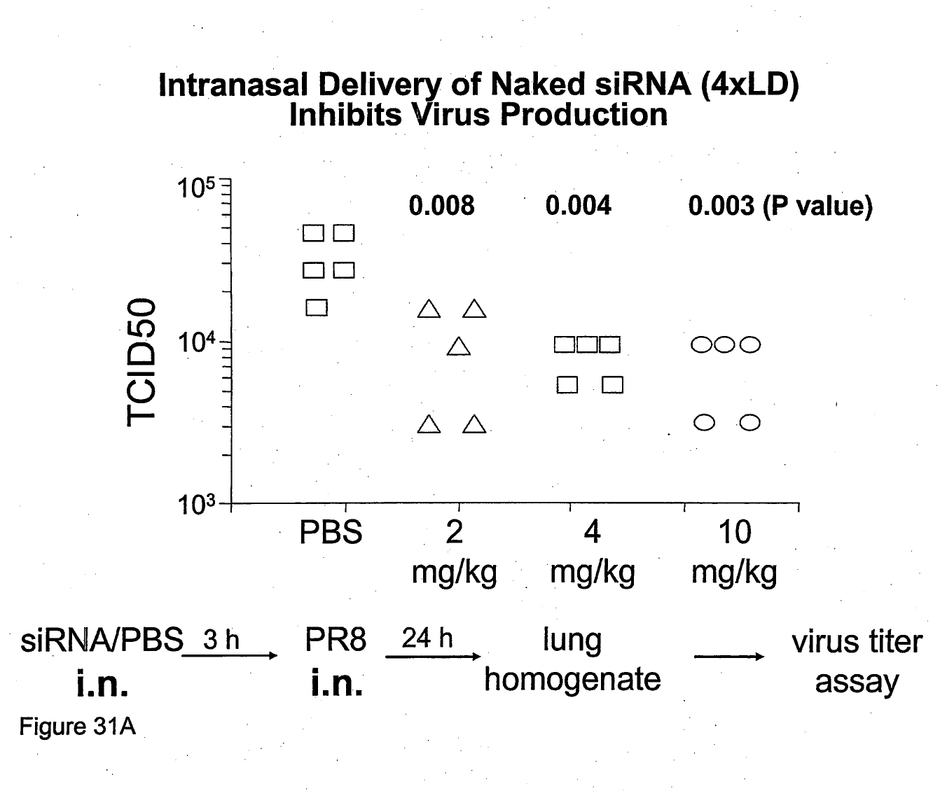

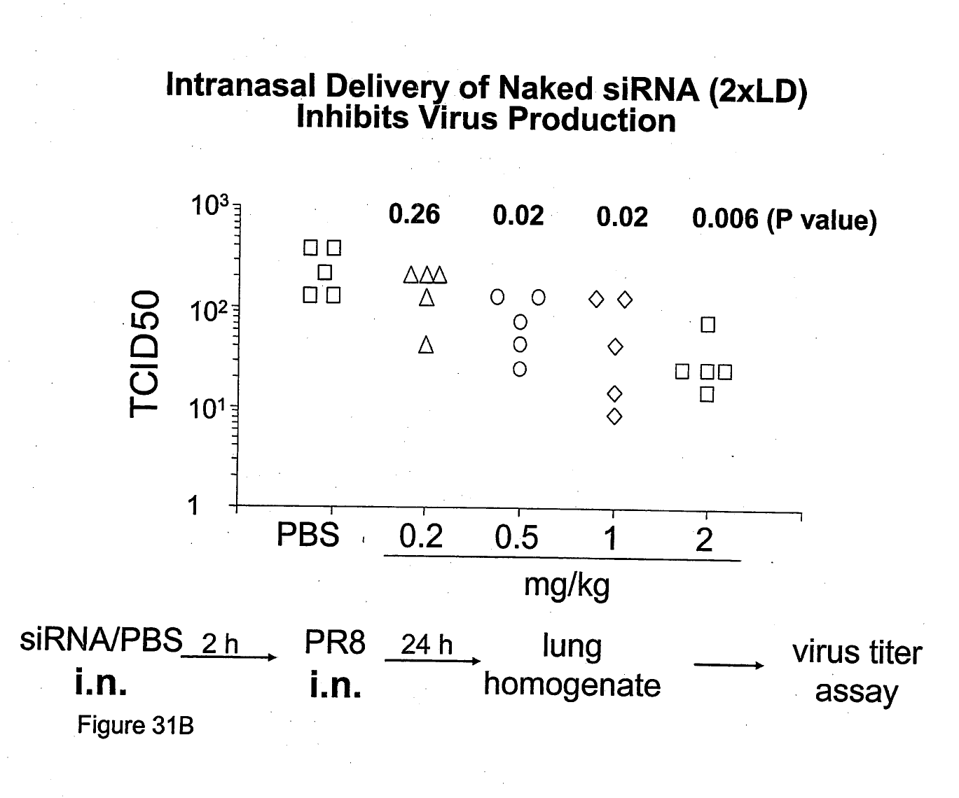

While cationic polymers have been shown to facilitate DNA plasmid transfection, given the considerable differences in structure and size between siRNA and shRNA molecules and DNA plasmids, whether cationic polymers would prove useful in enhancing uptake of siRNA was highly uncertain. However, as described in Example 12, the inventors have shown that compositions comprising PEI, PLL, or PLA and an siRNA that targets an influenza virus RNA significantly inhibit production of influenza virus in mice when administered intravenously either before or after influenza virus infection. The inhibition is dose-dependent and exhibits additive effects when two siRNAs targeted to different influenza virus RNAs were used. Thus siRNA, when combined with a cationic polymer such as PEI, PLL, or PLA, is able to reach the lung, to enter cells, and to effectively inhibit the viral replication cycle. While the presence of PEI significantly enhanced delivery to the lung, effective delivery occurred even in its absence (Examples 12; FIG. 22C), indicating that effective siRNA delivery to the respiratory system can be achieved using “naked” siRNA. It is believed that these findings represent the first report of efficacy in inhibiting production of infectious virus in a mammal using siRNA (as opposed, for example, to inhibiting production of viral transcripts or intermediates in a viral replicative cycle). As described in Example 16, pulmonary administration of a mixture of siRNA and a cationic polymer effectively inhibited a target transcript in lung cells. Examples 23 and 24 and FIG. 31 provide further evidence that naked siRNA delivered to the respiratory system of a mammalian subject effectively inhibits expression of a target transcript therein.

Other efforts to deliver siRNA intravenously to solid organs and tissues within the body (see, e.g., McCaffrey 2002; McCaffrey 2003; Lewis, D. L., 2002) have employed the technique known as hydrodynamic transfection, which involves rapid delivery of large volumes of fluid into the tail vein of mice and has been shown to result in accumulation of significant amounts of plasmid DNA in solid organs, particularly the liver (Liu 1999; Zhang 1999; Zhang 2000). This technique involves delivery of fluid volumes that are almost equivalent to the total blood volume of the animal, e.g., 1.6 ml for mice with a body weight of 18-20 grams, equivalent to approximately 8-12% of body weight, as opposed to conventional techniques that involve injection of approximately 200 μl of fluid (Liu 1999). In addition, injection using the hydrodynamic transfection approach takes place over a short time interval (e.g., 5 seconds), which is necessary for efficient expression of injected transgenes (Liu 1999). siRNA has also been delivered intravenously to subcutaneously implanted tumor cells in nude mice (Filleur 2003), but the relevance of this finding for intravenous delivery of RNAi-inducing agents to native organs and tissues is unclear given the distinctive features of this system. Furthermore, the present invention demonstrates effective intravenous delivery of an RNAi-inducing agent at doses of 5 mg/kg or less, e.g., 0.1-5 mg/kg of the body weight of a subject.

While the mechanism by which hydrodynamic transfection achieves transfer and high level expression of injected transgenes in the liver is not entirely clear, it is thought to be due to a reflux of DNA solution into the liver via the hepatic vein due to a transient cardiac congestion (Zhang 2000). A comparable approach for therapeutic purposes in humans seems unlikely to be feasible. The inventors, in contrast, have used conventional volumes of fluid (e.g., 200 μl) and have demonstrated effective delivery of siRNA to the lung under conditions that would be expected to lead to minimal expression of injected transgenes even in the liver, the site at which expression is most readily achieved using hydrodynamic transfection.

The invention therefore provides a method of inhibiting expression of a transcript, e.g., a viral transcript such as an influenza virus transcript, in a cell within a mammalian subject comprising the step of introducing a composition comprising an RNAi-inducing agent such as an siRNA or shRNA targeted to the target transcript into the vascular system of the subject using a conventional injection technique, e.g., a technique using conventional pressures and/or conventional volumes of fluid. In preferred embodiments of the invention the intravenous administration results in a therapeutically effective dose of the agent within a target organ, e.g., the lung. In certain embodiments of the invention the composition comprises a cationic polymer. In preferred embodiments of the invention the composition is introduced in a fluid volume equivalent to less than 10% of the subject's body weight. In certain embodiments of the invention the fluid volume is equivalent to less than 5%, less than 2%, less than 1%, or less than 0.1% of the subject's body weight. In certain embodiments of the invention the method achieves delivery of effective amounts of an RNAi-inducing agent in a cell in a body tissue or organ other than the liver, for example, the lung. In certain preferred embodiments of the invention the composition is introduced into a vein, e.g., by intravenous injection. However, the composition may also be administered into an artery, delivered using a device such as a catheter, indwelling intravenous line, etc. In certain preferred embodiments of the invention the RNAi-inducing agent inhibits production of a virus, e.g., in the lung.

As described in Example 15, the inventors have also shown that the cationic polymers PLL and PLA form complexes with siRNAs and promote uptake of functional siRNA in cultured cells. Transfection with complexes of PLL and NP-1496 or complexes of PLA and NP-1496 siRNA inhibited production of influenza virus in cells. These results and the results in mice discussed above demonstrate the advantages of using mixtures of cationic polymers and siRNA for delivery of siRNA to mammalian cells in the body of a subject. The approach described in Example 15 may be employed to test additional polymers, e.g., polymers modified by addition of groups (e.g., acyl, succinyl, acetyl, or imidazole groups) to reduce cytotoxicity, and to optimize those that are initially effective. Certain preferred modifications result in a reduction in the positive charge of the cationic polymer. Certain preferred modifications convert a primary amine into a secondary amine. Methods for modifying cationic polymers to incorporate such additional groups are well known in the art. (See, e.g., ref. 32). For example, the e-amino group of various residues may be substituted, e.g., by conjugation with a desired modifying group after synthesis of the polymer. In general, it is desirable to select a % substitution sufficient to achieve an appropriate reduction in cytotoxicity relative to the unsubstituted polymer while not causing too great a reduction in the ability of the polymer to enhance delivery of the RNAi-inducing agent. Accordingly, in certain embodiments of the invention between 5% and 75%, e.g., approximately 50% of the residues in the polymer are substituted. Similar effects may be achieved by initially forming copolymers of appropriately selected monomeric subunits, i.e., subunits some of which already incorporate the desired modification. Cationic polymers for use to facilitate delivery of RNAi-inducing agents may be modified so that they incorporate one or more residues other than the major monomeric subunit of which the polymer is comprised. For example, one or more alternate residues may be added to the end of a polymer, or polymers may be joined by a residue other than the major monomer of which the polymer is comprised.

A variety of additional cationic polymers may also be used. Examples include oly(β-amino ester) (PAE) polymers (such as those described in U.S. Ser. No. 09/969,431; 10/446,444; US Pub. 20020131951 and in refs. 34 and 93). While some poly(β-amino ester) (PAE) polymers have been shown to facilitate DNA plasmid transfection, given the considerable differences in structure and size between siRNA and shRNA molecules and DNA plasmids, whether cationic polymers would prove useful in enhancing uptake of siRNA was highly uncertain. However, as described in Example 12, the inventors showed that siRNA targeted to NP (NP-1496) inhibited influenza virus production in mice when administered intravenously together with a poly(beta amino ester). In addition, this siRNA inhibited influenza virus production in mice when administered intraperitoneally together with a second poly(beta amino ester).

Additional cationic polymers that may also be used to enhance delivery of inventive RNAi-inducing agents include polyamidoamine (PAMAM) dendrimers, poly(2-dimethylamino)ethyl methacrylate (pDMAEMA), and its quaternary amine analog poly(2-triemethylamino)ethyl methacrylate (pTMAEMA), poly [a-(4-aminobutyl)-L-glycolic acid (PAGA), and poly (4-hydroxy-1-proline ester). See Han (2000) for further description.

Modified cationic polymers, e.g., poly(L-histidine)-graft-poly(L-lysine) polymers (Berms 2000), polyhistidine-PEG (Putnam 2003), folate-PEG-graft-polyethyleneimine (Benns 2002), polyethylenimine-dextran sulfate (Tiyaboonchai 2003), etc., can be used. The polymers may be branched or linear and may be grafted or ungrafted. In certain embodiments the polymers form complexes with inventive RNAi-inducing entities, which are then administered to a subject. Any of the polymers may be modified to incorporate PEG or other hydrophilic polymers. Cationic polymers may be multiply modified.

The invention encompasses modification of any of the delivery agents described herein to incorporate a moiety that enhances delivery of the agent to cells and/or enhances the selective delivery of the agent to specific cells. Any of a variety of moieties may be used, e.g., (i) antibodies or antibody fragments that specifically bind to a molecule expressed by a cell in which inhibition is desired, (e.g., a respiratory epithelial cell); (ii) ligands that specifically bind to a molecule expressed by a cell in which inhibition is desired. Methods for conjugating antibodies, ligands, and/or delivery agents to nucleic acids or to the various delivery agents described herein are well known in the art. See e.g., “Cross-Linking”, Pierce Chemical Technical Library, available at the Web site having URL www.piercenet.com and originally published in the 1994-95 Pierce Catalog and references cited therein and Wong S S, Chemistry of Protein Conjugation and Crosslinking, CRC Press Publishers, Boca Raton, 1991; and G. T. Hermanson, Bioconjugate Techniques, Academic Press, Inc., 1995.

C. Additional Agents for Delivering RNAi-Inducing Entities to the Respiratory System

The invention encompasses compositions comprising any of a variety of additional agents and an RNAi-inducing entity, wherein the agent enhances delivery of the RNAi-inducing entity, e.g., to respiratory epithelial cells.

In certain embodiments peptide molecular transporters, which are peptides that can penetrate the plasma membrane from the cell surface, are included in a composition. They generally consist of 11-34 amino acid residues, are highly enriched for arginine, and are often referred to as arginine rich peptides (ARPs) or penetratins (see references 42-51, 120, 134-36).

In other embodiments a composition comprises a surfactant suitable for introduction into the lung. Examples include commercially available formulations Infasurf® (ONY, Inc., Amherst, N.Y.); Survanta® (Ross Labs, Abbott Park, Ill.), and Exosurf Neonatal® (GlaxoSmithKline, Research Triangle Park, N.C.). U.S. Pat. Nos. 4,338,301; 4,397,839; 4,312,860; 4,826,821; 5,110,806). U.S. Pat. No. 4,312,860; 4,826,821; and 5,110,806 describe additional surfactant compositions. In general, any lipid-containing material that does not cause substantial injury to the lung can be used as a surfactant.

Administration with detergents and thixotrophic solutions may also be used. Perfluorochemical liquids, e.g., heptacosafluorotributylamine (Fluorinert), and related molecules, may also be used. See (74, 126, 150) and U.S. Pat. No. 6,638,767 for further discussion. In addition, the invention encompasses the use of protein/polyethylenimine complexes incorporating inventive RNAi-inducing entities, for delivery, e.g., to the respiratory system, e.g., the lung. Other delivery agents that can be used include natural and synthetic cyclodextrins and mixtures of these with other delivery agents. See Singh, M, et al., Biotechnol Adv. 20(5-6):341-59, 2002; Eastburn, S D and Tao, B Y, Biotechnol Adv., 12(2):325-39, 1994) and U.S. Pub. No. 20030157030 for further information. Various non-cationic polymers such as poly(lactide) (PLA), poly(glycolide) (PLG), and poly(DL-lactide-co-glycolide) (PLGA) (Panyam 2002), polyvinyl alcohol, poly(N-ethyl-4-vinylpyridium bromide, Pluronics, poly(ether-anhydride) may be used. Combinations between cationic and non-cationic polymers such as poly(lactic-co-glycolic acid) (PLGA)-grafted poly(L-lysine) (Jeong 2002) and other combinations including PLA, PLG, or PLGA and any of the cationic polymers or modified cationic polymers such as those discussed above are employd in certain embodiments. Block copolymers, which may comprise cationic and/or non-cationic monomers, may also be used. Examples are described in U.S. Pat. Nos. 6,800,663; 6,692,770; 6,669,959; 6,616,941; 6,592,899; and 6,517,869.

VII. Compositions for Inhalational Delivery of RNAi-Inducing Entities

The invention provides compositions comprising RNAi-inducing entities, for administration by inhalation. Preferably the RNAi-inducing entity is an RNAi-inducing entioty such as an siRNA or shRNA. As mentioned above, RNAi-inducing agents can be administered directly to the respiratory system either in naked form or with a delivery agent by inhalation through the nose or mouth and into the lungs. In certain embodiments the RNAi-inducing agent is administered in an amount effective to treat or prevent a condition that affects the respiratory system, such as a respiratory virus infection, while resulting in minimal absorption into the blood and thus minimal systemic delivery of the RNAi-inducing agent. In certain embodiments of the invention the extent of absorption into the blood is such that no clinically significant effects are observed in an organ or tissue outside the respiratory system when the RNAi agent is administered at a dose that is effective in the lung.

In particular, the invention provides dry powder compositions comprising RNAi-inducing entities, preferably RNAi-inducing agents. The inventive agents are preferably delivered in the form of an aerosol spray from a pressured container or dispenser which contains a suitable propellant, e.g., a gas such as carbon dioxide, or a nebulizer. In certain embodiments the delivery system is suitable for delivering the composition into major airways (trachea and bronchi) of a subject and/or deeper into the lung (bronchioles and/or alveoli). In certain embodiments compositions comprising an RNAi-inducing entity are delivered using a nasal spray. Delivery agents may be included in the pharmaceutical composition. However, the inventors have also discovered that RNAi-inducing agents can effectively inhibit influenza virus when delivered to the respiratory system via the respiratory passages in the absence of specific delivery agents (Example 23). In certain embodiments RNAi-inducing agents are delivered to the lungs as a composition that consists essentially of the RNAi-inducing agent in dry form (e.g., dry powder) or in an aqueous medium that consists essentially of water, optionally also including a salt (e.g., NaCl, a phosphate salt), buffer, and/or an alcohol, e.g., as naked siRNA or shRNA.

Aerosol formulations for delivery to the airways and lung may comprise liquid or dry particles of various dimensions and properties. A dry particle composition containing particles smaller than about 1 mm in diameter is also referred to herein as a dry powder. By “dry” is meant that the composition has a relatively low liquid content, so that the particles are readily dispersible, e.g., in a dry powder inhalation device to form an aerosol or spray. By “powder” is meant a composition that consists largely or entirely of finely dispersed solid particles that are relatively free flowing and capable of being readily dispersed in an inhalation device and subsequently inhaled by a subject, e.g., a patient, preferably so that the particles can reach the alveoli of the lung, i.e., they are suitable for pulmonary delivery. Powder compositions may be characterized on the basis of various parameters such as the fine particle fraction (FPF), the emitted dose, the average particle density, and the mass median aerodynamic diameter (MMAD). Suitable methods are known in the art and are found, for example, in references 31 and 58 and in U.S. Publication Nos. 20020146373, 20030012742, and 20040092470. In certain embodiments of the invention particles having a mass mean aerodynamic diameter of between 1 μm and 25 μm, preferably between 1 μm and 10 μm, are used. In certain embodiments large porous particles having mean geometric diameters ranging between 3 and 15 μm and tap density between 0.04 and 0.6 g/cm³ are used (31, 58).

Methods for making dry particles are known in the art. Suitable methods include spray drying, spray-freeze drying, phase separation, single or double emulsion solvent evaporation, solvent extraction, and simple and complex coacervation. Particulate compositions can also be made using granulation, extrusion, and/or spheronization. Methods suitable for preparing dry powder oligonucleotide formulations are known in the art. See, e.g., U.S. Publication No. 20040092470. The methods employed preferably do not greatly reduce the physical integrity and ability of the nucleic acid to inhibit a target transcript. It is desirable to avoid extremes of temperature or pH that are known to result in significant degradation of nucleic acids. It will be appreciated that in general the extent of degradation may be a function of both the particular conditions and the time over which the nucleic acid is exposed to the conditions, such that minimizing the duration of exposure may be desirable and may allow more extreme conditions to be used. Compositions can be tested to determine whether the method selected is appropriate in terms of retaining sufficient efficacy. Preferably a selected formulation method results in a composition in which the portion consisting of the nucleic acid has at least 10% preferably at least 20%, 50%, or more of the level of activity of the input nucleic acid.

The conditions used in preparing the particles may be selected to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the particular active agents and other components included in the composition. If the particles prepared by any of the above methods have a size range outside of the desired range, the particles can be sized, for example, using a sieve, by milling, etc. Combinations of methods may be employed.

The dry powders may consist essentially of one or more RNAi-inducing agents. In certain embodiments the formulation includes one or more additional agents, e.g., stabilizing agents, delivery-enhancing agents such as those described above, excipients, etc. The term “excipient”, as used herein, refers to a substance that is present in a formulation of the invention other than an active agent or delivery-enhancing agent. Suitable excipients for pulmonary delivery are known in the art. Any of a large number of art-recognized compounds may be included in the inventive formulations. In general, compositions having concentrations of between 0.1% and 100% active agent (i.e., RNAi-inducing agent) by weight may be used.

Methods for testing particles, e.g., for ability to reduce target transcript levels and/or inhibit influenza virus production are described in Example 10. Similar methods may be used for any of the inventive aerosol formulations. Dry particle compositions may be dissolved in a suitable solvent and delivered as liquid aerosols or by other suitable delivery means.

Liquid particles can also be delivered, e.g., as aerosol formulations. In general, size ranges for such particles may be similar to those described above for dry particles. In certain embodiments the liquid particles are between approximately 0.5-5 μm for respiratory delivery, though smaller or larger particles could also be used. Suitable aqueous vehicles include water or saline, optionally including an alcohol. Additional considerations for pulmonary delivery are discussed in Bisgaard, H., et al., (eds.), Drug Delivery to the Lung, Vol. 26 in “Lung Biology in Health and Disease”, Marcel Dekker, New York, 2002.

Particles comprising the inventive RNAi-inducing agents can also be administered intravenously, if desired. For intravenous delivery, sizes of approximately 10 nm-50 μm are generally preferred.

VIII Therapeutic Applications

Compositions comprising the RNAi-inducing entites of the present invention may be used to inhibit or reduce respiratory virus infection or replication. In such applications, an effective amount of an inventive composition is delivered to a cell or organism prior to, simultaneously with, or after exposure to a virus, e.g., an influenza virus. Preferably, the amount of the RNAi-inducing entity is sufficient to reduce or delay one or more symptoms of infection. For purposes of description this section will often refer to inventive siRNAs, but the invention encompasses similar applications for other RNAi-inducing entities targeted to viral transcripts. It will also be appreciated that influenza virus is used herein as an example, but the methods may be applied to any of a wide range of other respiratory viruses.

Inventive compositions may comprise a single species of RNAi-inducing agents targeted to a single site in a single influenza transcript, or may comprise a plurality of different species, targeted to one or more sites in one or more influenza transcripts. In some embodiments of the invention, it will be desirable to utilize compositions containing collections of different RNAi-inducing agents (e.g., multiple different siRNAs) targeted to different influenza genes. For example, it may be desirable to attack the virus at multiple points in the viral life cycle using a variety of siRNAs directed against different viral transcripts. According to certain embodiments of the invention the composition contains an siRNAi targeted to each segment.

In certain embodiments the composition comprises 2, 3, 4, 5, 6, 7, 8, 9, or 10 different RNAi-inducing agent species, e.g., 2, 3, 4, 5, 6, 7, 8, 9, or 10 different siRNAs. In certain embodiments of the invention the RNAi-inducing agents are targeted to portions of the influenza virus genome having sequences selected from the group consisting of SEQ ID NOs: 272-380. In certain embodiments of the invention the RNAi-inducing agents are targeted portions of the influenza virus genome having sequences selected from the group consisting of any selected subset of SEQ ID NOs: 272-380. All such subsets are included herein for any and all purposes, even if not explicitly set forth.

In certain embodiments of the invention the composition comprises at least one RNAi-inducing agent targeted to PA and at least one RNAi-inducing agent targeted to another influenza virus gene, e.g., NP, PB1, or PB2. In certain embodiments of the invention the composition comprises at least one RNAi-inducing agent targeted to PB1 and at least one RNAi-inducing agent targeted to another influenza virus gene, e.g., NP, PA, or PB2. In certain embodiments of the invention the composition comprises at least one RNAi-inducing agent targeted to PB2 and at least one RNAi-inducing agent targeted to another influenza virus gene, e.g., NP, PB1, or PA. In certain embodiments of the invention the composition comprises at least one RNAi agent targeted to NP and at least one RNAi-inducing agent targeted to another influenza virus gene, e.g., PA, PB 1, or PB2.

According to certain embodiments of the invention, inventive siRNA compositions may contain more than one siRNA species targeted to a single viral transcript. To give but one example, it may be desirable to include at least one siRNA targeted to coding regions of a target transcript and at least one siRNA targeted to the 3′ UTR. This strategy may provide extra assurance that products encoded by the relevant transcript will not be generated because at least one siRNA in the composition will target the transcript for degradation while at least one other inhibits the translation of any transcripts that avoid degradation.

Thus the invention encompasses combinations of inventive RNAi-inducing entities including, but not limited to, approaches in which multiple RNAi-inducing agents, e.g., multiple siRNAs or shRNAs are administered and approaches in which a single vector directs synthesis of siRNAs that inhibit multiple influenza virus transcripts or of RNAs that may be processed to yield a plurality of siRNAs. See Example 11 for further details. According to certain embodiments of the invention the composition includes an RNAi-inducing agent targeted to at least one influenza virus A transcript and an RNAi-inducing agent targeted to at least one influenza virus B transcript. In certain embodiments the composition comprises an RNAi-inducing agent targeted to both an influenza A virus transcript and an influenza B virus transcript. According to certain embodiments of the invention the composition comprises multiple siRNAs having different sequences that target the same portion of a particular segment. According to certain embodiments of the invention the composition comprises multiple RNAi-inducing agents that inhibit different influenza virus strains or subtypes

Influenza viruses undergo both antigenic shift and antigenic drift, and resistance to therapeutic agents may arise. It may expected that, after an inventive composition has been in use for some time, mutation and/or reassortment may occur so that a variant that is not inhibited by the particular RNAi-inducing agents provided may emerge. The present invention therefore contemplates evolving therapeutic regimes. For example, one or more new RNAi-inducing agents can be selected in a particular case in response to a particular mutation or reassortment. For instance, it would often be possible to design a new RNAi-inducing agent identical to the original except incorporating whatever mutation had occurred or targeting a newly acquired RNA segment; in other cases, it will be desirable to target a new sequence within the same transcript; in yet other cases, it will be desirable to target a new transcript entirely.

It will often be desirable to combine the administration of inventive RNAi-inducing agents with one or more other anti-viral agents in order to inhibit, reduce, or prevent one or more symptoms or characteristics of infection. In certain preferred embodiments of the invention, the inventive RNAi-inducing agents are combined with one or more other antiviral agents such as NA inhibitors, M inhibitors, etc. Examples include amantadine or rimantadine and/or zanamivir, oseltamivir, peramivir (BCX-1812, RWJ-270201) Ro64-0796 (GS 4104) or RWJ-270201. However, the administration of the inventive RNAi-inducing agent compositions may also be combined with one or more of any of a variety of agents including, for example, influenza vaccines (e.g., conventional vaccines employing influenza viruses or viral antigens as well as DNA vaccines) of which a variety are known. See Palese, P. and Garcia-Sastre, 2002; Cheung and Lieberman, 2002, Lèuscher-Mattli, 2000; and Stiver, 2003, for further information.

In different embodiments of the invention the RNAi-inducing agents are present in the same mixture as the other agent(s) or the treatment regimen for an individual includes both RNAi-inducing agents and the other agent(s), not necessarily delivered in the same mixture or at the same time. Thus, as used herein, the term “combination” is not intended to indicate that compounds must be present in, or administered to a subject as, a single composition of matter, e.g., as part of the same dosage unit (e.g., in the same aerosol formulation, particle composition, tablet, capsule, pill, solution, etc.) although they may be. Instead, in certain embodiments of the invention the agents are administered individually but concurrently. As used herein the term “coadministration” or “concurrent administration” of two or more compounds is not intended to indicate that the compounds must be administered at precisely the same time. In general, compounds are coadministered or administered concurrently if they are present within the body at the same time in less than de minimis quantities. Accordingly, the compounds may, but need not be, administered together as part of a single composition. In addition, the compounds may, but need not be, administered simultaneously (e.g., within less than 5 minutes, or within less than one minute) or within a short time of one another (e.g., less than an hour, less than 30 minutes, less than 10 minutes, approximately 5 minutes apart). According to various embodiments of the invention compounds administered within such time intervals may be considered to be administered at substantially the same time. One of ordinary skill in the art will be able to readily determine an appropriate time interval between administration of the compounds so that they will each be present at more than de minimis levels within the body or, preferably, at effective concentrations within the body.

The inventive RNAi-inducing agents and vectors offer a complementary strategy to vaccination and may be administered to individuals who have or have not been vaccinated with any of the various vaccines currently available or under development (reviewed in Palese, P. and Garcia-Sastre, A., J. Clin. Invest., 110(1): 9-13, 2002). Current vaccine formulations in the United States contain inactivated virus and must be administered by intramuscular injection. The vaccine is tripartite and contains representative strains from both subtypes of influenza A that are presently circulating (H3N2 and H1N1), in addition to an influenza B type. Each season specific recommendations identify particular strains for use in that season's vaccines. Other vaccine approaches include cold-adapted live influenza virus, which can be administered by nasal spray; genetically engineered live influenza virus vaccines containing deletions or other mutations in the viral genome; replication-defective influenza viruses, and DNA vaccines, in which plasmid DNA encoding one or more of the viral proteins is administered either intramuscularly or topically (see, e.g., Macklin, M. D., et al., J Virol. 72(2):1491-6, 1998; Ilium, L., et al., Adv Drug Deliv Rev, 51(1-3):81-96, 2001; Ulmer, J., Vaccine, 20:S74-S76, 2002). Immunocompromised patients and elderly individuals may gain particular benefit from RNAi-based therapeutics since they may experience reduced efficacy of influenza virus vaccines.

In some embodiments of the invention, it may be desirable to target administration of inventive compositions to cells infected with influenza virus, or at least to cells susceptible of influenza virus infection (e.g., cells expressing sialic acid-containing receptors). In other embodiments, it will be desirable to have available the greatest breadth of delivery options.

As noted above, inventive therapeutic protocols involve administering an effective amount of an RNAi-inducing agent or vector prior to, simultaneously with, or after exposure to influenza virus. For example, uninfected individuals may be “immunized” with an inventive composition prior to exposure to influenza; at risk individuals (e.g., the elderly, immunocompromised individuals, persons who have recently been in contact with someone who is suspected, likely, or known to be infected with influenza virus, etc.) can be treated substantially contemporaneously with exposure, e.g., within 2 hours or less following exposure. In other embodiments a subject is treated at a later time, e.g., within 2-12, 12-24, 24-36, or 36-48 hours, following a suspected or known exposure. The subject may be symptomatic or asymptomatic. In certain embodiments the subject is protected by administration of an RNAi-inducing agent or vector up to 48 hours, up to 24 hours, up to 12 hours, up to 3 hours, etc., before an exposure. Of course individuals suspected or known to be infected may receive inventive treatment at any time.

Certain preferred influenza virus inhibitors inhibit viral replication, so that the level of replication is lower in a cell containing the inhibitor than in a control cell not containing the inhibitor by at least about 2 fold, preferably at least about 4 fold, more preferably at least about 8 fold, 16 fold, 64 fold, 100 fold, 200 fold, or to an even greater degree. Certain preferred influenza virus inhibitors prevent (e.g., reduce to undetectable levels) or significantly reduce viral replication (e.g., 10% or less, 25% of less, 50% or less, 75%, or less, relative to the level that would occur in the absence of the RNAi-inducing agent) for at least 24 hours, at least 36 hours, at least 48 hours, or about 60 hours following administration of the agent and/or infection.

In certain embodiments of the invention a sustained release preparation is used for prophylactic purposes, e.g., a formulation that releases a sufficient amount of active agent to protect a subject from influenza virus infection, or to lessen the symptoms of such infection over a period of time. For example, the formulation may release an effective amount of agent over a period of several days, a week, 1-2 weeks, or longer. Biodegradable polymeric delivery systems comprising the RNAi-inducing agent or vector can be used.

Thus the RNAi-inducing entities of the invention are therapeutically useful in at least 3 distinct situations: (i) An RNAi-inducing entity may be administered to a subject who is not suspected or known to have been exposed to influenza virus. In such a situation the RNAi-inducing entity preferably prevents the development of a clinically significant infection, or lessens its severity; (ii) An RNAi-inducing entity may be administered to a subject who is suspected or known to have been exposed to influenza virus, e.g., within a preceding time interval of up to a week. The RNAi-inducing entity preferably prevents the development of a clinically significant infection, or lessens its severity. (iii) An RNAi-inducing entity may be administered to a subject who has become clinically ill. The RNAi-inducing entity inhibits influenza virus replication and preferably lessens the severity and/or duration of at least one symptom of influenza virus infection. Subjects who have an infection of the upper respiratory tract, lower respiratory tract, or both, can be treated. In certain embodiments of the invention the subject has viral pneumonia as a result of influenza virus infection.

In certain embodiments of the invention gene therapy is used to prevent influenza or to treat an individual who is already ill. Gene therapy protocols may involve administering an effective amount of a gene therapy vector capable of directing expression of an inhibitory RNAi-inducing agent to a subject either before, substantially contemporaneously, with, or after influenza virus infection.

As mentioned above, influenza viruses infect a wide variety of species in addition to humans. The present invention includes the use of the inventive compositions for the treatment of nonhuman species, particularly species such as chickens, swine, and horses.

IX. Pharmaceutical Formulations

As discussed above, inhalational delivery of the RNAi-inducing entities is preferred in certain embodiments of the invention, while intravenous delivery is preferred in other embodiments of the invention. While inhalational delivery may be more suitable for patients who are in relatively good health, intravenous delivery may be more suitable for individuals who are unable to mount an adequate inspiratory effort and/or suffer from conditions that may impede effective delivery via the respiratory route (e.g., excessive mucus production; situations in which portions of the lung are consolidated due to bacterial infection or occluded by scar tissue, etc.) or in which it is desired to maintain a relatively constant concentration of the agent.

However, inventive compositions may be formulated for delivery by any available route including, but not limited to parenteral (e.g., intravenous), intradermal, subcutaneous, oral, nasal, bronchial, ophthalmic, transdermal (topical), transmucosal, rectal, and vaginal routes. Preferred routes of delivery include parenteral, transmucosal, nasal, bronchial, and oral. Inventive pharmaceutical compositions typically include an RNAi-inducing agent or a vector that will result in production of an RNAi-inducing agent after delivery, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

Systemic administration of any of the RNAi-inducing entities of the invention can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In addition to the delivery agents described above, in certain embodiments of the invention, the active entities are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Sustained release formulations, which may release active agents over a period of hours, days, weeks, or even longer, may be particularly useful for prophylactic purposes. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an RNAi-inducing entity as described herein, can include a single treatment or, in many cases, can include a series of treatments.

Exemplary doses include milligram or microgram amounts of the inventive nucleic acid, e.g., siRNA, per kg of subject or sample weight (e.g., about 1 μg/kg per kilogram to about 500 mg/kg per kilogram, about 100 mg/kg to about 5 mg/kg, or about 1 mg/kg to about 50 mg/kg) For local administration (e.g., intranasal), doses much smaller than these may be used.

It is furthermore understood that appropriate doses of an RNAi-inducing agent depend upon the potency of the agent and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

The present invention includes the use of inventive nucleic acids, e.g., siRNA or shRNA-containing compositions for treatment of nonhuman animals including, but not limited to, horses, swine, and birds. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001.

Inventive pharmaceutical compositions can be included in a container, pack, or dispenser together with instructions for administration.

### Exemplification

### Example 1

### Design of siRNAs to Inhibit Influenza A Virus

Genomic sequences from a set of influenza virus strains were compared in their positive sense form, and regions of each segment that were most conserved were identified. This group of viruses included viruses derived from bird, swine, horse, and human. To perform the comparison the sequences of individual segments from 12 to 15 strains of influenza A virus from different animal (nonnhuman) species isolated in different years and from 12 to 15 strains from humans isolated in different years were aligned. The strains were selected to encompass a wide variety of HA and NA subtypes. Regions that differed either by 0, 1, or 2 nucleotides among the different strains were selected. For example, the following strains were used for selection of siRNAs that target the NP transcript, accession number before each strain name refers to the accession number of the NP sequence and the lengths of the sequence that were compared are indicated by nucleotide number.

The order of the entries in the following list is: accession number, strain name, length of sequence compared, year, subtype. Accession numbers for the other genome segments differ but may be found readily in databases mentioned above. Strains compared were:

| | | | | |
|---|---|---|---|---|
| NC_002019 | A/Puerto Rico/8/34 | 1565 | 1934 | H1N1 |
| M30746 | A/Wilson-Smith/33 | 1565 | 1933 | H1N1 |
| M81583 | A/Leningrad/134/47/57 | 1566 | 1957 | H2N2 |
| AF348180 | A/Hong Kong/1/68 | 1520 | 1968 | H3N2 |
| L07345 | A/Memphis/101/72 | 1565 | 1972 | H3N2 |
| D00051 | A/Udorn/307/72 | 1565 | 1972 | H3N2 |
| L07359 | A/Guangdong/38/77 | 1565 | 1977 | H3N2 |
| M59333 | A/Ohio/201/83 | 1565 | 1983 | H1N1 |
| L07364 | A/Memphis/14/85 | 1565 | 1985 | H3N2 |
| M76610 | A/Wisconsin/3623/88 | 1565 | 1988 | H1N1 |
| U71144 | A/Akita/1/94 | 1497 | 1994 | H3N2 |
| AF084277 | A/Hong Kong/483/97 | 1497 | 1997 | H5N1 |
| AF036359 | A/Hong Kong/156/97 | 1565 | 1997 | H5N1 |
| AF250472 | A/Aquatic bird/Hong Kong/ | 1497 | 1998 | H11N1 |
| | M603/98 | | | |
| ISDN13443 | A/Sydney/274/2000 | 1503 | 2000 | H3N2 |
| M63773 | A/Duck/Manitoba/1/53 | 1565 | 1953 | H10N7 |
| M63775 | A/Duck/Pennsylvania/1/69 | 1565 | 1969 | H6N1 |
| M30750 | A/Equine/London/1416/73 | 1565 | 1973 | H7N7 |
| M63777 | A/Gull/Maryland/5/77 | 1565 | 1977 | H11N9 |
| M30756 | A/gull/Maryland/1815/79 | 1565 | 1979 | H13N6 |
| M63785 | A/Mallard/Astrakhan(Gurjev)/ | 1565 | 1982 | H14N5 |
| | 263/82 | | | |
| M27520 | A/whale/Maine/328/84 | 1565 | 1984 | H13N2 |
| M63768 | A/Swine/Iowa/17672/88 | 1565 | 1988 | H1N1 |
| Z26857 | A/turkey/Germany/3/91 | 1554 | 1991 | H1N1 |
| U49094 | A/Duck/Nanchang/1749/92 | 1407 | 1992 | H11N2 |
| AF156402 | A/Chicken/Hong Kong/G9/97 | 1536 | 1997 | H9N2 |
| AF285888 | A/Swine/Ontario/01911-1/99 | 1532 | 1999 | H4N6 |

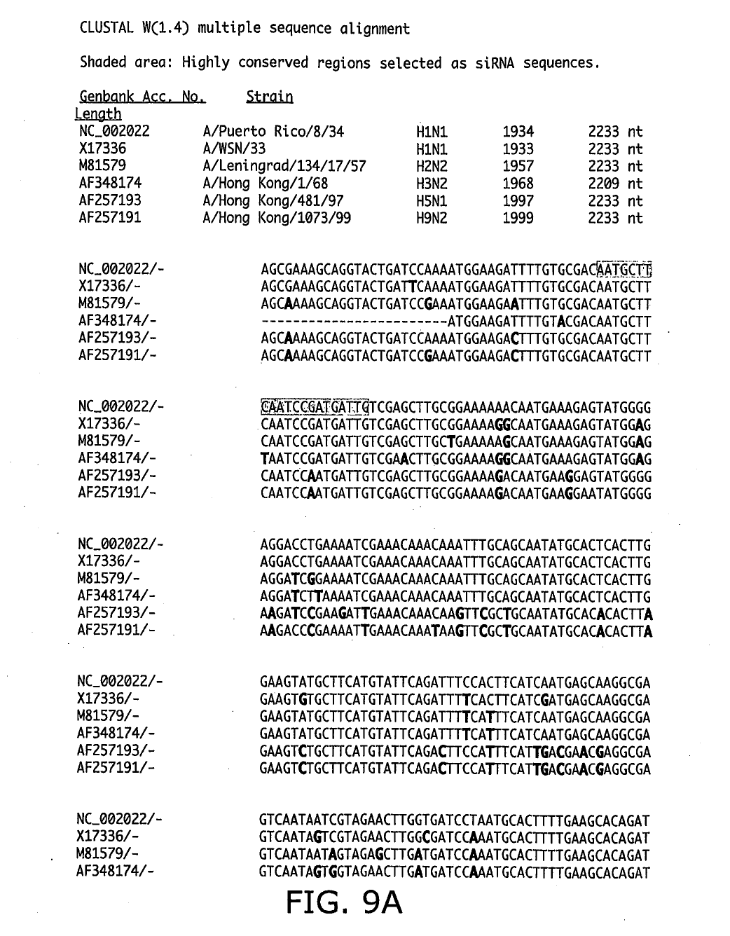

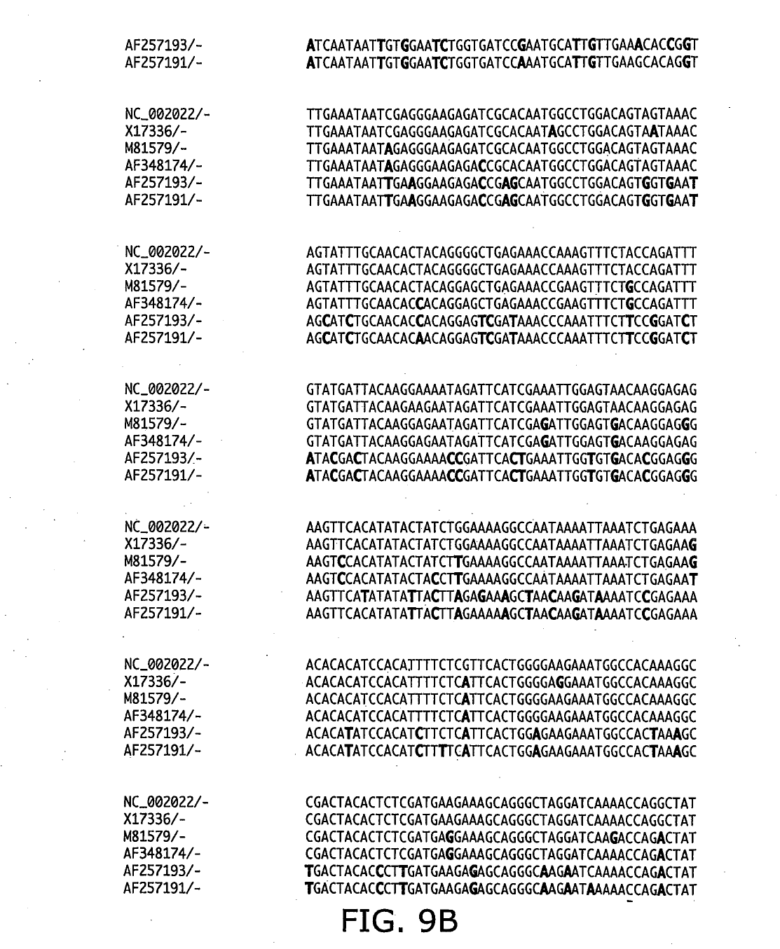

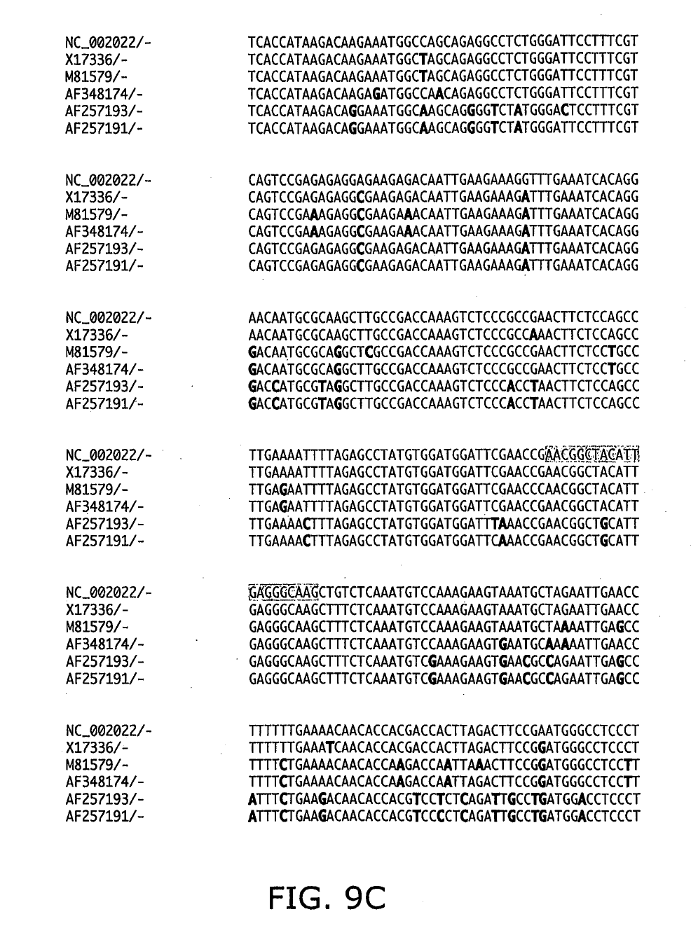

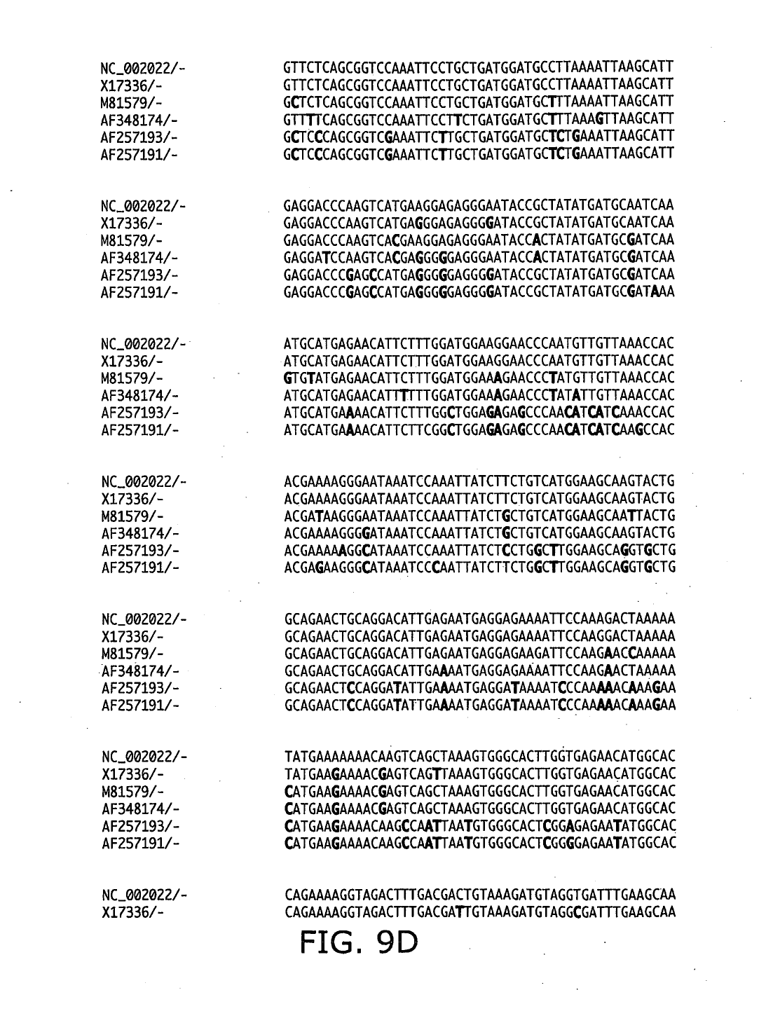

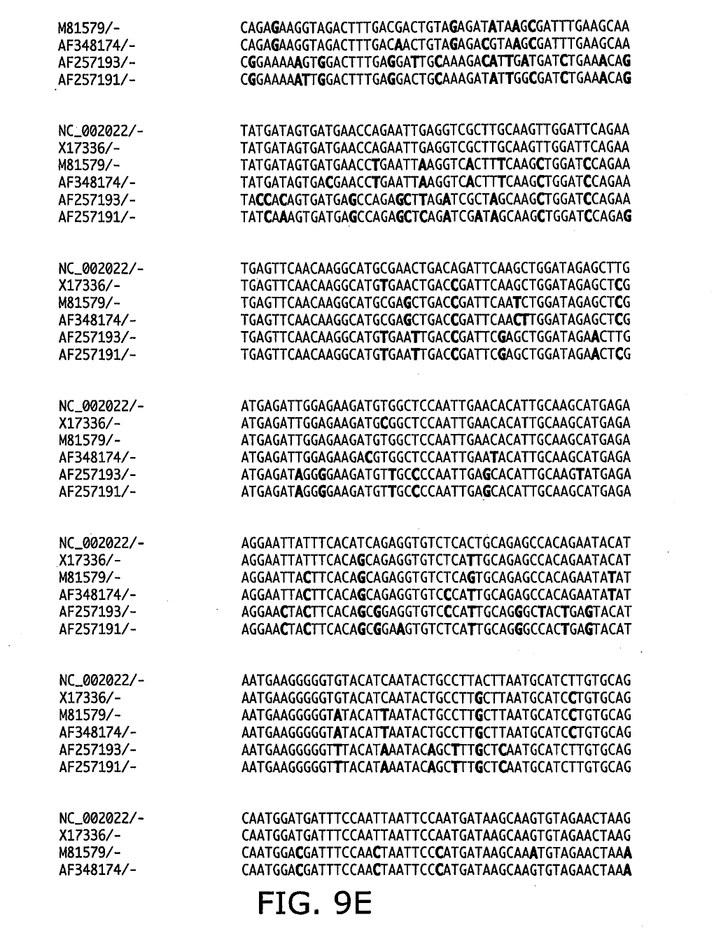

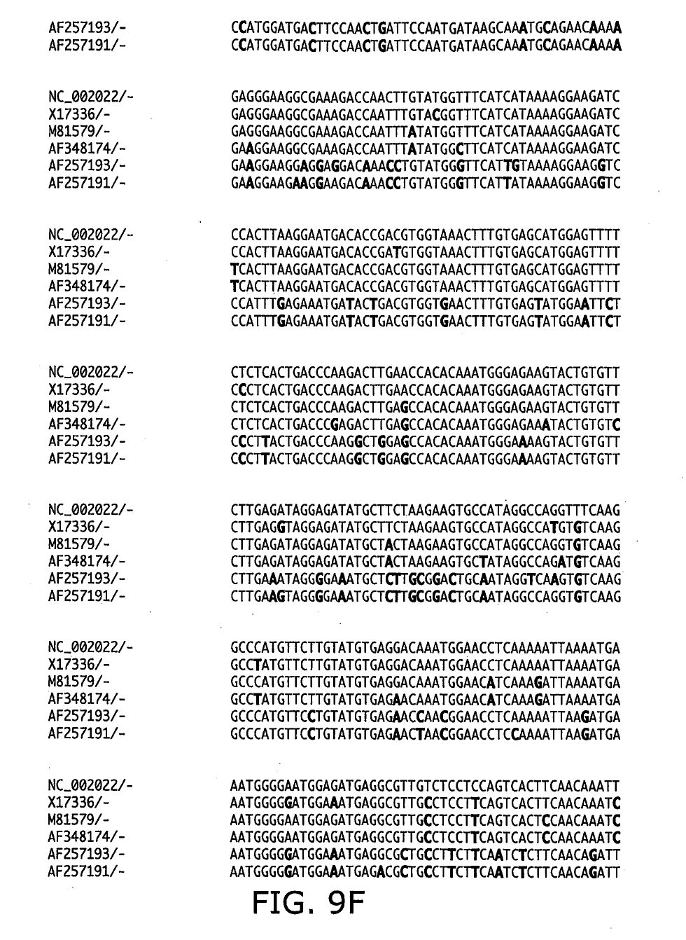

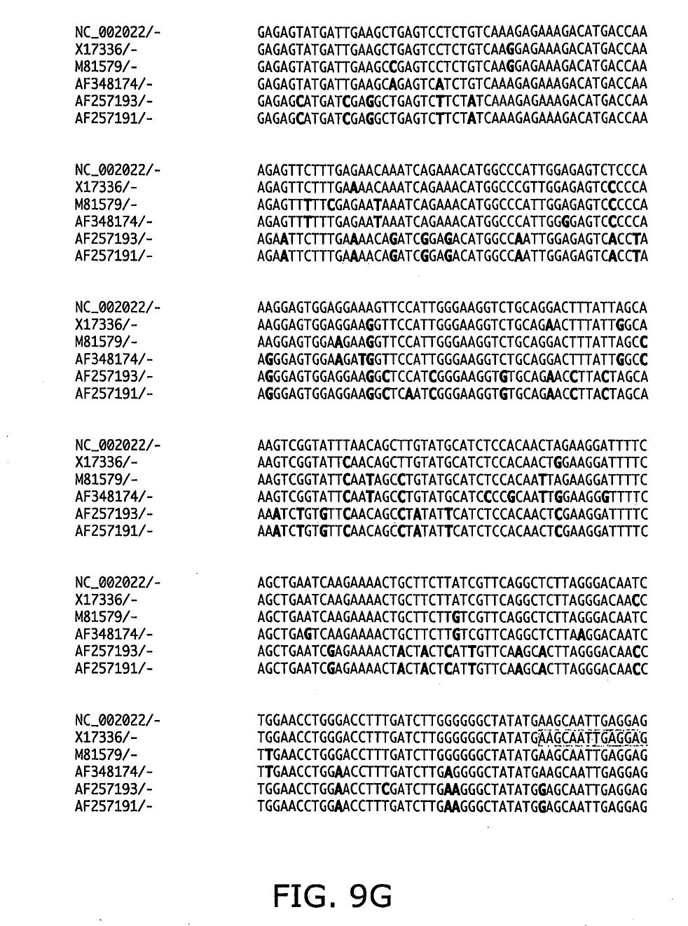

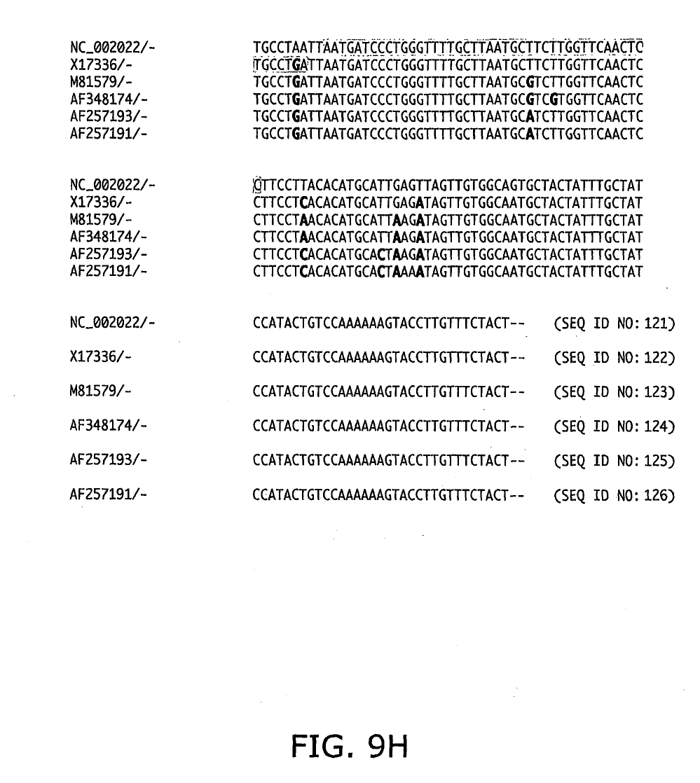

FIG. 9 shows an example of the selection of certain regions of the PA transcript that are highly conserved among six influenza A variants (all of which have a human host of origin), in which regions are considered highly conserved if they differ by either 0, 1, or 2 nucleotides. (Note that the sequences are listed as DNA rather than RNA and therefore contain T rather than U.) The sequence of strain A/Puerto Rico/8/34 (H1N1) was selected as the base sequence, i.e., the sequence with which the other sequences were compared. The other members of the set were A/WSN/33 (H1N1), A/Leningrad/134/17/57 (H2N2), A/Hong Kong/1/68 (H3N2), A/Hong Kong/481/97 (H5N1), and A/Hong Kong/1073/99 (H9N2). The figure presents a multiple sequence alignment produced by the computer program CLUSTAL W (1.4). Nucleotides that differ from the base sequence are shaded.

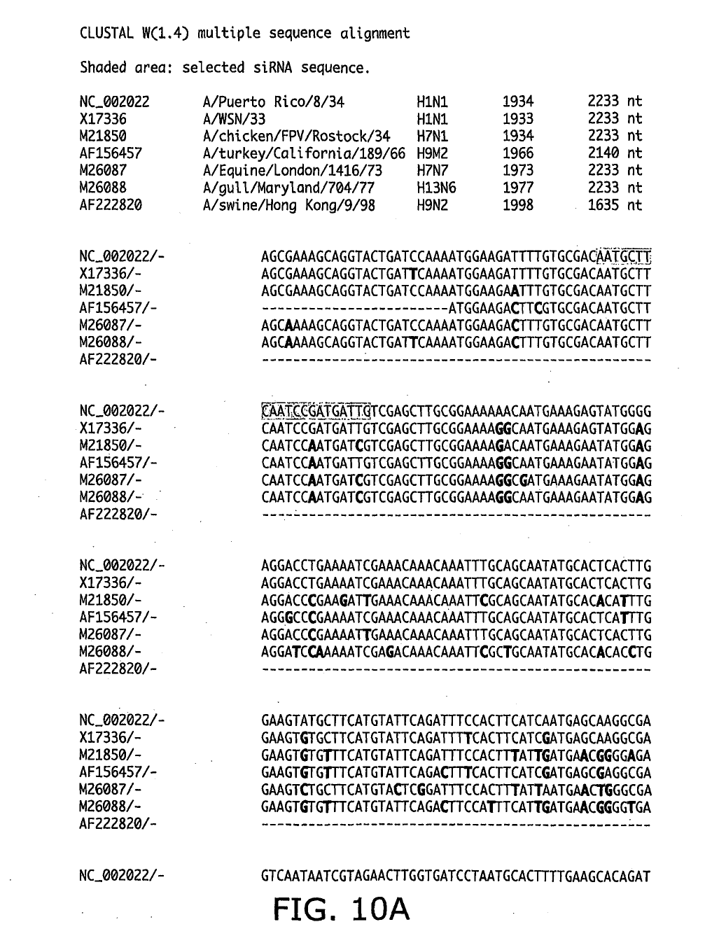

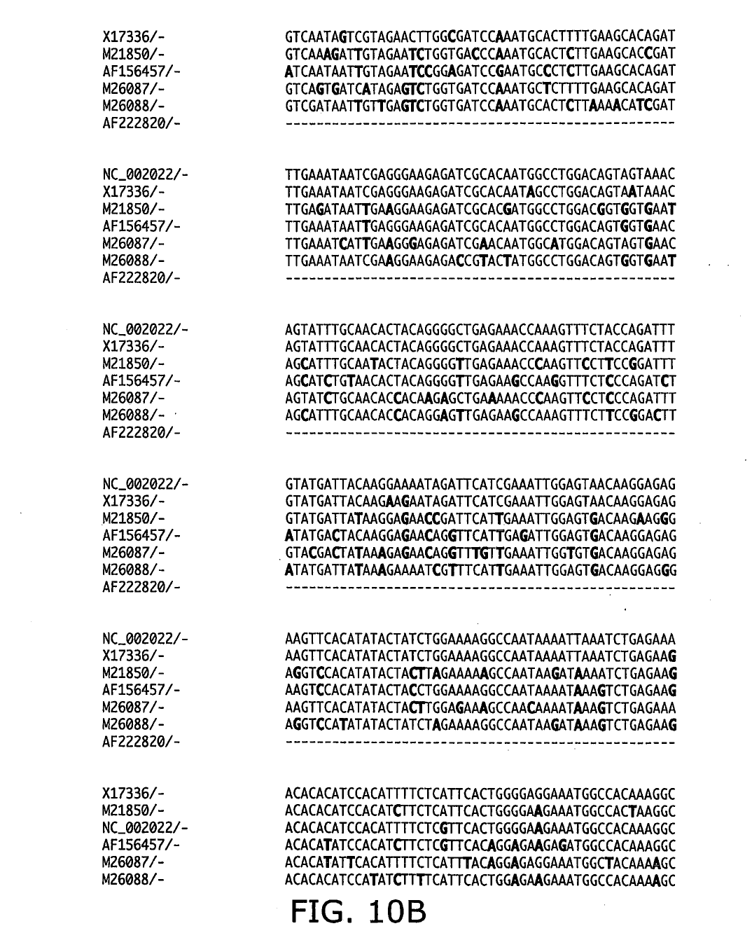

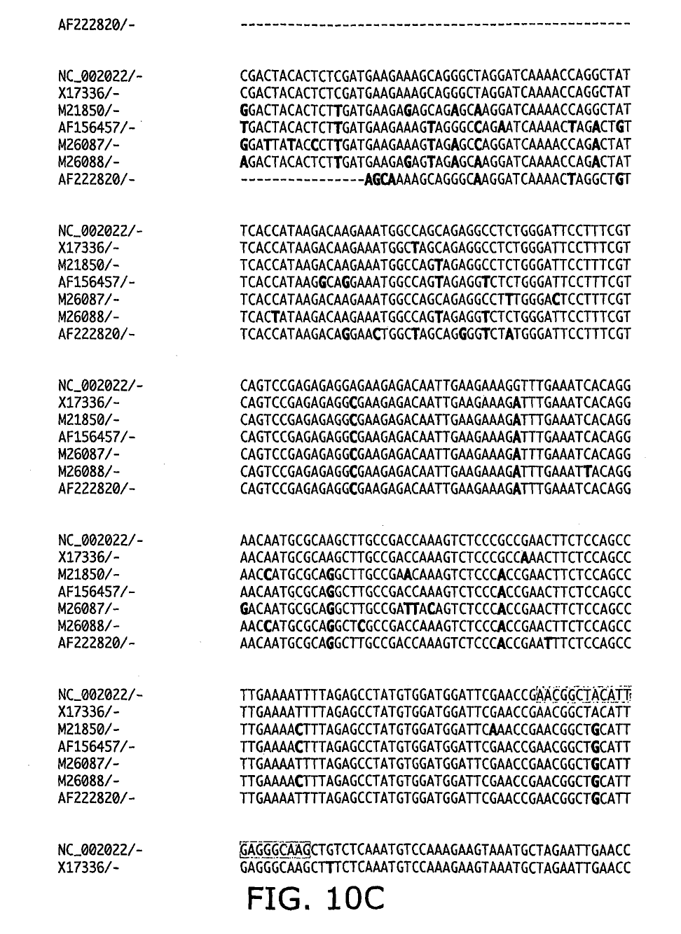

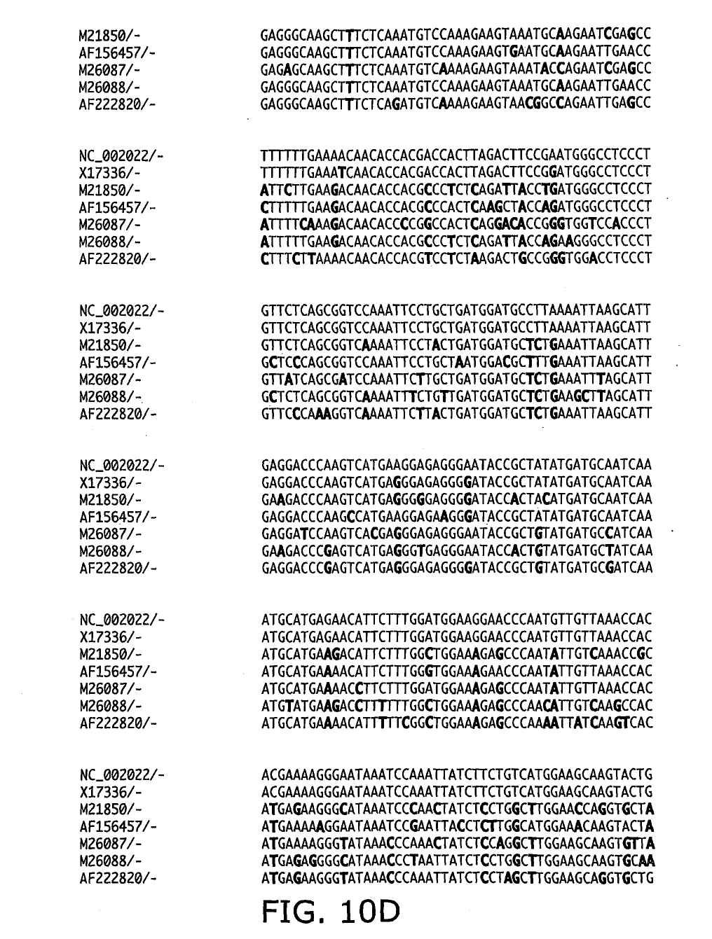

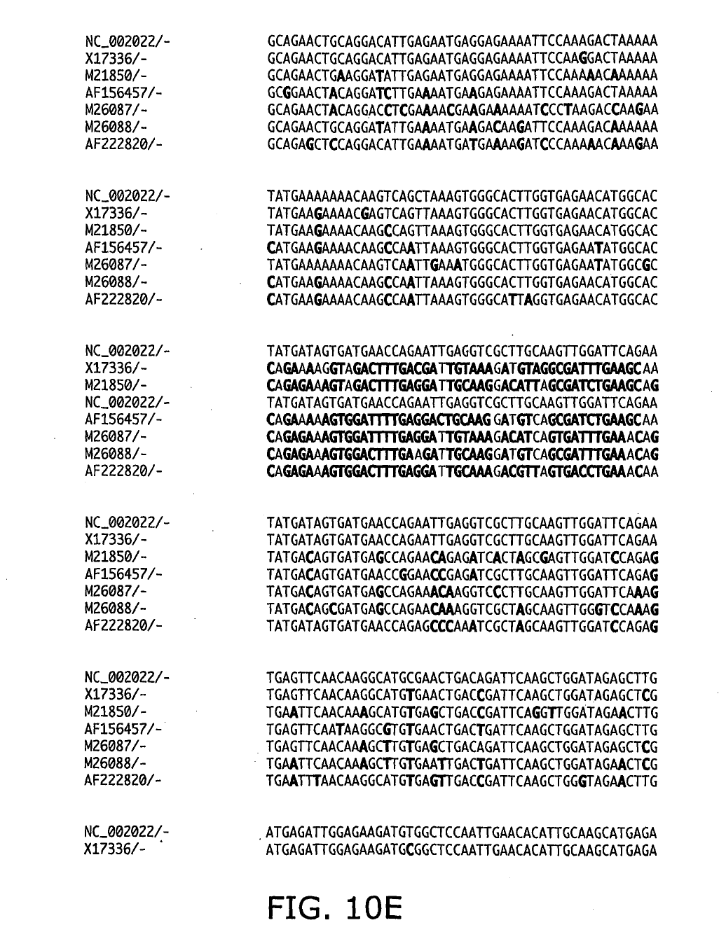

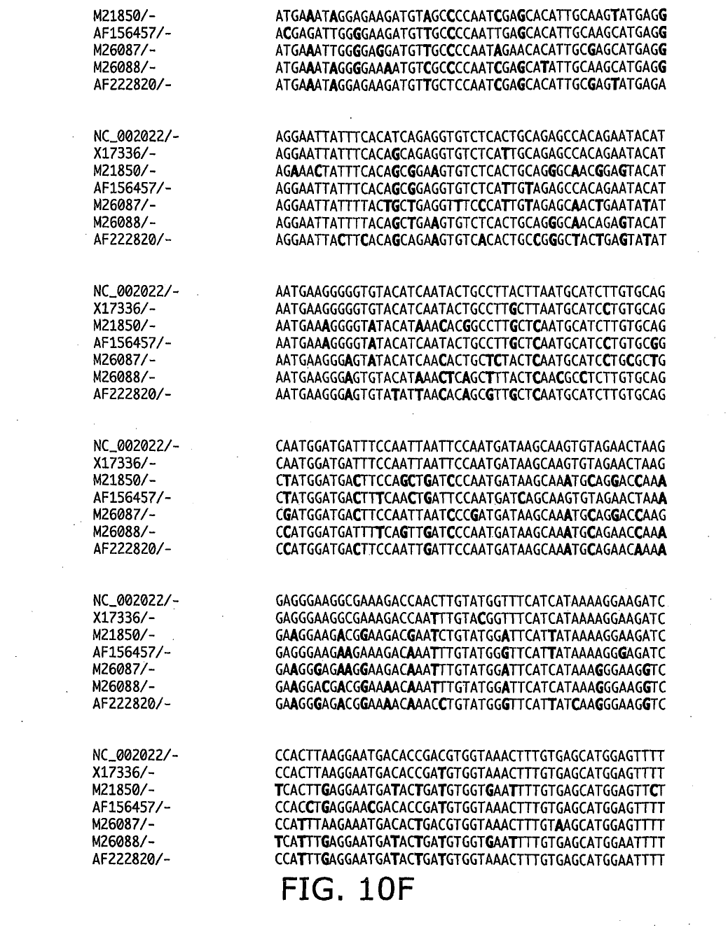

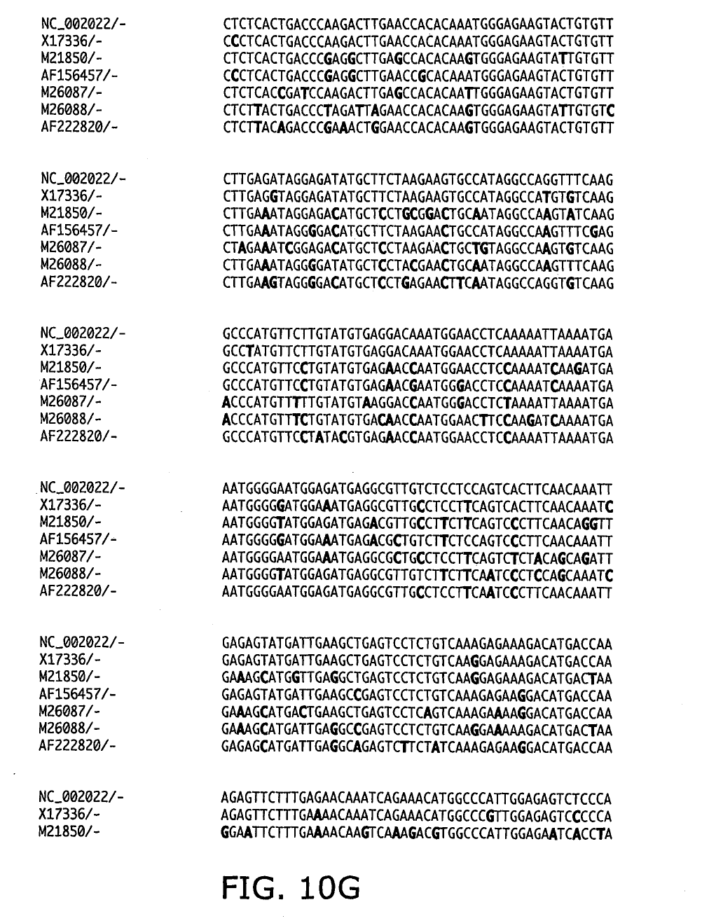

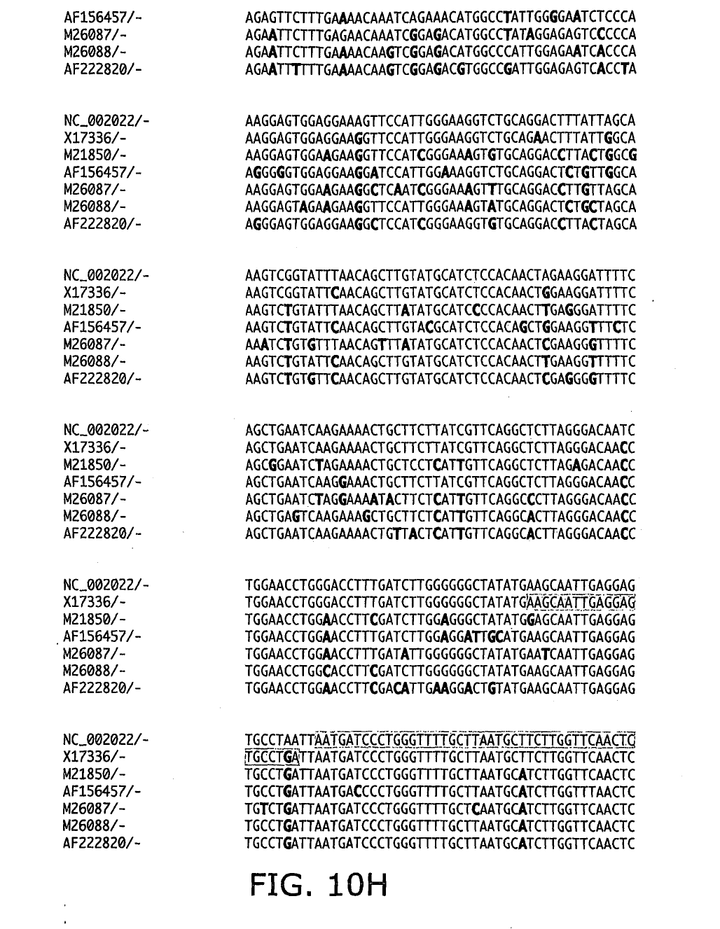

FIG. 10 shows an example of the selection of certain regions of the PA transcript that are highly conserved among five influenza A variants (all of which have different animal hosts of origin) and also among two strains that have a human host of origin, in which regions are considered highly conserved if they differ by either 0, 1, or 2 nucleotides. (Note that the sequences are listed as DNA rather than RNA and therefore contain T rather than U.) The sequence of strain A/Puerto Rico/8/34 (H1N1) was selected as the base sequence, i.e., the sequence with which the other sequences were compared. The other members of the set were A/WSN/33 (H1N1), A/chicken/FPV/Rostock/34 (H7N1), A/turkey/California/189/66 (H9M2), A/Equine/London/1416/73 (H7N7), A/gull/Maryland/704/77 (H13N6), and A/swine/Hong Kong/9/98 (H9N2). Nucleotides that differ from the base sequence are shaded.

Note that in the sequence comparisons in FIGS. 9 and 10 many different highly conserved regions can be selected since large portions of the sequence meet the criteria for being highly conserved. However, sequences that have AA at the 5′ end provide for a 19 nucleotide core sequence and a 2 nucleotide 3′ UU overhang in the complementary (antisense) siRNA strand. Therefore regions that were highly conserved were scanned to identify 21 nucleotide portions that had AA at their 5′ end so that the complementary nucleotides, which are present in the antisense strand of the siRNA, are UU. For example, each of the shaded sequences has AA at its 5′ end. Note that the UU 3′ overhang in the antisense strand of the resulting siRNA molecule may be replaced by TT or dTdT as shown in Table 2. However, it is not necessary that the 2 nt 3′ overhang of the antisense strand is UU.

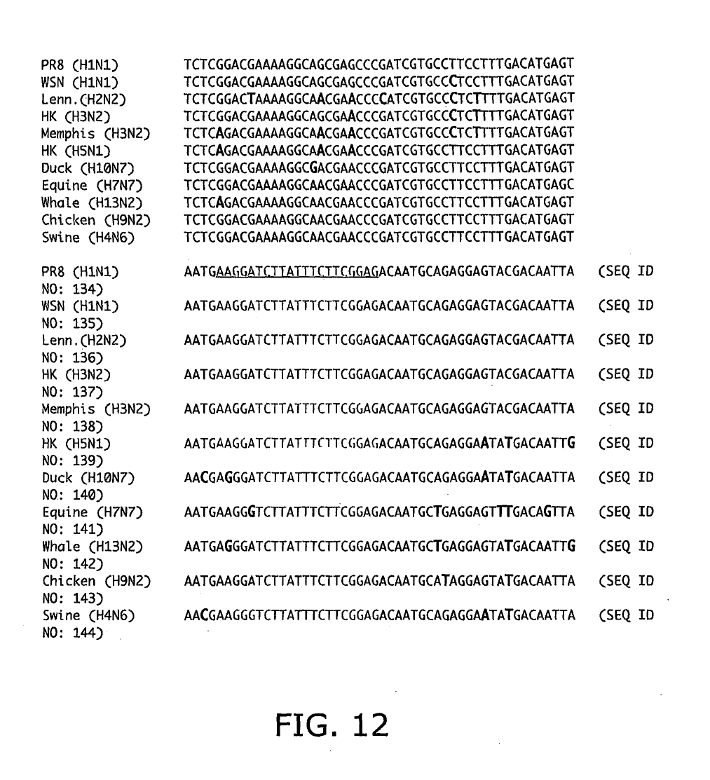

Further illustrating the method, FIG. 12 shows a sequence comparison between a portion of the 3′ region of NP sequences among twelve influenza A virus subtypes or isolates that have either a human or animal host of origin. The underlined sequence and the corresponding portions of the sequences below the underlined sequence were used to design siRNA NP-1496 (see below). These sequences are indicated in FIG. 12. The base sequence is the sequence of strain A/Puerto Rico/8/34. Shaded letters indicate nucleotides that differ from the base sequence.

Table 1A lists 21-nucleotide regions that are highly conserved among a set of influenza virus sequences for each of the viral gene segments. The sequences in Table 1A are listed in 5′ to 3′ direction according to the sequence present in viral mRNA except that T is used instead of U. The numbers indicate the locations of the sequences in the viral genome. For example, PB2-117/137 denotes a sequence extending from position 117 to position 137 in segment PB2. Many of the sequences meet the additional criterion that they have AA at their 5′ end so as to result in a 3′ UU overhang in the complementary strand. For the PA segment, in cases where a one or two nucleotide difference existed, the sequences of the siRNAs were based on the A/PR8/34 (H1N1) strain except for sequence PA-2087/2107 AAGCAATTGAGGAGTGCCTGA (SEQ ID NO: 30), which was based on the A/WSN/33(H1N1) strain. Note that at position 20 five of the six sequences contain a G while the base sequence (accession number NC_{—}002019) contains an A. Thus in this case the sequence of the base sequence was not used for siRNA design. The terms PA-2087 and PA-2087(G) are used interchangeably herein.

To design siRNAs based on the sequences listed in Table 1A, nucleotides 3-21 were selected as the core regions of siRNA sense strand sequences, and a two nt 3′ overhang consisting of dTdT was added to each resulting sequence. A sequence complementary to nucleotides 1-21 of each sequence was selected as the corresponding antisense strand. For example, to design an siRNA based on the highly conserved sequence PA-44/64, i.e., AATGCTTCAATCCGATGATTG (SEQ ID NO: 22) a 19 nt core region having the sequence TGCTTCAATCCGATGATTG (SEQ ID NO: 109) was selected. A two nt 3′ overhang consisting of dTdT was added, resulting (after replacement of T by U) in the sequence 5′-UGCUUCAAUCCGAUGAUUGdTdT-3′ (SEQ ID NO: 79), which was the sequence of the siRNA sense strand. The sequence of the corresponding antisense siRNA strand sequence is complementary to SEQ ID NO: 22, i.e., CAAUCAUCGGAUUGAAGCAdTdT (SEQ ID NO: 80) where T has been replaced by U except for the 2 nt 3′ overhang.

Table 1B lists siRNAs designed based on additional highly conserved regions of influenza virus transcripts. The first 19 nt sequences of the sequences indicated as “sense strand” in Table 1B are sequences of highly conserved regions. The sense strand siRNA sequences are shown with a dTdT overhang at the 3′ end, which does not correspond to influenza virus sequences and is an optional feature of the siRNA. Corresponding antisense strands are also shown, also incorporating a dTdT overhang at the 3′ end as an optional feature. Nomenclature is as in Table 1B. For example, PB2-4/22 sense indicates an siRNA whose sense strand has the sequence of nucleotides 4-22 of the PB2 transcript. PB2-4/22 antisense indicates the complementary antisense strand corresponding to PB2-4/22 sense. For siRNA that target sites in a transcript that span a splice site, the positions within the unspliced transcript are indicated. For example, M-44-52/741-750 indicates that nucleotides corresponding to 44-52 and 741-750 of the genomic sequences are targeted in the spliced mRNA.

Shaded areas in FIGS. 9 and 10 indicate some of the 21 nucleotide regions that meet the criteria for being highly conserved. siRNAs were designed based on these sequences as described above. The actual siRNA sequences that were tested are listed in Table 2.

TABLE 1A
Conserved regions for design of siRNA to interfere
with influenza A virus infection
Segment 1: PB2

| | | |
|---|---|---|
| PB2-117/137 | AATCAAGAAGTACACATCAGG | (SEQ ID NO: 1) |
| PB2-124/144 | AAGTACACATCAGGAAGACAG | (SEQ ID NO: 2) |
| PB2-170/190 | AATGGATGATGGCAATGAAAT | (SEQ ID NO: 3) |
| PB2-195/215 | AATTACAGCAGACAAGAGGAT | (SEQ ID NO: 4) |
| PB2-1614/1634 | AACTTACTCATCGTCAATGAT | (SEQ ID NO: 5) |
| PB2-1942/1962 | AATGTGAGGGGATCAGGAATG | (SEQ ID NO: 6) |
| PB2-2151/2171 | AAGCATCAATGAACTGAGCAA | (SEQ ID NO: 7) |
| PB2-2210/2230 | AAGGAGACGTGGTGTTGGTAA | (SEQ ID NO: 8) |
| PB2-2240/2260 | AACGGGACTCTAGCATACTTA | (SEQ ID NO: 9) |
| PB2-2283/2303 | AAGAATTCGGATGGCCATCAA | (SEQ ID NO: 10) |

Segment 2: PB1

| | | |
|---|---|---|
| PB1-6/26 | AAGCAGGCAAACCATTTGAAT | (SEQ ID NO: 11) |
| PB1-15/35 | AACCATTTGAATGGATGTCAA | (SEQ ID NO: 12) |
| PB1-34/54 | AATCCGACCTTACTTTTCTTA | (SEQ ID NO: 13) |
| PB1-56/76 | AAGTGCCAGCACAAAATGCTA | (SEQ ID NO: 14) |
| PB1-129/149 | AACAGGATACACCATGGATAC | (SEQ ID NO: 15) |
| PB1-1050/1070 | AATGTTCTCAAACAAAATGGC | (SEQ ID NO: 16) |
| PB1-1242/1262 | AATGATGATGGGCATGTTCAA | (SEQ ID NO: 17) |
| PB1-2257/2277 | AAGATCTGTTCCACCATTGAA | (SEQ ID NO: 18) |

Segment 3: PA

| | | |
|---|---|---|
| PA-6/26 | AAGCAGGTACTGATCCAAAAT | (SEQ ID NO: 19) |
| PA-24/44 | AATGGAAGATTTTGTGCGACA | (SEQ ID NO: 20) |
| PA-35/55 | TTGTGCGACAATGCTTCAATC | (SEQ ID NO: 21) |
| PA-44/64 | AATGCTTCAATCCGATGATTG | (SEQ ID NO: 22) |
| PA-52/72 | AATCCGATGATTGTCGAGCTT | (SEQ ID NO: 23) |
| PA-121/141 | AACAAATTTGCAGCAATATGC | (SEQ ID NO: 24) |
| PA-617/637 | AAGAGACAATTGAAGAAAGGT | (SEQ ID NO: 25) |
| PA-711/731 | TAGAGCCTATGTGGATGGATT | (SEQ ID NO: 26) |
| PA-739/759 | AACGGCTACATTGAGGGCAAG | (SEQ ID NO: 27) |
| PA-995/1015 | AACCACACGAAAAGGGAATAA | (SEQ ID NO: 28) |
| PA-2054/2074 | AACCTGGGACCTTTGATCTTG | (SEQ ID NO: 29) |
| PA-2087/2107 | AAGCAATTGAGGAGTGCCTGA | (SEQ ID NO: 30) |
| PA-2110/2130 | AATGATCCCTGGGTTTTGCTT | (SEQ ID NO: 31) |
| PA-2131/2151 | AATGCTTCTTGGTTCAACTCC | (SEQ ID NO: 32) |

Segment 4: HA

| | | |
|---|---|---|
| HA-1119/1139 | TTGGAGCCATTGCCGGTTTTA | (SEQ ID NO: 33) |
| HA-1121/1141 | GGAGCCATTGCCGGTTTTATT | (SEQ ID NO: 34) |
| HA-1571/1591 | AATGGGACTTATGATTATCCC | (SEQ ID NO: 35) |

Segment 5: NP

| | | |
|---|---|---|
| NP-19/39 | AATCACTCACTGAGTGACATC | (SEQ ID NO: 36) |
| NP-42/62 | AATCATGGCGTCCCAAGGCAC | (SEQ ID NO: 37) |
| NP-231/251 | AATAGAGAGAATGGTGCTCTC | (SEQ ID NO: 38) |
| NP-390/410 | AATAAGGCGAATCTGGCGCCA | (SEQ ID NO: 39) |
| NP-393/413 | AAGGCGAATCTGGCGCCAAGC | (SEQ ID NO: 40) |
| NP-708/728 | AATGTGCAACATTCTCAAAGG | (SEQ ID NO: 41) |
| NP-1492/1512 | AATGAAGGATCTTATTTCTTC | (SEQ ID NO: 42) |
| NP-1496/1516 | AAGGATCTTATTTCTTCGGAG | (SEQ ID NO: 43) |
| NP-1519/1539 | AATGCAGAGGAGTACGACAAT | (SEQ ID NO: 44) |

Segment 6: NA

| | | |
|---|---|---|
| NA-20/40 | AATGAATCCAAATCAGAAAAT | (SEQ ID NO: 45) |
| NA704/724 | GAGGACACAAGAGTCTGAATG | (SEQ ID NO: 46) |
| NA-861/881 | GAGGAATGTTCCTGTTACCCT | (SEQ ID NO: 47) |
| NA-901/921 | GTGTGTGCAGAGACAATTGGC | (SEQ ID NO: 48) |

Segment 7: M

| | | |
|---|---|---|
| M-156/176 | AATGGCTAAAGACAAGACCAA | (SEQ ID NO: 49) |
| M-175/195 | AATCCTGTCACCTCTGACTAA | (SEQ IDNO: 50) |
| M-218/238 | ACGCTCACCGTGCCCAGTGAG | (SEQ ID NO: 51) |
| M-244/264 | ACTGCAGCGTAGACGCTTTGT | (SEQ ID NO: 52) |
| M-373/393 | ACTCAGTTATTCTGCTGGTGC | (SEQ ID NO: 53) |
| M-377/397 | AGTTATTCTGCTGGTGCACTT | (SEQ ID NO: 54) |
| M-480/500 | AACAGATTGCTGACTCCCAGC | (SEQ ID NO: 55) |
| M-584/604 | AAGGCTATGGAGCAAATGGCT | (SEQ ID NO: 56) |
| M-598/618 | AATGGCTGGATCGAGTGAGCA | (SEQ ID NO: 57) |
| M-686/706 | ACTCATCCTAGCTCCAGTGCT | (SEQ ID NO: 58) |
| M-731/751 | AATTTGCAGGCCTATCAGAAA | (SEQ ID NO: 59) |
| M-816/836 | ATTGTGGATTCTTGATCGTCT | (SEQ ID NO: 60) |
| M-934/954 | AAGAATATCGAAAGGAACAGC | (SEQ ID NO: 61) |
| M-982/1002 | ATTTTGTCAGCATAGAGCTGG | (SEQ ID NO: 62) |

Segment 8: NS

| | | |
|---|---|---|
| NS-101/121 | AAGAACTAGGTGATGCCCCAT | (SEQ ID NO: 63) |
| NS-104/124 | AACTAGGTGATGCCCCATTCC | (SEQ ID NO: 64) |
| NS-128/148 | ATCGGCTTCGCCGAGATCAGA | (SEQ ID NO: 65) |
| NS-137/157 | GCCGAGATCAGAAATCCCTAA | (SEQ ID NO: 66) |
| NS-562/582 | GGAGTCCTCATCGGAGGACTT | (SEQ ID NO: 67) |
| NS-589/609 | AATGATAACACAGTTCGAGTC | (SEQ ID NO: 68) |

TABLE 1B

Conserved regions for design
of siRNA to interfere with influenza A virus infection

| | |
|---|---|
| Segment 1: PB2 | |
| PB2-4/22 sense | GAAAGCAGGUCAAUUAUAUdTdT (SEQ ID NO: 190) |
| PB2-4/22 antisense | AUAUAAUUGACCUGCUUUCdTdT (SEQ ID NO: 191) |
| PB2-12/30 sense | GUCAAUUAUAUUCAAUAUGdTdT (SEQ ID NO: 192) |
| PB2-12/30 antisense | CAUAUUGAAUAUAAUUGACdTdT (SEQ ID NO: 193) |
| PB2-68/86 sense | CUCGCACCCGCGAGAUACUdTdT (SEQ ID NO: 194) |
| PB2-68/86 antisense | AGUAUCUCGCGGGUGCGAGdTdT (SEQ ID NO: 195) |
| PB2-115/133 sense | AUAAUCAAGAAGUACACAUdTdT (SEQ ID NO: 196) |
| PB2-115/133 antisense | AUGUGUACUUCUUGAUUAUdTdT (SEQ ID NO: 197) |
| PB2-167/185 sense | UGAAAUGGAUGAUGGCAAUdTdT (SEQ ID NO: 198) |
| PB2-167/185 antisense | AUUGCCAUCAUCCAUUUCAdTdT (SEQ ID NO: 199) |
| PB2-473/491 sense | CUGGUCAUGCAGAUCUCAGdTdT (SEQ ID NO: 200) |
| PB2-473/491 antisense | CUGAGAUCUGCAUGACCAGdTdT (SEQ ID NO: 201) |
| PB2-956/974 sense | UAUGCAAGGCUGCAAUGGGdTdT (SEQ ID NO: 202) |
| PB2-956/974 antisense | CCCAUUGCAGCCUUGCAUAdTdT (SEQ ID NO: 203) |
| PB2-1622/1640 sense | CAUCGUCAAUGAUGUGGGAdTdT (SEQ ID NO: 204) |
| PB2-1622/1640 antisense | UCCCACAUCAUUGACGAUGdTdT (SEQ ID NO: 205) |
| Segment 2: PB1 | |
| PB1-1124/1142 sense | AAAUACCUGCAGAAAUGCUdTdT (SEQ ID NO: 206) |
| PB1-1124/1142 antisense | AGCAUUUCUGCAGGUAUUUdTdT (SEQ ID NO: 207) |
| PB1-1618/1636 sense | AACAAUAUGAUAAACAAUGdTdT (SEQ ID NO: 208) |
| PB1-1618/1636 antisense | CAUUGUUUAUCAUAUUGUUdTdT (SEQ ID NO: 209) |
| Segment 3: PA | |
| PA-3/21 sense | CGAAAGCAGGUACUGAUCCdTdT (SEQ ID NO: 210) |
| PA-3/21 antisense | GGAUCAGUACCUGCUUUCGdTdT (SEQ ID NO: 211) |
| PA-544/562 sense | AGGCUAUUCACCAUAAGACdTdT (SEQ ID NO: 212) |
| PA-544/562 antisense | GUCUUAUGGUGAAUAGCCUdTdT (SEQ ID NO: 213) |
| PA-587/605 sense | GGGAUUCCUUUCGUCAGUCdTdT (SEQ ID NO: 214) |
| PA-587/605 antisense | GACUGACGAAAGGAAUCCCdTdT (SEQ ID NO: 215) |
| PA-1438/1466 sense | GCAUCUUGUGCAGCAAUGGdTdT (SEQ ID NO: 216) |
| PA-1438/1466 antisense | CCAUUGCUGCACAAGAUGCdTdT (SEQ ID NO: 217) |
| PA-2175/2193 sense | GUUGUGGCAGUGCUACUAUdTdT (SEQ ID NO: 218) |
| PA-2175/2193 antisense | AUAGUAGCACUGCCACAACdTdT (SEQ ID NO: 219) |
| PA-2188/2206 sense | UACUAUUUGCUAUCCAUACdTdT (SEQ ID NO: 220) |
| PA-2188/2206 antisense | GUAUGGAUAGCAAAUAGUAdTdT (SEQ ID NO: 221) |
| Segment 5: NP | |
| NP-14/32 sense | UAGAUAAUCACUCACUGAGdTdT (SEQ ID NO: 222) |
| NP-14/32 antisense | CUCAGUGAGUGAUUAUCUAdTdT (SEQ ID NO: 223) |
| NP-50/68 sense | CGUCCCAAGGCACCAAACGdTdT (SEQ ID NO: 224) |
| NP-50/68 antisense | CGUUUGGUGCCUUGGGACGdTdT (SEQ ID NO: 225) |
| NP-1505/1523 sense | AUUUCUUCGGAGACAAUGCdTdT (SEQ ID NO: 226) |
| NP-1505/1523 antisense | GCAUUGUCUCCGAAGAAAUdTdT (SEQ ID NO: 227) |
| NP-1521/1539 sense | UGCAGAGGAGUACGACAAUdTdT (SEQ ID NO: 228) |
| NP-1521/1539 antisense | AUUGUCGUACUCCUCUGCAdTdT (SEQ ID NO: 229) |
| NP-1488/1506 sense | GAGTAATGAAGGATCTTATdTdT (SEQ ID NO: 230) |
| NP-1488/1506 antisense | ATAAGATCCTTCATTACTCdTdT (SEQ ID NO: 231) |
| Segment 7: M | |
| M-3/21 sense | CGAAAGCAGGUAGAUAUUGdTdT (SEQ ID NO: 232) |
| M-3/21 antisense | CAAUAUCUACCUGCUUUCGdTdT (SEQ ID NO: 233) |
| M-13/31 sense | UAGAUAUUGAAAGAUGAGUdTdT (SEQ ID NO: 234) |
| M-13/31 antisense | ACUCAUCUUUCAAUAUCUAdTdT (SEQ ID NO: 235) |
| M-150/158 sense | UCAUGGAAUGGCUAAAGACdTdT (SEQ ID NO: 236) |
| M-150/158 antisense | GUCUUUAGCCAUUCCAUGAdTdT (SEQ ID NO: 237) |
| M-172/190 sense | ACCAAUCCUGUCACCUCUGdTdT (SEQ ID NO: 238) |
| M-172/190 antisense | CAGAGGUGACAGGAUUGGUdTdT (SEQ ID NO: 239) |
| M-211/229 sense | UGUGUUCACGCUCACCGUGdTdT (SEQ ID NO: 240) |
| M-211/229 antisense | CACGGUGAGCGUGAACACAdTdT (SEQ ID NO: 241) |
| M-232/250 sense | CAGUGAGCGAGGACUGCAGdTdT (SEQ ID NO: 242) |
| M-232/250 antisense | CUGCAGUCCUCGCUCACUGdTdT (SEQ ID NO: 243) |
| M-255/273 sense | GACGCUUUGUCCAAAAUGCdTdT (SEQ ID NO: 244) |
| M-255/273 antisense | GCAUUUUGGACAAAGCGUCdTdT (SEQ ID NO: 245) |
| M-645/663 sense | GUCAGGCUAGGCAAAUGGUdTdT (SEQ ID NO: 246) |
| M-645/663 antisense | ACCAUUUGCCUAGCCUGACdTdT (SEQ ID NO: 247) |
| M-723/741 sense | UUCUUGAAAAUUUGCAGGCdTdT (SEQ ID NO: 248) |
| M-723/741 antisense | GCCUGCAAAUUUUCAAGAAdTdT (SEQ ID NO: 249) |
| M-808/826 sense | UCAUUGGGAUCUUGCACUUdTdT (SEQ ID NO: 250) |
| M-808/826 antisense | AAGUGCAAGAUCCCAAUGAdTdT (SEQ ID NO: 251) |
| M-832/850 sense | UGUGGAUUCUUGAUCGUCUdTdT (SEQ ID NO: 252) |
| M-832/850 antisense | AGACGAUCAAGAAUCCACAdTdT (SEQ ID NO: 253) |
| M-986/1004 sense | UGUCAGCAUAGAGCUGGAGdTdT (SEQ ID NO: 254) |
| M-986/1004 antisense | CUCCAGCUCUAUGCUGACAdTdT (SEQ ID NO: 255) |
| M-44-52/741-750 sense | GTCGAAACGCCTATCAGAAdTdT (SEQ ID NO: 256) |
| M-44-52/741-750 antisense | UUCUGAUAGGCGUUUCGACdTdT (SEQ ID NO: 257) |
| Segment 8: NS | |
| NS-5/23 sense | AAAAGCAGGGUGACAAAGAdTdT (SEQ ID NO: 258) |
| NS-5/23 antisense | UCUUUGUCACCCUGCUUUUdTdT (SEQ ID NO: 259) |
| NS-9/27 sense | GCAGGGUGACAAAGACAUAdTdT (SEQ ID NO: 260) |
| NS-9/27 antisense | UAUGUCUUUGUCACCCUGCdTdT (SEQ ID NO: 261) |
| NS-543/561 sense | GGAUGUCAAAAAUGCAGUUdTdT (SEQ ID NO: 262) |
| NS-543/561 antisense | AACUGCAUUUUUGACAUCCdTdT (SEQ ID NO: 263) |
| NS-623/641 sense | AGAGAUUCGCUUGGAGAAGdTdT (SEQ ID NO: 264) |
| NS-623/641 antisense | CUUCUCCAAGCGAAUCUCUdTdT (SEQ ID NO: 265) |
| NS-642/660 sense | CAGUAAUGAGAAUGGGAGAdTdT (SEQ ID NO: 266) |
| NS-642/660 antisense | UCUCCCAUUCUCAUUACUGdTdT (SEQ ID NO: 267) |
| NS-831/849 sense | UUGUGGAUUCUUGAUCGUCdTdT (SEQ ID NO: 268) |
| NS-831/839 antisense | GACGAUCAAGAAUCCACAAdTdT (SEQ ID NO: 269) |

### Example 2

### siRNAs that Target Viral RNA Polymerase or Nucleoprotein Inhibit Influenza A Virus Production

Materials and Methods

Cell Culture. Madin-Darby canine kidney cells (MDCK), a kind gift from Dr. Peter Palese, Mount Sinai School of Medicine, New York, N.Y., were grown in DMEM medium containing 10% heat-inactivated FCS, 2 mM L-glutamine, 100 units/ml penicillin, and 100 μg/ml streptomycin. Cells were grown at 37° C., 5% CO₂. For electroporation, the cells were kept in serum-free RPMI 1640 medium. Virus infections were done in infection medium (DMEM, 0.3% bovine serum albumin (BSA, Sigma, St. Louis, Mo.), 10 mM Hepes, 100 units/ml penicillin, and 100 μg/ml streptomycin).

Viruses. Influenza viruses A/PR/8/34 (PR8) and A/WSN/33 (WSN), subtypes H1N1, kind gifts from Dr. Peter Palese, Mount Sinai School of Medicine, were grown for 48 h in 10-day-embryonated chicken eggs (Charles River laboratories, MA) at 37° C. Allantoic fluid was harvested 48 h after virus inoculation and stored at −80° C. The same virus strains and methods were used throughout the examples described herein.

siRNAs. siRNAs were designed as described above. In addition to conforming to the selection criteria described in Example 1, the siRNAs were generally designed in accordance with principles described in Technical Bulletin #003—Revision B, “siRNA Oligonucleotides for RNAi Applications”, available from Dharmacon Research, Inc., Lafayette, Colo. 80026. Technical Bulletins #003 and #004 from Dharmacon contain a variety of information relevant to siRNA design parameters, synthesis, etc., and are incorporated herein by reference. Sense and antisense sequences that were tested are listed in Table 2.

TABLE 2
siRNA Sequences

| | |
|---|---|
| Name | siRNA sequence (5′-3′) |

| | |
|---|---|
| PB2-2210/2230 (sense) | GGAGACGUGGUGUUGGUAAdTdT (SEQ ID NO: 69) |
| PB2-2210/2230 (antisense) | UUACCAACACCACGUCUCCdTdT (SEQ ID NO: 70) |
| PB2-2240/2260 (sense) | CGGGACUCUAGCAUACUUAdTdT (SEQ ID NO: 71) |
| PB2-2240/2260 (antisense) | UAAGUAUGCUAGAGUCCCGdTdT (SEQ ID NO: 72) |
| PB1-6/26 (sense) | GCAGGCAAACCAUUUGAAUdTdT (SEQ ID NO: 73) |
| PB1-6/26 (antisense) | AUUCAAAUGGUUUGCCUGCdTdT (SEQ ID NO: 74) |
| PB1-129/149 (sense) | CAGGAUACACCAUGGAUACdTdT (SEQ ID NO: 75) |
| PB1-129/149 (antisense) | GUAUCCAUGGUGUAUCCUGdTdT (SEQ ID NO: 76) |
| PB1-2257/2277 (sense) | GAUCUGUUCCACCAUUGAAdTdT (SEQ ID NO: 77) |
| PB1-2257/2277 (antisense) | UUCAAUGGUGGAACAGAUCdTdT (SEQ ID NO: 78) |
| PA-44/64 (sense) | UGCUUCAAUCCGAUGAUUGdTdT (SEQ ID NO: 79) |
| PA-44/64 (antisense) | CAAUCAUCGGAUUGAAGCAdTdT (SEQ ID NO: 80) |
| PA-739/759 (sense) | CGGCUACAUUGAGGGCAAGdTdT (SEQ ID NO: 81) |
| PA-739/759 (antisense) | CUUGCCCUCAAUGUAGCCGdTdT (SEQ ID NO: 82) |
| PA-2087/2107 (G) (sense) | GCAAUUGAGGAGUGCCUGAdTdT (SEQ ID NO: 83) |
| PA-2087/2107 (G) (antisense) | UCAGGCACUCCUCAAUUGCdTdT (SEQ ID NO: 84) |
| PA-2110/2130 (sense) | UGAUCCCUGGGUUUUGCUUdTdT (SEQ ID NO: 85) |
| PA-2110/2130 (antisense) | AAGCAAAACCCAGGGAUCAdTdT (SEQ ID NO: 86) |
| PA-2131/2151 (sense) | UGCUUCUUGGUUCAACUCCdTdT (SEQ ID NO: 87) |
| PA-2131/2151 (antisense) | GGAGUUGAACCAAGAAGCAdTdT (SEQ ID NO: 88) |
| NP-231/251 (sense) | UAGAGAGAAUGGUGCUCUCdTdT (SEQ ID NO: 89) |
| NP-231/251 (antisense) | GAGAGCACCAUUCUCUCUAdTdT (SEQ ID NO: 90) |
| NP-390/410 (sense) | UAAGGCGAAUCUGGCGCCAdTdT (SEQ ID NO: 91) |
| NP-390/410 (antisense) | UGGCGCCAGAUUCGCCUUAdTdT (SEQ ID NO: 92) |
| NP-1496/1516 (sense) | GGAUCUUAUUUCUUCGGAGdTdT (SEQ ID NO: 93) |
| NP-1496/1516 (antisense) | CUCCGAAGAAAUAAGAUCCdTdT (SEQ ID NO: 94) |
| NP-1496/1516a (sense) | GGAUCUUAUUUCUUCGGAGAdTdT (SEQ ID NO: 188) |
| NP-1496/1516a (antisense) | UCUCCGAAGAAAUAAGAUCCdTdT (SEQ ID NO: 189) |
| M-37/57 (sense) | CCGAGGUCGAAACGUACGUdTdT (SEQ ID NO: 95) |
| M-37/57 (antisense) | ACGUACGUUUCGACCUCGGdTdT (SEQ ID NO: 96) |
| M-480/500 (sense) | CAGAUUGCUGACUCCCAGCdTdT (SEQ ID NO: 97) |
| M-480/500 (antisense) | GCUGGGAGUCAGCAAUCUGdTdT (SEQ ID NO: 98) |
| M-598/618 (sense) | UGGCUGGAUCGAGUGAGCAdTdT (SEQ ID NO: 99) |
| M-598/618 (antisense) | UGCUCACUCGAUCCAGCCAdTdT (SEQ ID NO: 100) |
| M-934/954 (sense) | GAAUAUCGAAAGGAACAGCdTdT (SEQ ID NO: 101) |
| M-934/954 (antisense) | GCUGUUCCUUUCGAUAUUCdTdT (SEQ ID NO: 102) |
| NS-128/148 (sense) | CGGCUUCGCCGAGAUCAGAdAdT (SEQ ID NO: 103) |
| NS-128/148 (antisense) | UCUGAUCUCGGCGAAGCCGdAdT (SEQ ID NO: 104 |
| NS-562/582 (R) (sense) | GUCCUCCGAUGAGGACUCCdTdT (SEQ ID NO: 105) |
| NS-562/582 (R) (antisense) | GGAGUCCUCAUCGGAGGACdTdT (SEQ ID NO: 106) |
| NS-589/609 (sense) | UGAUAACACAGUUCGAGUCdTdT (SEQ ID NO: 107) |
| NS-589/609 (antisense) | GACUCGAACUGUGUUAUCAdTdT (SEQ ID NO: 108) |

All siRNAs were synthesized by Dharmacon Research (Lafayette, Colo.) using 2′ACE protection chemistry. The siRNA strands were deprotected according to the manufacturer's instructions, mixed in equimolar ratios and annealed by heating to 95° C. and slowly reducing the temperature by 1° C. every 30 s until 35° C. and 1° C. every min until 5° C.

siRNA electroporation. Log-phase cultures of MDCK cells were trypsinized, washed and resuspended in serum-free RPMI 1640 at 2×10⁷ cells per ml. 0.5 ml of cells were placed into a 0.4 cm cuvette and were electroporated using a Gene Pulser apparatus (Bio-Rad) at 400 V, 975 μF with 2.5 nmol siRNAs. Electroporation efficiencies were approximately 30-40% of viable cells. Electroporated cells were divided into 3 wells of a 6-well plate in DMEM medium containing 10% FCS and incubated at 37° C., 5% CO₂.

Viral infection. Six to eight h following electroporation, the serum-containing medium was washed away and 100 μl of PR8 or WSN virus at the appropriate multiplicity of infection was inoculated into the wells, each of which contained approximately 10⁶ cells. Cells were infected with either 1,000 PFU (one virus per 1,000 cells; MOI=0.001) or 10,000 PFU (one virus per 100 cells; MOI=0.01) of virus. After 1 h incubation at room temperature, 2 ml of infection medium with 4 μg/ml of trypsin was added to each well and the cells were incubated at 37° C., 5% CO₂. At indicated times, supernatants were harvested from infected cultures and the titer of virus was determined by hemagglutination of chicken erythrocytes (50 μl, 0.5%, Charles River laboratories, MA).

Measurement of Viral Titer. Supernatants were harvested at 24, 36, 48, and 60 hours after infection. Viral titer was measured using a standard hemagglutinin assay as described in Knipe D M, Howley, P M, Fundamental Virology, 4th edition, p. 34-35. The hemagglutination assay was done in V-bottomed 96-well plates. Serial 2-fold dilutions of each sample were incubated for 1 h on ice with an equal volume of a 0.5% suspension of chicken erythrocytes (Charles River Laboratories). Wells containing an adherent, homogeneous layer of erythrocytes were scored as positive. For plaque assays, serial 10-fold dilutions of each sample were titered for virus as described in Fundamental Virology, 4^{th} edition, p. 32, as well known in the art.

Results

To investigate the feasibility of using siRNA to suppress influenza virus replication, various influenza virus A RNAs were targeted. Specifically, the MDCK cell line, which is readily infected and widely used to study influenza virus, was utilized. Each siRNA was individually introduced into populations of MDCK cells by electroporation. siRNA targeted to GFP (sense: 5′-GGCUACGUCCAGGAGCGCAUU-3′ (SEQ ID NO: 110); antisense: 5′-UGCGCUCCUGGACGUAGCCUU-3′ (SEQ ID NO: 111)) was used as control. This siRNA is referred to as GFP-949. In subsequent experiments (described in examples below) the UU overhang at the 3′ end of both strands was replaced by dTdT with no effect on results. A mock electroporation was also performed as a control. Eight hours after electroporation cells were infected with either influenza A virus PR8 or WSN at an MOI of either 0.1 or 0.01 and were analyzed for virus production at various time points (24, 36, 48, 60 hours) thereafter using a standard hemagglutination assay. GFP expression was assayed by flow cytometry using standard methods.

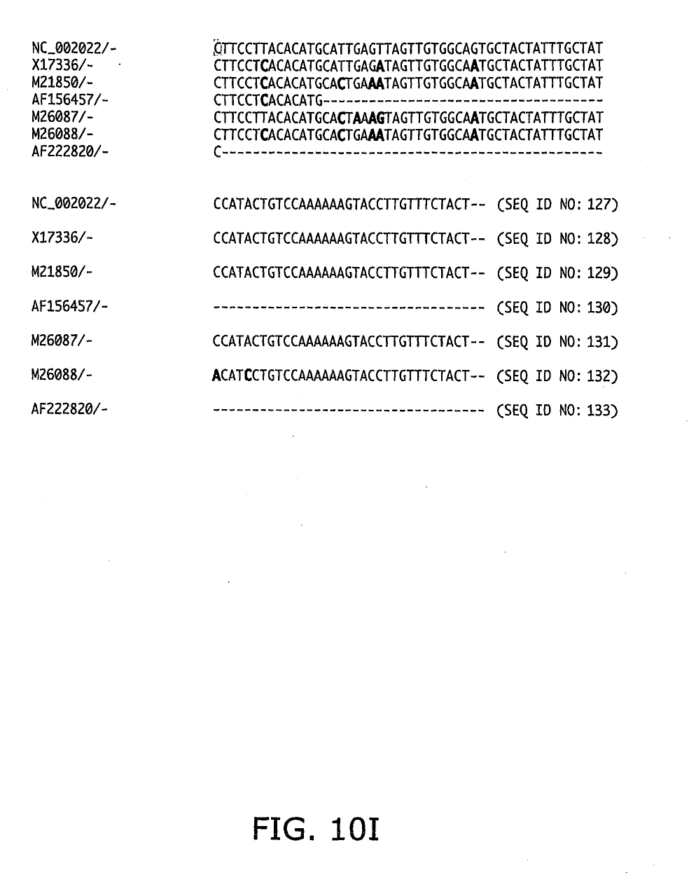

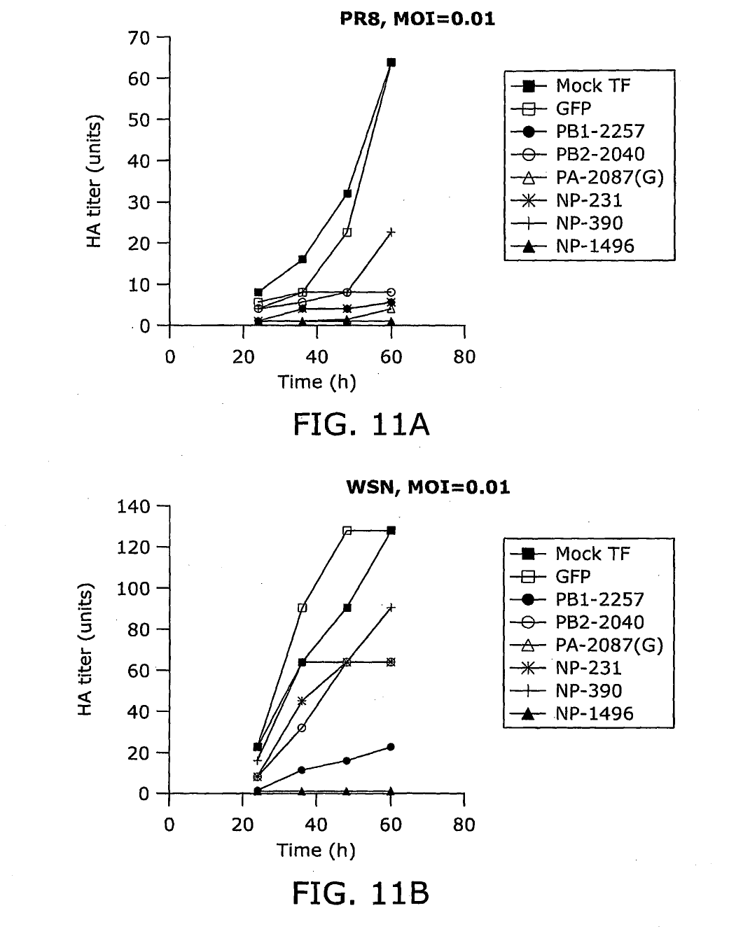

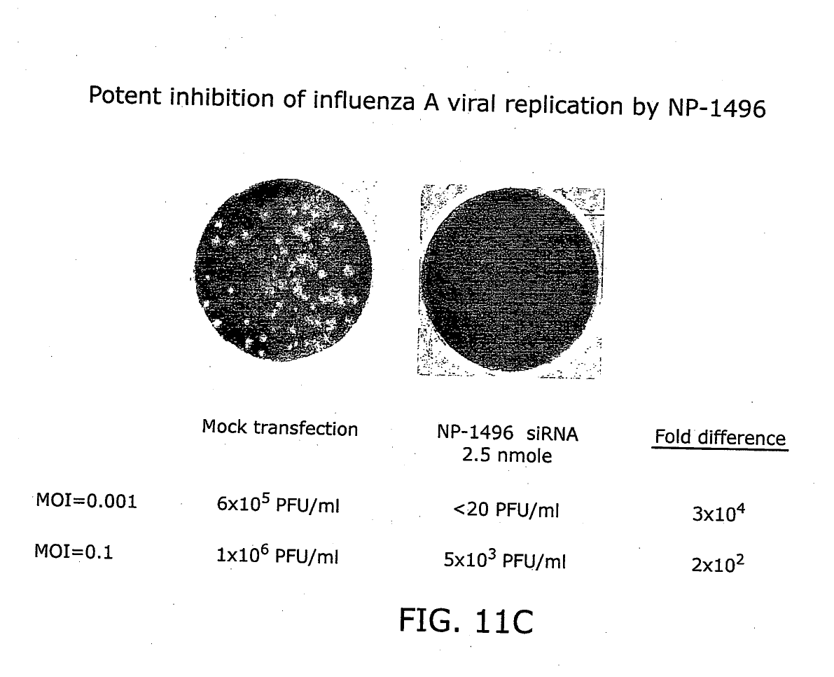

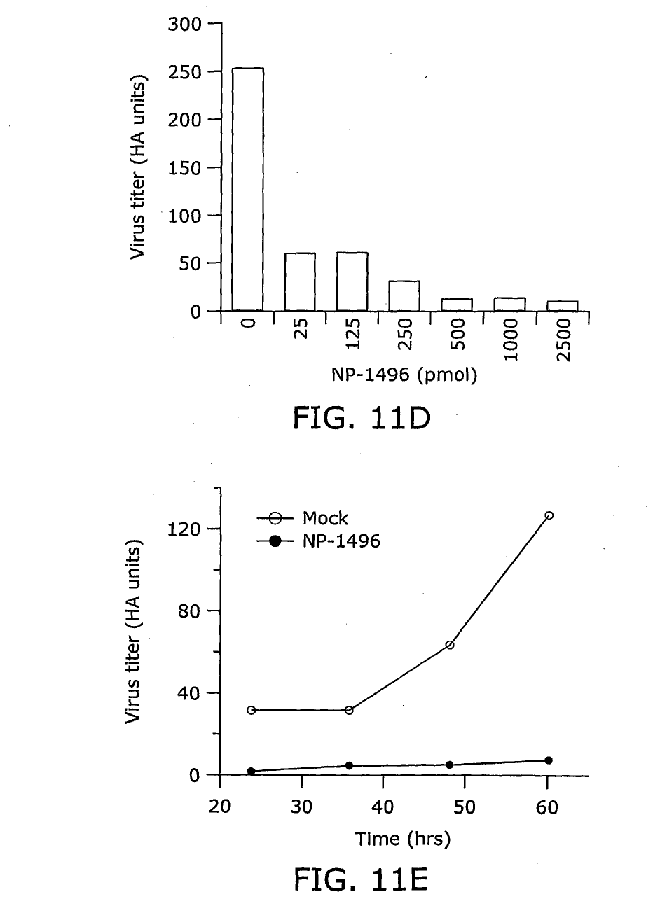

FIGS. 11A and 11B compare results of experiments in which the ability of individual siRNAs to inhibit replication of influenza virus A strain A/Puerto Rico/8/34 (H1N1) (FIG. 11A) or influenza virus A strain A/WSN/33 (H1N1) (FIG. 11B) was determined by measuring HA titer. Thus a high HA titer indicates a lack of inhibition while a low HA titer indicates effective inhibition. MDCK cells were infected at an MOI of 0.01. For these experiments one siRNA that targets the PB1 segment (PB1-2257/2277), one siRNA that targets the PB2 segment (PB2-2240/2260), one siRNA that targets the PA segment (PA-2087/2107 (G)), and three different siRNAs that target the NP genome and transcript (NP-231/251, NP-390/410, and NP-1496/1516) were tested. Note that the legends on FIGS. 11A and 11B list only the 5′ nucleotide of the siRNAs.

Symbols in FIGS. 11A and 11B are as follows: Filled squares represents control cells that did not receive siRNA. Open squares represents cells that received the GFP control siRNA. Filled circles represent cells that received siRNA PB1-2257/2277. Open circles represent cells that received siRNA PB2-2240/2260. Open triangles represent cells that received siRNA PA-2087/2107 (G). The X symbol represents cells that received siRNA NP-231/251. The + symbol represents cells that received siRNA NP-390/410. Closed triangles represent cells that received siRNA NP-1496/1516. Note that in the graphs certain symbols are sometimes superimposed. For example, in FIG. 11B the open and closed triangles are superimposed. Tables 3 and 4, which list the numerical values for each point, may be consulted for clarification.

As shown in FIGS. 11A and 11B (Tables 3 and 4), in the absence of siRNA (mock TF) or the presence of control (GFP) siRNA, the titer of virus increased over time, reaching a peak at approximately 48-60 hours after infection. In contrast, at 60 hours the viral titer was significantly lower in the presence of any of the siRNAs. For example, in strain WSN the HA titer (which reflects the level of virus) was approximately half as great in the presence of siRNAs PB2-2240 or NP-231 than in the controls. In particular, the level of virus was below the detection limit (10,000 PFU/ml) in the presence of siRNA NP-1496 in both strains. This represents a decrease by a factor of more than 60-fold in the PR8 strain and more than 120-fold in the WSN strain. The level of virus was also below the detection limit (10,000 PFU/ml) in the presence of siRNA PA-2087(G) in strain WSN and was extremely low in strain PR8. Suppression of virus production by siRNA was evident even from the earliest time point measured. Effective suppression, including suppression of virus production to undetectable levels (as determined by HA titer) has been observed at time points as great as 72 hours post-infection.

Table 5 summarizes results of siRNA inhibition assays at 60 hours in MDCK cells expressed in terms of fold inhibition. Thus a low value indicates lack of inhibition while a high value indicates effective inhibition. The location of siRNAs within a viral gene is indicated by the number that follows the name of the gene. As elsewhere herein, the number represents the starting nucleotide of the siRNA in the gene. For example, NP-1496 indicates an siRNA specific for NP, the first nucleotide starting at nucleotide 1496 of the NP sequence. Values shown (fold-inhibition) are calculated by dividing hemagglutinin units from mock transfection by hemagglutinin units from transfection with the indicated siRNA; a value of 1 means no inhibition.

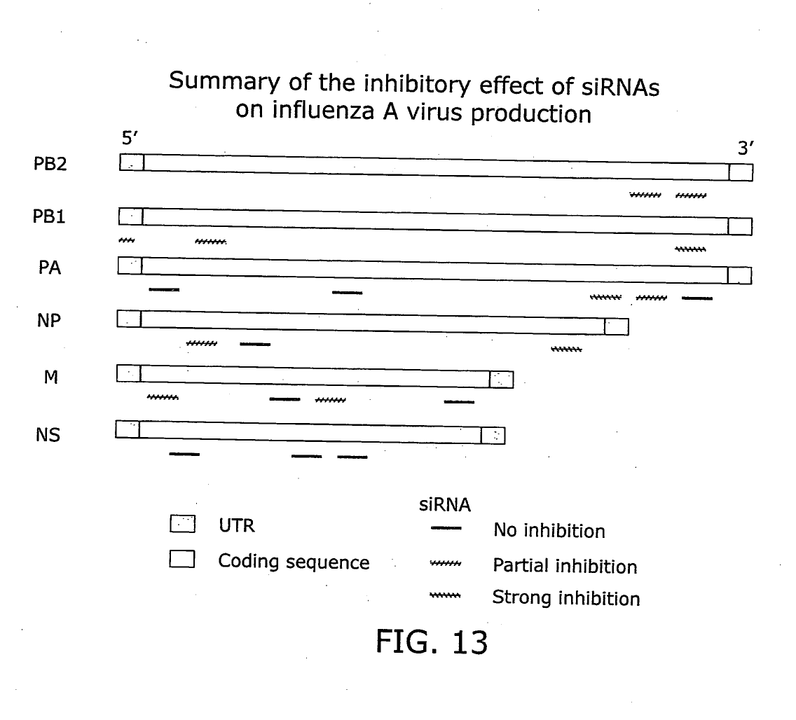

A total of twenty siRNAs, targeted to 6 segments of the influenza virus genome (PB2, PB1, PA, NP, M and NS), were tested in the MDCK cell line system (Table 5). About 15% of the siRNA (PB1-2257, PA-2087G and NP-1496) tested displayed a strong effect, inhibiting viral production by more than 100 fold in most cases at MOI=0.001 and by 16 to 64 fold at MOI=0.01, regardless of whether PR8 or WSN virus was used. In particular, when siRNA NP-1496 or PA-2087 was used, inhibition was so pronounced that culture supernatants lacked detectable hemagglutinin activity. These potent siRNAs target 3 different viral gene segments: PB1 and PA, which are involved in the RNA transcriptase complex, and NP which is a single-stranded RNA binding nucleoprotein. Consistent with findings in other systems, the sequences targeted by these siRNAs are all positioned relatively close to the 3-prime end of the coding region (FIG. 13).

Approximately 40% of the siRNAs significantly inhibited virus production, but the extent of inhibition varied depending on certain parameters. Approximately 15% of siRNAs potently inhibited virus production regardless of whether PR8 or WSN virus was used. However, in the case of certain siRNAs, the extent of inhibition varied somewhat depending on whether PR8 or WSN was used. Some siRNAs significantly inhibited virus production only at early time points (24 to 36 hours after infection) or only at lower dosage of infection (MOI=0.001), such as PB2-2240, PB1-129, NP-231 and M-37. These siRNAs target different viral gene segments, and the corresponding sequences are positioned either close to 3-prime end or 5-prime end of the coding region (FIG. 13). Tables 5A and 5B present results of the assays. Approximately 45% of the siRNAs had no discernible effect on the virus titer, indicating that they were not effective in interfering with influenza virus production in MDCK cells. In particular, none of the four siRNAs which target the NS gene segment showed any inhibitory effect.

To estimate virus titers more precisely, plaque assays with culture supernatants were performed (at 60 hrs) from culture supernatants obtained from virus-infected cells that had undergone mock transfection or transfection with NP-1496. Approximately 6×10⁵ pfu/ml was detected in mock supernatant, whereas no plaques were detected in undiluted NP-1496 supernatant (FIG. 11C). As the detection limit of the plaque assay is about 20 pfu (plaque forming unit)/ml, the inhibition of virus production by NP-1496 is at least about 30,000 fold. Even at an MOI of 0.1, NP-1496 inhibited virus production about 200-fold.

To determine the potency of siRNA, a graded amount of NP-1496 was transfected into MDCK cells followed by infection with PR8 virus. Virus titers in the culture supernatants were measured by hemagglutinin assay. As the amount of siRNA decreased, virus titer increased in the culture supernatants as shown in FIG. 11D. However, even when as little as 25 pmol of siRNA was used for transfection, approximately 4-fold inhibition of virus production was detected as compared to mock transfection, indicating the potency of NP-1496 siRNA in inhibiting influenza virus production.

For therapy, it is desirable for siRNA to be able to effectively inhibit an existing virus infection. In a typical influenza virus infection, new virions are released beginning at about 4 hours after infection. To determine whether siRNA could reduce or eliminate infection by newly released virus in the face of an existing infection, MDCK cells were infected with PR8 virus for 2 hours and then transfected with NP-1496 siRNA. As shown in FIG. 11E, virus titer increased steadily over time following mock transfection, whereas virus titer increased only slightly in NP-1496 transfected cells. Thus administration of siRNA after virus infection is effective.

Together, these results show that (i) certain siRNAs can potently inhibit influenza virus production; (ii) influenza virus production can be inhibited by siRNAs specific for different viral genes, including those encoding NP, PA, and PB 1 proteins; and (iii) siRNA inhibition occurs in cells that were infected previously in addition to cells infected simultaneously with or following administration of siRNAs.

TABLE 3
Inhibition of Virus Strain A/Puerto Rico/8/34 (H1N1) Production
by siRNAs
siRNA

| | | | PB1- | PB2- | PA- | | | NP- |
|---|---|---|---|---|---|---|---|---|
| | Mock | GFP | 2257 | 2040 | 2087(G) | NP-231 | NP-390 | 1496 |
| 24 hr | 8 | 8 | 1 | 4 | 1 | 1 | 4 | 1 |
| 36 hr | 16 | 8 | 4 | 8 | 1 | 4 | 8 | 1 |
| 48 hr | 32 | 32 | 4 | 8 | 2 | 4 | 8 | 1 |
| 60 hr | 64 | 64 | 8 | 8 | 4 | 8 | 32 | 1 |

TABLE 4
Inhibition of Virus Strain A/WSN/33 (H1N1) Production by siRNAs
siRNA

| | | | PB1- | PB2- | PA- | | | NP- |
|---|---|---|---|---|---|---|---|---|
| | Mock | GFP | 2257 | 2040 | 2087(G) | NP-231 | NP-390 | 1496 |
| 24 hr | 32 | 32 | 1 | 8 | 1 | 8 | 16 | 1 |
| 36 hr | 64 | 128 | 16 | 32 | 1 | 64 | 64 | 1 |
| 48 hr | 128 | 128 | 16 | 64 | 1 | 64 | 64 | 1 |
| 60 hr | 128 | 128 | 32 | 64 | 1 | 64 | 128 | 1 |

TABLE 5A
Effects of 20 siRNAs on influenza virus production in MDCK cells

| | | Infecting virus (MOI) | | | | |
|---|---|---|---|---|---|---|
| | | PR8 | PR8 | PR8 | WSN | WSN |
| | siRNA | (0.001) | (0.01) | (0.1) | (0.001) | (0.01) |
| Exp. 1 | GFP-949 | 2 | 1 | | | |
| | PB2-2210 | 16 | 8 | | | |
| | PB2-2240 | 128 | 16 | | | |
| | PB1-6 | 4 | 4 | | | |
| | PB1-129 | 128 | 16 | | | |
| | PB1-2257 | 256 | 64 | | | |
| Exp. 2 | GFP-949 | 2 | 1 | | | |
| | PA-44 | 2 | 1 | | | |
| | PA-739 | 4 | 2 | | | |
| | PA-2087 | 128 | 16 | | | |
| | PA-2110 | 8 | 4 | | | |
| | PA-2131 | 4 | 2 | | | |
| Exp. 3 | NP-231 | 16 | 4 | | 4 | |
| | NP-390 | 4 | 2 | | 2 | |
| | NP-1496 | 16 | 64 | | 128 | |
| | M-37 | 2 | 2 | | 128 | |
| Exp. 4 | M-37 | | 2 | 1 | 128 | |
| | M-480 | | 2 | 1 | 4 | |
| | M-598 | | 2 | 1 | 128 | |
| | M-934 | | 1 | 1 | 4 | |
| | NS-128 | | 2 | 1 | 2 | |
| | NS-562 | | 1 | 1 | 1 | |
| | NS-589 | | 1 | 1 | 1 | |
| | NP-1496 | | 64 | 16 | 128 | |
| Exp. 5 | GFP-949 | | 1 | | | 1 |
| | PB2-2240 | | 8 | | | 2 |
| | PB1-2257 | | 8 | | | 4 |
| | PA-2087 | | 16 | | | 128 |
| | NP-1496 | | 64 | | | 128 |
| | NP-231 | | 8 | | | 2 |

TABLE 5B
Effects of siRNAs on influenza virus production in MDCK cells

| 10\hat{ }3 pfu/well | | | | | 10\hat{ }4 pfu/well | | | | |
|---|---|---|---|---|---|---|---|---|---|
| (MOI.001) | | | | | (MOI.01) | | | | |
| | # | | | | | # | | | |
| | WELLS | | HA UNITS | | | WELLS | | HA UNITS | |
| | 24 HR | 48 HR | 24 HR | 48 HR | | 24 HR | 48 HR | 24 HR | 48 HR |
| Exp. 1 | | | | | | | | | |
| NT | 2 | 5 | 4 | 32 | NT | 6 | 7 | 64 | 128 |
| NP 1496 | 0 | 0 | 1 | 1 | NP 1496 | 3 | 5 | 8 | 32 |
| M 3 | 1 | 5 | 2 | 32 | M 3 | 5 | 7 | 32 | 128 |
| M 150 | 1 | 3 | 2 | 8 | M 150 | 6 | 7 | 64 | 128 |
| M 172 | 0 | 2 | 1 | 4 | M 172 | 6 | 7 | 64 | 128 |
| PB2 4 | 0 | 3 | 1 | 8 | PB2 4 | 5 | 7 | 32 | 128 |
| PB2 68 | 1 | 5 | 2 | 32 | PB2 68 | 6 | 7 | 64 | 128 |
| PB2 115 | 2 | 5 | 4 | 32 | PB2 115 | 6 | 7 | 64 | 128 |
| Exp. 2 | | | | | | | | | |
| NT | 1 | 4 | 2 | 16 | NT | 7 | >8 | 128 | >256 |
| NP 1496 | 0 | 1 | 1 | 2 | NP 1496 | 0 | 1 | 1 | 2 |
| NS 5 | 0 | 3 | 1 | 8 | NS 5 | 6 | >8 | 64 | >256 |
| NS 9 | 1 | 4 | 2 | 16 | NS 9 | 6 | >8 | 64 | >256 |
| M 211 | 1 | 4 | 2 | 16 | M 211 | 7 | >8 | 128 | >256 |
| M 232 | 0 | 2 | 1 | 4 | M 232 | 5 | >8 | 32 | >256 |
| PB2 167 | 0 | 2 | 1 | 4 | PB2 167 | 5 | 7 | 32 | 128 |
| PB2 473 | 1 | 4 | 2 | 16 | PB2 473 | 7 | >8 | 128 | >256 |
| Exp. 3 | | | | | | | | | |
| NT | 1 | 5 | 2 | 32 | NT | 4 | 7 | 16 | 128 |
| NP 1496 | 0 | 1 | 1 | 2 | NP 1496 | 0 | 3 | 1 | 8 |
| NS 543 | 1 | 5 | 2 | 32 | NS 543 | 2 | 6 | 4 | 64 |
| NS 623 | 1 | 5 | 2 | 32 | NS 623 | 4 | 7 | 16 | 128 |
| M 723 | 1 | 5 | 2 | 32 | M 723 | 2 | 7 | 4 | 128 |
| Exp. 4 | | | | | | | | | |
| NT | 3 | 6 | 8 | 64 | NT | >8 | 9 | >256 | 512 |
| NP 1496 | 0 | 1 | 1 | 2 | NP 1496 | 1 | 4 | 2 | 16 |
| NP 1501 | 0 | 4 | 1 | 16 | NP 1501 | 6 | 8 | 64 | 256 |
| NS 642 | 3 | 5 | 8 | 32 | NS 642 | 8 | 9 | 256 | 512 |
| NS 831 | 3 | 5 | 8 | 32 | NS 831 | 8 | 9 | 256 | 512 |
| PB1 1618 | 3 | 6 | 8 | 64 | PB1 1618 | 7 | 9 | 128 | 512 |
| PB2 12 | 3 | 5 | 8 | 32 | PB2 12 | 7 | 9 | 128 | 512 |
| M 232 | 2 | 5 | 4 | 32 | M 232 | 6 | 8 | 64 | 256 |
| Exp. 5 | | | | | | | | | |
| NT | 1 | 4 | 2 | 16 | NT | 5 | 7 | 32 | 128 |
| NP 1496 | 0 | 1 | 1 | 2 | NP 1496 | 0 | 1 | 1 | 2 |
| NP 14 | 1 | 4 | 2 | 16 | NP 14 | 5 | 7 | 32 | 128 |
| NP 1488 | 0 | 1 | 1 | 2 | NP 1488 | 0 | 3 | 1 | 8 |
| PB2 2283 | 0 | 2 | 1 | 4 | PB2 2283 | 4 | 5 | 16 | 32 |
| PA 2188 | 1 | 4 | 2 | 16 | PA 2188 | 4 | 6 | 16 | 64 |
| Exp. 6 | | | | | | | | | |
| NT | 2 | 4 | 4 | 16 | NT | 7 | 8 | 128 | 256 |
| NP 1496 | 0 | 0 | 1 | 1 | NP 1496 | 1 | 1 | 2 | 2 |
| PB2 956 | 2 | 4 | 4 | 16 | PB2 956 | 7 | 8 | 128 | 256 |
| M 13 | 2 | 4 | 4 | 16 | M 13 | 7 | 8 | 128 | 256 |
| M 255 | 1 | 2 | 2 | 4 | M 255 | 5 | 6 | 32 | 64 |
| M 645 | 1 | 2 | 2 | 4 | M 645 | 5 | 6 | 32 | 64 |
| M 808 | 1 | 3 | 2 | 8 | M 808 | 6 | 7 | 64 | 128 |
| M 832 | 1 | 1 | 2 | 2 | M 832 | 5 | 6 | 32 | 64 |
| M 986 | 2 | 3 | 4 | 8 | M 986 | 7 | 7 | 128 | 128 |
| NP 1488 | 0 | 0 | 1 | 1 | NP 1488 | 1 | 4 | 2 | 16 |
| Exp. 7 | | | | | | | | | |
| NT | 2 | 5 | 4 | 32 | NT | 6 | 8 | 64 | 256 |
| NP 1496 | 0 | 0 | 1 | 1 | NP 1496 | 2 | 3 | 4 | 8 |
| NP 1501 | 0 | 4 | 1 | 16 | NP 1501 | 4 | 6 | 16 | 64 |
| PB2 4 | 0 | 2 | 1 | 4 | PB2 4 | 5 | 6 | 32 | 64 |
| PB2 2283 | 0 | 3 | 1 | 8 | PB2 2283 | 4 | 6 | 16 | 64 |
| M 172 | 2 | 5 | 4 | 32 | M 172 | 5 | 7 | 32 | 128 |
| M 255 | 0 | 2 | 1 | 4 | M 255 | 4 | 6 | 16 | 64 |
| M 645 | 0 | 3 | 1 | 8 | M 645 | 4 | 6 | 16 | 64 |
| M 832 | 0 | 1 | 1 | 2 | M 832 | 4 | 6 | 16 | 64 |

### Example 3

### siRNAs that Target Viral RNA Polymerase or Nucleoprotein Inhibit Influenza A Virus Production in Chicken Embryos

Materials and Methods

SiRNA-oligofectamine complex formation and chicken embryo inoculation.

SiRNAs were prepared as described above. Chicken eggs were maintained under standard conditions. 30 μl of Oligofectamine (product number: 12252011 from Life Technologies, now Invitrogen) was mixed with 30 μl of Opti-MEM I (Gibco) and incubated at RT for 5 min. 2.5 nmol (10 μl) of siRNA was mixed with 30 μl of Opti-MEM I and added into diluted oligofectamine. The siRNA and oligofectamine was incubated at RT for 30 min. 10-day old chicken eggs were inoculated with siRNA-oligofectamine complex together with 100 μl of PR8 virus (5000 pfu/ml). The eggs were incubated at 37° C. for indicated time and allantoic fluid was harvested. Viral titer in allantoic fluid was tested by HA assay as described above.

Results

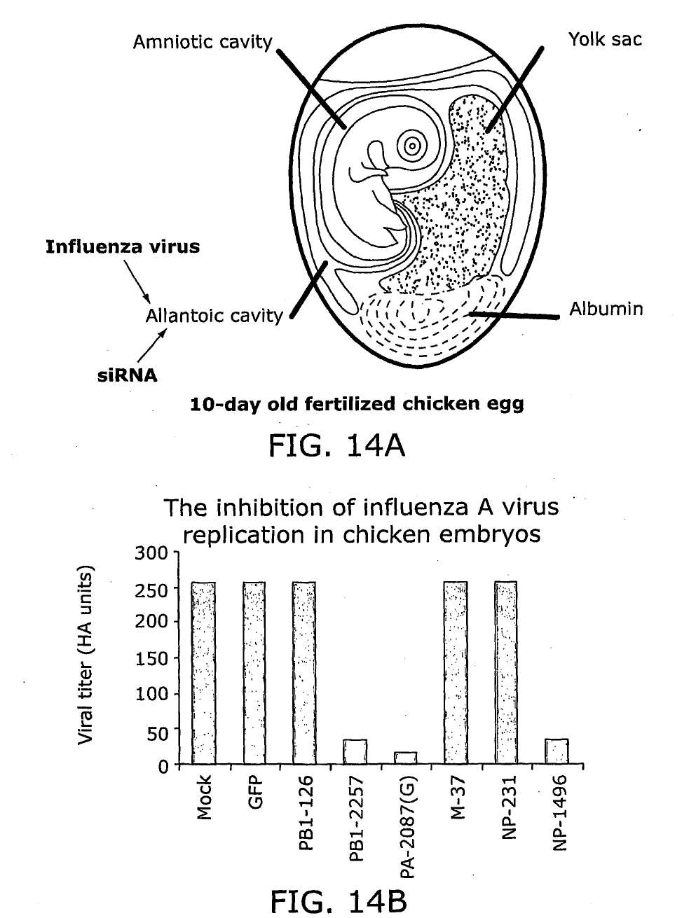

To confirm the results in MDCK cells, the ability of siRNA to inhibit influenza virus production in fertilized chicken eggs was also assayed. Because electroporation cannot be used on eggs, Oligofectamine, a lipid-based agent that has been shown to facilitate intracellular uptake of DNA oligonucleotides as well as siRNAs in vitro was used (25). Briefly, PR8 virus alone (500 pfu) or virus plus siRNA-oligofectamine complex was injected into the allantoic cavity of 10-day old chicken eggs as shown schematically in FIG. 14A. Allantoic fluids were collected 17 hours later for measuring virus titers by hemagglutinin assay. As shown in FIG. 14B, when virus was injected alone (in the presence of Oligofectamine), high virus titers were readily detected. Co-injection of GFP-949 did not significantly affect the virus titer. (No significant reduction in virus titer was observed when Oligofectamine was omitted.)

The injection of siRNAs specific for influenza virus showed results consistent with those observed in MDCK cells: The same siRNAs (NP-1496, PA2087 and PB1-2257) that inhibited influenza virus production in MDCK cells also inhibited virus production in chicken eggs, whereas the siRNAs (NP-231, M-37 and PB1-129) that were less effective in MDCK cells were ineffective in fertilized chicken eggs. Thus, siRNAs are also effective in interfering with influenza virus production in fertilized chicken eggs.

### Example 4

### SiRNA Inhibits Influenza Virus Production at the mRNA Level

Materials and Methods

SiRNA preparation was performed as described above.

RNA extraction, reverse transcription and real time PCR. 1×10⁷ MDCK cells were electroporated with 2.5 nmol of NP-1496 or mock electroporated (no siRNA). Eight hours later, influenza A PR8 virus was inoculated into the cells at MOI=0.1. At times 1, 2, and 3-hour post-infection, the supernatant was removed, and the cells were lysed with Trizol reagent (Gibco). RNA was purified according to the manufacturer's instructions. Reverse transcription (RT) was carried out at 37° C. for 1 hr, using 200 ng of total RNA, specific primers (see below), and Omniscript Reverse transcriptase kit (Qiagen) in a 20-μl reaction mixture according to the manufacturer's instructions. Primers specific for either mRNA, NP vRNA, NP cRNA, NS vRNA, or NS cRNA were as follows:

(SEQ ID NO: 112)

mRNA, dT₁₈ = 5′-TTTTTTTTTTTTTTTTTT-3′

(SEQ ID NO: 113)

NP vRNA, NP-367: 5′-CTCGTCGCTTATGACAAAGAAG-3′.

(SEQ ID NO: 114)

NP cRNA, NP-1565R: 5′-ATATCGTCTCGTATTAGTAGAAACAAGG
GTATTTTT-3′.

(SEQ ID NO: 115)

NS vRNA, NS-527: 5′-CAGGACATACTGATGAGGATG-3′.

(SEQ ID NO: 116)

NS cRNA, NS-890R: 5′-ATATCGTCTCGTATTAGTAGAAACAAGG
GTGTTTT-3′.

1 μl of RT reaction mixture (i.e., the sample obtained by performing reverse transcription) and sequence-specific primers were used for real-time PCR using SYBR Green PCR master mix (AB Applied Biosystems) including SYBR Green I double-stranded DNA binding dye. PCRs were cycled in an ABI PRISM 7000 sequence detection system (AB applied Biosystem) and analyzed with ABI PRISM 7000 SDS software (AB Applied Biosystems). The PCR reaction was carried out at 50° C., 2 min, 95° C., 10 min, then 95° C., 15 sec and 60° C., 1 min for 50 cycles. Cycle times were analyzed at a reading of 0.2 fluorescence units. All reactions were done in duplicate. Cycle times that varied by more than 1.0 between the duplicates were discarded. The duplicate cycle times were then averaged and the cycle time of β-actin was subtracted from them for a normalized value.

PCR primers were as follows.

For NP RNAs:

(SEQ ID NO: 117)

NP-367: 5′-CTCGTCGCTTATGACAAAGAAG-3′.

(SEQ ID NO: 118)

NP-460R: 5′-AGATCATCATGTGAGTCAGAC-3′.
For NS RNAs:

(SEQ ID NO: 119)

NS-527: 5′-CAGGACATACTGATGAGGATG-3′.

(SEQ ID NO: 120)

NS-617R: 5′-GTTTCAGAGACTCGAACTGTG-3′.

Results

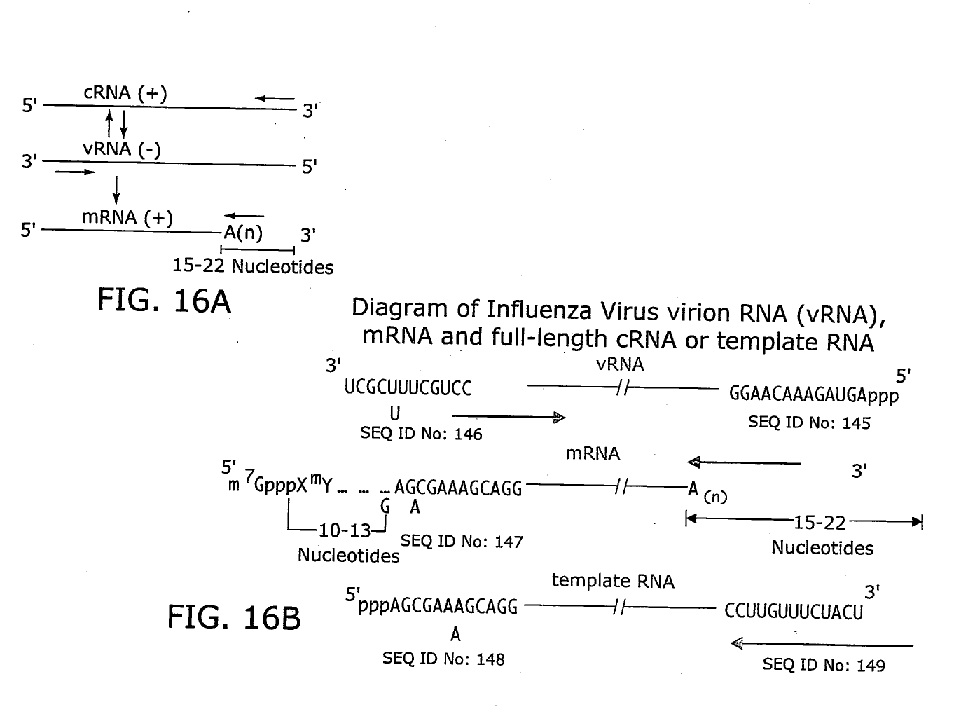

As described above, during replication of influenza virus, vRNA is transcribed to produce cRNA, which serves as a template for more vRNA synthesis, and mRNA, which serves as a template for protein synthesis (1). Although RNAi is known to target the degradation of mRNA in a sequence-specific manner (16-18), there is a possibility that vRNA and cRNA are also targets for siRNA since vRNA of influenza A virus is sensitive to nuclease (1). To investigate the effect of siRNA on the degradation of various RNA species, reverse transcription using sequence-specific primers followed by real time PCR was used to quantify the levels of vRNA, cRNA and mRNA. FIG. 16 shows the relationship between influenza virus vRNA, mRNA, and cRNA. As shown in FIGS. 16A and 16B, cRNA is the exact complement of vRNA, but mRNA contains a cap structure at the 5′ end plus the additional 10 to 13 nucleotides derived from host cell mRNA, and mRNA contains a polyA sequence at the 3′ end, beginning at a site complementary to a site 15-22 nucleotides downstream from the 5′ end of the vRNA segment. Thus compared to vRNA and cRNA, mRNA lacks 15 to 22 nucleotides at the 3′ end. To distinguish among the three viral RNA species, primers specific for vRNA, cRNA and mRNA were used in the first reverse transcription reaction (FIG. 16B). For mRNA, poly dT18 was used as primer. For cRNA, a primer complementary to the 3′ end of the RNA that is missing from mRNA was used. For vRNA, the primer can be almost anywhere along the RNA as long as it is complementary to vRNA and not too close to the 5′ end. The resulting cDNA transcribed from only one of the RNAs was amplified by real time PCR.

Following influenza virus infection, new virions are starting to be packaged and released by about 4 hrs. To determine the effect of siRNA on the first wave of mRNA and cRNA transcription, RNA was isolated early after infection. Briefly, NP-1496 was electroporated into MDCK cells. A mock electroporation (no siRNA) was also performed). Six to eight hours later, cells were infected with PR8 virus at MOI=0.1. The cells were then lysed at 1, 2 and 3 hours post-infection and RNA was isolated. The levels of mRNA, vRNA and cRNA were assayed by reverse transcription using primers for each RNA species, followed by real time PCR.

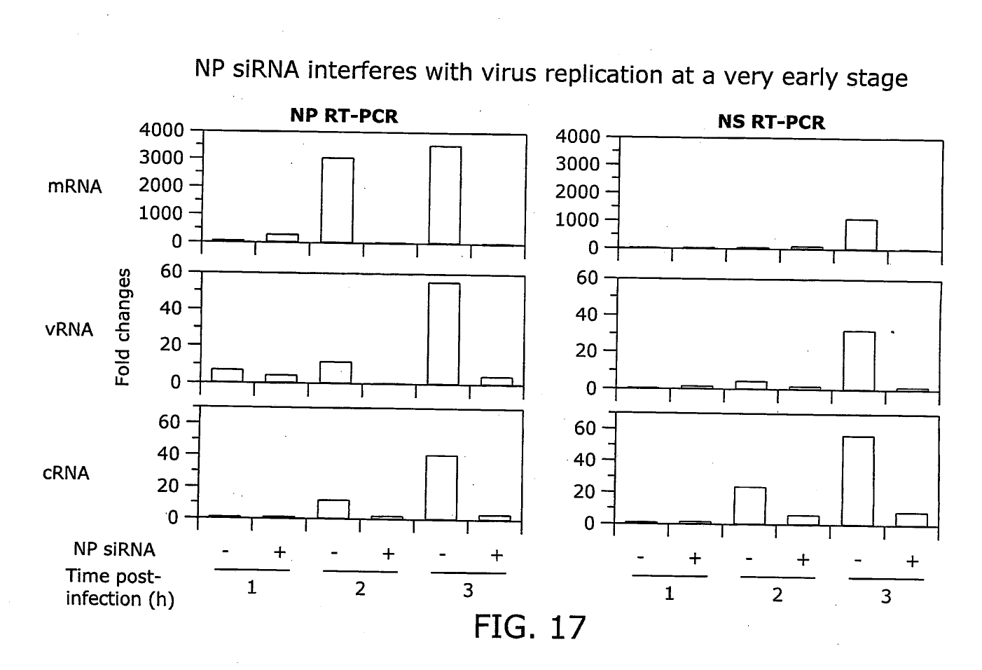

FIG. 17 shows amounts of viral NP and NS RNA species at various times following infection with virus, in cells that were mock transfected or transfected with siRNA NP-1496 approximately 6-8 hours prior to infection. As shown in FIG. 17, 1 hour after infection, there was no significant difference in the amount of NP mRNA between samples with or without NP siRNA transfection. As early as 2 hours post-infection, NP mRNA increased by 38 fold in the mock transfection group, whereas the levels of NP mRNA did not increase (or even slightly decreased) in cells transfected with siRNA. Three hours post-infection, mRNA transcript levels continued to increase in the mock transfection whereas a continuous decrease in the amount of NP mRNA was observed in the cells that received siRNA treatment. NP vRNA and cRNA displayed a similar pattern except that the increase in the amount of vRNA and cRNA in the mock transfection was significant only at 3 hrs post-infection. While not wishing to be bound by any theory, this is probably due to the life cycle of the influenza virus, in which an initial round of mRNA transcription occurs before cRNA and further vRNA synthesis.

These results indicate that, consistent with the results of measuring intact, live virus by hemagglutinin assay or plaque assay, the amounts of all NP RNA species were also significantly reduced by the treatment with NP siRNA. Although it is known that siRNA mainly mediates degradation of mRNA, the data from this experiment does not exclude the possibility of siRNA-mediated degradation of NP cRNA and vRNA although the results described below suggest that reduction in NP protein levels as a result of reduction in NP mRNA results in decreased stability of NP cRNA and/or vRNA.

### Example 5

### Identification of the Target of RNA Interference

Materials and Methods

SiRNA preparation of unmodified siRNAs was performed as described above. Modified RNA oligonucleotides, in which the 2′-hydroxyl group was substituted with a 2′-O-methyl group at every nucleotide residue of either the sense or antisense strand, or both, were also synthesized by Dharmacon. Modified oligonucleotides were deprotected and annealed to the complementary strand as described for unmodified oligonucleotides. siRNA duplexes were analyzed for completion of duplex formation by gel electrophoresis.

Cell culture, transfection with siRNAs, and infection with virus. These were performed essentially as described above. Briefly, for the experiment involving modified NP-1496 siRNA, MDCK cells were first transfected with NP-1496 siRNAs (2.5 nmol) formed from wild type (wt) and modified (m) strands and infected 8 hours later with PR8 virus at a MOI of 0.1. Virus titers in the culture supernatants were assayed 24 hours after infection. For the experiment involving M-37 siRNA, MDCK cells were transfected with M-37 siRNAs (2.5 nmol), infected with PR8 virus at an MOI of 0.01, and harvested for RNA isolation 1, 2, and 3 hours after infection. See Table 2 for M-37 sense and antisense sequences.

RNA extraction, reverse transcription and real time PCR were performed essentially as described above. Primers specific for either mRNA, M-specific vRNA, and M-specific cRNA, used for reverse transcription, were as follows:

(SEQ ID NO: 112)

mRNA, dT₁₈ = 5′-TTTTTTTTTTTTTTTTTT-3′

(SEQ ID NO: 161)

M vRNA: 5′-CGCTCAGACATGAGAACAGAATGG-3′

(SEQ ID NO: 162)

M cRNA: 5′-ATATCGTCTCGTATTAGTAGAAACAAGGTAGTTTTT-
3′.

PCR primers for M RNAs were as follows:

(SEQ ID NO: 163)

M forward: 5′-CGCTCAGACATGAGAACAGAATGG-3′

(SEQ ID NO: 164)

M reverse: 5′-TAACTAGCCTGACTAGCAACCTC-3′

Results

To investigate the possibility that siRNA might interfere with vRNA and/or cRNA in addition to mRNA, NP-1496 siRNAs in which either the sense (S or +) or antisense (AS or −) strand was modified were synthesized. The modification, which substitutes a 2′-O-methyl group for the 2′-hydroxyl group in every nucleotide residue, does not affect base-pairing for duplex formation, but the modified RNA strand no longer supports RNA interference. In other words, an siRNA in which the sense strand is modified but the antisense strand is wild type (mS:wtAS) will support degradation of RNAs having a sequence complementary to the antisense strand but not a sequence complementary to the sense strand. Conversely, an siRNA in which the sense strand is wild type but the antisense strand is modified (wtS:mAS) will support degradation of RNAs having a sequence complementary to the sense strand but will not support degradation of RNAs having a sequence complementary to the sense strand.

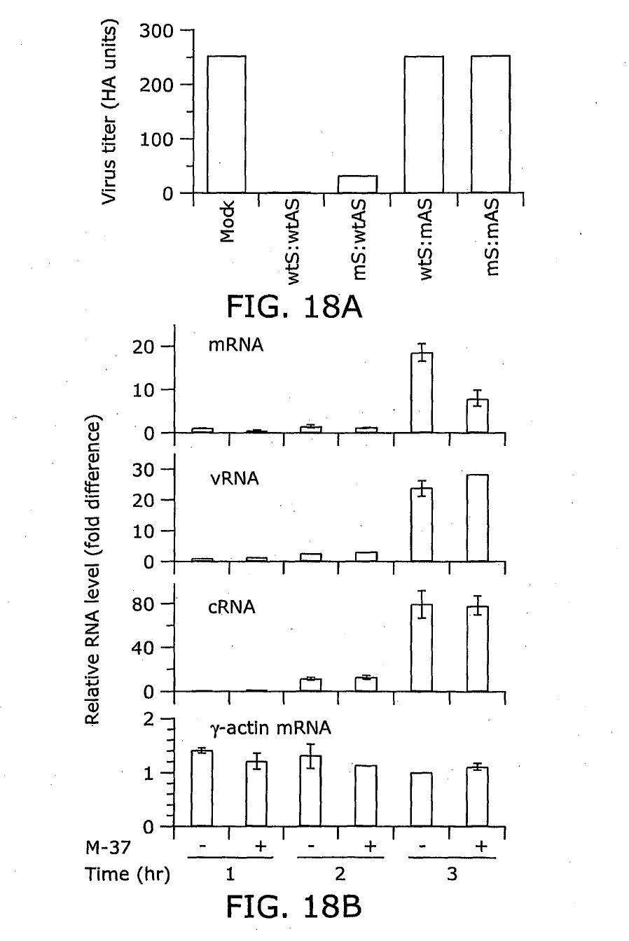

MDCK cells were either mock transfected or transfected with NP-1496 siRNAs in which either the sense strand (mS:wtAS) or the antisense strand (wtS:mAS), was modified while the other strand was wild type. Cells were also transfected with NP-1496 siRNA in which both strands were modified (mS:mAS). Cells were then infected with PR8 virus, and virus titer in supernatants was measured. As shown in FIG. 18A, high virus titers were detected in cultures subjected to mock transfection. As expected, very low virus titers were detected in cultures transfected with wild type siRNA (wtS:wtAS), but high virus titers were detected in cultures transfected with siRNA in which both strands were modified (mS:mAS). Virus titers were high in cultures transfected with siRNA in which the antisense strand was modified (wtAS:mAS), whereas the virus titers were low in cultures transfected with siRNA in which the sense strand only was modified (mS:wtAS). While not wishing to be bound by any theory, the inventors suggest that the requirement for a wild type antisense (−) strand of siRNA duplex to inhibit influenza virus production suggests that the target of RNA interference is either mRNA (+) or cRNA (+) or both.

To further distinguish these possibilities, the effect of siRNA on the accumulation of corresponding mRNA, vRNA, and cRNA was examined. To follow transcription in a cohort of simultaneously infected cells, siRNA-transfected MDCK cells were harvested for RNA isolation 1, 2, and 3 hours after infection (before the release and re-infection of new virions). The viral mRNA, vRNA, and cRNA were first independently converted to cDNA by reverse transcription using specific primers. Then, the level of each cDNA was quantified by real time PCR. As shown in FIG. 18B, when M-specific siRNA M-37 was used, little M-specific mRNA was detected one or two hours after infection. Three hours after infection, M-specific mRNA was readily detected in the absence of M-37. In cells transfected with M-37, the level of M-specific mRNA was reduced by approximately 50%. In contrast, the levels of M-specific vRNA and cRNA were not inhibited by the presence of M-37. While not wishing to be bound by any theory, these results indicate that viral mRNA is probably the target of siRNA-mediated interference.

### Example 6

### Effects of Certain siRNAs on Viral RNA Accumulation

Materials and Methods

SiRNA preparation was performed as described above.

RNA extraction, reverse transcription and real time PCR were performed as described in Example 3. Primers specific for either mRNA, NP vRNA, NP cRNA, NS vRNA, NS cRNA, M vRNA, or M cRNA were as described in Examples 4 and 5. Primers specific for PB1 vRNA, PB1 cRNA, PB2 vRNA, PB2 cRNA, PA vRNA, or PA cRNA, used for reverse transcription, were as follows:

(SEQ ID NO: 165)

PB1 vRNA: 5′-GTGCAGAAATCAGCCCGAATGGTTC-3′

(SEQ ID NO: 166)

PB1 cRNA: 5′-ATATCGTCTCGTATTAGTAGAAACAAGGCATTT-3′

(SEQ ID NO: 167)

PB2 vRNA: 5′-GCGAAAGGAGAGAAGGCTAATGTG-3′

(SEQ ID NO: 168)

PB2 cRNA: 5′-ATATGGTCTCGTATTAGTAGAAACAAGGTCGTTT-3′

(SEQ ID NO: 169)

PA vRNA: 5′-GCTTCTTATCGTTCAGGCTCTTAGG-3′

(SEQ ID NO: 170)

PA cRNA: 5′-ATATCGTCTCGTATTAGTAGAAACAAGGTACTT-3′

PCR primers for PB1, PB2, and PA RNAs were as follows:

(SEQ ID NO: 171)

PB1 forward: 5′-CGGATTGATGCACGGATTGATTTC-3′

(SEQ ID NO: 172)

PB1 reverse: 5′-GACGTCTGAGCTCTTCAATGGTGGAAC-3′

(SEQ ID NO: 173)

PB2 forward: 5′-GCGAAAGGAGAGAAGGCTAATGTG-3′

(SEQ ID NO: 174)

PB2 reverse: 5′-AATCGCTGTCTGGCTGTCAGTAAG-3′

(SEQ ID NO: 175)

PA forward: 5′-GCTTCTTATCGTTCAGGCTCTTAGG-3′

(SEQ ID NO: 176)

PA reverse: 5′-CCGAGAAGCATTAAGCAAAACCCAG-3′

Results

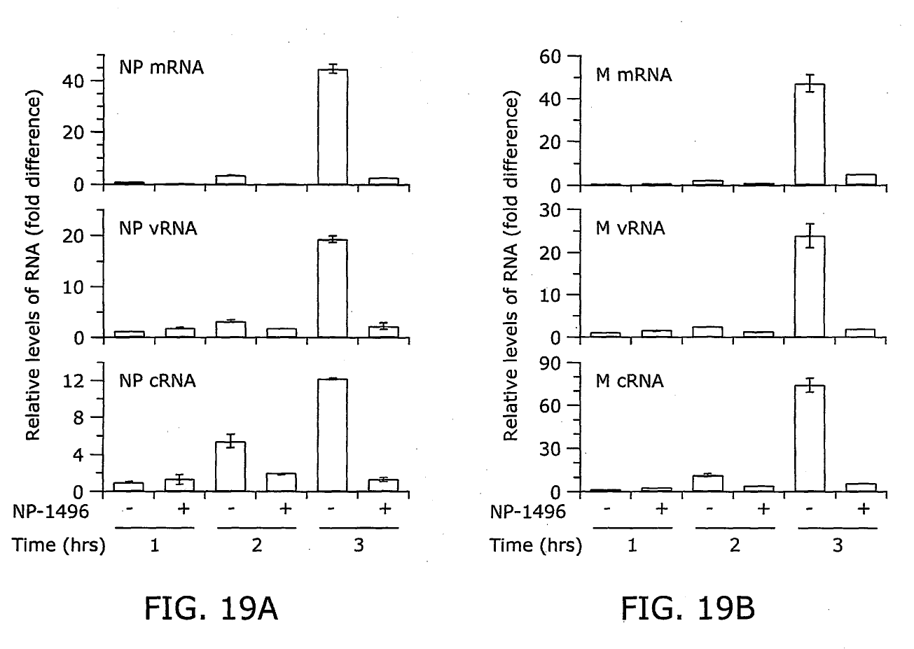

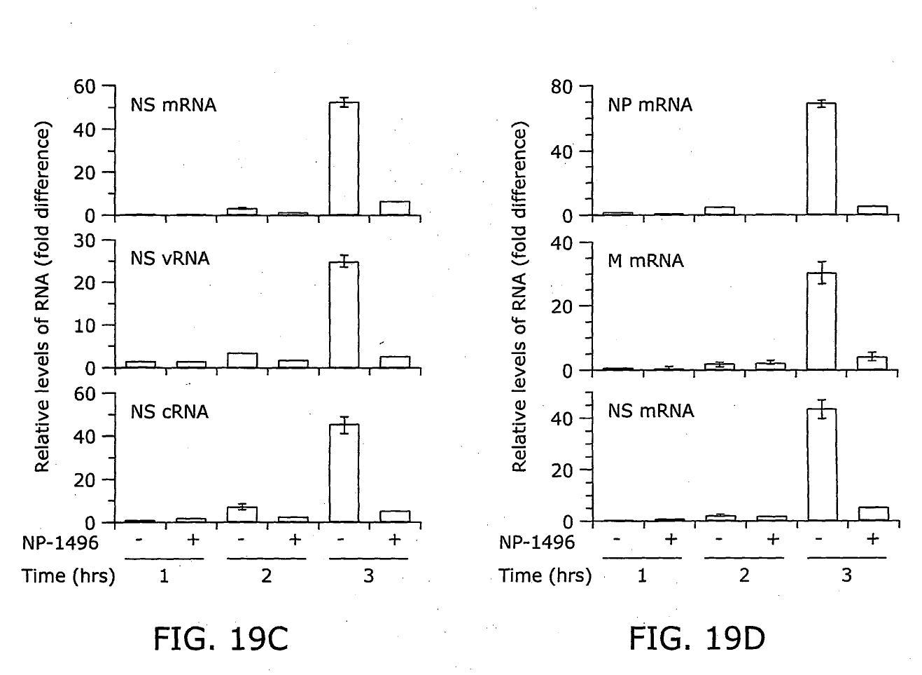

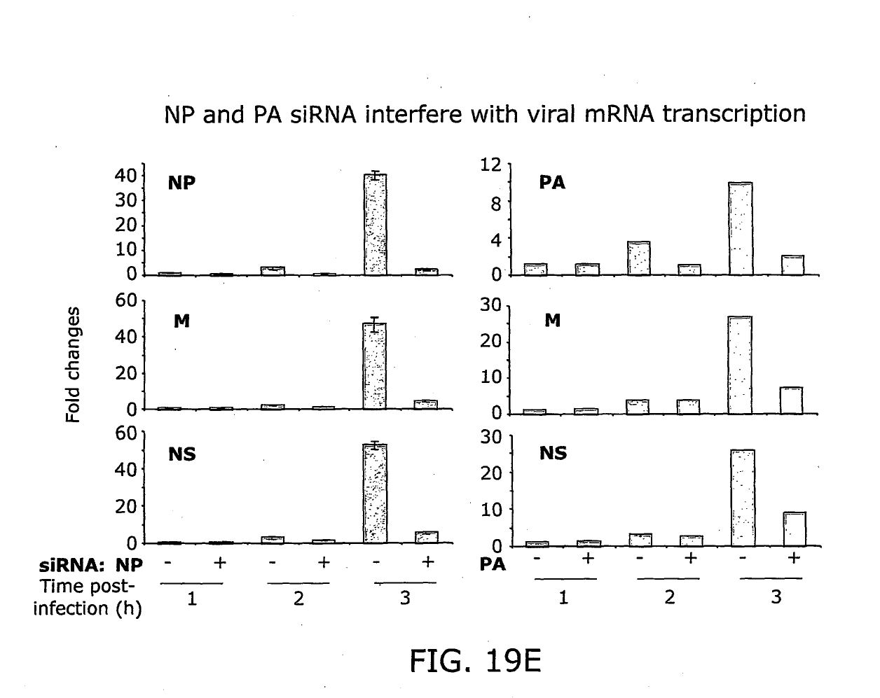

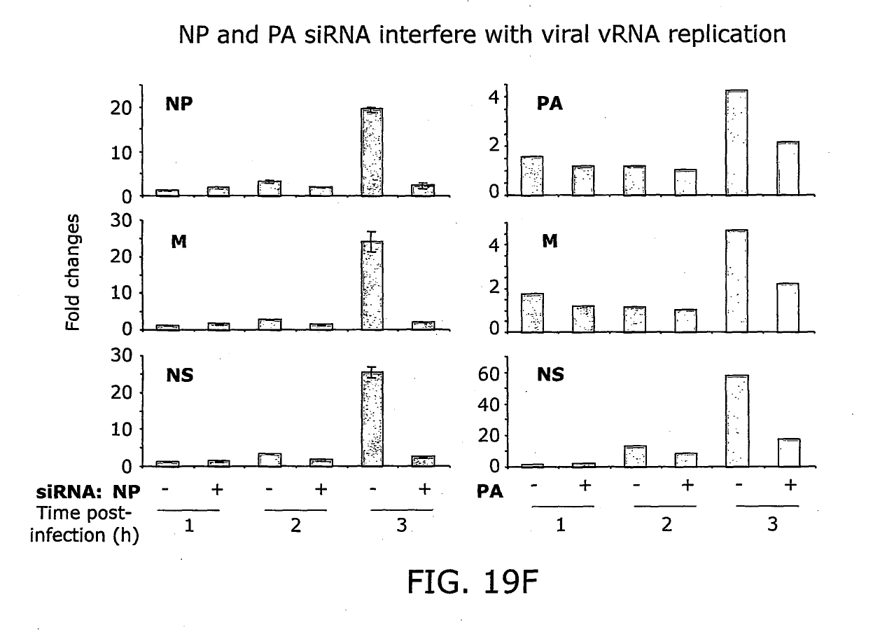

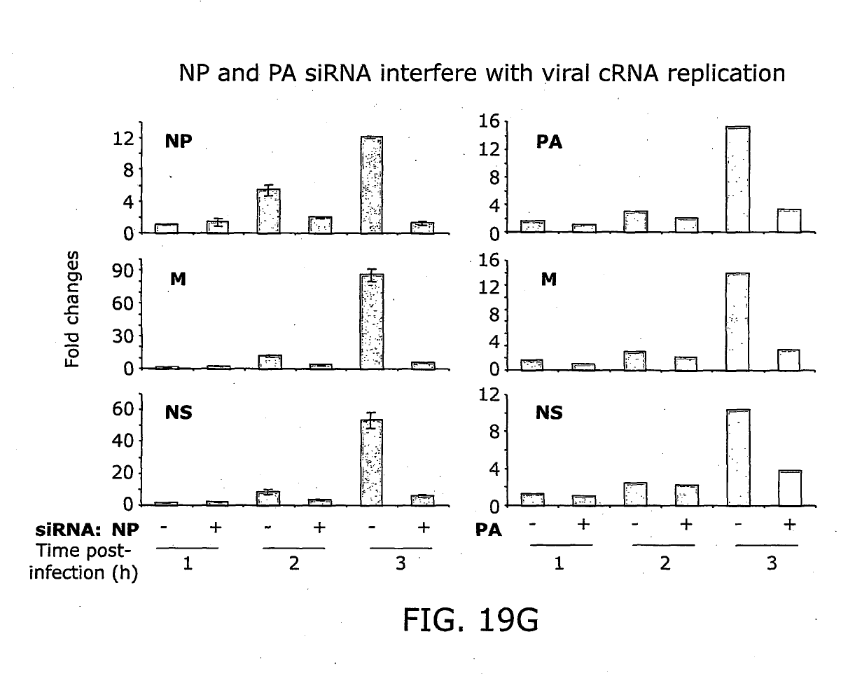

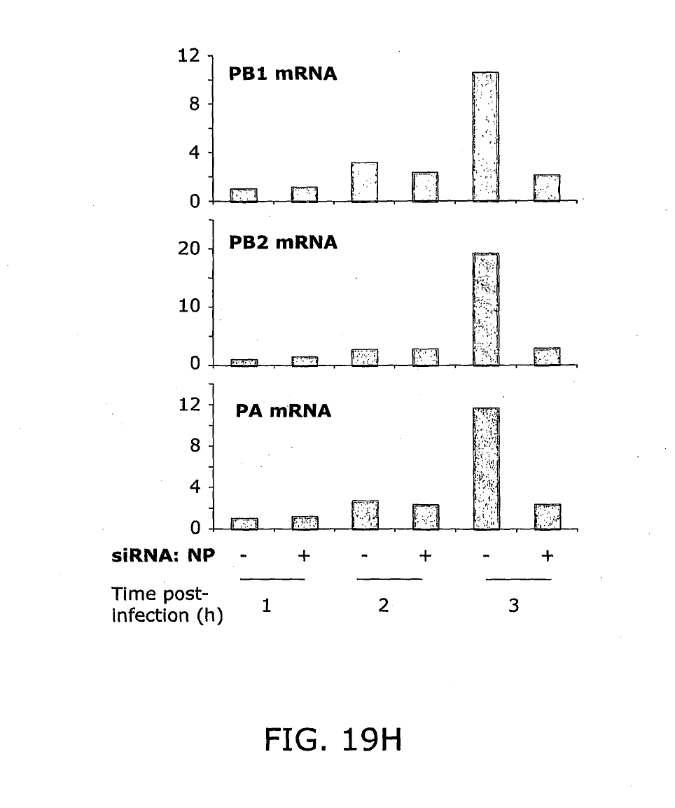

To determine whether NP-1496 targets the degradation of the NP gene segment specifically or whether the levels of viral RNAs other than NP are also affected, primers specific for NS were used for RT and real time PCR to measure the amount of different NS RNA species (mRNA, vRNA, cRNA) as described above (Example 4). As shown in FIG. 19, the changes in NS mRNA, vRNA and cRNA showed the same pattern as that observed for NP RNAs. At 3 hours post-infection, a significant increase in all NS RNA species could be seen in mock transfected cells, whereas no significant changes in NS RNA levels were seen in the cells that received NP-1496 siRNA. This result indicates that the transcription and replication of different viral RNAs are coordinately regulated, at least with respect to NP RNAs. By coordinately regulated is meant that levels of one transcript affect levels of another transcript, either directly or indirectly. No particular mechanism is implied. When NP transcripts are degraded by siRNA treatment the levels of other viral RNAs are also reduced.

To further explore the effect of NP siRNAs on other viral RNAs, accumulation of mRNA, vRNA, and cRNA of all viral genes was measured in cells that had been treated with NP-1496. As shown in FIG. 19A (top panel), NP-specific mRNA was low one or two hours after infection. Three hours after infection, NP mRNA was readily detected in the absence of NP-1496, whereas in the presence of NP-1496, the level of NP mRNA remained at the background level, indicating that siRNA inhibited the accumulation of specific mRNA. As shown in FIG. 19A (middle and bottom panels) levels of NP-specific and NS-specific vRNA and cRNA were greatly inhibited by the presence of NP-1496. These results confirm the results described in Example 4. In addition, in the NP-1496-treated cells, the accumulation of mRNA, vRNA, and cRNA of the M, NS, PB1, PB2, and PA genes was also inhibited (FIGS. 19B, 19C, and 19H). Furthermore, the broad inhibitory effect was also observed for PA-2087. The top, middle, and bottom panels on the left side in FIGS. 19E, 19F, and 19G display the same results as presented in FIGS. 19A, 19B, and 19C, showing the inhibition of viral mRNA transcription and of viral vRNA and cRNA replication by NP-1496 siRNA. The top, middle, and bottom panels on the right side in FIGS. 19E, 19F, and 19G present results of the same experiment performed with PA-2087 siRNA at the same concentration. As shown in FIG. 19E, right upper, middle, and lower panels respectively, at three hours after infection PA, M, and NS mRNA were readily detected in the absence of PA-2087, whereas the presence of PA-2087 inhibited transcription of PA, M, and NS mRNA. As shown in FIG. 19F, right upper, middle, and lower panels respectively, at three hours after infection PA, M, and NS vRNA were readily detected in the absence of PA-2087, whereas the presence of PA-2087 inhibited accumulation of PA, M, and NS vRNA. As shown in FIG. 19G, right upper, middle, and lower panels respectively, at three hours after infection PA, M, and NS cRNA were readily detected in the absence of PA-2087, whereas the presence of PA-2087 inhibited accumulation of PA, M, and NS cRNA. In addition, FIG. 19H shows that NP-specific siRNA inhibits the accumulation of PB1—(top panel), PB2—(middle panel) and PA—(lower panel) specific mRNA.

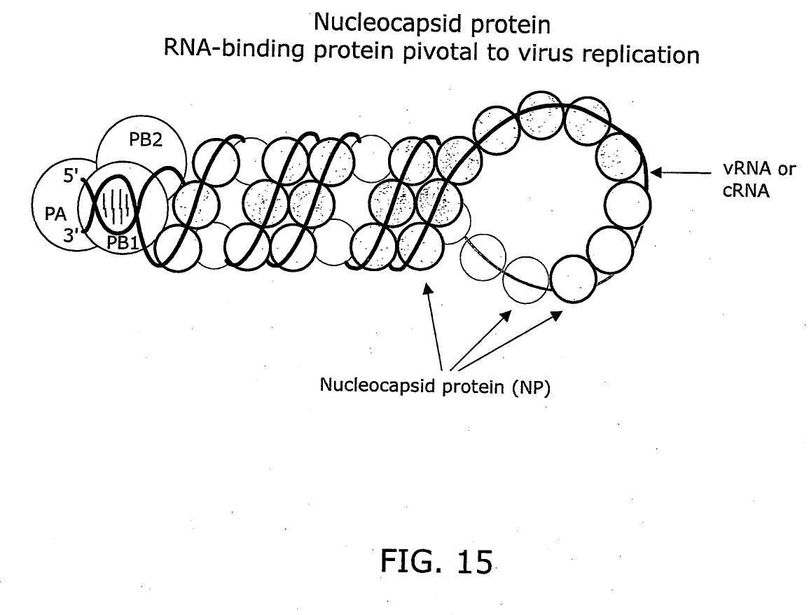

While not wishing to be bound by any theory, the inventors suggest that the broad effect of NP siRNA is probably a result of the importance of NP in binding and stabilizing vRNA and cRNA, and not because NP-specific siRNA targets RNA degradation non-specifically. The NP gene segment in influenza virus encodes a single-stranded RNA-binding nucleoprotein, which can bind to both vRNA and cRNA (see FIG. 15). During the viral life cycle, NP mRNA is first transcribed and translated. The primary function of the NP protein is to encapsidate the virus genome for the purpose of RNA transcription, replication and packaging. In the absence of NP protein, the full-length synthesis of both vRNA and cRNA is strongly impaired. When NP siRNA induces the degradation of NP RNA, NP protein synthesis is impaired and the resulting lack of sufficient NP protein subsequently affects the replication of other viral gene segments. In this way, NP siRNA could potently inhibit virus production at a very early stage.

The number of NP protein molecules in infected cells has been hypothesized to regulate the levels of mRNA synthesis versus genome RNA (vRNA and cRNA) replication (1). Using a temperature-sensitive mutation in the NP protein, previous studies have shown that cRNA, but not mRNA, synthesis was temperature sensitive both in vitro and in vivo (70, 71). NP protein was shown to be required for elongation and antitermination of the nascent cRNA and vRNA transcripts (71, 72). The results presented above show that NP-specific siRNA inhibited the accumulation of all viral RNAs in infected cells. While not wishing to be bound by any theory, it appears probable that in the presence of NP-specific siRNA, the newly transcribed NP mRNA is degraded, resulting in the inhibition of NP protein synthesis following virus infection. Without newly synthesized NP, further viral transcription and replication, and therefore new virion production is inhibited.

Similarly, in the presence of PA-specific, the newly transcribed PA mRNA is degraded, resulting in the inhibition of PA protein synthesis. Despite the presence of 30-60 copies of RNA transcriptase per influenza virion (1), without newly synthesized RNA transcriptase, further viral transcription and replication are likely inhibited. Similar results were obtained using siRNA specific for PB 1. In contrast, the matrix (M) protein is not required until the late phase of virus infection (1). Thus, M-specific siRNA inhibits the accumulation of M-specific mRNA but not vRNA, cRNA, or other viral RNAs. Taken together, these findings demonstrate a critical requirement for newly synthesized nucleoprotein and polymerase proteins in influenza viral RNA transcription and replication. Both mRNA- and virus-specific mechanisms by which NP-, PA-, and PB1-specific siRNAs interfere with mRNA accumulation and other viral RNA transcription suggest that these siRNAs may be especially potent inhibitors of influenza virus infection.

### Example 7

### Broad Inhibition of Influenza Virus RNA Accumulation by Certain siRNAs is Not Due to the Interferon Response or to Virus-induced RNA Degradation

Materials and Methods

Measurement of RNA levels. RNA levels were measured using PCR under standard conditions. The following PCR primers were used for measurement of γ-actin RNA.

(SEQ ID NO: 177)

65 -actin forward: 5′-TCTGTCAGGGTTGGAAAGTC-3′

(SEQ ID NO: 178)

γ-actin reverse: 5′-AAATGCAAACCGCTTCCAAC-3′

Culture of Vero cells and measurements of phosphorylated PKR were performed according to standard techniques described in the references cited below.

Results

One possible cause for the broad inhibition of viral RNA accumulation described in Example 6 is an interferon response of the infected cells in the presence of siRNA (23, 65, 66). Thus, the above experiments were repeated in Vero cells in which the entire IFN locus, including all α, β, and ω genes, are deleted (67, 68) (Q. G. and J. C. unpublished data). Just as in MDCK cells, the accumulation of NP-, M-, and NS-specific mRNAs were all inhibited by NP-1496 (FIG. 19D). In addition, the effect of siRNA on the levels of transcripts from cellular genes, including β-actin, γ-actin, and GAPDH, was assayed using PCR. No significant difference in the transcript levels was detected in the absence or presence of siRNA (FIG. 18C bottom panel, showing lack of effect of M-37 siRNA on γ-actin mRNA, and data not shown), indicating that the inhibitory effect of siRNA is specific for viral RNAs. These results suggest that the broad inhibition of viral RNA accumulation by certain siRNAs is not a result of a cellular interferon response.

Following influenza virus infection, the presence of dsRNA also activates a cellular pathway that targets RNA for degradation (23). To examine the effect of siRNA on the activation of this pathway, we assayed the levels of phosphorylated protein kinase R (PKR), the most critical component of the pathway (23). Transfection of MDCK cells with NP-1496 in the absence of virus infection did not affect the levels of activated PKR (data not shown). Infection by influenza virus resulted in an increased level of phosphorylated PKR, consistent with previous studies (65, 66, 69). However, the increase was the same in the presence or absence of NP-1496 (data not shown). Thus, the broad inhibition of viral RNA accumulation is not a result of enhanced virus-induced degradation in the presence of siRNA.

### Example 8

### Systematic Identification of siRNAs with Superior Ability to Inhibit Influenza Virus Production Either Alone or in Combination

A high throughput screen (Example 18) was conducted to identify siRNAs with superior ability to inhibit influenza virus production. The siRNAs were tested individually in cell culture, and a number were further tested in mice. Certain combinations were also tested and demonstrated an additive effect. Systematic testing of additional combinations is performed to identify combinations with synergistic (i.e., greater than additive) effects. The siRNAs and other RNAi-inducing entitities comprising the same antisense strands are further tested against additional influenza virus strains, including major human and avian pathogens.

### Example 9

### Evaluation of Non-viral Delivery Agents that Facilitate Cellular Uptake of siRNA

A variety of non-viral delivery agents were tested for their ability to enhance cellular uptake of siRNA. Subsequent examples provide data showing positive results (e.g., inhibition of influenza virus production) with a number of the polymers in both cell culture and in animals. Additional delivery agents are tested using similar approaches.

### Example 10

### Testing of Compositions Containing RNAi-Inducing Agents in Mice

Dry particles comprising an RNAi-inducing agent targeted to an influenza virus transcript are prepared as described (58). In this procedure, water-soluble excipients (i.e. lactose, albumin, etc.) and therapeutics were dissolved in distilled water. The solution was fed to a Niro Atomizer Portable Spray Dryer (Niro, Inc., Colombus, Md.) to produce the dry powders, which have a mean geometric diameters ranged between 3 and 15 μm and tap density between 0.04 and 0.6 g/cm³. The dry powders are administered to the respiratory system of mice by inhalation or intratracheal administration. Inhalational delivery of a dry powder aerosol is accomplished by forced ventilation on anesthetized mice. For intratracheal administration, a solution containing therapeutics is injected via a tube into the lungs of anesthetized mice (54). In other experiments, for delivery of liquids, liquid aerosols are produced by a nebulizer into a sealed plastic cage, where the mice are placed (52). Insufflators such as those available from Penn Century (URLwww.penncentury.com), e.g., Model IA-IC may be used for pulmonary delivery of dry powders to small animals.

### Example 11

### Inhibition of Influenza Virus Infection by siRNAs Transcribed from Templates Provided by DNA Vectors or Lentiviruses

As an alternative to the approaches described above, the use of DNA vectors from which siRNA precursors can be transcribed and processed into effective siRNAs was explored as described in Examples 13 and 14. FIGS. 20A-20C show vectors that were used for previous studies (27, 59), results of which are shown in FIG. 20D and further described in U.S. Ser. No. 10/674,159. FIGS. 21A-21C show additional constructs that can be used to test the efficacy of hairpin precursors that include precursors for multiple different siRNAs.

### Example 12

### Inhibition of Influenza Virus Production in Mice by siRNAs

This example describes experiments showing that administration of siRNAs targeted to influenza virus NP or PA transcripts inhibit production of influenza virus in mice when administered either prior to or following infection with influenza virus. The inhibition is dose-dependent and shows additive effects when two siRNAs each targeted to a transcript expressed from a different influenza virus gene were administered together.

Materials and Methods

SiRNA preparation. This was performed as described above.

SiRNA delivery. siRNAs (30 or 60 μg of GFP-949, NP-1496, or PA-2087) were incubated with jetPEI™ for oligonucleotides cationic polymer transfection reagent, N/P ratio=5 (Qbiogene, Inc., Carlsbad, Calif.; Cat. No. GDSP20130; N/P refers to the number of nitrogens per nucleotide phosphate in the jetPEI/siRNA mixture) or with poly-L-lysine (MW (vis) 52,000; MW (LALLS) 41,800, Sigma Cat. No. P2636) for 20 min at room temperature in 5% glucose. The mixture was injected into mice intravenously, into the retro-orbital vein, 200 μl per mouse, 4 mice per group. 200 μl 5% glucose was injected into control (no treatment) mice. The mice were anesthetized with 2.5% Avertin before siRNA injection or intranasal infection.

Viral infection. B6 mice (maintained under standard laboratory conditions) were intranasally infected with PR8 virus by dropping virus-containing buffer into the mouse's nose with a pipette, 30 ul (12,000 pfu) per mouse.

Determination of viral titer. Mice were sacrificed at various times following infection, and lungs were harvested. Lungs were homogenized, and the homogenate was frozen and thawed twice to release virus. PR8 virus present in infected lungs was titered by infection of MDCK cells. Flat-bottom 96-well plates were seeded with 3×10⁴ MDCK cells per well, and 24 hrs later the serum-containing medium was removed. 25 μl of lung homogenate, either undiluted or diluted from 1×10⁻¹ to 1×10⁻⁷, was inoculated into triplicate wells. After 1 h incubation, 175 μl of infection medium with 4 μg/ml of trypsin was added to each well. Following a 48 h incubation at 37° C., the presence or absence of virus was determined by hemagglutination of chicken RBC by supernatant from infected cells. The hemagglutination assay was carried out in V-bottom 96-well plates. Serial 2-fold dilutions of supernatant were mixed with an equal volume of a 0.5% suspension (vol/vol) of chicken erythrocytes (Charles River Laboratories) and incubated on ice for 1 h. Wells containing an adherent, homogeneous layer of erythrocytes were scored as positive. The virus titers were determined by interpolation of the dilution end point that infected 50% of wells by the method of Reed and Muench (TCID₅₀), thus a lower TCID₅₀ reflects a lower virus titer. The data from any two groups were compared by Student t test, which was used throughout the experiments described herein to evaluate significance.

Results

FIG. 22A shows results of an experiment demonstrating that siRNA targeted to viral NP transcripts inhibits influenza virus production in mice when administered prior to infection. 30 or 60 μg of GFP-949 or NP-1496 siRNAs were incubated with jetPEI and injected intravenously into mice as described above in Materials and Methods. Three hours later mice were intranasally infected with PR8 virus, 12000 pfu per mouse. Lungs were harvested 24 hours after infection. As shown in FIG. 22A, the average log₁₀TCID₅₀ of the lung homogenate for mice that received no siRNA treatment (NT; filled squares) or received an siRNA targeted to GFP (GFP 60 μg; open squares) was 4.2. In mice that were pretreated with 30 μg siRNA targeted to NP (NP 30 μg; open circles) and jetPEI, the average log₁₀TCID₅₀ of the lung homogenate was 3.9. In mice that were pretreated with 60 μg siRNA targeted to NP (NP 60 μg; filled circles) and jetPEI, the average log₁₀TCID₅₀ of the lung homogenate was 3.2. The difference in virus titer in the lung homogenate between the group that received no treatment and the group that received 60 μg NP siRNA was significant with P=0.0002. Data for individual mice are presented in Table 6A (NT=no treatment).

FIG. 22B shows results of another experiment demonstrating that siRNA targeted to viral NP transcripts inhibits influenza virus production in mice when administered intravenously prior to infection in a composition containing the cationic polymer PLL. 30 or 60 μg of GFP-949 or NP-1496 siRNAs were incubated with PLL and injected intravenously into mice as described above in Materials and Methods. Three hours later mice were intranasally infected with PR8 virus, 12000 pfu per mouse. Lungs were harvested 24 hours after infection. As shown in FIG. 22B, the average log₁₀TCID₅₀ of the lung homogenate for mice that received no siRNA treatment (NT; filled squares) or received an siRNA targeted to GFP (GFP 60 μg; open squares) was 4.1. In mice that were pretreated with 60 μg siRNA targeted to NP (NP 60 μg; filled circles) and PLL, the average log₁₀TCID₅₀ of the lung homogenate was 3.0. The difference in virus titer in the lung homogenate between the group that received 60 μg GFP and the group that received 60 μg NP siRNA was significant with P=0.001. Data for individual mice are presented in Table 6A (NT=no treatment). These data indicate that siRNA targeted to the influenza NP transcript reduced the virus titer in the lung when administered prior to virus infection. They also indicate that a mixtures of an siRNA with a cationic polymer effectively inhibits influenza virus in the lung when administered by intravenous injection, not requiring techniques such as hydrodynamic transfection.

TABLE 6A
Inhibition of influenza virus production in mice by siRNA with
cationic polymers

| | |
|---|---|
| Treatment | log₁₀TCID50 |

| | | | | |
|---|---|---|---|---|
| NT (jetPEI experiment) | 4.3 | 4.3 | 4.0 | 4.0 |
| GFP (60 μg) + jetPEI | 4.3 | 4.3 | 4.3 | 4.0 |
| NP (30 μg) + jetPEI | 4.0 | 4.0 | 3.7 | 3.7 |
| NP (60 μg) + jetPEI | 3.3 | 3.3 | 3.0 | 3.0 |
| NT (PLL experiment) | 4.0 | 4.3 | 4.0 | 4.0 |
| GFP (60 μg) + PLL | 4.3 | 4.0 | 4.0 | (not done) |
| NP (60 μg) + PLL | 3.3 | 3.0 | 3.0 | 2.7 |

FIG. 22C shows results of a third experiment demonstrating that siRNA targeted to viral NP transcripts inhibits influenza virus production in mice when administered prior to infection and demonstrates that the presence of a cationic polymer significantly increases the inhibitory efficacy of siRNA. 60 μg of GFP-949 or NP-1496 siRNAs were incubated with phosphate buffered saline (PBS) or jetPEI and injected intravenously into mice as described above in Materials and Methods. Three hours later mice were intranasally infected with PR8 virus, 12000 pfu per mouse. Lungs were harvested 24 hours after infection. As shown in FIG. 22C, the average log₁₀TCID₅₀ of the lung homogenate for mice that received no siRNA treatment (NT; open squares) was 4.1, while the average log₁₀TCID₅₀ of the lung homogenate for mice that received an siRNA targeted to GFP in PBS (GFP PBS; open triangles) was 4.4. In mice that were pretreated with 60 μg siRNA targeted to NP in PBS (NP PBS; closed triangles) the average log₁₀TCID₅₀ of the lung homogenate was 4.2, showing only a modest increase in efficacy relative to no treatment or treatment with an siRNA targeted to GFP. In mice that were pretreated with 60 μg siRNA targeted to GFP in jetPEI (GFP PEI; open circles), the average log₁₀TCID₅₀ of the lung homogenate was 4.2. However, in mice that received 60 μg siRNA targeted to NP in jetPEI (NP PEI; closed circles), the average log₁₀TCID₅₀ of the lung homogenate was 3.2. The difference in virus titer in the lung homogenate between the group that received GFP siRNA in PBS and the group that received NP siRNA in PBS was significant with P=0.04, while the difference in virus titer in the lung homogenate between the group that received GFP siRNA with jetPEI and the group that received NP siRNA with jetPEI was highly significant with P=0.003. Data for individual mice are presented in Table 6B (NT=no treatment).

TABLE 6B
Inhibition of influenza virus production in mice by siRNA showing
increased efficacy with cationic polymer

| | |
|---|---|
| Treatment | log₁₀TCID50 |

| | | | | |
|---|---|---|---|---|
| NT | 4.3 | 4.3 | 4.0 | 3.7 |
| GFP (60 μg) + PBS | 4.3 | 4.3 | 4.7 | 4.3 |
| NP (60 μg) + PBS | 3.7 | 4.3 | 4.0 | 4.0 |
| GFP (60 μg) + jetPEI | 4.3 | 4.3 | 4.0 | 3.0 |
| NT (60 μg) + jetPEI | 3.3 | 3.0 | 3.7 | 3.0 |

Additional experiments were performed to assess the ability of siRNA to inhibit influenza virus production at various times after infection, when administered at various time points prior to or following infection.

siRNA was administered as described above except that 120 ug siRNA was administered 12 hours before virus infection. Table 6C shows the results expresesed as log₁₀TCID₅₀. The P value comparing NP-treated with control group was 0.049

TABLE 6C

| | Mouse | | | |
|---|---|---|---|---|
| | 1 | Mouse 2 | Mouse 3 | Mouse 4 |
| NT | 4.3 | 4 | 4 | 4 |
| GFP-949 | 4.3 | 4 | 4 | 4 |
| NP-1496 | 4 | 3.7 | 3.7 | 3.3 |

In another experiment, siRNA (60 ug) was administered 3 hours before infection. 1500 pfu of PR8 virus was administered intranasally. The infected lung was harvested 48 h after infection. Table 6D shows the results expressed as log₁₀TCID₅₀. The P value comparing NP-treated with control group was 0.03.

TABLE 6D

| | Mouse | | | |
|---|---|---|---|---|
| | 1 | Mouse 2 | Mouse 3 | Mouse 4 |
| NT | 4 | 4 | 4 | 4 |
| GFP-949 | 4.3 | 4 | 4 | 3.7 |
| NP-1496 | 3 | 3.7 | 3.7 | 3.3 |

In another experiment, siRNA (120 ug) was administered 24 hours after PR8 (1500 pfu) infection. 52 hours post-infection, the lung was harvested and virus titer was measured. Table 6D shows the results expressed as log₁₀TCID₅₀. The P value comparing NP-treated with control group was 0.03.

TABLE 6E

| | Mouse | | | |
|---|---|---|---|---|
| | 1 | Mouse 2 | Mouse 3 | Mouse 4 |
| GFP-949 | 2.3 | 2.7 | 2 | 2.7 |
| NP-1496 | 2 | 2 | 1.7 | 2 |

Other polymers were also shown to be effective siRNA delivery agents. FIG. 22D is a plot showing that siRNA targeted to NP (NP-1496) inhibits influenza virus production in mice when administered intravenously together with a poly(beta amino ester) (J28). FIG. 22E is a plot showing that siRNA targeted to NP (NP-1496) inhibits influenza virus production in mice when administered intraperitoneally together with a poly(beta amino ester) (J28 or C32) while a control RNA (GFP) has no significant effect. The experiments were performed essentially as described above except that the ratio of polymer to siRNA was a weight/weight ratio (for instance, 60:1 w/w). Polymers and siRNA were mixed and administered to mice either intravenously or intraperitoneally 3 hours prior to intranasal infection with 12,000 pfu of PR8 virus. Lungs were harvested 24 hours later and HA assays were performed. The amine and bis(acrylate ester) monomers present in J28 and C32 are described and depicted in U.S. Ser. No. 10/446,444. The polymers were a kind gift of Dr. Robert Langer.

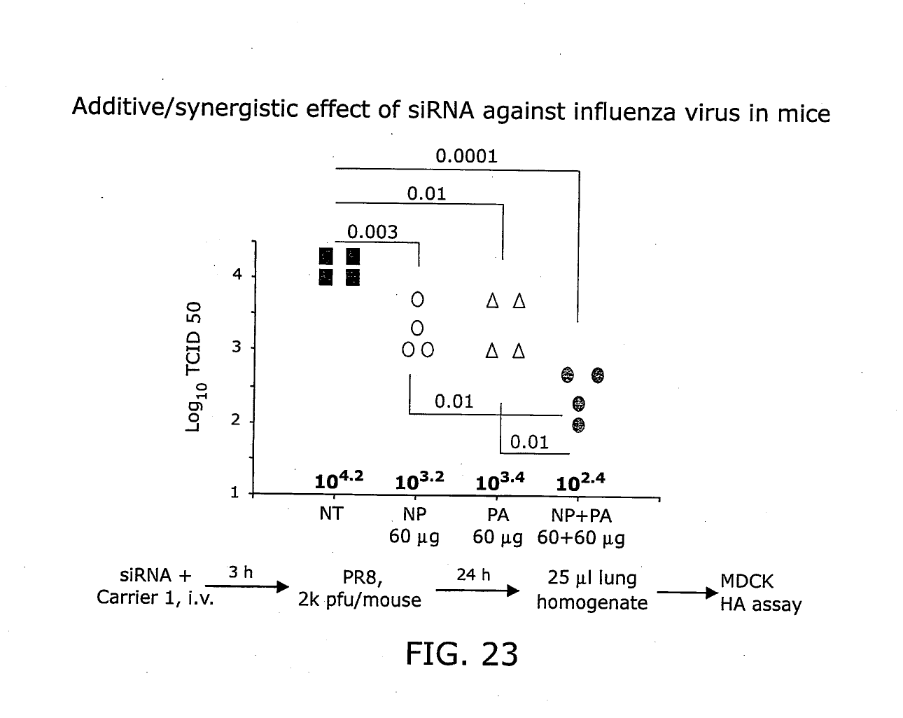

FIG. 23 shows results of an experiment demonstrating that siRNAs targeted to different influenza virus transcripts exhibit an additive effect. Sixty μg of NP-1496 siRNA, 60 μg PA-2087 siRNA, or 60 μg NP-1496 siRNA+60 μg PA-2087 siRNA were incubated with jetPEI and injected intravenously into mice as described above in Materials and Methods. Three hours later mice were intranasally infected with PR8 virus, 12000 pfu per mouse. Lungs were harvested 24 hours after infection. As shown in FIG. 23, the average log₁₀TCID₅₀ of the lung homogenate for mice that received no siRNA treatment (NT; filled squares) was 4.2. In mice that received 60 μg siRNA targeted to NP (NP 60 μg; open circles), the average log₁₀TCID₅₀ of the lung homogenate was 3.2. In mice that received 60 μg siRNA targeted to PA (PA 60 μg; open triangles), the average log₁₀TCID₅₀ of the lung homogenate was 3.4. In mice that received 60 μg siRNA targeted to NP+60 μg siRNA targeted to PA (NP+PA; filled circles), the average log₁₀TCID₅₀ of the lung homogenate was 2.4. The differences in virus titer in the lung homogenate between the group that received no treatment and the groups that received 60 μg NP siRNA, 60 μg PA siRNA, or 60 μg NP siRNA+60 μg PA siRNA were significant with P=0.003, 0.01, and 0.0001, respectively. The differences in lung homogenate between the groups that received 60 μg NP siRNA or 60 μg NP siRNA and the group that received 60 μg NP siRNA+60 μg PA siRNA were significant with P=0.01. Data for individual mice are presented in Table 7 (NT=no treatment). These data indicate that pretreatment with siRNA targeted to the influenza NP or PA transcript reduced the virus titer in the lungs of mice subsequently infected with influenza virus. The data further indicate that a combination of siRNA targeted to different viral transcripts exhibit an additive effect, suggesting that therapy with a combination of siRNAs targeted to different transcripts may allow a reduction in dose of each siRNA, relative to the amount of a single siRNA that would be needed to achieve equal efficacy.

TABLE 7
Additive effect of siRNA against influenza virus in mice

| | |
|---|---|
| Treatment | log₁₀TCID50 |

| | | | | |
|---|---|---|---|---|
| NT | 4.3 | 4.3 | 4.0 | 4.0 |
| NP (60 μg) | 3.7 | 3.3 | 3.0 | 3.0 |
| PA (60 μg) | 3.7 | 3.7 | 3.0 | 3.0 |
| NP + PA (60 μg | 2.7 | 2.7 | 2.3 | 2.0 |
| each) | | | | |

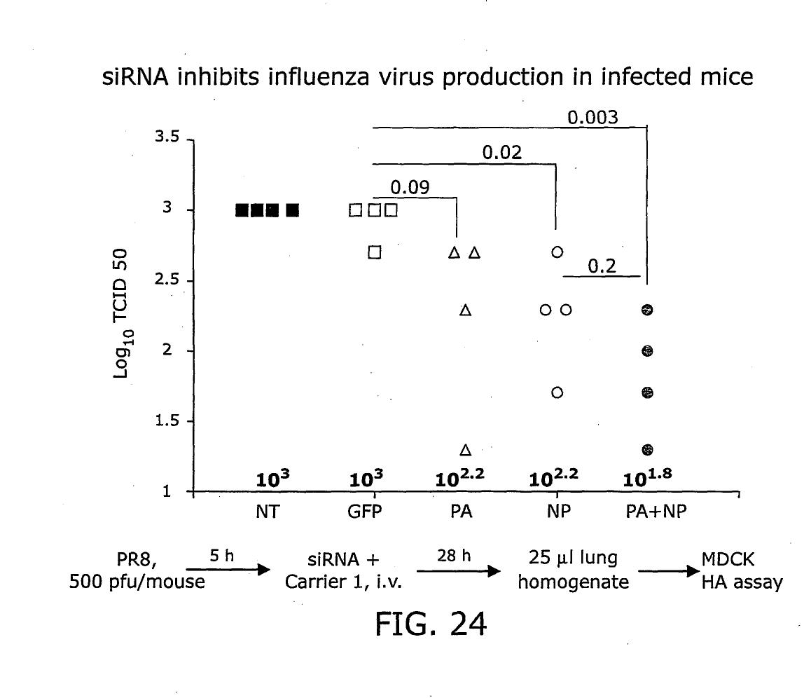

FIG. 24 shows results of an experiment demonstrating that siRNA targeted to viral NP transcripts inhibits influenza virus production in mice when administered following infection. Mice were intranasally infected with PR8 virus, 500 pfu. Sixty μg of GFP-949 siRNA, 60 μg PA-2087 siRNA, 60 μg NP-1496 siRNA, or 60 μg NP siRNA+60 μg PA siRNA were incubated with jetPEI and injected intravenously into mice 5 hours later as described above in Materials and Methods. Lungs were harvested 28 hours after administration of siRNA. As shown in FIG. 24, the average log₁₀TCID₅₀ of the lung homogenate for mice that received no siRNA treatment (NT; filled squares) or received the GFP-specific siRNA GFP-949 (GFP; open squares) was 3.0. In mice that received 60 μg siRNA targeted to PA (PA 60 μg; open triangles), the average log₁₀TCID₅₀ of the lung homogenate was 2.2. In mice that received 60 μg siRNA targeted to NP (NP 60 μg; open circles), the average log₁₀TCID₅₀ of the lung homogenate was 2.2. In mice that received 60 μg NP siRNA+60 μg PA siRNA (PA+NP; filled circles), the average log₁₀TCID₅₀ of the lung homogenate was 1.8. The differences in virus titer in the lung homogenate between the group that received no treatment and the groups that received 60 μg PA, NP siRNA, or 60 μg NP siRNA+60 μg PA siRNA were significant with P=0.09, 0.02, and 0.003, respectively. The difference in virus titer in the lung homogenate between the group that received NP siRNA and PA+NP siRNAs had a P value of 0.2. Data for individual mice are presented in Table 8 (NT=no treatment). These data indicate that siRNA targeted to the influenza NP and/or PA transcripts reduced the virus titer in the lung when administered following virus infection.

TABLE 8
Inhibition of influenza virus production in infected mice by siRNA

| | |
|---|---|
| Treatment | log₁₀TCID50 |

| | | | | |
|---|---|---|---|---|
| NT | 3.0 | 3.0 | 3.0 | 3.0 |
| GFP (60 μg) | 3.0 | 3.0 | 3.0 | 2.7 |
| PA (60 μg) | 2.7 | 2.7 | 2.3 | 1.3 |
| NP (60 μg) | 2.7 | 2.3 | 2.3 | 1.7 |
| NP + PA (60 μg | 2.3 | 2.0 | 1.7 | 1.3 |
| each) | | | | |

### Example 13

### Inhibition of Influenza Virus Production in Cells by Administration of a Lentivirus that Provides a Template for Production of shRNA

Materials and Methods

Cell culture. Vero cells were seeded in 24-well plates at 4×10⁵ cells per well in 1 ml of DMEM-10% FCS and were incubated at 37° C. under 5% CO₂.

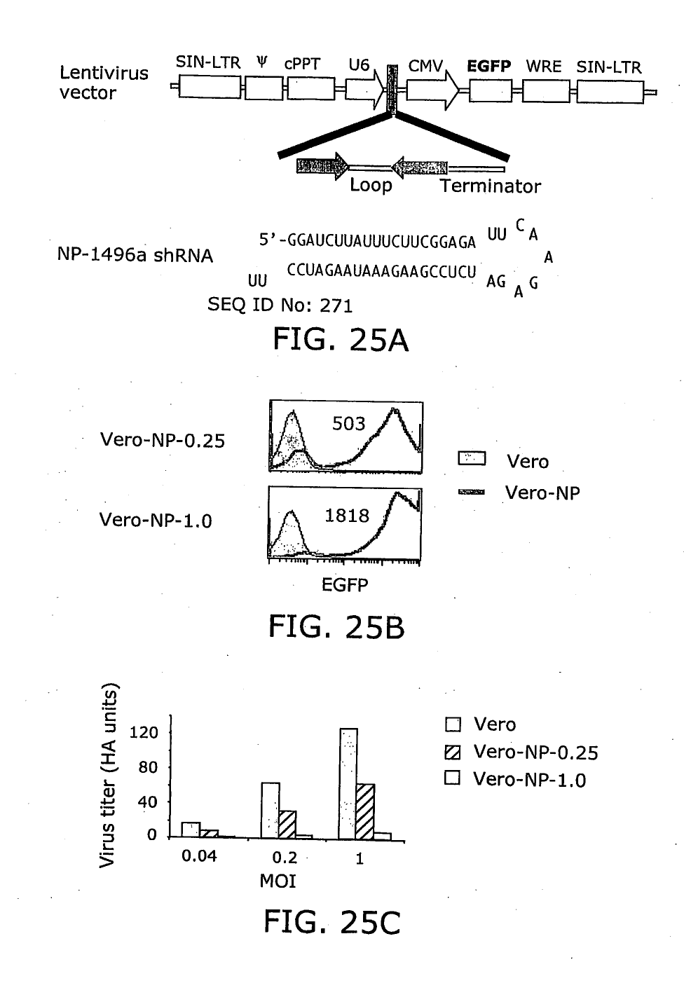

Production of lentivirus that provides a template for shRNA production. An oligonucleotide that serves as a template for synthesis of an NP-1496a shRNA (see FIG. 25A) was cloned between the U6 promoter and termination sequence of lentiviral vector pLL3.7 (Rubinson, D., et al, Nature Genetics, Vol. 33, pp. 401-406, 2003), as depicted schematically in FIG. 25A. The oligonucleotide was inserted between the HpaI and XhoI restriction sites within the multiple cloning site of pLL3.7. This lentiviral vector also expresses EGFP for easy monitoring of transfected/infected cells. Lentivirus was produced by co-transfecting the DNA vector comprising a template for production of NP-1496a shRNA and packaging vectors into 293T cells. Forty-eight h later, culture supernatant containing lentivirus was collected, spun at 2000 rpm for 7 min at 4° C. and then filtered through a 0.45 um filter. Vero cells were seeded at 1×10⁵ per well in 24-well plates. After overnight culture, culture supernatants containing that contained the insert (either 0.25 ml or 1.0 ml) were added to wells in the presence of 8 ug/ml polybrene. The plates were then centrifuged at 2500 rpm, room temperature for 1 h and returned to culture. Twenty-four h after infection, the resulting Vero cell lines (Vero-NP-0.25, and Vero-NP-1.0) were analyzed for GFP expression by flow cytometry along with parental (non-infected) Vero cells. It is noted that NP-1496a differs from NP-1496 due to the inadvertent inclusion of an additional nucleotide (A) at the 3′ end of the sense portion and a complementary nucleotide (U) at the 5′ end of the antisense portion, resulting in a duplex portion that is 20 nt in length rather than 19 as in NP-1496. (See Table 2). According to other embodiments of the invention NP-1496 sequences rather than NP-1496a sequences are used. In addition, the loop portion of NP-1496a shRNA differs from that of NP-1496 shRNA shown in FIG. 21.

Influenza virus infection and determination of viral titer. Control Vero cells and Vero cells infected with lentivirus containing the insert (Vero-NP-0.25 and Vero-NP-1.0) were infected with PR8 virus at MOI of 0.04, 0.2 and 1. Influenza virus titers in the supernatants were determined by HA assay 48 hrs after infection as described in Example 12.

Results

Lentivirus containing templates for production of NP-1496a shRNA were tested for ability to inhibit influenza virus production in Vero cells. The NP-1496a shRNA includes two complementary regions capable of forming a stem-loop structure containing a double-stranded portion that has the same sequence as the NP-1496a siRNA described above. As shown in FIG. 25B, incubation of lentivirus-containing supernatants with Vero cells overnight resulted in expression of EGFP, indicating infection of Vero cells by lentivirus. The shaded curve represents mean fluorescence intensity in control cells (uninfected). When 1 ml of supernatant was used, almost all cells became EGFP positive and the mean fluorescence intensity was high (1818) (Vero-NP-1.0). When 0.25 ml of supernatant was used, most cells (˜95%) were EGFP positive and the mean fluorescence intensity was lower (503) (Vero-NP-0.25).

Parental Vero cells and lentivirus-infected Vero cells were then infected with influenza virus at MOI of 0.04, 0.2, and 0.1, and virus titers were assayed 48 hrs after influenza virus infection. With increasing MOI, the virus titers increased in the supernatants of parental Vero cell cultures (FIG. 25C). In contrast, the virus titers remained very low in supernatants of Vero-NP-1.0 cell cultures, indicating influenza virus production was inhibited in these cells. Similarly, influenza virus production in Vero-NP-0.25 cell cultures was also partially inhibited. The viral titers are presented in Table 9. These results suggest that NP-1496 shRNA expressed from lentivirus vectors can be processed into siRNA to inhibit influenza virus production in Vero cells. The extent of inhibition appears to be proportional to the extent of virus infection per cell (indicated by EGFP level).

TABLE 9
Inhibition of influenza virus production by siRNA expressed in cells
in tissue culture

| Cell Line | Viral Titer | | |
|---|---|---|---|
| Vero | 16 | 64 | 128 |
| Vero-NP-0.25 | 8 | 32 | 64 |
| Vero-NP-1.0 | 1 | 4 | 8 |

### Example 14

### Inhibition of Influenza Production in Mice by Intranasal Administration of a DNA Vector From Which siRNA Precursors Are Transcribed

Materials and Methods

Construction of plasmids that serves as template for shRNA. Construction of a plasmid from which NP-1496a shRNA is expressed is described in Example 13. Oligonucleotides that serve as templates for synthesis of PB1-2257 shRNA or RSV-specific shRNA were cloned between the U6 promoter and termination sequence of lentiviral vector pLL3.7 as described in Example 13 and depicted schematically in FIG. 25A for NP-1496a shRNA. The sequences of the oligonucleotides were as follows:

NP-1496a sense:

(SEQ ID NO: 179)

5′-
TGGATCTTATTTCTTCGGAGATTCAAGAGATCTCCGAAGAAATAAGATCC
TTTTTTC-3′
NP-1496a antisense:

(SEQ ID NO: 180)

5′-TCGAGAAAAAAGGATCTTATTTCTTCGGAGATCTCTTGAATCTCCGA
AGAAATAAGATCCA-3′
PB1-2257 sense:

(SEQ ID NO: 181)

5′-TGATCTGTTCCACCATTGAATTCAAGAGATTCAATGGTGGAACAGAT
CTTTTTTC-3′
PB1-2257 antisense:

(SEQ ID NO: 182)

5′-
TCGAGAAAAAAGATCTGTTCCACCATTGAATCTCTTGAATTCAATGGTGG
AACAGATCA-3′
RSV sense:

(SEQ ID NO: 183)

5′-TGCGATAATATAACTGCAAGATTCAAGAGATCTTGCAGTTATATTAT
CGTTTTTTC-3′
RSV antisense:

(SEQ ID NO: 184)

5′-TCGAGAAAAAACGATAATATAACTGCAAGATCTCTTGAATCTTGCAG
TTATATTATCGCA-3′

The RSV shRNA expressed from the vector comprising the above oligonucleotide is processed in vivo to generate an siRNA having sense and antisense strands with the following sequences:

(SEQ ID NO: 185)

Sense: 5′-CGATAATATAACTGCAAGA-3′

(SEQ ID NO: 186)

Antisense: 5′-TCTTGCAGTTATATTATCG-3′

A PA-specific hairpin may be similarly constructed using the following oligonucleotides:

PA-2087 sense:

(SEQ ID NO: 187)

5′-TGCAATTGAGGAGTGCCTGATTCAAGAGATCAGGCACTCCTCAATTG
CTTTTTC-3′
PA-2087 antisense:

(SEQ ID NO: 270)

5′-
TCGAGAAAAAAGCAATTGAGGAGTGCCTGATCTCTTGAATCAGGCACTCC
TCAATTGCA-3′

Viral infection and determination of viral titer. These were performed as described in Example 12.

DNA Delivery. Plasmid DNAs capable of serving as templates for expression of NP-1496a shRNA, PB 1-2257 shRNA, or RSV-specific shRNA (60 μg each) were individually mixed with 40 μl Infasurf (ONY, Inc., Amherst N.Y.) and 20 μl of 5% glucose and were administered intranasally to groups of mice, 4 mice each group, as described above. A mixture of 40 μl Infasurf and 20 μl of 5% glucose was administered to mice in the no treatment (NT) group. The mice were intranasally infected with PR8 virus, 12000 pfu per mouse, 13 hours later, as described above. Lungs were harvested and viral titer determined 24 hours after infection.

Results

The ability of shRNAs expressed from DNA vectors to inhibit influenza virus infection in mice was tested. For these experiments, plasmid DNA was mixed with Infasurf, a natural surfactant extract from calf lung similar to vehicles previously shown to promote gene transfer in the lung (74). The DNA/Infasurf mixtures were instilled into mice by dropping the mixture into the nose using a pipette. Mice were infected with PR8 virus, 12000 pfu per mouse, 13 hours later. Twenty-four hrs after influenza virus infection, lungs were harvested and virus titers were measured by MDCK/hemagglutinin assay.

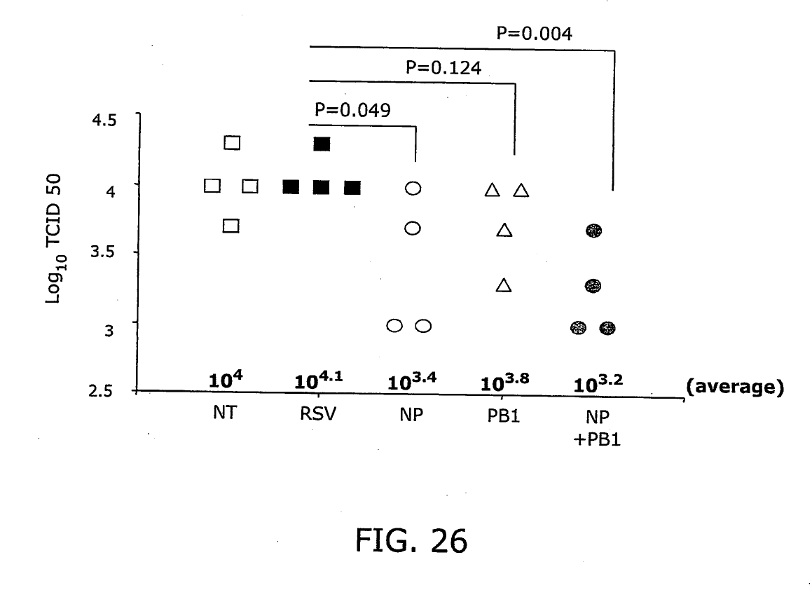

As shown in FIG. 26, virus titers were high in mice that were not given any plasmid DNA or were given a DNA vector expressing a respiratory syncytial virus (RSV)-specific shRNA. Lower virus titers were observed when mice were given plasmid DNA that expresses either NP-1496a shRNA or PB1-2257 shRNA. The virus titers were more significantly decreased when mice were given both influenza-specific plasmid DNAs together, one expressing NP-1496a shRNA and the other expressing PB1-2257 shRNA.

The average log₁₀TCID₅₀ of the lung homogenate for mice that received no treatment (NT; open squares) or received a plasmid encoding an RSV-specific shRNA (RSV; filled squares) was 4.0 or 4.1, respectively. In mice that received plasmid capable of serving as a template for NP-1496a shRNA (NP; open circles), the average log₁₀TCID₅₀ of the lung homogenate was 3.4. In mice that received plasmid capable of serving as a template for PB1-2257 shRNA (PB; open triangles), the average log₁₀TCID₅₀ of the lung homogenate was 3.8. In mice that received plasmids capable of serving as templates for NP and PB shRNAs (NP+PB1; filled circles), the average log₁₀TCID₅₀ of the lung homogenate was 3.2. The differences in virus titer in the lung homogenate between the group that received no treatment or RSV-specific shRNA plasmid and the groups that received NP shRNA plasmid, PB1 shRNA plasmid, or NP and PB1 shRNA plasmids had P values of 0.049, 0.124, and 0.004 respectively. Data for individual mice are presented in Table 10 (NT=no treatment). These results show that shRNA expressed from DNA vectors can be processed into siRNA to inhibit influenza virus production in mice and demonstrate that Infasurf is a suitable vehicle for the delivery of plasmids from which shRNA can be expressed. In particular, these data indicate that shRNA targeted to the influenza NP and/or PB1 transcripts reduced the virus titer in the lung when administered following virus infection.

TABLE 10
Inhibition of influenza virus production by shRNA expressed in mice

| | |
|---|---|
| Treatment | log₁₀TCID50 |

| | | | | |
|---|---|---|---|---|
| NT | 4.3 | 4.0 | 4.0 | 4.3 |
| RSV (60 μg) | 4.3 | 4.0 | 4.0 | 4.0 |
| NP (60 μg) | 4.0 | 3.7 | 3.0 | 3.0 |
| PB1 (60 μg) | 4.0 | 4.0 | 3.7 | 3.3 |
| NP + PB1 (60 μg | 3.7 | 3.3 | 3.0 | 3.0 |
| each) | | | | |

### Example 15

### Cationic Polymers Promote Cellular Uptake of siRNA

Materials and Methods

Reagents. Poly-L-lysines of two different average molecular weights [poly-L-lysine (MW (vis) 52,000; MW (LALLS) 41,800, Cat. No. P2636) and poly-L-lysine (MW (vis) 9,400; MW (LALLS) 8,400, Cat. No. P2636], poly-L-arginine (MW 15,000-70,000 Cat. No. P7762) and 3-(4,5-dimethylthiazol-2-yl)-2,5-diphenyl tetrazolium bromide (MTT) were purchased from Sigma. For purposes of description molecular weights obtained using the LALLS method will be assumed, but it is to be understood that molecular weights are approximate since the polymers display some heterogeneity in size.

Gel retardation assay. siRNA-polymer complexes were formed by mixing 10 μl of siRNA (10 pmol in 10 mM Hepes buffer, pH 7.2) with 10 μl of polymer solution containing varying amounts of polymer. Complexes were allowed to form for 30 min at room temperature, after which 20 μl was run on a 4% agarose gel. Bands were visualized with ethidium-bromide staining.

Cytotoxicity assay. siRNA-polymer complexes were formed by mixing equal amounts (50 pmol) of siRNA in 10 mM Hepes buffer, pH 7.2 with polymer solution containing varying amounts of polymer for 30 min at room temperature. Cytotoxicity was evaluated by MTT assay. Cells were seeded in 96-well plates at 30,000 cells per well in 0.2 ml of DMEM containing 10% fatal calf serum (FCS). After overnight incubation at 37° C., the medium was removed and replaced with 0.18 ml OPTI-MEM (GIBCO/BRL). siRNA-polymer complexes in 20 μl of Hepes buffer were added to the cells. After a 6-h incubation at 37° C., the polymer-containing medium was removed and replaced with DMEM-10% FCS. The metabolic activity of the cells was measured 24 h later using the MTT assay according to the manufacturer's instructions. Experiments were performed in triplicate, and the data was averaged.

Cell culture, transfection, siRNA polymer complex formation, and viral titer determination. Vero cells were grown in DMEM containing 10% heat-inactivated FCS, 2 mM L-glutamine, 100 units/ml penicillin, and 100 μg/ml streptomycin at 37° C. under a 5% CO2/95% air atmosphere. For transfection experiments, logarithmic-phase Vero cells were seeded in 24-well plates at 4×10⁵ cells per well in 1 ml of DMEM-10% FCS. After overnight incubation at 37° C., siRNA-polymer complexes were formed by adding 50 μl of siRNA (400 pmol (about 700 ng) in 10 mM Hepes buffer, pH 7.2) to 50 μl of polymer vortexing. Different concentrations of polymer were used in order to achieve complete complex formation between the siRNA and polymer. The mixture was incubated at room temperature for 30 min to complete complex formation. The cell-growth medium was removed and replaced with OPTI-MEM I (Life Technologies) just before the complexes were added.

After incubating the cells with the complexes for 6 h at 37° C. under 5% CO₂, the complex-containing medium was removed and 200 μl of PR8 virus in infection medium, MOI=0.04, consisting of DMEM, 0.3% BSA (Sigma), 10 mM Hepes, 100 units/ml penicillin, and 100 μg/ml streptomycin, was added to each well. After incubation for 1 h at room temperature with constant rocking, 0.8 ml of infection medium containing 4 μg/ml trypsin was added to each well and the cells were cultured at 37° C. under 5% CO₂. At different times after infection, supernatants were harvested from infected cultures and the virus titer was determined by hemagglutination (HA) assay as described above.

Transfection of siRNA by Lipofectamine 2000 (Life Technology) was carried out according to the manufacturer's instruction for adherent cell lines. Briefly, logarithmic-phase Vero cells were seeded in 24-well plate at 4×10⁵ cells per well in 1 ml of DMEM-10% FCS and were incubated at 37° C. under 5% CO₂. On the next day, 50 μl of diluted Lipofectamine 2000 in OPTI-MEM I were added to 50 μl of siRNA (400 pmol in OPTI-MEM I) to form complexes. The cell were washed and incubated with serum-free medium. The complexes were applied to the cells and the cells were incubated at 37° C. for 6 h before being washed and infected with influenza virus as described above. At different times after infection, supernatants were harvested from infected cultures and the virus titer was determined by hemagglutination (HA) assay as described above.

Results

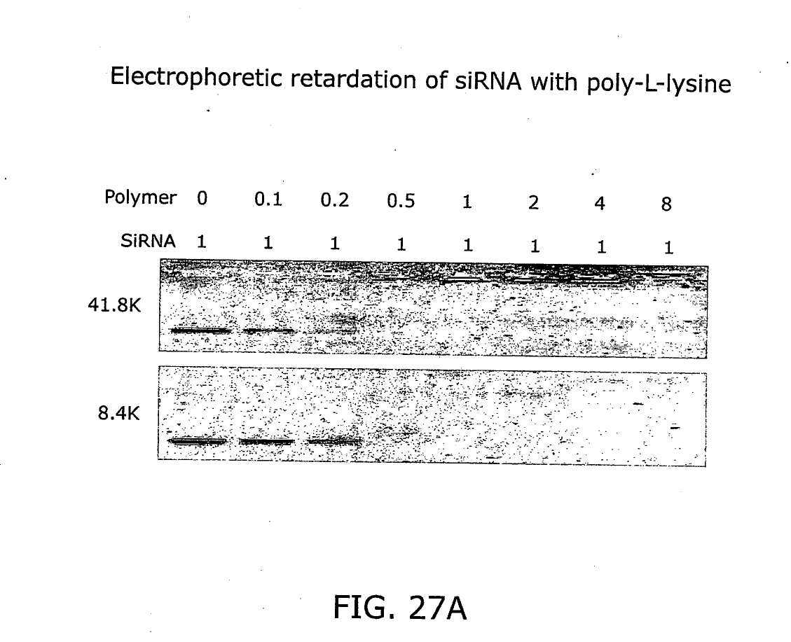

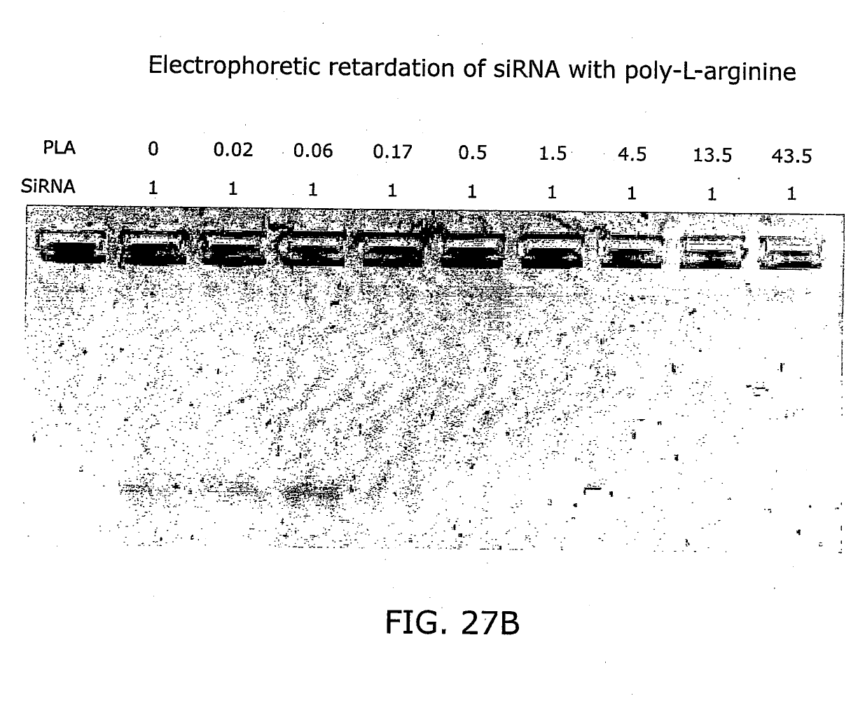

The ability of poly-L-lysine (PLL) and poly-L-arginine (PLA) to form complexes with siRNA and promote uptake of siRNA by cultured cells was tested. To determine whether PLL and/or PLA form complexes with siRNA, a fixed amount of NP-1496 siRNA was mixed with increasing amounts of polymer. Formation of polymer/siRNA complexes was then visualized by electrophoresis in a 4% agarose gel. With increasing amounts of polymer, electrophoretic mobility of siRNA was retarded (FIGS. 27A and 27B), indicating complex formation. FIGS. 27A and 27B represent complex formation between siRNAs and PLL (41.8K) or PLA, respectively. The amount of polymer used in each panel increases from left to right. In FIGS. 27A and 27B in each panel, a band can be seen in the lanes on the left, indicating lack of complex formation and hence entry of the siRNA into the gel and subsequent migration. As one moves to the right, the band disappears, indicating complex formation and failure of the complex to enter the gel and migrate.

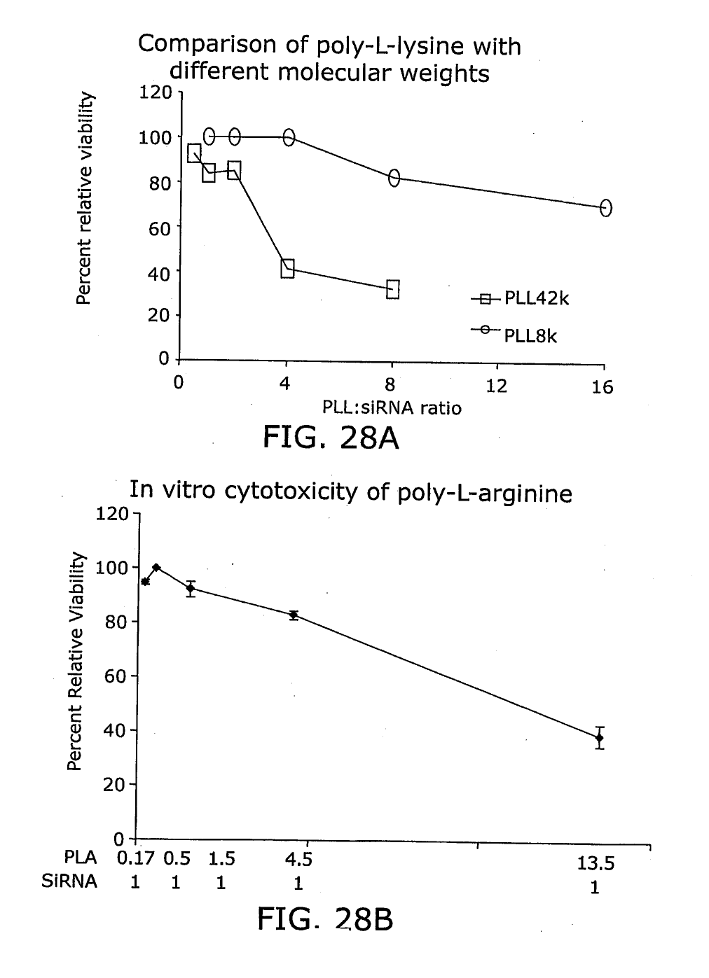

To investigate cytotoxicity of siRNA/polymer complexes, mixtures of siRNA and PLL or PLA at different ratios were added to Vero cell cultures in 96-well plates. The metabolic activity of the cells were measured by MIT assay (74). Experiments were performed in triplicate, and data was averaged. Cell viability was significantly reduced with increasing amounts of PLL (MW ˜42K) whereas PLL (˜8K) showed significantly lower toxicity, exhibiting minimal or no toxicity at PLL/siRNA ratios as high as 4:1 (FIG. 28A; circles=PLL (MW˜8K); squares=PLL (MW ˜42K)). Cell viability was reduced with increasing PLA/siRNA ratios as shown in FIG. 28B, but viability remained above 80% at PLA/siRNA ratios as high as 4.5:1. The polymer/siRNA ratio is indicated on the x-axis in FIGS. 28A and 28B. The data plotted in FIGS. 28A and 28B are presented in Tables 11 and 12. In Table 11 the numbers indicate % viability of cells following treatment with polymer/siRNA complexes, relative to % viability of untreated cells. ND=Not done. In Table 12 the numbers indicate PLA/siRNA ratio, % survival, and standard deviation as shown.

TABLE 11
Cytotoxicity of PLL/siRNA complexes (% survival)

| | polymer/siRNA ratio | | | | | |
|---|---|---|---|---|---|---|
| Treatment | 0.5 | 1.0 | 2.0 | 4.0 | 8.0 | 16.0 |
| PLL ~8.4K | 92.26 | 83.57 | 84.39 | 41.42 | 32.51 | ND |
| PLL ~41.8K | ND | 100 | 100 | 100 | 82.55 | 69.63 |

TABLE 12
Cytotoxicity of PLA/siRNA complexes (% survival)

| | polymer/siRNA ratio | | | | |
|---|---|---|---|---|---|
| | 0.17 | 0.5 | 1.5 | 4.5 | 13.5 |
| % survival | 94.61 | 100 | 92.33 | 83 | 39.19 |
| Standard | .74 | 1.91 | 2.92 | 1.51 | 4.12 |
| deviation | | | | | |

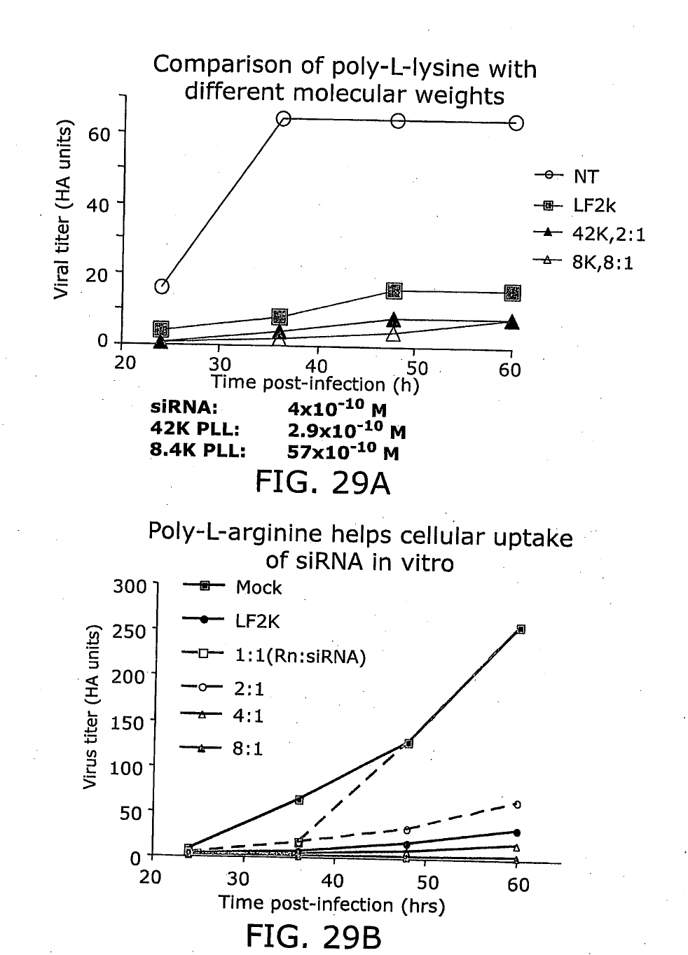

To determine whether PLL or PLA promotes cellular uptake of siRNA, various amounts of polymer and NP-1496 were mixed at ratios at which all, siRNA was complexed with polymer. Equal amounts of siRNA were used in each case. A lower polymer/siRNA ratio was used for ˜42K PLL than for ˜8K PLL since the former proved more toxic to cells. The complexes were added to Vero cells, and 6 hrs later the cultures were infected with PR8 virus. At different times after infection, culture supernatants were harvested and assayed for virus by HA assay. FIG. 29A is a plot of virus titers over time in cells receiving various transfection treatments (circles=no treatment; squares=Lipofectamine; filled triangles=PLL (˜42K at PLL/siRNA ratio=2); open triangles=PLL (˜8K at PLL/siRNA ratio=8). As shown in FIG. 29A, virus titers increased with time in the non-transfected cultures. Virus titers were significantly lower in cultures that were transfected with NP-1496/Lipofectamine and were even lower in cultures treated with PLL/NP-1496 complexes. The data plotted in FIG. 29A are presented in Table 13A (NT=no treatment; LF2K=Lipofectamine. The PLL:siRNA ratio is indicated in parentheses.

PLA was similarly tested over a range of polymer/siRNA ratios. FIG. 29B is a plot of virus titers over time in cells receiving various transfection treatments (filled squares=mock transfection; filled circles=Lipofectamine; open squares=PLA at PLA/siRNA ratio=1; open circles=PLA at PLA/siRNA ratio=2; open triangles=PLA at PLA/siRNA ratio=4; filled triangles=PLA at PLA/siRNA ratio=8). As shown in FIG. 29B, virus titers increased with time in the control (mock-transfected) culture and in the culture treated with PLA/siRNA at a 1:1 ratio. Virus titers were significantly lower in cultures that were transfected with NP-1496/Lipofectamine and were even lower in cultures treated with PLA/siRNA complexes containing complexes at PLA/siRNA ratios of 4:1 or higher. Increasing amounts of polymer resulted in greater reduction in viral titer. The data plotted in FIG. 29B are presented in Table 13B.

TABLE 13A
Inhibition of influenza virus production by polymer/siRNA complexes

Time (hours)

| Treatment | 24 | 36 | 48 | 60 |
|---|---|---|---|---|
| mock transfection | 16 | 64 | 64 | 64 |
| LF2K | 4 | 8 | 16 | 16 |
| PLL ~42K (2:1) | 1 | 4 | 8 | 8 |
| PLL ~8K (8:1) | 1 | 2 | 4 | 8 |

TABLE 13B
Inhibition of influenza virus production by polymer/siRNA complexes

Time (hours)

| Treatment | 24 | 36 | 48 | 60 |
|---|---|---|---|---|
| mock transfection | 8 | 64 | 128 | 256 |
| LF2K | 2 | 6 | 16 | 32 |
| PLA (1:1) | 4 | 16 | 128 | 256 |
| PLA (2:1) | 4 | 16 | 32 | 64 |
| PLA (4:1) | 1 | 4 | 8 | 16 |
| PLA (8:1) | 1 | 1 | 1 | 2 |

Thus, cationic polymers promote cellular uptake of siRNA and inhibit influenza virus production in a cell line and are more effective than the widely used transfection reagent Lipofectamine. These results also suggest that additional cationic polymers may readily be identified to stimulate cellular uptake of siRNA and describe a method for their identification. PLL and PLA can serve as positive controls for such efforts.

### Example 16A

### Inhibition of Luciferase Activity in the Lung by Delivery of siRNA to the Vascular System or the Respiratory Tract

Materials and Methods

siRNAs were obtained from Dharmacon and were deprotected and annealed as described above. siRNA sequences for NP (NP-1496), PA (PA-2087), PB1 (PB1-2257), and GFP were as given above. Luc-specific siRNA was as described in (McCaffrey, A P, et al., Nature, 418:38-39)

PEI-mediated DNA transfection in mice. pCMV-luc DNA (Promega) was mixed with PEI (Qbiogene, Carlsbad, Calif.) at a nitrogen/phosphorus molar ratio (N/P ratio) of 10 at room temperature for 20 min. For i.v. administration, 200 μl of the mixture containing 60 μg of DNA was injected retroorbitally into 8 week old male C57BL/6 mice (Taconic Farms). For intratracheal (i.t.) adminstration, 50 μl of the mixture containing 30 μg or 60 μg of DNA was administered into the lungs of anesthetized mice using a Penn Century Model IA-IC insufflator.

PEI-mediated siRNA delivery in mice. siRNA-PEI compositions were formed by mixing 60 μg of luc-specific or GFP-specific siRNA with jetPEI at an N/P ratio of 5 at room temperature for 20 min. For i.v. administration, 200 μl of the mixture containing the indicated amounts of siRNA was injected retroorbitally. For pulmonary administration, 50 μl was delivered intratracheally.

Luc assay. At various times after pCMV-luc DNA administration, lungs, spleen, liver, heart, and kidney were harvested and homogenized in Cell Lysis Buffer (Marker Gene Technologies, Eugene, Oreg.). Luminescence was analyzed with the Luciferase Assay System (Promega) and measured with an Optocomp® I luminometer (MGM Instruments, Hamden, Conn.). The protein concentrations in homogenates were measured by the BCA assay (Pierce).

Results

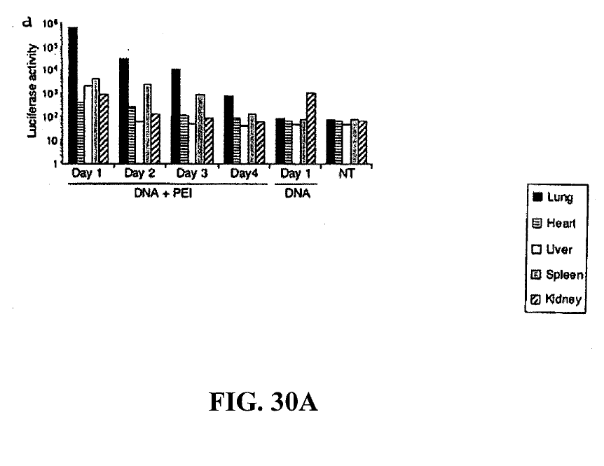

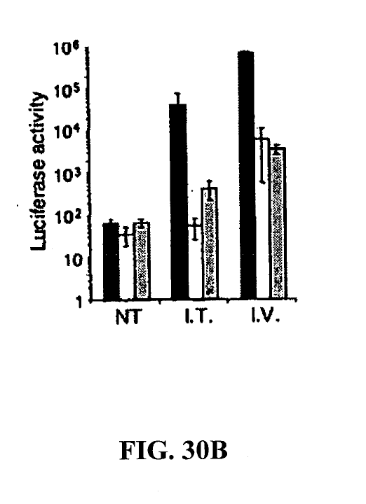

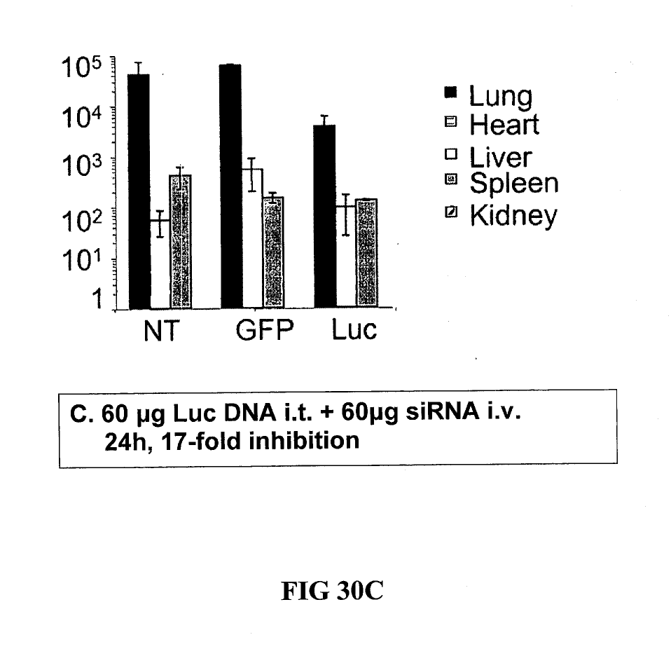

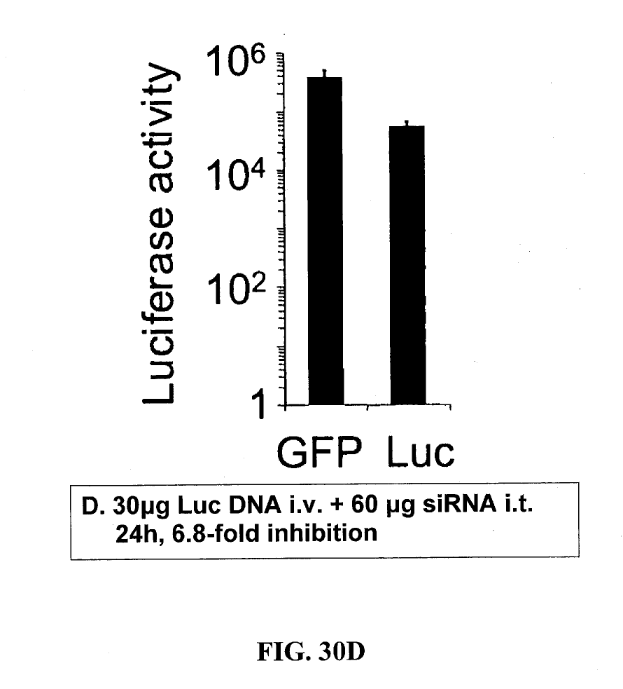

To determine the tissue distribution of PEI-mediated nucleic acid delivery in mice, pCMV-luc DNA-PEI complexes were injected i.v., and 24 hr later, Luc activity was measured in various organs. Activity was highest in the lungs, where Luc activity was detected for at least 4 days (FIG. 30A), whereas in heart, liver, spleen, and kidney, levels were 100-1,000 times lower and were detected for a shorter time after injection. When DNA-PEI complexes were instilled i.t., significant Luc activity was also detected in the lungs, although at a lower level than after i.v. adminstration (FIG. 30B).

To test the ability of PEI to promote uptake of siRNAs by the lungs following i.v. administration, mice were first given pCMV-luc DNA-PEI complexes i.t., followed by i.v. injection of Luc-specific siRNA complexed with PEI, control GFP-specific siRNA complexed with PEI, or the same volume of 5% glucose. Twenty-four hours later, Luc activity in the lungs was 17-fold lower in mice that received Luc siRNA than in those given GFP siRNA or no treatment (FIG. 30C). Because Luc siRNA can inhibit Luc expression only in the same lung cells that were transfected with the DNA vector, these results indicate that i.v. injection of a siRNA-PEI mixture achieves effective inhibition of a target transcript in the lung.

To test the ability of PEI to promote uptake of siRNAs by the lungs following pulmonary administration, mice were first given pCMVDNA-PEI complexes i.v., followed immediately by i.t. administration of Luc-specific siRNA mixed with PEI, control GFP-specific siRNA mixed with PEI, or the same volume of 5% glucose. Twenty-four hours later, luciferase activities were assayed in lung homogenates. FIG. 30D shows luciferase activity in lung homogenates from mice given either luciferase-specific or GFP-specific siRNA, normalized to protein amounts. Luciferase activity was 6.8-fold lower in mice that were treated with luciferase siRNA than those treated with GFP siRNA. These results indicate that pulmonary administration of an siRNA-PEI mixture achieves effective inhibition of a target transcript in lung cells.

### Example 16B

### Inhibition of Cyclophilin B in the Lung by Delivery of siRNA to the Respiratory System

Cyclophilin B is an endogenous gene that is widely expressed in mammals. To assess the ability of siRNA delivered directly to the respiratory system to inhibit expression of an endogenous gene, outbred Blackswiss mice (around 30 g or more body weight) were anesthetized by isofluorane/oxygen, and siRNA targeted to cyclophilin B (Dharmacon, D-001136-01-20 siCONTROL Cyclophilin B siRNA (Human/Mouse/Rat) or control GFP-949 siRNA (2 mg/kg) was administered intranasally to groups of 2 mice for each siRNA. Lungs were harvested 24 hours after administration. RNA was extracted from the lung and reverse transcription was done using a random primer. Real time PCR was then performed using cyclophilin B and GAPDH Taqman gene expression assay (Applied Biosystems). Results (Table 14) showed 70% silencing of cyclophilin B by siRNA targeted to cyclophilin B.

TABLE 14
Inhibition of Cyclophilin B in the Lung

| | | Ave | |
|---|---|---|---|
| Average | Normalized | normal | silencing % |
| PBS-1 | 5.395406 | 4.288984 | |
| PBS-2 | 3.182562 | | |
| GFP-1 | 2.547352 | 3.752446 | 12.50968 |
| GFP-2 | 4.957539 | | |
| Cyclo-1 | 1.173444 | 1.256672 | 70.7 |
| Cyclo-2 | 1.339901 | | |

### Example 17

### Selection of Favorably Conserved Target Portions

To identify favorably conserved regions of various influenza virus A transcripts for use as target portions against which to target RNAi-inducing agents to inhibit expression in a wide variety of strains, genome segments from a set of virus strains isolated from humans were aligned (in their positive sense form, i.e., the sequences found in mRNA). The strains included a number of strains in addition to those listed in Example 1. Tables 15A-15H list the Genbank accession number (left column), strain name (middle column), and serotype (right column) of the influenza A virus genome segments that were used to identify favorably conserved regions. The entire sequence of each segment was aligned and compared, with the exception of introns. 5′ and 3′ untranslated regions were included. The set of strains that was aligned differed for different segments, but each set included at least 19 strains isolated in various years spanning the time between 1934 and 2004. The strains included all HA and NA types known to circulate in humans (H1, H2, H3, H5, H9, N1, N2).

TABLE 15A
Influenza A virus NP segment (strains isolated from humans)

| | | |
|---|---|---|
| AF389119 | A/Puerto Rico/8/34/Mount Sinai | H1N1 |
| M63752 | A/Singapore/1/57 | H2N2 |
| M23976 | A/Ann Arbor/6/60 | H2N2 |
| AY210103 | A/Korea/426/68 | H2N2 |
| M76606 | A/New Jersey/8/76 | H1N1 |
| L07351 | A/Memphis/18/78 | H3N2 |
| AJ628066 | A/Fiji/15899/83 | H1N1 |
| L07369 | A/Memphis/3/88 | H3N2 |
| M63755 | A/Wisconsin/3523/88 | H1N1 |
| L07373 | A/Guangdong/38/89 | H3N2 |
| L07357 | A/Shanghai/6/90 | H3N2 |
| L24394 | A/MD/12/91 | H1N1 |
| AF038254 | A/Kitakyushu/159/93 | H3N2 |
| AB019358 | A/Nagasaki/48/95 | H3N2 |
| AJ291400 | A/Hong Kong/156/97 | H5N1 |
| AF255749 | A/Hong Kong/498/97 | H3N2 |
| AF255752 | A/Hong Kong/542/97 | H5N1 |
| AF038259 | A/Shiga/25/97 | H3N2 |
| AF255753 | A/Hong Kong/97/98 | H5N1 |
| AF342819 | A/Wisconsin/10/98 | H1N1 |
| AJ289871 | A/Hong Kong/1073/99 | H9N2 |
| AJ458276 | A/Switzerland/9243/99 | H3N2 |
| ISDN13443 | A/Sydney/274/2000 | H3N2 |
| AB126624 | A/Yokohama/22/2002 | H1N2 |
| AY575905 | A/Hong Kong/212/03 | H5N1 |
| AY526749 | A/Viet Nam/1196/04 | H5N1 |

TABLE 15B
Influenza A virus PA transcripts (strains isolated from humans)

| | | |
|---|---|---|
| AF389117 | A/Puerto Rico/8/34/Mount Sinai | H1N1 |
| M26078 | A/Singapore/1/57 | H2N2 |
| M23974 | A/Ann Arbor/6/60 | H2N2 |
| M26079 | A/Korea/426/68 | H2N2 |
| AJ605762 | A/Fiji/15899/83 | H1N1 |
| AF037424 | A/Kitakyushu/159/93 | H3N2 |
| U71138 | A/Shiga/20/95 | H3N2 |
| AJ289874 | A/Hong Kong/156/97 | H5N1 |
| AF257198 | A/Hong Kong/498/97 | H3N2 |
| AF257201 | A/Hong Kong/542/97 | H5N1 |
| AF037429 | A/Shiga/25/97 | H3N2 |
| AF258519 | A/Hong Kong/427/98 | H1N1 |
| AF257202 | A/Hong Kong/97/98 | H5N1 |
| AY043028 | A/Guangzhou/333/99 | H9N2 |
| AF257191 | A/Hong Kong/1073/99 | H9N2 |
| AJ293922 | A/Hong Kong/1774/99 | H3N2 |
| AB126627 | A/Yokohama/22/2002 | H1N2 |
| AY576404 | A/Hong Kong/212/03 | H5N1 |
| AY526750 | A/Viet Nam/1196/04 | H5N1 |

TABLE 15C
Influenza A virus PB1 transcripts (strains isolated from humans)

| | | |
|---|---|---|
| AF389116 | A/Puerto Rico/8/34/Mount Sinai | H1N1 |
| M25924 | A/Singapore/1/57 | H2N2 |
| M23972 | A/Ann Arbor/6/60 | H2N2 |
| M25935 | A/Korea/426/68 | H2N2 |
| AJ564807 | A/Fiji/15899/83 | H1N1 |
| AF037418 | A/Kitakyushu/159/93 | H3N2 |
| U71130 | A/Shiga/20/95 | H3N2 |
| AJ404633 | A/Hong Kong/156/97 | H5N1 |
| AF258823 | A/Hong Kong/498/97 | H3N2 |
| AF258826 | A/Hong Kong/542/97 | H5N1 |
| AF037423 | A/Shiga/25/97 | H3N2 |
| AF258526 | A/Hong Kong/427/98 | H1N1 |
| AF258827 | A/Hong Kong/97/98 | H5N1 |
| AY043029 | A/Guangzhou/333/99 | H9N2 |
| AF258816 | A/Hong Kong/1073/99 | H9N2 |
| AJ293921 | A/Hong Kong/1774/99 | H3N2 |
| AJ489539 | A/England/627/01 | H1N2 |
| AB126625 | A/Yokohama/22/2002 | H1N2 |
| AY576392 | A/Hong Kong/212/03 | H5N1 |
| AY526751 | A/Viet Nam/1196/04 | H5N1 |

TABLE 15D
Influenza A virus PB2 transcripts (strains isolated from humans)

| | | |
|---|---|---|
| AF389115 | A/Puerto Rico/8/34/Mount Sinai | H1N1 |
| M73521 | A/Singapore/1/57 | H2N2 |
| M23970 | A/Ann Arbor/6/60 | H2N2 |
| M73524 | A/Korea/426/68 | H2N2 |
| M91712 | A/Udorn/307/72 | H3N2 |
| AJ564805 | A/Fiji/15899/83 | H1N1 |
| AF037412 | A/Kitakyushu/159/93 | H3N2 |
| U53158 | A/Wisconsin/4754/94 | H1N1 |
| U71134 | A/Shiga/20/95 | H3N2 |
| AF036363 | A/Hong Kong/156/97 | H5N1 |
| AF258842 | A/Hong Kong/498/97 | H3N2 |
| AF258845 | A/Hong Kong/542/97 | H5N1 |
| AF037417 | A/Shiga/25/97 | H3N2 |
| AF258525 | A/Hong Kong/427/98 | H1N1 |
| AF258846 | A/Hong Kong/97/98 | H5N1 |
| AF342824 | A/Wisconsin/10/98 | H1N1 |
| AY043030 | A/Guangzhou/333/99 | H9N2 |
| AJ404630 | A/Hong Kong/1073/99 | H9N2 |
| AJ293920 | A/Hong Kong/1774/99 | H3N2 |
| AJ489485 | A/England/627/01 | H1N2 |
| AB126626 | A/Yokohama/22/2002 | H1N2 |
| AY576380 | A/Hong Kong/212/03 | H5N1 |
| AY526752 | A/Viet Nam/1196/04 | H5N1 |

TABLE 15E
Influenza A virus M transcripts (strains isolated from humans)

| | | |
|---|---|---|
| AF389121 | A/Puerto Rico/8/34/Mount Sinai | H1N1 |
| X08093 | A/Singapore/1/57 | H2N2 |
| M23978 | A/Ann Arbor/6/60 | H2N2 |
| M63531 | A/Korea/426/68 | H2N2 |
| J02167 | A/udorn/72 | H3N2 |
| AJ298947 | A/Fiji/15899/83 | H1N1 |
| U65562 | A/Kitakyushu/159/93 | H3N2 |
| U53168 | A/Wisconsin/4754/94 | H1N1 |
| U65573 | A/Shiga/20/95 | H3N2 |
| AJ458306 | A/Sauth Africa/1147/96 | H3N2 |
| AF036358 | A/Hong Kong/156/97 | H5N1 |
| AF255370 | A/Hong Kong/498/97 | H3N2 |
| AF255373 | A/Hong Kong/542/97 | H5N1 |
| AF038274 | A/Shiga/25/97 | H3N2 |
| AF258523 | A/Hong Kong/427/98 | H1N1 |
| AF255374 | A/Hong Kong/97/98 | H5N1 |
| AF342818 | A/Wisconsin/10/98 | H1N1 |
| AY043025 | A/Guangzhou/333/99 | H9N2 |
| AF255363 | A/Hong Kong/1073/99 | H9N2 |
| AJ293925 | A/Hong Kong/1774/99 | H3N2 |
| AJ458304 | A/Saudi Arabia/7971/2000 | H1N1 |
| AJ489530 | A/England/627/01 | H1N2 |
| AB126629 | A/Yokohama/22/2002 | H1N2 |
| AY575893 | A/Hong Kong/212/03 | H5N1 |
| AY526748 | A/Viet Nam/1196/04 | H5N1 |

TABLE 15F
Influenza A virus NS transcripts (strains isolated from humans)

| | | |
|---|---|---|
| AF389122 | A/Puerto Rico/8/34/Mount Sinai | H1N1 |
| AY210151 | A/Singapore/1/57 | H2N2 |
| AY210161 | A/Ann Arbor/6/60 | H2N2 |
| AY210191 | A/Korea/426/68 | H2N2 |
| V01102 | A/UDORN/72 | H3N2 |
| AJ298950 | A/Fiji/15899/83 | H1N1 |
| D30676 | A/Kitakyushu/159/93 | H3N2 |
| U53170 | A/Wisconsin/4754/94 | H1N1 |
| U65673 | A/Shiga/20/95 | H3N2 |
| AF036360 | A/Hong Kong/156/97 | H5N1 |
| AF256183 | A/Hong Kong/498/97 | H3N2 |
| AF256187 | A/Hong Kong/542/97 | H5N1 |
| AF038279 | A/Shiga/25/97 | H3N2 |
| AF258521 | A/Hong Kong/427/98 | H1N1 |
| AF256188 | A/Hong Kong/97/98 | H5N1 |
| AF342817 | A/Wisconsin/10/98 | H1N1 |
| AY043027 | A/Guangzhou/333/99 | H9N2 |
| AF256177 | A/Hong Kong/1074/99 | H9N2 |
| AJ293941 | A/Hong Kong/1774/99 | H3N2 |
| AJ519463 | A/Saudi Arabia/7971/2000 | H1N1 |
| AJ489552 | A/England/627/01 | H1N2 |
| AB126628 | A/Yokohama/22/2002 | H1N2 |
| AY576368 | A/Hong Kong/212/03 | H5N1 |
| AY526747 | A/Viet Nam/1196/04 | H5N1 |

TABLE 15G
Influenza A virus NA transcripts (strains isolated from humans)

| | | |
|---|---|---|
| AF389120 | A/Puerto Rico/8/34/Mount Sinai | H1N1 |
| AY209895 | A/Singapore/1/57 | H2N2 |
| AY209903 | A/Ann Arbor/6/60 | H2N2 |
| AY209932 | A/Korea/426/68 | H2N2 |
| M27970 | A/New Jersey/8/76 | H1N1 |
| K01017 | A/Memphis/10/78 | H1N1 |
| AJ006954 | A/Fiji/15899/83 | H1N1 |
| U42634 | A/England/427/88 | H3N2 |
| U47816 | A/Wisconsin/3523/88 | H1N1 |
| U42636 | A/Shanghai/24/90 | H3N2 |
| U42774 | A/California/271/92 | H3N2 |
| AF038260 | A/Kitakyushu/159/93 | H3N2 |
| AJ457945 | A/Nanchang/933/95 | H3N2 |
| AF036357 | A/Hong Kong/156/97 | H5N1 |
| AF102670 | A/Hong Kong/542/97 | H5N1 |
| AF038264 | A/Shiga/25/97 | H3N2 |
| AF102661 | A/Hong Kong/97/98 | H5N1 |
| AF533749 | A/Montevideo/2728/98 | H3N2 |
| AY043022 | A/Shaoguan/408/98 | H9N2 |
| AF342820 | A/Wisconsin/10/98 | H1N1 |
| AJ404629 | A/Hong Kong/1073/99 | H9N2 |
| AJ293923 | A/Hong Kong/1774/99 | H3N2 |
| AJ307628 | A/Montreal/MTL516/00 | H3N2 |
| AF503466 | A/Singapore/63/2001 | H1N2 |
| AJ457947 | A/Ireland/1092/2002 | H3N2 |
| AB126623 | A/Yokohama/22/2002 | H1N2 |
| AY575881 | A/Hong Kong/212/03 | H5N1 |
| AY555151 | A/Thailand/1-KAN-1/2004 | H5N1 |
| AY526746 | A/Viet Nam/1196/04 | H5N1 |

TABLE 15H
Influenza A virus HA transcripts (strains isolated from humans)

| | | |
|---|---|---|
| AF389118 | A/Puerto Rico/8/34/Mount Sinai | H1N1 |
| L20410 | A/Singapore/1/57 | H2N2 |
| AF270721 | A/Ann Arbor/6/60 | H2N2 |
| L11133 | A/Korea/426/68 | H2N2 |
| AY661044 | A/Bilthoven/628/76 | H3N2 |
| AJ289702 | A/Fiji/15899/83 | H1N1 |
| AY661055 | A/England/427/88 | H3N2 |
| L33755 | A/Finland/75/88 | H1N1 |
| AY661074 | A/Shanghai/24/90 | H3N2 |
| AF008817 | A/California/271/92 | H3N2 |
| D30669 | A/Kitakyushu/159/93 | H3N2 |
| AF008725 | A/Nanchang/933/95 | H3N2 |
| AF036356 | A/Hong Kong/156/97 | H5N1 |
| AF102678 | A/Hong Kong/542/97 | H5N1 |
| AJ311466 | A/Sydney/5/97 | H3N2 |
| AF102676 | A/Hong Kong/97/98 | H5N1 |
| AY271794 | A/Memphis/31/98 | H3N2 |
| AY043017 | A/Shaoguan/408/98 | H9N2 |
| AF342821 | A/Wisconsin/10/98 | H1N1 |
| AJ404626 | A/Hong Kong/1073/99 | H9N2 |
| AJ293926 | A/Hong Kong/1774/99 | H3N2 |
| ISDNOS0022 | A/Oslo/6391/2000 | H3N2 |
| AF503481 | A/Singapore/63/2001 | H1N2 |
| AY661030 | A/Netherlands/120/02 | H3N2 |
| AB126622 | A/Yokohama/22/2002 | H1N2 |
| AY575869 | A/Hong Kong/212/03 | H5N1 |
| AY555150 | A/Thailand/1-KAN-1/2004 | H5N1 |
| AY526745 | A/Viet Nam/1196/04 | H5N1 |

In order to identify a set of preferred target portions among a large set of influenza A virus strains, we selected the sequence of the PR8 strain as a base sequence. We used the entire sequence of each segment, excluding introns. The sequences are shown in FIGS. 32A-32J (SEQ ID NOs: 383-392). These sequences were concatenated to form a single long sequence in the following order: NP, PB2, PB1, PA, M1, M2, NS1, NS2, HA, NA. Portions of the sequence having a length of 19 nucleotides were identified by starting at the 5′ end, applying a 19 nucleotide “window” to select the first potential target portion, and then shifting the window in the 3′ direction by 1 nucleotide at a time in order to select the next potential target portion. This process was continued until the 3′ end of the transcript was reached. Every 19 nucleotide region was considered to represent a potential target portion, though it was recognized that shorter or longer target portions could also have been selected. Regions that encompassed portions of two different transcripts were excluded. The sequences of the 13,472 potential target portions that resulted from this process may readily be determined by referring to SEQ ID NOs: 383-392 in FIG. 32. The target portions extend, for example, from positions 1-19, 2-20, 3-21, 4-22, 5-23, 6-24, etc., in each sequence. In the NP transcript (SEQ ID NO: 383), the target portions are 1-19, 2-20, 3-21, 4-22 . . . 1547-1565. In the PB2 (SEQ ID NO: 384) transcript, the target portions are 1-19, 2-20, 3-21, 4-22 . . . 2323-2341. In the PB1 transcript (SEQ ID NO: 385), the target portions are 1-19, 2-20, 3-21, 4-22 . . . 2323-2341. In the PA transcript (SEQ ID NO: 386), the target portions are 1-19, 2-20, 3-21, 4-22 . . . 2215-2233. In the M1 transcript (SEQ ID NO: 387), the target portions are 1-19, 2-20, 3-21, 4-22 . . . 1009-1027. In the M2 transcript (SEQ ID NO: 388), the target portions are 1-19, 2-20, 3-21, 4-22 . . . 321-339. In the NS1 transcript (SEQ ID NO: 389), the target portions are 1-19, 2-20, 3-21, 4-22 . . . 872-890. In the NS2 transcript (SEQ ID NO: 390), the target portions are 1-19, 2-20, 3-21, 4-22 . . . 400-418. In the HA transcript (SEQ ID NO: 391), the target portions are 1-19, 2-20, 3-21, 4-22 . . . 1767-1775. In the NA transcript, the target portions are 1-19, 2-20, 3-21, 4-22 . . . 1395-1413 (SEQ ID NO: 392). For illustrative purposes the first 6 target portions of the NP transcript are listed in Table 16.

TABLE 16
Target Portions in NP Gene

| ID | | Nucleotide |
|---|---|---|
| Number | Sequence | Position |
| 1 | AGCAAAAGCAGGGTAGATA (SEQ ID NO: | 1-19 |
| | 394) | |
| 2 | GCAAAAGCAGGGTAGATAA (SEQ ID NO: | 2-20 |
| | 395) | |
| 3 | CAAAAGCAGGGTAGATAAT (SEQ ID NO: | 3-21 |
| | 396) | |
| 4 | AAAAGCAGGGTAGATAATC (SEQ ID NO: | 4-22 |
| | 397) | |
| 5 | AAAGCAGGGTAGATAATCA (SEQ ID NO: | 5-23 |
| | 398) | |
| 6 | AAGCAGGGTAGATAATCAC (SEQ ID NO: | 6-24 |
| | 399) | |

Potential target portions were subjected to a selection step to identify target portions having preferred features for inhibition by RNAi-inducing agents. Criteria applied in the selection step included a filter based on GC content, and a filter based on the presence or absence of consecutive stretches G or C nucleotides. For example, preferred RNAi-inducing agent inhibitory regions preferably contain less than 70% G or C nucleotides and preferably do not contain continuous stretches of more than 3 G nucleotides or more than 3 C nucleotides. Therefore preferred target portions contain less than 70% G or C nucleotides and preferably do not contain continuous stretches of more than 3 G nucleotides or more than 3 C nucleotides. The target portions that remained after application of the filter are referred to as “functional target portions”. The 2,244 functional target portions are listed in Table 17.

The sequences of the functional target portions from PR8 were compared with the sequences of the corresponding target portions of the other strains listed in Tables 15A-15H. The corresponding target portions in the other strains were readily identified based on their position in the segments and on their sequences, which were generally substantially identical with the target portion in PR8.

We recognized that in accordance with the “wobble rules”, GU base pairing can occur. We also recognized that the importance of maintaining complementarity between the antisense strand and the target differs at different positions. We therefore identified target portions for which at least 80% of the strains compared with PR8 met the following criteria when the target portion in PR8 was compared with the corresponding target portion in the strain, with both being aligned in the 5′ to 3′ direction: (1) An A to G or C to U difference between the PR8 sequence and the corresponding sequence is allowed at any position; (2) A G to A or C to A difference between the PR8 sequence and the corresponding sequence is allowed at only one or more of positions 1, 18, and 19; (3) There are 0, 1, 2, or 3 differences between the PR8 sequence and the corresponding sequence between positions 1 and 9; (4) There are no more than 2 consecutive differences between the PR8 sequence and the corresponding sequence; and (5) There is at most 1 difference between the PR8 sequence and the corresponding sequence between positions 11 and 17.

Target portions that meet the foregoing criteria were designated as “favorably conserved target portions”. The sequences of the 220 target portions that are favorably conserved among influenza strains derived from humans are listed in Table 18. The target portions were from transcripts NP, PB2, PB1, PA, M, NS, and HA. These favorably conserved target portions are preferred targets for inventive RNAi-inducing agents for inhibition of a plurality of different human derived influenza A virus strains and are perfectly complementary to the inhibitory region of certain preferred antisense strands.

Following selection of the favorably conserved target portions, corresponding target portions from influenza A virus strains isolated avians (including duck, chicken, gull, teal, tern, quail, pheasant, turkey, goose, pigeon, falcon, and various other birds), or from the environment (presumed to be of avian origin) were aligned and compared with the target portions identified from human-derived strains. The strains included a large number of strains in addition to those listed in Example 1. Tables 19A-19F list the Genbank accession number (left column), strain name (middle column), and serotype (right column) of the influenza A virus genome segments that were used (in their positive sense form) to identify favorably conserved target portions. The set of strains that was aligned differed for different segments but that each set included at least 30 strains isolated in various years spanning 1934-2004. The same selection criteria used for identification of favorably conserved target portions from human derived strains were applied. The result was 138 target portions that are favorably conserved among both human and avian derived strains. The target portions were from transcripts NP, PB2, PB1, PA, M, and NS. These favorably conserved target portions are listed in Table 20. These target portions are preferred targets for inventive RNAi-inducing agents for inhibition of a plurality of different human derived and avian derived influenza A virus strains and are perfectly complementary to the inhibitory region of certain preferred antisense strands of inventive RNAi-inducing agents.

TABLE 19A
Influenza A virus PA transcripts (strains isolated from avians)

| | | |
|---|---|---|
| M21850 | A/chicken/FPV/Rostock/34 | H7N1 |
| M26088 | A/Gull/Maryland/704/77 | H13N6 |
| M26085 | A/pintail/Alberta/119/79 | H4N6 |
| AY633193 | A/mallard/Alberta/203/93 | H6N5 |
| AY633305 | A/mallard/Alberts/76/94 | H6N8 |
| AF098606 | A/Chicken/Hong Kong/728/97 | H5N1 |
| AF156444 | A/Chicken/Hong Kong/G9/97 | H9N2 |
| AY633393 | A/teal/Alberta/16/97 | H2N9 |
| AF536676 | A/Chicken/Henan/98 | H9N2 |
| AJ291397 | A/Chicken/Pakistan/2/99 | H9N2 |
| AF216731 | A/Environment/Hong Kong/437-8/99 | H5N1 |
| AY633353 | A/pintail/Alberta/207/99 | H4N8 |
| AY180690 | A/Bantam/Nanchang/9-366/2000 | H3N3 |
| AF508676 | A/Chicken/Henan/62/00 | H9N2 |
| AY059530 | A/Duck/Hong Kong/ww461/2000 | H5N1 |
| AY180683 | A/Duck/Nanchang/4-191/2000 | H4N6 |
| AF523462 | A/Duck/Shantou/2030/00 | H9N1 |
| AY180693 | A/Quail/Nanchang/4-034/2000 | H4N6 |
| AY180696 | A/Quail/Nanchang/4-040/2000 | H9N2 |
| AF457716 | A/chicken/California/1002a/00 | H6N2 |
| AY633153 | A/mallard/Alberta/127/00 | H3N8 |
| AF509210 | A/Chicken/Hong Kong/866.3/01 | H5N1 |
| AY180709 | A/Quail/Nanchang/2-040/2001 | H3N6 |
| AF523454 | A/Wild Duck/Shantou/4808/01 | H9N2 |
| AF457683 | A/chicken/California/6643/01 | H6N2 |
| AY180678 | A/chicken/Nanchang/3-201/01 | H3N6 |
| AY422033 | A/duck/Hokkaido/86/01 | H2N3 |
| AY303660 | A/chicken/Chile/176822/02 | H7N3 |
| AY576411 | A/chicken/Hong Kong/61.9/02 | H5N1 |
| AY586433 | A/turkey/Italy/220158/2002 | H7N3 |
| AY342420 | A/chicken/Netherlands/1/03 | H7N7 |
| AY616764 | A/chicken/British Columbia/04 | H7N3 |
| AY609311 | A/chicken/Guangdong/174/04 | H5N1 |

TABLE 19B
Influenza A virus PB1 transcripts (strains isolated from avians)

| | | |
|---|---|---|
| U48280 | A/Duck/hong Hong/62/76 | H11N2 |
| M25933 | A/gull/Maryland/704/77 | H13N6 |
| M25925 | A/Turkey/Minnesota/833/80 | H4N2 |
| AY633210 | A/Mallard/Alberta/206/96 | H6N8 |
| AY633186 | A/mallard/Alberta/202/96 | H2N5 |
| AF098592 | A/Chicken/Hong Kong/728/97 | H5N1 |
| AF156416 | A/Chicken/Hong Kong/G9/97 | H9N2 |
| AY633394 | A/teal/Alberta/16/97 | H2N9 |
| AF536666 | A/Chicken/Henan/98 | H9N2 |
| AF508626 | A/Chicken/Korea/99029/99 | H9N2 |
| AF216732 | A/Environment/Hong Kong/437-8/99 | H5N1 |
| AY633354 | A/pintail/Alberta/207/99 | H4N8 |
| AY180888 | A/Bantam/Nanchang/9-366/2000 | H3N3 |
| AY180885 | A/Duck/Nanchang/4-191/2000 | H4N6 |
| AF523445 | A/Duck/Shantou/2030/00 | H9N1 |
| AY180891 | A/Quail/Nanchang/4-034/2000 | H4N6 |
| AY180892 | A/Quail/Nanchang/4-040/2000 | H9N2 |
| AF457698 | A/chicken/California/431/00 | H6N2 |
| AY633154 | A/mallard/Alberta/127/00 | H3N8 |
| AF509184 | A/Chicken/Hong Kong/866.3/01 | H5N1 |
| AY180893 | A/Chicken/Nanchang/1-101/2001 | H3N6 |
| AY180864 | A/Quail/Nanchang/2-040/2001 | H3N6 |
| AF523431 | A/Wild Duck/Shantou/4808/01 | H9N2 |
| AY180872 | A/chicken/Nanchang/3-201/01 | H3N6 |
| AY422037 | A/duck/Hokkaido/86/01 | H2N3 |
| AY303664 | A/chicken/Chile/4957/02 | H7N3 |
| AY576400 | A/chicken/Hong Kong/YU777/02 | H5N1 |
| AY586436 | A/turkey/Italy/220158/2002 | H7N3 |
| AY340085 | A/chicken/Netherlands/1/03 | H7N7 |
| AY616765 | A/chicken/British Columbia/04 | H7N3 |
| AY609310 | A/chicken/Guangdong/174/04 | H5N1 |
| AY590582 | A/chicken/Nakorn-Patom/Thailand/CU-K2/2004 | H5N1 |

TABLE 19C
Influenza A virus PB2 transcripts (strains isolated from avians)

| | | |
|---|---|---|
| M73514 | A/Turkey/Minnesota/833/80 | H4N2 |
| M73516 | A/Gull/Astrakhan/227/84 | H13N6 |
| AF268115 | A/Ruddy Turnstone/Delaware/168/94 | H3N4 |
| AY633243 | A/mallard/Alberta/232/94 | H6N8 |
| AF268119 | A/Shorebird/Delaware/23/96 | H10N9 |
| AF508651 | A/Chicken/Guangdong/11/97 | H9N2 |
| AF098579 | A/Chicken/Hong Kong/728/97 | H5N1 |
| AF156430 | A/Chicken/Hong Kong/G9/97 | H9N2 |
| AF536686 | A/Chicken/Henan/98 | H9N2 |
| AJ410602 | A/duck/Hong Kong/323/98 | H6N2 |
| AF508642 | A/Chicken/Pakistan/5/99 | H9N2 |
| AF216733 | A/Environment/Hong Kong/437-8/99 | H5N1 |
| AY633355 | A/pintail/Alberta/207/99 | H4N8 |
| AY180775 | A/Bantam/Nanchang/9-366/2000 | H3N3 |
| AY180770 | A/Duck/Nanchang/4-191/2000 | H4N6 |
| AF523481 | A/Duck/Shantou/2030/00 | H9N1 |
| AY180772 | A/Quail/Nanchang/4-034/2000 | H4N6 |
| AY180773 | A/Quail/Nanchang/4-040/2000 | H9N2 |
| AF457689 | A/chicken/California/465/00 | H6N2 |
| AY633155 | A/mallard/Alberta/127/00 | H3N8 |
| AF509158 | A/Chicken/Hong Kong/866.3/01 | H5N1 |
| AY180759 | A/Quail/Nanchang/2-040/2001 | H3N6 |
| AF523464 | A/Wild Duck/Shantou/4808/01 | H9N2 |
| AF457681 | A/chicken/California/6643/01 | H6N2 |
| AY180763 | A/chicken/Nanchang/3-201/01 | H3N6 |
| AY422041 | A/duck/Hokkaido/86/01 | H2N3 |
| AY576388 | A/chicken/Hong Kong/YU777/02 | H5N1 |
| AJ627496 | A/turkey/Italy/220158/2002 | H7N3 |
| AY342413 | A/avian/Netherlands/219/03 | H7N7 |
| AY342414 | A/chicken/Netherlands/1/03 | H7N7 |
| AY616766 | A/chicken/British Columbia/04 | H7N3 |
| AY609309 | A/chicken/Guangdong/174/04 | H5N1 |
| AY590681 | A/chicken/Nakorn-Patom/Thailand/CU-K2/2004 | H5N1 |

TABLE 19D
Influenza A virus M transcripts (strains isolated from avians)

| | | |
|---|---|---|
| M23917 | A/chicken/FPV/Weybridge | H7N7 |
| M63538 | A/Gull/Massachusetts/26/80 | H13N6 |
| AJ427302 | A/curlew sandpiper/Hong Kong/208/89 | H10N5 |
| U49117 | A/Duck/Nanchang/1749/92 | H11N2 |
| AF073180 | A/Chicken/New Jersey/15086-3/94 | H7N3 |
| AF098562 | A/Chicken/Hong Kong/728/97 | H5N1 |
| AF156458 | A/Chicken/Hong Kong/G9/97 | H9N2 |
| AF250487 | A/Duck/Hong Kong/P169/97 | H3N8 |
| AF250489 | A/Duck/Hong Kong/P54/97 | H11N9 |
| AF250490 | A/Duck/Hong Kong/T25/97 | H11N8 |
| AF250492 | A/Duck/Hong Kong/Y264/97 | H4N8 |
| AY633389 | A/teal/Alberta/16/07 | H2N9 |
| AF536726 | A/Chicken/Henan/98 | H9N2 |
| AY241600 | A/Chicken/NY/12273-11/99 | H7N3 |
| AF216727 | A/Environment/Hong Kong/437-8/99 | H5N1 |
| AJ427299 | A/aquatic bird/Hong Kong/399/99 | H3N8 |
| AY633349 | A/pintail/Alberta/207/99 | H3N3 |
| AY180518 | A/Bantam/Nanchang/9-366/2000 | H3N3 |
| AY180509 | A/Duck/Nanchang/4191/2000 | H4N6 |
| AF523500 | A/Duck/Shantou/2030/00 | H9N1 |
| AY180507 | A/Quail/Nanchang/4-034/2000 | H4N6 |
| AY180504 | A/Quail/Nanchang/4-040/2000 | H9N2 |
| AY300962 | A/avian/NY/53726/00 | H5N2 |
| AY633149 | A/mallard/Alberta/127/00 | H3N8 |
| AF474055 | A/Chicken/California/6643/2001 | H6N2 |
| AF509055 | A/Chicken/Hong Kong/866.3/01 | H5N1 |
| AY180523 | A/Quail/Nanchang/2-040/2001 | H3N6 |
| AF523484 | A/Wild Duck/Shantou/4804/01 | H9N2 |
| AY300975 | A/blue-winged teal/TX/2/01 | H7N3 |
| AY180505 | A/chicken/Nanchang/3-201/01 | H3N6 |
| AY422020 | A/duck/Hokkaido/86/01 | H2N3 |
| AY241624 | A/Turkey/VA/67/02 | H7N2 |
| AY300974 | A/duck/NY/191255-59/02 | H5N8 |
| AY300971 | A/turkey/CA/D0208651-C/02 | H5N2 |
| AJ627497 | A/turkey/Italy/220168/2002 | H7N3 |
| AY340091 | A/chicken/Netherlands/1/03 | H7N7 |
| AY611525 | A/chicken/British Columbia/04 | H7N3 |
| AY609315 | A/chicken/Guangdong/174/04 | H5N1 |
| AY590578 | A/chicken/Nakorn-Patom/Thailand/CU-K2/2004 | H5N1 |

TABLE 19E
Influenza A virus NS transcripts (strains isolated from avians)

| | | |
|---|---|---|
| J02105 | A/duck/Alberta/60/76 | H12N5 |
| U96743 | A/Gull/Maryland/1824/78 | H13N9 |
| U96739 | A/Chicken/Pennsylvania/1370/83 | H13N6 |
| AF007035 | A/Duck/Ohio/421/87 | H7N8 |
| AF074267 | A/Chicken/New Jersey/15086-3/94 | H7N3 |
| AF156472 | A/Chicken/Hong Kong/G9/97 | H9N2 |
| AF319651 | A/Chicken/Italy/9097/97 | H5N9 |
| AF250495 | A/Duck/Hong Kong/P169/97 | H3N8 |
| AF250498 | A/Duck/Hong Kong/P54/97 | H11N9 |
| AF250499 | A/Duck/Hong Kong/T25/97 | H11N8 |
| AF250501 | A/Duck/Hong Kong/Y264/97 | H4N8 |
| AY633392 | A/teal/Alberta/16/97 | H2N9 |
| AF536736 | A/Chicken/Henan/98 | H9N2 |
| AJ410587 | A/duck/Hong Kong/324/98 | H6N2 |
| AY241629 | A/Chicken/NY/12273-11/99 | H7N3 |
| AF216726 | A/Environment/Hong Kong/437-8/99 | H5N1 |
| AJ427300 | A/aquatic bird/Hong Kong/399/99 | H3N8 |
| AY633352 | A/pintail/Alberta/207/99 | H4N8 |
| AY180647 | A/Bantam/Nanchang/9-366/2000 | H3N3 |
| AY180606 | A/duck/Nanchang/4-191/2000 | H4N6 |
| AF523502 | A/Duck/Shantou/2030/00 | H9N1 |
| AY180614 | A/Quail/Nanchang/4-034/2000 | H4N6 |
| AY180617 | Å/Quail/Nanchang/4-040/2000 | H9N2 |
| AY300986 | A/avain/NY/53726/00 | H5N2 |
| AY180620 | A/mallard/Alberta/127/00 | H3N8 |
| AY300999 | A/blue-winged teal/TX/2/01 | H7N3 |
| AF457675 | A/chicken/California/905/01 | H6N2 |
| AY259220 | A/chicken/Hebei/1/01 | H9N2 |
| AY180600 | A/chicken/Nanchang/3-201/01 | H3N6 |
| AY422029 | A/duck/Hokkaido/86/01 | H2N3 |
| AY241662 | A/Turkey/VA/67/02 | H7N2 |
| AY300998 | A/duck/NY/191255-59/02 | H5N8 |
| AY300995 | A/turkey/CA/D0208651-C/02 | H5N2 |
| AY303645 | A/chicken/Chile/4322/03 | H7N3 |
| AY342424 | A/chicken/Netherlands/1/03 | H7N7 |
| AY611528 | A/chicken/British Columbia/04 | H7N3 |
| AY609316 | A/chicken/Guangdong/174/04 | H5N1 |
| AY590580 | A/chicken/Nakorn-Patom/Thailand/CU-K2/2004 | H5N1 |

TABLE 19F
Influenza A virus NP transcripts (strains isolated from avians)

| | | |
|---|---|---|
| M63779 | A/FPV/Dobson/‘Dutch’/27 | H7N7 |
| AJ243993 | A/FPV/ROSTOCK/34 | H7N1 |
| M22576 | A/FPV/Rostock/34 | H7N1 |
| M21937 | A/FPV/Rostock/34 | H7N1 |
| M24660 | A/FPV/Rostock/34 (mutant ts19) | H7N1 |
| M24556 | A/FPV/Rostock/34 (revertant 19R) | H7N1 |
| M24557 | A/FPV/Rostock/34 (revertant 81R) | H7N1 |
| M24453 | A/Chicken/Germany/N/49 | H10N7 |
| M63773 | A/Duck/Manitoba/1/53 | H10N7 |
| M63780 | A/Duck/England/1/56 | H11N6 |
| M30762 | A/duck/Czechoslovakia/56 | H4N6 |
| M30763 | A/duck/Ukraine/2/60 | H11N8 |
| M30767 | A/Tern/South Africa/61 | H5N3 |
| M63781 | A/Duck/England/1/62 | H4N6 |
| AF156415 | A/Turkey/California/189/66 | H9N2 |
| M63774 | A/Turkey/Ontario/7732/66 | H5N9 |
| M63775 | A/Duck/Pennsylvania/1/69 | H6N1 |
| M27298 | A/shearwater/Australia/72 | H6N5 |
| M27519 | A/tern/Turkmenia/18/72 | H3N3 |
| M22344 | A/parrot/Ulster/73 | H7N1 |
| M63776 | A/Duck/Memphis/928/74 | H3N8 |
| M22573 | A/duck/Hong Kong/7/75 | H3N2 |
| M36812 | A/Anas acuta/Primorje/695/76 | H2N3 |
| AF523423 | A/Duck/Hong Kong/86/76 | H9N2 |
| U49093 | A/Goose/Hong Kong/8/76 | H1N1 |
| M30760 | A/duck/New Zealand/31/76 | H4N6 |
| U49097 | A/Duck/Hong Kong/193/77 | H1N2 |
| M63777 | A/Gull/Maryland/5/77 | H11N9 |
| M30765 | A/budgerigar/Hokkaido/1/77 | H4N6 |
| M22574 | A/duck/Bavaria/2/77 | H1N1 |
| M27521 | A/gull/Maryland/704/77 | H13N6 |
| M76603 | A/turkey/England/647/77 | H1N1 |
| M63782 | A/Duck/Beijing/1/78 | H3N6 |
| AF523421 | A/Duck/Hong Kong/289/78 | H9N2 |
| AF523424 | A/Duck/Hong Kong/366/78 | H9N2 |
| D00050 | A/Mallard/New York/6750/78 | H2N2 |
| M30755 | A/gull/Maryland/1824/78 | H13N9 |
| AF523422 | A/Duck/Hong Kong/552/79 | H9N2 |
| U49095 | A/Duck/Hong Kong/717/79 | H1N3 |
| M30761 | A/grey teal/Australia/2/79 | H4N4 |
| M30756 | A/gull/Maryland/1815/79 | H13N6 |
| M63783 | A/Duck/Australia/749/80 | H1N1 |
| AF079571 | A/Duck/Hokkaido/8/80 | H3N8 |
| M30752 | A/Gull/Massachussetts/26/80 | H13N6 |
| M30757 | A/Gull/Minnesota/945/80 | H13N6 |
| M63784 | A/Teal/Iceland/29/80 | H7N7 |
| M30769 | A/turkey/Minnesota/833/80 | H4N2 |
| M63778 | A/Turkey/Minnesota/1661/81 | H1N1 |
| M63785 | A/Mallard/Astrakhan(Gurjev)/263/82 | H14N5 |
| M30764 | A/mallard/Astrakhan/244/82 | H14 |
| M30768 | A/chicken/Pennsylvania/1/83 | H5N2 |
| AY633119 | A/mallard/Alberta/743/83 | H9N1 |
| M30753 | A/gull/Astrakhan/227/84 | H13N6 |
| AY633311 | A/mallard/Alberta/98/85 | H6N2 |
| AY633319 | A/pintail/Alberta/113/85 | H6N2 |
| M30766 | A/ruddy turnstone/New Jersey/47/85 | H4N6 |
| Z26855 | A/oystercatcher/Germany/87 | H1N1 |
| AY633279 | A/mallard/Alberta/321/88 | H9N2 |
| M76609 | A/turkey/North Carolina/1790/88 | H1N1 |
| AJ427301 | A/curlew sandpiper/Hong Kong/208/89 | H10N5 |
| AY633295 | A/mallard/Alberta/11/91 | H9N2 |
| AY633167 | A/mallard/Alberta/17/91 | H9N2 |
| Z26857 | A/turkey/Germany/3/91 | H1N1 |
| U49094 | A/Duck/Nanchang/1749/92 | H11N2 |
| AF156410 | A/Quail/Hong Kong/AF157/92 | H9N2 |
| AY633191 | A/mallard/Alberta/203/92 | H6N5 |
| AF156414 | A/Quail/Arkansas/29209-1/93 | H9N2 |
| AY633335 | A/pintail/Alberta/179/93 | H6N1 |
| AF156409 | A/Chicken/Beijing/1/94 | H9N2 |
| AY497120 | A/Chicken/Hidalgo/232/94 | H5N2 |
| AF098624 | A/Chicken/Hidalgo/26654-1368/94 | H5N2 |
| AF156408 | A/Chicken/Hong Kong/739/94 | H9N2 |
| AF098625 | A/Chicken/Mexico/26654-1374/94 | H5N2 |
| AY497113 | A/Chicken/Mexico/31381-7/94 | H5N2 |
| AF098627 | A/Chicken/Puebla/14585-622/94 | H5N2 |
| AY497114 | A/Chicken/Puebla/8623-607/94 | H5N2 |
| AF098626 | A/Chicken/Puebla/8623-607/94 | H5N2 |
| AY633383 | A/Redhead/Alberta/291/94 | H6N8 |
| AY633239 | A/mallard/Alberta/232/94 | H6N8 |
| AY633303 | A/mallard/Alberta/76/94 | H6N8 |
| AY633327 | A/pintail/Alberta/155/94 | H6N8 |
| AF536699 | A/Chicken/Beijing/1/95 | H9N2 |
| AF213906 | A/Chicken/Italy/24/95 | H1N1 |
| AY497117 | A/Chicken/Puebla/28159-474/95 | H5N2 |
| AF098628 | A/Chicken/Queretaro/14588-19/95 | H5N2 |
| AF508600 | A/Duck/Germany/113/95 | H9N2 |
| AF213905 | A/Mallard/Italy/24/95 | H1N1 |
| AF508596 | A/Ostrich/South Africa/9508103/95 | H9N2 |
| AB020778 | A/Chicken/Beijing/1/96 | H9N2 |
| AF536703 | A/Chicken/Hebei/1/96 | H9N2 |
| AF156412 | A/Chicken/Korea/25232-96006/96 | H9N2 |
| AF156411 | A/Chicken/Korea/38349-p96323/96 | H9N2 |
| AF203787 | A/Chicken/Korea/MS96/96 | H9N2 |
| AF508613 | A/Chicken/Shandong/6/96 | H9N2 |
| AF144303 | A/Goose/Guangdong/1/96 | H5N1 |
| AY633207 | A/Mallard/Alberta/206/96 | H6N8 |
| AF508617 | A/Quail/Shanghai/8/96 | H9N2 |
| AF156413 | A/Shorebird/Delaware/9/96 | H9N2 |
| AY633183 | A/mallard/Alberta/202/96 | H2N5 |
| AF536700 | A/Chicken/Beijing/2/97 | H9N2 |
| AF508607 | A/Chicken/Guangdong/11/97 | H9N2 |
| AF536702 | A/Chicken/Guangdong/97 | H9N2 |
| AF508609 | A/Chicken/Heilongjiang/10/97 | H9N2 |
| AF046084 | A/Chicken/Hong Kong/220/97 | H5N1 |
| AF098618 | A/Chicken/Hong Kong/728/97 | H5N1 |
| AF098619 | A/Chicken/Hong Kong/786/97 | H5N1 |
| AF098620 | A/Chicken/Hong Kong/915/97 | H5N1 |
| AF156403 | A/Chicken/Hong Kong/G23/97 | H9N2 |
| AF156402 | A/Chicken/Hong Kong/G9/97 | H9N2 |
| AF098617 | A/Chicken/Hong Kong/y388/97 | H5N1 |
| AF319644 | A/Chicken/Italy/312/97 | H5N2 |
| AF319645 | A/Chicken/Italy/330/97 | H5N2 |
| AF319646 | A/Chicken/Italy/367/97 | H5N2 |
| AF319647 | A/Chicken/Italy/9097/97 | H5N9 |
| AF508615 | A/Chicken/Shenzhen/9/97 | H9N2 |
| AF508612 | A/Chicken/Sichuan/5/97 | H9N2 |
| AF250473 | A/Duck/Hong Kong/P185/97 | H3N8 |
| AF250474 | A/Duck/Hong Kong/P54/97 | H11N9 |
| AF250470 | A/Duck/Hong Kong/T25/97 | H11N8 |
| AF250471 | A/Duck/Hong Kong/T37/97 | H11N8 |
| AF156405 | A/Duck/Hong Kong/Y280/97 | H9N2 |
| AF156406 | A/Duck/Hong Kong/Y439/97 | H9N2 |
| AF098621 | A/Duck/Hong Kong/p46/97 | H5N1 |
| AF098622 | A/Duck/Hong Kong/y283/97 | H5N1 |
| AF508616 | A/Duck/Nanjing/1/97 | H9N2 |
| ISDN22474 | A/Duck/Singapore/645/97 | H5N3 |
| AF370122 | A/Goose/Guangdong/3/97 | H5N1 |
| AF250475 | A/Goose/Hong Kong/W217/97 | H6N9 |
| AF098623 | A/Goose/Hong Kong/w355/97 | H5N1 |
| AF508603 | A/Pheasant/Ireland/PV18/97 | H9N2 |
| AF156404 | A/Pigeon/Hong Kong/Y233/97 | H9N2 |
| AF156407 | A/Quail/Hong Kong/G1/97 | H9N2 |
| AF250480 | A/Teal/Hong Kong/W312/97 | H6N1 |
| AF057293 | A/chicken/Hong Kong/258/97 | H5N1 |
| AY633135 | A/mallard/Alberta/117/97 | H3N8 |
| AB049161 | A/parakeet/Chiba/1/97 | H9N2 |
| AY633343 | A/pintail/Alberta/156/97 | H3N8 |
| AY633367 | A/pintail/Alberta/22/97 | H2N9 |
| AY633391 | A/teal/Alberta/16/97 | H2N9 |
| AF250472 | A/Aquatic bird/Hong Kong/M603/98 | H11N1 |
| AF508605 | A/Chicken/Beijing/8/98 | H9N2 |
| AF508599 | A/Chicken/Germany/R45/98 | H9N2 |
| AF536704 | A/Chicken/Hebei/2/98 | H9N2 |
| AF536705 | A/Chicken/Hebei/3/98 | H9N2 |
| AF508608 | A/Chicken/Hebei/4/98 | H9N2 |
| AF536706 | A/Chicken/Henan/98 | H9N2 |
| AY497116 | A/Chicken/Puebla/231-5284/98 | H5N2 |
| AF536708 | A/Chicken/Shandong/98 | H9N2 |
| AY253753 | A/Chicken/Shanghai/F/98 | H9N2 |
| AY497115 | A/chicken/Aguascalientes/124-3705/98 | H5N2 |
| AY633199 | A/mallard/Alberta/205/98 | H2N3 |
| AY633215 | A/mallard/Alberta/211/98 | H1N1 |
| AY633231 | A/mallard/Alberta/226/98 | H2N3 |
| AY633247 | A/mallard/Alberta/242/98 | H3N8 |
| AY633255 | A/mallard/Alberta/279/98 | H3N8 |
| AY633263 | A/mallard/Alberta/295/98 | H4N6 |
| AY633271 | A/mallard/Alberta/30/98 | H4N6 |
| AY633287 | A/mallard/Alberta/47/98 | H4N1 |
| AB049162 | A/parakeet/Narita/92A/98 | H9N2 |
| AF536701 | A/Chicken/Beijing/3/99 | H9N2 |
| AF222619 | A/Chicken/Hong Kong/FY20/99 | H9N2 |
| AF222620 | A/Chicken/Hong Kong/KC12/99 | H9N2 |
| AF222616 | A/Chicken/Hong Kong/NT16/99 | H9N2 |
| AF186272 | A/Chicken/Hong Kong/SF2/99 | H9N2 |
| AF508601 | A/Chicken/Iran/11T/99 | H9N2 |
| AF508604 | A/Chicken/Korea/99029/99 | H9N2 |
| AF536707 | A/Chicken/Liaoning/99 | H9N2 |
| AF508611 | A/Chicken/Ningxia/5/99 | H9N2 |
| AJ291394 | A/Chicken/Pakistan/2/99 | H9N2 |
| AF508597 | A/Chicken/Pakistan/4/99 | H9N2 |
| AF508598 | A/Chicken/Pakistan/5/99 | H9N2 |
| AF508602 | A/Chicken/Saudi Arabia/532/99 | H9N2 |
| AF508614 | A/Chicken/Shijiazhuang/2/99 | H9N2 |
| AF216736 | A/Environment/Hong Kong/437-10/99 | H5N1 |
| AF216712 | A/Environment/Hong Kong/437-4/99 | H5N1 |
| AF216720 | A/Environment/Hong Kong/437-6/99 | H5N1 |
| AF216728 | A/Environment/Hong Kong/437-8/99 | H5N1 |
| AF222618 | A/Pheasant/Hong Kong/SSP11/99 | H9N2 |
| AF222615 | A/Pigeon/Hong Kong/FY6/99 | H9N2 |
| AF222614 | A/Quail/Hong Kong/A17/99 | H9N2 |
| AF186270 | A/Quail/Hong Kong/NT28/99 | H9N2 |
| AF222617 | A/Quail/Hong Kong/SSP10/99 | H9N2 |
| AF186271 | A/Silky Chicken/Hong Kong/SF43/99 | H9N2 |
| AF222621 | A/Silky Chicken/Hong Kong/SF44/99 | H9N2 |
| AY038019 | A/Turkey/MO/24093/99 | H1N2 |
| AJ427298 | A/aquatic bird/Hong Kong/399/99 | H3N8 |
| AF261750 | A/chicken/Taiwan/7-5/99 | H6N1 |
| AY585429 | A/duck/Guangxi/07/1999 | H5N1 |
| AJ410555 | A/duck/Hong Kong/3096/99 | H6N2 |
| AJ410556 | A/duck/Hong Kong/3461/99 | H6N1 |
| AY633127 | A/mallard/Alberta/111/99 | H4N6 |
| AY633175 | A/mallard/Alberta/199/99 | H3N6 |
| AY633223 | A/mallard/Alberta/215/99 | H6N8 |
| AJ410548 | A/pheasant/Hong Kong/SH39/99 | H6N1 |
| AY633351 | A/pintail/Alberta/207/99 | H4N8 |
| AY633359 | A/pintail/Alberta/210/99 | H4N6 |
| AY633375 | A/pintail/Alberta/37/99 | H3N8 |
| AJ410549 | A/quail/Hong Kong/1721-20/99 | H6N1 |
| AJ410550 | A/quail/Hong Kong/1721-30/99 | H6N1 |
| AJ627488 | A/turkey/Italy/4603/1999 | H7N1 |
| AY180580 | A/Bantam/Nanchang/9-366/2000 | H3N3 |
| AF474069 | A/Chicken/California/650/00 | H6N2 |
| AF508606 | A/Chicken/Guangdong/10/00 | H9N2 |
| AF508610 | A/Chicken/Henan/62/00 | H9N2 |
| AY180581 | A/Chicken/Nanchang/1-0016/2000 | H9N2 |
| AY180545 | A/Chicken/Nanchang/12-220/2000 | H3N6 |
| AY180527 | A/Chicken/Nanchang/12-301/2000 | H3N6 |

| | | |
|---|---|---|
| AY180554 | A/Chicken/Nanchang/2-0527/2000 | H4N6 |
| AY180539 | A/Chicken/Nanchang/3-0128/2000 | H4N6 |
| AY180531 | A/Chicken/Nanchang/4-008/2000 | H4N6 |
| AY180562 | A/Chicken/Nanchang/4-010/2000 | H9N2 |
| AY180529 | A/Chicken/Nanchang/7-010/2000 | H3N6 |
| AY180535 | A/Chicken/Nanchang/9-220/2000 | H3N6 |
| AY059497 | A/Duck/Hong Kong/2986.1/2000 | H5N1 |
| AY059494 | A/Duck/Hong Kong/ww381/2000 | H5N1 |
| AY059495 | A/Duck/Hong Kong/ww461/2000 | H5N1 |
| AY180584 | A/Duck/Nanchang/1-0070/2000 | H9N2 |
| AY180549 | A/Duck/Nanchang/10-096/2000 | H3N6 |
| AY180553 | A/Duck/Nanchang/10-383/2000 | H3N6 |
| AY180583 | A/Duck/Nanchang/10-389/2000 | H9N2 |
| AY180542 | A/Duck/Nanchang/11-197/2000 | H9N2 |
| AY180544 | A/Duck/Nanchang/11-290/2000 | H9N2 |
| AY180537 | A/Duck/Nanchang/11-392/2000 | H9N2 |
| AY180586 | A/Duck/Nanchang/12-280/2000 | H3N6 |
| AY180524 | A/Duck/Nanchang/2-0147/2000 | H4N6 |
| AY180557 | A/Duck/Nanchang/2-0485/2000 | H2N9 |
| AY180558 | A/Duck/Nanchang/2-0486/2000 | H2N9 |
| AY180573 | A/Duck/Nanchang/2-0492/2000 | H2N9 |
| AY180574 | A/Duck/Nanchang/3-090/2000 | H2N9 |
| AY180572 | A/Duck/Nanchang/4-165/2000 | H4N6 |
| AY180559 | A/Duck/Nanchang/4-173/2000 | H4N6 |
| AY180568 | A/Duck/Nanchang/4-184/2000 | H2N9 |
| AY180571 | A/Duck/Nanchang/4-190/2000 | H2N9 |
| AY180570 | A/Duck/Nanchang/4-191/2000 | H4N6 |
| AY180534 | A/Duck/Nanchang/7-092/2000 | H9N2 |
| AY180547 | A/Duck/Nanchang/8-174/2000 | H3N6 |
| AY180546 | A/Duck/Nanchang/8-197/2000 | H3N6 |
| AY180532 | A/Duck/Nanchang/8-198/2000 | H3N6 |
| AY180526 | A/Duck/Nanchang/9-091/2000 | H3N6 |
| AY180579 | A/Duck/Nanchang/9-385/2000 | H3N6 |
| AF523413 | A/Duck/Shantou/1042/00 | H9N2 |
| AF523410 | A/Duck/Shantou/1043/00 | H9N2 |
| AF523425 | A/Duck/Shantou/1588/00 | H9N1 |
| AF523420 | A/Duck/Shantou/1881/00 | H9N2 |
| AF523426 | A/Duck/Shantou/2030/00 | H9N1 |
| AF523415 | A/Duck/Shantou/2102/00 | H9N2 |
| AF523411 | A/Duck/Shantou/2134/00 | H9N2 |
| AF523419 | A/Duck/Shantou/2143/00 | H9N2 |
| AF523417 | A/Duck/Shantou/2144/00 | H9N2 |
| AF523416 | A/Duck/Shantou/830/00 | H9N2 |
| AY059498 | A/Goose/Hong Kong/3014.8/2000 | H5N1 |
| AF398419 | A/Goose/Hong Kong/385.3/2000 | H5N1 |
| AF398420 | A/Goose/Hong Kong/385.5/2000 | H5N1 |
| AY059492 | A/Goose/Hong Kong/ww26/2000 | H5N1 |
| AY059493 | A/Goose/Hong Kong/ww28/2000 | H5N1 |
| AY059496 | A/Goose/Hong Kong/ww491/2000 | H5N1 |
| AY180530 | A/Pigeon/Nanchang/11-045/2000 | H3N6 |
| AY180538 | A/Pigeon/Nanchang/11-145/2000 | H9N2 |
| AY180525 | A/Pigeon/Nanchang/2-0461/2000 | H9N2 |
| AY180560 | A/Pigeon/Nanchang/7-058/2000 | H9N2 |
| AY180536 | A/Pigeon/Nanchang/8-142/2000 | H3N6 |
| AY180582 | A/Pigeon/Nanchang/9-058/2000 | H3N3 |
| AY180550 | A/Quail/Nanchang/10-028/2000 | H3N6 |
| AY180543 | A/Quail/Nanchang/12-340/2000 | H1N1 |
| AY180575 | A/Quail/Nanchang/2-0460/2000 | H9N2 |
| AY180576 | A/Quail/Nanchang/2-0579/2000 | H4N6 |
| AY180540 | A/Quail/Nanchang/4-026/2000 | H4N6 |
| AY180541 | A/Quail/Nanchang/4-034/2000 | H4N6 |
| AY180563 | A/Quail/Nanchang/4-040/2000 | H9N2 |
| AY180548 | A/Quail/Nanchang/7-026/2000 | H3N6 |
| AY180564 | A/Wild Duck/Nanchang/2-0480/2000 | H9N2 |
| AF457701 | A/chicken/California/431/00 | H6N2 |
| AF457693 | A/chicken/California/465/00 | H6N2 |
| AY496851 | A/chicken/Mudanjiang/0823/2000 | H9N2 |
| AJ410554 | A/chukka/Hong Kong/FY295/00 | H6N1 |
| AJ410553 | A/chukka/Hong Kong/NT261/00 | H6N1 |
| AY585423 | A/duck/Fujian/19/2000 | H5N1 |
| AY585425 | A/duck/Guangdong/07/2000 | H5N1 |
| AY585426 | A/duck/Guangdong/12/2000 | H5N1 |
| AY585428 | A/duck/Guangdong/40/2000 | H5N1 |
| AY585439 | A/duck/Zhejiang/11/2000 | H5N1 |
| AY585440 | A/duck/Zhejiang/52/2000 | H5N1 |
| AY633143 | A/mallard/Alberta/119/00 | H4N6 |
| AY633151 | A/mallard/Alberta/127/00 | H3N8 |
| AY633159 | A/mallard/Alberta/136/00 | H4N6 |
| AJ421064 | A/pheasant/Hong Kong/FY294/00 | H6N1 |
| AJ427309 | A/pheasant/Hong Kong/FY294/00 | H6N1 |
| AJ427864 | A/quail/Hong Kong/FY298/00 | H6N1 |
| AJ410551 | A/quail/Hong Kong/SF550/00 | H6N1 |
| AJ410552 | A/quail/Hong Kong/SF595/00 | H6N1 |
| AF474070 | A/Chicken/California/139/01 | H6N2 |
| AY497118 | A/Chicken/El Salvador/102711-1/01 | H5N2 |
| AF509126 | A/Chicken/Hong Kong/715.5/01 | H5N1 |
| AF509127 | A/Chicken/Hong Kong/751.1/01 | H5N1 |
| AF509128 | A/Chicken/Hong Kong/822.1/01 | H5N1 |
| AF509129 | A/Chicken/Hong Kong/829.2/01 | H5N1 |
| AF509130 | A/Chicken/Hong Kong/830.2/01 | H5N1 |
| AF509131 | A/Chicken/Hong Kong/858.3/01 | H5N1 |
| AF509132 | A/Chicken/Hong Kong/866.3/01 | H5N1 |
| AF509133 | A/Chicken/Hong Kong/867.1/01 | H5N1 |
| AF509135 | A/Chicken/Hong Kong/873.3/01 | H5N1 |
| AF509136 | A/Chicken/Hong Kong/876.1/01 | H5N1 |
| AF509134 | A/Chicken/Hong Kong/879.1/01 | H5N1 |
| AF509137 | A/Chicken/Hong Kong/891.1/01 | H5N1 |
| AF509138 | A/Chicken/Hong Kong/893.2/01 | H5N1 |
| AF509120 | A/Chicken/Hong Kong/FY150/01 | H5N1 |
| AY221551 | A/Chicken/Hong Kong/FY150/01 | H5N1 |
| AY221550 | A/Chicken/Hong Kong/FY150/01- | H5N1 |
| AF509117 | A/Chicken/Hong Kong/FY77/01 | H5N1 |
| AY221549 | A/Chicken/Hong Kong/NT873.3/01 | H5N1 |
| AY221548 | A/Chicken/Hong Kong/NT873.3/01- | H5N1 |
| AF509125 | A/Chicken/Hong Kong/SF219/01 | H5N1 |
| AY221556 | A/Chicken/Hong Kong/YU562/01 | H5N1 |
| AF509118 | A/Chicken/Hong Kong/YU562/01 | H5N1 |
| AF509119 | A/Chicken/Hong Kong/YU563/01 | H5N1 |
| AY221555 | A/Chicken/Hong Kong/YU822.2/01 | H5N1 |
| AY221554 | A/Chicken/Hong Kong/YU822.2/01- | H5N1 |
| AY180585 | A/Chicken/Nanchang/1-020/2001 | H3N6 |
| AY180565 | A/Chicken/Nanchang/1-101/2001 | H3N6 |
| AY180533 | A/Chicken/Nanchang/2-120/2001 | H3N6 |
| AY180566 | A/Chicken/Nanchang/2-220/2001 | H3N6 |
| AY180555 | A/Chicken/Nanchang/3-120/2001 | H3N2 |
| AY180578 | A/Chicken/Nanchang/4-301/2001 | H9N2 |
| AY180551 | A/Chicken/Nanchang/4-361/2001 | H9N2 |
| AF468842 | A/Duck/Anyang/AVL-1/2001 | H5N1 |
| AF509141 | A/Duck/Hong Kong/573.4/01 | H5N1 |
| AF509142 | A/Duck/Hong Kong/646.3/01 | H5N1 |
| AY233394 | A/Duck/NC/91347/01 | H1N2 |
| AY180577 | A/Duck/Nanchang/1-100/2001 | H3N6 |
| AY180528 | A/Duck/Nanchang/1-161/2001 | H3N6 |
| AY180552 | A/Duck/Nanchang/1-181/2001 | H3N6 |
| AY180556 | A/Duck/Nanchang/2-182/2001 | H3N6 |
| AF523414 | A/Duck/Shantou/1605/01 | H9N2 |
| AF523418 | A/Duck/Shantou/2088/01 | H9N2 |
| AF509139 | A/Goose/Hong Kong/76.1/01 | H5N1 |
| AF509140 | A/Goose/Hong Kong/ww100/01 | H5N1 |
| AF509121 | A/Pheasant/Hong Kong/FY155/01 | H5N1 |
| AY221553 | A/Pheasant/Hong Kong/FY155/01 | H5N1 |
| AY221552 | A/Pheasant/Hong Kong/FY155/01- | H5N1 |
| AF509124 | A/Pigeon/Hong Kong/SF215/01 | H5N1 |
| AF509123 | A/Quail/Hong Kong/SF203/01 | H5N1 |
| AY180569 | A/Quail/Nanchang/2-040/2001 | H3N6 |
| AY180567 | A/Quail/Nanchang/3-140/2001 | H3N6 |
| AF509122 | A/Silky Chicken/Hong Kong/SF189/01 | H5N1 |
| AF523412 | A/Wild Duck/Shantou/4808/01 | H9N2 |
| AF457709 | A/chicken/California/139/01 | H6N2 |
| AF457685 | A/chicken/California/6643/01 | H6N2 |
| AF457676 | A/chicken/California/905/01 | H6N2 |
| AY180561 | A/chicken/Nanchang/3-201/01 | H3N6 |
| AY268949 | A/chicken/Wangcheng/4/2001 | H9N2 |
| AY585422 | A/duck/Fujian/17/2001 | H5N1 |
| AY585424 | A/duck/Guangdong/01/2001 | H5N1 |
| AY585430 | A/duck/Guangxi/22/2001 | H5N1 |
| AY585431 | A/duck/Guangxi/35/2001 | H5N1 |
| AY585432 | A/duck/Guangxi/50/2001 | H5N1 |
| AY422023 | A/duck/Hokkaido/107/01 | H2N3 |
| AY422024 | A/duck/Hokkaido/17/01 | H2N3 |
| AY422025 | A/duck/Hokkaido/86/01 | H2N3 |
| AY422026 | A/duck/Hokkaido/95/01 | H2N2 |
| AY585434 | A/duck/Shanghai/08/2001 | H5N1 |
| AY585435 | A/duck/Shanghai/13/2001 | H5N1 |
| AY585438 | A/duck/Shanghai/38/2001 | H5N1 |
| AY586423 | A/mallard/Italy/33/01 | H7N3 |
| AY586424 | A/mallard/Italy/43/01 | H7N3 |
| AY497119 | A/Chicken/Guatemala/194573/02 | H5N2 |
| AY651511 | A/Ck/HK/31.2/2002 | H5N1 |
| AY651522 | A/Ck/HK/3169.1/2002 | H5N1 |
| AY651521 | A/Ck/HK/3176.3/2002 | H5N1 |
| AY651512 | A/Ck/HK/37.4/2002 | H5N1 |
| AY651514 | A/Ck/HK/YU22/2002 | H5N1 |
| AY575908 | A/Eg/Hong Kong/757.3/02 | H5N1 |
| AY575909 | A/G.H/Hong Kong/793.1/02 | H5N1 |
| AY651510 | A/Gf/HK/38/2002 | H5N1 |
| AY651513 | A/SCk/HK/YU100/2002 | H5N1 |
| AY303658 | A/chicken/Chile/176822/02 | H7N3 |
| AY303659 | A/chicken/Chile/4957/02 | H7N3 |
| AY575911 | A/chicken/Hong Kong/31.4/02 | H5N1 |
| AY575915 | A/chicken/Hong Kong/409.1/02 | H5N1 |
| AY575912 | A/chicken/Hong Kong/61.9/02 | H5N1 |
| AY575914 | A/chicken/Hong Kong/96.1/02 | H5N1 |
| AY575913 | A/chicken/Hong Kong/YU777/02 | H5N1 |
| AY585420 | A/duck/Fujian/01/2002 | H5N1 |
| AY585421 | A/duck/Fujian/13/2002 | H5N1 |
| AY585427 | A/duck/Guangdong/22/2002 | H5N1 |
| AY585433 | A/duck/Guangxi/53/2002 | H5N1 |
| AY575910 | A/duck/Hong Kong/821/02 | H5N1 |
| AY585436 | A/duck/Shanghai/35/2002 | H5N1 |
| AY585437 | A/duck/Shanghai/37/2002 | H5N1 |
| AY651524 | A/feral pigeon/HK/862.7/2002 | H5N1 |
| AY575907 | A/goose/Hong Kong/739.2/02 | H5N1 |
| AY651526 | A/grey heron/HK/861.1/2002 | H5N1 |
| AY575916 | A/pheasant/Hong Kong/sv674.15/02 | H5N1 |
| AY651527 | A/teal/China/2978.1/2002 | H5N1 |
| AY651525 | A/tree sparrow/HK/864/2002 | H5N1 |
| AY586426 | A/turkey/Italy/214845/02 | H7N3 |
| AJ627486 | A/turkey/Italy/214845/2002 | H7N3 |
| AJ627495 | A/turkey/Italy/220158/2002 | H7N3 |
| AY586425 | A/turkey/Italy/220158/2002 | H7N3 |
| AY651515 | A/Ck/HK/2133.1/2003 | H5N1 |
| AY651519 | A/Ck/HK/FY157/2003 | H5N1 |
| AY651516 | A/Ck/HK/NT93/2003 | H5N1 |
| AY651517 | A/Ck/HK/SSP141/2003 | H5N1 |
| AY651518 | A/Ck/HK/WF157/2003 | H5N1 |
| AY651520 | A/Ck/HK/YU324/2003 | H5N1 |
| AY651490 | A/Ck/Indonesia/2A/2003 | H5N1 |
| AY651485 | A/Ck/Indonesia/BL/2003 | H5N1 |
| AY651487 | A/Ck/Indonesia/PA/2003 | H5N1 |
| AY651532 | A/Ck/ST/4231/2003 | H5N1 |
| AY651529 | A/Dk/HN/5806/2003 | H5N1 |
| AY651534 | A/Dk/ST/4003/2003 | H5N1 |
| AY651535 | A/Dk/YN/6255/2003 | H5N1 |
| AY651536 | A/Dk/YN/6445/2003 | H5N1 |
| AY342426 | A/avian/Netherlands/033/03 | H7N7 |
| AY342425 | A/avian/Netherlands/219/03 | H7N7 |
| AY651523 | A/black headed gull/HK/12.1/2003 | H5N1 |
| AJ620352 | A/chicken/Germany/R28/03 | H7N7 |
| AY342427 | A/chicken/Netherlands/1/03 | H7N7 |
| AY518364 | A/duck/China/E319-2/03 | H5N1 |
| AY576929 | A/Chicken/Vietnam/CM/2004 | H5N1 |
| AY651488 | A/Ck/Indonesia/4/2004 | H5N1 |
| AY651489 | A/Ck/Indonesia/5/2004 | H5N1 |
| AY651491 | A/Ck/Thailand/1/2004 | H5N1 |
| AY651492 | A/Ck/Thailand/73/2004 | H5N1 |
| AY651493 | A/Ck/Thailand/9.1/2004 | H5N1 |
| AY651502 | A/Ck/Viet Nam/33/2004 | H5N1 |
| AY651503 | A/Ck/Viet Nam/35/2004 | H5N1 |
| AY651504 | A/Ck/Viet Nam/36/2004 | H5N1 |
| AY651505 | A/Ck/Viet Nam/37/2004 | H5N1 |
| AY651506 | A/Ck/Viet Nam/38/2004 | H5N1 |
| AY651507 | A/Ck/Viet Nam/39/2004 | H5N1 |
| AY651508 | A/Ck/Viet Nam/C57/2004 | H5N1 |
| AY651538 | A/Ck/YN/115/2004 | H5N1 |
| AY651537 | A/Ck/YN/374/2004 | H5N1 |
| AY651531 | A/Dk/HN/101/2004 | H5N1 |
| AY651530 | A/Dk/HN/303/2004 | H5N1 |
| AY651486 | A/Dk/Indonesia/MS/2004 | H5N1 |
| AY651496 | A/Dk/Thailand/71.1/2004 | H5N1 |
| AY651509 | A/Dk/Viet Nam/11/2004 | H5N1 |
| AY650273 | A/GSC_chicken/British Columbia/04 | H7N3 |
| AY648290 | A/GSC_chicken_B/British Columbia/04 | H7N3 |
| AY651497 | A/Gs/Thailand/79/2004 | H5N1 |
| AY651533 | A/Ph/ST/44/2004 | H5N1 |
| AY651494 | A/Qa/Thailand/57/2004 | H5N1 |
| AY651495 | A/bird/Thailand/3.1/2004 | H5N1 |
| AY611527 | A/chicken/British Columbia/04 | H7N3 |
| AY646081 | A/chicken/British Columbia/GSC_human_B/04 | H7N3 |
| AY609313 | A/chicken/Guangdong/174/04 | H5N1 |
| AY684707 | A/chicken/Hubei/327/2004 | H5N1 |
| AY653196 | A/chicken/Jilin/9/2004 | H5N1 |
| AY590579 | A/chicken/Nakorn-Patom/Thailand/CU-K2/2004 | H5N1 |
| AY574189 | A/chicken/Vietnam/HD1/2004 | H5N1 |
| AY574192 | A/chicken/Vietnam/HD2/2004 | H5N1 |
| AY576931 | A/muscovy duck/Vietnam/MdGL/2004 | H5N1 |
| AY651528 | A/peregrine falcon/HK/D0028/2004 | H5N1 |

### Example 18

### High Throughput Screen to Identify Highly Effective siRNAs

Cell Culture. Human lung epithelial cells A-549 (ATCC) were grown in DMEM medium containing 10% heat inactivated fetal calf serum (FCS), 2 mM L-glutamine, 100 units/ml penicillin and 100 ug/ml streptomycin. Other cell lines derived from the airway such as Calu-3 or HBE cells (ATCC), or other cell lines, could also be used. Cells were grown at 37° C. in a humidified incubator with 5% CO₂.

siRNAs. Various siRNAs each containing an antisense strand with a 19 nucleotide inhibitory region perfectly complementary to a highly conserved target portion listed in Table 16 and a sense strand complementary to the antisense strand were designed and synthesized. The siRNAs contained a 3′ dTdT overhang on both strands. All siRNAs were synthesized by Dharmacon Research (Lafayette, Colo.) using 2′ACE protection chemistry. The siRNAs were deprotected, desalted, and annealed by the manufacturer.

Dual luciferase assay to identify highly effective siRNAs. Full length cDNAs corresponding to the relevant influenza virus transcript; i.e., from NP, PA, PB1, PB2, M, NS, NA or HA gene from PR8 virus were cloned into the psiCHECK™-2 vector (Cat. No. C8021, Promega, Madison, Wis.) according to the directions of the manufacturer (see Promega Technical Bulletin No. 329, available at www.promega.com/tbs/tb329/tb329.pdf. The cDNAs were inserted into the 3′ UTR of a synthetic Renilla luciferase gene (hLuc) optimized for expression in mammalian cells. The DNA and siRNA targeted to the cDNA insert (or nonspecific sicontrol, Dharmacon) were co-transfected into A-549 cells using Lipofectamine 2000 (Invitrogen) according to the manufacturer's instructions. Following transfection, a fusion of the Renilla gene and the influenza virus gene was transcribed. Luciferase activity was measured as a function of the amount of each siRNA 24 hours after transfection. In this assay, RNAi mediated by the siRNA or shRNA hybridizing to the targeted portion of the fusion transcript (i.e., the influenza-specific portion of the fusion transcript) causes in degradation of the fused Renilla:influenza virus gene trancript resulting in a decreased luciferase signal. Firefly luciferase expressed from the same vector was used as a transfection control and specificity control as described in Promega Technical Bulletin 329. Specifically, 24hrs after transfection, the lyse and substrate buffer (Dual-Glo Luciferase Assay System) were added to cells, and luciferase activity was read. 10 min later, Stop and Glo for Renilla was added and Renilla luciferase activity was read. Triplicates of each sample were done. The firefly luciferase activity from each well was used as transfection control for renilla in the same well. The ratio of Renilla/firefly luciferase activities was used as the final value of luciferase activity from each well. The average of the ratio from influenza siRNA triplicates was compared to that of sicontrol triplicates. Silencing % was computed as 100×(1−influenzasiRNA/sicontrol).

Table 26 presents data for a number of highly effective siRNAs identified in the screen. The table lists target gene (NP, PA, PB1, PB2), siRNA concentration, and ID numbers of the siRNA. Average silencing % at each concentration tested is listed under the siRNA ID. Blank spaces indicate experiment not done. The results indicate that a number of siRNAs silenced transcript expression by at least 80% even at concentrations as low as 0.6 nM.

TABLE 26
siRNA ID
Target gene: NP

| Average | Conc. | | | | | | |
|---|---|---|---|---|---|---|---|
| Silencing | (nM) | 154 | 758 | 1121 | 1313 | NP-1496 | 1499 |
| (%) | 0.0048 | 9.32 | 0 | 10.88 | 11.72 | | |
| | 0.024 | 48.2 | 10.56 | 34.72 | 46.93 | | |
| | 0.12 | 65.71 | 35.25 | 66.54 | 75.28 | 29.23 | 12.08 |
| | 0.6 | 84.37 | 66.76 | 84.75 | 87.43 | 68.62 | 66.13 |
| | 3 | 90.09 | 71.96 | 89.08 | 91.05 | 84.74 | 78.99 |
| | 15 | 90.46 | 74.21 | 89.13 | 91.67 | 90.41 | 84.71 |
| | 75 | 90.03 | 69.94 | 86.96 | 89.82 | 90.96 | 85.97 |

Target gene: PA

| Average | Conc. | | | | |
|---|---|---|---|---|---|
| Silencing (%) | (nM) | 7736 | 7803 | 8282 | 8286 |
| | 0.0048 | 11.4 | 27.27 | 11.94 | 11.62 |
| | 0.024 | 29.3 | 44.88 | 18.78 | 37.57 |
| | 0.12 | 60.14 | 77.37 | 40.34 | 69.03 |
| | 0.6 | 78.25 | 85.13 | 85.95 | 83.23 |
| | 3 | 85.76 | 86.78 | 92.18 | 89 |
| | 15 | 85.74 | 87.82 | 93.34 | 89.8 |
| | 75 | 85.42 | 86 | | 90.64 |

Target gene: PB1

| Average | Conc. | | | | | | PB1- |
|---|---|---|---|---|---|---|---|
| Silencing (%) | (nM) | 4276 | 5018 | 5457 | 5773 | 6124 | 2257 |
| | 0.0048 | | 0 | 9.11 | 0 | 8.4 | 13.7 |
| | 0.024 | | 22.98 | 23.92 | 22.21 | 27.03 | 34.2 |
| | 0.12 | 4.61 | 72.99 | 60.67 | 53.46 | 57.67 | 66.32 |
| | 0.6 | 60.81 | 85.83 | 82.63 | 75.09 | 76.65 | 81.29 |
| | 3 | 87.98 | 89.12 | 87.63 | 84.76 | 80.84 | 84.88 |
| | 15 | 92.17 | 89.07 | 89.81 | 84.94 | 83.93 | 86.05 |
| | 75 | 92.07 | 93.4 | 88.57 | 87.56 | | 82.29 |

Target gene: PB2

| Average | Conc. | | | | | PB2- |
|---|---|---|---|---|---|---|
| Silencing (%) | (nM) | 2327 | 3276 | 3807 | 3817 | 2240 |
| | 0.0048 | 7.79 | 0 | 0 | 14.51 | 5.76 |
| | 0.024 | 51.49 | 30.49 | 17.15 | 45.26 | 35.44 |
| | 0.12 | 81.92 | 66.53 | 42.36 | 73.04 | 55.83 |
| | 0.6 | 88.79 | 82.09 | 70.44 | 80.35 | 77.48 |
| | 3 | 90.84 | 87.19 | 79.65 | 83.76 | 80.66 |
| | 15 | 90.82 | 87.04 | 85.08 | 83.53 | 82.65 |
| | 75 | 90.95 | 88.39 | 83.26 | 82.06 | 85.19 |

Certain of the siRNAs and/or shRNAs that were identified as highly effective using the above screen were further tested against influenza virus in tissue culture as described in Example 19 and/or animal models, as described elsewhere herein.

### Example 19

### Screens to Identify Highly Effective siRNAs that Inhibit Viral Replication

Cell Culture. Vero cells (ATCC) were grown in DMEM medium containing 10% heat inactivated fetal calf serum (FCS), 2 mM L-glutamine, 100 units/ml penicillin and 100 ug/ml streptomycin. Cells are grown at 37° C. in a humidified incubator with 5%. CO₂. Virus infections were done in DMEM containing 0.3% bovine serum albumin (BSA, Sigma, St. Louis, Mo.), 10 mM HEPES, 100 units/ml penicillin, and 100 μg/ml streptomycin.

siRNAs. siRNAs containing an antisense strand with a 19 nucleotide inhibitory region perfectly complementary to each of the highly conserved target portions listed in Table 18 and a sense strand complementary to the antisense strand were designed and synthesized. The siRNAs contained a 3′ dTdT overhang on both strands. All siRNAs were synthesized by Dharmacon Research (Lafayette, Colo.) using 2′ACE protection chemistry. The siRNAs were deprotected, desalted and annealed by the manufacturer.

siRNA transfection. Log-phase cultures of Vero cells were trypsinized, washed and seeded in 96-well plates at 20,000 cells per well and incubated at 37° C. in a humidified incubator with 5% CO₂ overnight. For the first screen, 10 pmol of siRNA (19 by duplex region with dTdT 3′ overhang) was added to 25 μl of Opti-MEM I (Invitrogen). 0.75 μl of Lipofectamine 2000 (Invitrogen) and 0.19 μl of SuperRNAsin (Ambion) were diluted in 25 μl of Opti-MEM I and mixed gently. Diluted lipid was combined with the diluted siRNA (total volume is 50 μl) and the mixture was incubated for 20 min at room temperature to allow the siRNA-Lipofectamine 2000 complexes to form. At the end of incubation, 50 μl of transfection complex were pipetted into each well containing cells and 50 μl DMEM medium containing 10% FCS. Final concentration of each siRNA was 100 nM. Each siRNA was tested in triplicate, and results were averaged. NP-1496 (100 nM) and sicontrol (Dharmacon) served as positive and negative controls, respectively. siRNA transfection was performed in the same way for the second screen except that a concentration of 1 nM siRNA was used.

Viral Infection. After incubation at 37° C. in a humidified incubator with 5% CO₂ for 6 hours, the supernatant was removed and the cells in each well were infected with PR8 virus in 25 μl of PBS containing 0.3% BSA. Infection was performed at an MOI of 0.1 for the first and second screens. The plates were rocked gently at room temperature for 1 h before 175 μl of DMEM containing 0.3% BSA, 4 μg trypsin, 10 mM HEPES was added to each well. The plates were then incubated at 37° C. in a humidified incubator with 5% CO₂.

Measurement of Viral Titer. Supernatants were harvested at 24 hours after infection. Viral titer was measured as described above.

Results

In order to identify those siRNAs with higher activity from among a set of 215 selected siRNAs targeted to highly conserved target portions of the influenza genome, we performed a series of high throughput screens (HTS). Rather than directly testing whether the siRNA can suppress expression of its specific target gene, we decided to focus on evaluating the capability of the siRNAs to inhibit influenza virus production in the cells. Therefore, we used the virus titer as the readout, while recognizing that certain siRNAs which cannot reduce the virus titer significantly may still possess the capability to degrade target mRNAs and are thus of use for certain purposes.

Among the 215 siRNAs tested in the first HTS, 90 showed at least a 4-fold decrease in PR8 virus titer in Vero cell culture relative to either no treatment (NT) or the sicontrol. No non-specific inhibition was seen when vero cells were transfected with 100 nM of sicontrol. The transfection efficiency was about 80%. Results are summarized in Table 21 and are expressed in terms of fold inhibition of virus titer. The actual extent of inhibition is likely to be greater than presented in the table.

TABLE 21
Results of High Throughput Screen #1

| Virus Titer | |
|---|---|
| Inhibition (fold) | siRNA ID |
| >8 | 752, 754, 758, 1121, 1122, 1223, 1225, 1254, 1313, 1316, 1381, 1383, |
| | 1488, 1499, 1527, NP1496 |
| ≧4 | 109, 119, 154, 222, 224, 301, 302, 359, 375, 688, 692, 1490, 1568, |
| | 1714, 1720, 1728, 2054, 2283, 2327, 3225, 3231, 3276, 3277, 3693, |
| | 3700, 3880, 3882, 3887, 4120, 4163, 4276, 4283, 4913, 4915, 4980, |
| | 5018, 5359, 5360, 5457, 5460, 5574, 5578, 5580, 5584, 5680, 5773, |
| | 5791, 5793, 5795, 5902, 5942, 5945, 5947, 5997, 6081, 6087, 6089, |
| | 6351, 6436, 6559, 6696, 6732, 6744, 7202, 7424, 75177580, 7581, |
| | 7648, 7736, 7803, 7807, 8172, 8271, 8282, 8285, 8286, 8289, 8299, |
| | 8300, 8709, 9331 |

We performed a second high throughput screen using a lower concentration of siRNA (1 nM) to more accurately identify the highly effective siRNAs from among those that showed, at least a 4-fold decrease in virus titer in the first HTS. Thirty siRNAs showed around a 3- to 4-fold decrease in PR8 virus titer in Vero cell culture when tested at this lower concentration. Results of the second HTS are presented in Table 22.

TABLE 22
Results of High Throughput Screen #2

| siRNA ID | Average | siRNA ID | Average | siRNA ID | Average |
|---|---|---|---|---|---|
| 109 | 2 | 1122 | 1 | 3276 | 1.33 |
| 119 | 1.67 | 1223 | 1.67 | 3277 | 1.67 |
| 154 | 1 | 1225 | 1.33 | 3693 | 4 |
| 222 | 2 | 1254 | 1.33 | 3700 | 3.33 |
| 224 | 1.67 | 1313 | 1 | 3880 | 4 |
| 301 | 1.67 | 1316 | 1 | 3882 | 2 |
| 302 | 1.67 | 1381 | 2 | 3887 | 2 |
| 359 | 2 | 1383 | 1.67 | 4120 | 2 |
| 375 | 1.67 | 1488 | 1.67 | 4163 | 2 |
| 688 | 1.67 | 1499 | 1.67 | 4276 | 1 |
| 692 | 2.67 | 1527 | 1.67 | 4283 | 1.33 |
| 752 | 2 | 1590 | 2 | 4913 | 2 |
| 754 | 1.67 | 1714 | 1.67 | 4915 | 1.33 |
| 758 | 1.33 | 2283 | 1.67 | 4980 | 2 |
| 1121 | 1 | 2327 | 1.33 | 5018 | 1.33 |
| sicontrol | 4 | NT | 4 | | |
| 5359 | 1.67 | 5947 | 1.67 | 7648 | 1 |
| 5360 | 1.67 | 5997 | 1.67 | 7736 | 1.67 |
| 5457 | 1.33 | 6081 | 1.33 | 7803 | 1 |
| 5460 | 1.33 | 6087 | 1.33 | 7807 | 1.67 |
| 5574 | 1.67 | 6089 | 1.33 | 8172 | 1.33 |
| 5578 | 1.67 | 6351 | 1.67 | 8271 | 3.33 |
| 5580 | 1.67 | 6436 | 2 | 8282 | 1 |
| 5584 | 1 | 6559 | 2 | 8285 | 4 |
| 5680 | 3.33 | 6696 | 1.67 | 8286 | 1 |
| 5773 | 1 | 6732 | 1.67 | 8289 | 1 |
| 5791 | 2 | 6744 | 1.67 | 8299 | 4 |
| 5793 | 1.67 | 7202 | 1.67 | 8300 | 1.67 |
| 5795 | 2.67 | 7424 | 4 | 9331 | 4 |
| 5902 | 2 | 7517 | 1.67 | sicontrol | 4 |
| 5942 | 2.67 | 7580 | 1.33 | NT | 4 |
| 5945 | 2 | 7581 | 1.33 | | |

### Example 20

### Confirmatory siRNA Screen

Materials and Methods

Thirty siRNAs selected from the second HTS and some siRNAs from the first HTS were tested in MDCK cells to confirm the results. 10 million MDCK cells in serum-free RPMI 1640 medium were mixed with siRNA at final concentration of 100 nM and electroporated at 400 V and 975 μF using a Gene Pulser apparatus (Bio-Rad). Electroporated cells were divided into three wells of a six-well plate and cultured in DMEM containing 10% fetal calf serum for 6 hours. The culture media was then removed and 100 microliters of PR8 virus in infection medium, consisting of DMEM, 0.3% BSA, 10 mM Hepes, 100 units/ml penicillin and 100 μg/ml streptomycin, was added to each well. Infection was performed at an MOI of 0.2, 0.02 and 0.002 for each of the three wells, respectively. After incubation for 1 h at room temperature, 2 ml of infection medium containing 4 μg/ml trypsin was added to each well, and the cells were cultured at 37 C under 5% CO2. At various times after infection, supernatants were harvested from infected cultures and the virus titer was determined by Hemagglutinin assay.

Results

To confirm the ability of the highly effective siRNAs identified in the first and second screens to inhibit influenza virus production, a third screen was performed in MDCK cells. MDCK cells are sensitive to even smaller amount of infection by laboratory strains of influenza virus than Vero cells. The virions replicate much faster in MDCK cells than in Vero cells. This third screen in MDCK cells further compared the efficiency of siRNAs in virus inhibition and confirmed the results of the first two screens with an accuracy of ˜85-90%.

At an MOI=0.2, 3 NP siRNAs reduced virus titer by at least 16-fold at 24 hours post-infection. 4 NP siRNAs (including 3 NP above), 2 PB2, 3 PB1 and 4 PA siRNAs reduced viral titer at least 8-fold. The following siRNAs represent the 13 most effective siRNAs tested: 154, 758, 1121, 1313, 2327, 3276, 4276, 5018, 5457, 7736, 7803, 8282, and 8286. At an MOI=0.02, 8 NP, 2 PB2, 3 PB1 and 8 PA siRNAs (including the siRNAs that were effective at MOI=0.2) reduced virus titer by at least 8-fold at 24 hours post-infection. At an MOI=0.002, 10 NP, 8 PB2, 11 PB1, 10 PA siRNAs (including the effective siRNAs effective at MOI=0.02) reduce virus titer by at least 4-fold at 24 hours post-infection. No virus inhibition was observed by sicontrol siRNA.

Results of the screen are presented in Table 23. The columns show HA units at times 24, 36, 48, and in some cases 60 hours post-infection at MOI=0.2/0.02/0.002, respectively. Plates were tested in sets, and sicontrol represents the control siRNA tested with each set. NT=no treatment. The most effective siRNAs are shown in bold.

TABLE 23
Results of High Throughput Screen #3

| siRNA | 24 h | 36 h | 48 h | 60 h |
|---|---|---|---|---|
| sicontrol | 512/256/8 | 1024/512/32 | 1024/512/32 | 1024/512/64 |
| 1313 | 32/4/1 | 128/16/1 | 256/64/2 | 512/64/4 |
| 1488 | 128/32/1 | 256/64/2 | 512/256/8 | 1024/256/16 |
| 2283 | 128/64/2 | 256/128/2 | 256/256/4 | 256/256/8 |
| 2327 | 64/32/2 | 256/64/2 | 256/128/4 | 512/256/8 |
| 3276 | 64/32/1 | 128/64/2 | 256/128/8 | 256/128/16 |
| 3277 | 128/64/2 | 256/128/4 | 256/128/16 | 512/128/16 |

| siRNA | 24 h | 36 h | 48 h |
|---|---|---|---|
| sicontrol | 1024/256/16 | 2148/1024/32 | 2148/1024/64 |
| 154 | 32/4/1 | 128/8/1 | 128/16/1 |
| 1121 | 64/4/1 | 128/16/1 | 256/32/2 |
| 1122 | 256/16/1 | 512/64/1 | 1024/64/4 |
| 1316 | 256/32/2 | 1024/128/16 | 1024/128/32 |
| 1499 | 256/32/1 | 1024/128/4 | 2148/128/8 |
| 1590 | 512/256/4 | 1024/256/16 | 2148/256/32 |
| 1714 | 512/128/2 | 1024/256/8 | 1024/256/8 |
| 3693 | 512/256/2 | 1024/512/8 | 1024/512/16 |
| 3700 | 512/256/2 | 1024/512/16 | 1024/512/16 |
| 8289 | 512/128/2 | 1024/128/16 | 1024/256/32 |
| sicontrol | 1024/256/4 | 2148/512/16 | 2148/1024/32 |
| 758 | 128/16/1 | 512/64/1 | 512/64/4 |
| 5018 | 128/16/1 | 512/64/1 | 512/64/4 |
| 6732 | 256/32/1 | 512/64/4 | 1024/512/8 |
| 7580 | 256/16/1 | 512/64/4 | 1024/512/8 |
| 7581 | 256/32/1 | 512/64/4 | 1024/512/16 |
| 7648 | 256/32/1 | 512/64/4 | 1024/512/32 |
| 7736 | 128/32/1 | 256/64/4 | 1024/512/16 |
| 7803 | 64/16/1 | 256/64/1 | 1024/512/8 |
| 8282 | 128/16/1 | 512/32/2 | 1024/512/8 |
| 8286 | 128/32/1 | 256/128/8 | 1024/512/16 |
| 7807 | 256/64/1 | 512/128/8 | 1024/512/32 |
| NT | 1024/256/16 | 1024/512/32 | 1024/512/64 |
| sicontrol | 1024/256/16 | 1024/512/32 | 1024/512/64 |
| 4276 | 128/32/1 | 256/64/8 | 256/64/8 |
| 4283 | 512/64/2 | 512/128/16 | 512/128/16 |
| 4915 | 512/128/4 | 512/128/16 | 512/128/16 |
| 5457 | 128/32/1 | 256/64/4 | 256/64/8 |
| 5460 | 512/64/4 | 512/128/16 | 512/128/16 |
| 5584 | 512/128/2 | 512/128/16 | 512/128/16 |
| 5773 | 256/32/1 | 256/64/4 | 256/64/4 |
| 6081 | 512/128/2 | 512/128/16 | 512/128/16 |
| 6087 | 512/64/2 | 512/128/16 | 512/128/16 |
| 6089 | 512/128/4 | 512/256/32 | 512/256/32 |
| 8172 | 512/128/8 | 512/256/16 | 512/256/32 |
| 1225 | 512/64/4 | 512/64/16 | 512/64/32 |
| 1254 | 256/64/4 | 256/128/32 | 512/128/32 |

### Example 21

### siRNA Dose Response Test

Materials and Methods

Thirteen siRNAs selected from the third HTS and some siRNAs from the second HTS were tested for dose response in MDCK cells. 10 million MDCK cells in serum-free RPMI 1640 medium were mixed with siRNA at various siRNA concentrations ranging from 0.8 nM to 100 nM. siRNA was electroporated into cells at 400 V and 975 μF by using a Gene Pulser apparatus (Bio-Rad). Electroporated cells were divided into three wells of a six-well plate and cultured in DMEM containing 10% fetal calf serum for 6 hours. The culture media was then removed and 100 microliters of PR8 virus in infection medium, consisting of DMEM, 0.3% BSA, 10 mM Hepes, 100 units/ml penicillin and 100 μg/ml streptomycin, was added to each well, to attain an MOI of 0.2, 0.02 and 0.002 for each of the three wells. After incubation for 1 h at room temperature, 2 ml of infection medium containing 4 μg/ml trypsin was added to each well and the cells were cultured at 37 C under 5% CO₂. At different times after infection, supernatants were harvested from infected cultures and the virus titer was determined by Hemagglutinin assay as described above.

Results: At MOI=0.2 and MOI=0.02, the minimum concentration of siRNAs that inhibit virus production by 2-fold at 24 hours post-infection are as follows, where asterisks indicate siRNAs that were identified as described in Example 1.

### NP Gene Segment as Target:

| | |
|---|---|
| 154 | 0.8 nM |
| 758 | 1 nM |
| 1121 | 1 nM |
| 1313 | 0.8 nM |
| 1499 | 5 nM |
| NP1496* | 4 nM |

### PB2 Gene Segment as Target:

| | |
|---|---|
| 2327 | 1 nM |
| 3276 | 1 nM |

### PB1 Gene Segment as Target:

| | |
|---|---|
| 4276 | 1 nM |
| 5018 | 5 nM |
| 5457 | 5 nM |
| 5773 | 5 nM |
| PB1-2257* | 5 nM |

### PA Gene Segment as Target:

| | |
|---|---|
| 7736 | 5 nM |
| 7803 | 1 nM |
| 8282 | 5 nM |
| 8286 | 5 nM |

Results are shown in Table 24 in terms of HA units at 24, 36, or 48 hours post-infection at MOI of 0.2/0.02/0.002 for various siRNA concentrations.

TABLE 24
Results of Dose Response Screen

| siRNA | 24 h | 36 h | 48 h |
|---|---|---|---|
| NT | 1024/128/2 | 2048/256/4 | 2048/256/8 |
| sicontrol 100 nM | 1024/128/2 | 2048/256/4 | 2048/256/8 |
| NP1496 0.8 nM | 1024/128/2 | 1024/256/4 | 2048/256/8 |
| NP1496 4 nM | 512/64/1 | 1024/256/4 | 2048/256/4 |
| NP1496 20 nM | 512/64/1 | 1024/256/4 | 2048/256/4 |
| 1313 0.8 nM | 512/64/1 | 1024/256/2 | 2048/256/4 |
| 1313 4 nM | 512/64/1 | 1024/128/2 | 2048/256/4 |
| 1313 20 nM | 256/32/1 | 512/64/1 | 1024/128/2 |
| 1313 25 nM | 128/8/1 | 256/32/1 | 512/64/1 |
| 154 0.8 nM | 512/64/1 | 1024/256/2 | 2048/256/4 |
| 154 4 nM | 512/64/1 | 1024/128/2 | 2048/128/2 |
| 154 20 nM | 256/32/1 | 512/64/1 | 1024/128/2 |
| 154 25 nM | 128/8/1 | 256/32/1 | 512/64/1 |
| 8172 100 nM | 512/128/1 | 1024/256/4 | 2048/256/8 |
| 8289 100 nM | 256/32/1 | 512/128/2 | 2048/256/4 |
| NT | 1024/128/2 | 2048/256/4 | 2048/256/8 |
| Sicontrol 100 nM | 1024/128/2 | 2048/256/4 | 2048/256/8 |
| 7803 1 nM | 512/64/1 | 1024/64/2 | 2048/128/4 |
| 7803 5 nM | 512/32/1 | 1024/64/2 | 1024/128/4 |
| 7803 25 nM | 256/32/1 | 512/64/2 | 1024/128/4 |
| 8282 1 nM | 1024/64/2 | 1024/128/4 | 2048/256/8 |
| 8282 5 nM | 512/64/1 | 1024/128/4 | 2048/256/8 |
| 8282 25 nM | 512/64/1 | 1024/128/4 | 1024/128/4 |
| 6696 100 nM | 512/32/1 | 1024/128/4 | 2048/256/8 |
| NT | 1024/128/2 | 2048/256/4 | 2048/256/8 |
| Sicontrol 25 nM | 1024/128/2 | 2048/256/4 | 2048/256/8 |
| 2327 1 nM | 512/64/1 | 1024/128/2 | 2048/128/8 |
| 2327 5 nM | 512/64/1 | 1024/128/2 | 2048/128/8 |
| 2327 25 nM | 512/32/1 | 1024/64/2 | 1024/64/8 |
| 3276 1 nM | 1024/64/2 | 1024/128/4 | 2048/128/8 |
| 3276 5 nM | 512/64/1 | 1024/128/2 | 2048/128/8 |
| 3276 25 nM | 512/64/1 | 1024/64/2 | 2048/64/4 |
| 758 1 nm | 512/64/1 | 1024/128/2 | 1024/128/4 |
| 758 5 nM | 512/64/1 | 1024/128/2 | 2048/128/8 |
| 758 25 nM | 256/32/1 | 1024/64/2 | 2048/64/8 |
| NT | 1024/128/2 | 2048/256/4 | 2048/512/8 |
| Sicontrol 25 nM | 1024/128/2 | 2048/256/4 | 2048/512/8 |
| 4276 1 nM | 512/64/1 | 1024/128/2 | 1024/128/4 |
| 4276 5 nM | 512/32/1 | 1024/64/2 | 1024/128/4 |
| 4276 25 nM | 256/16/1 | 512/32/1 | 512/64/2 |
| 5018 1 nM | 1024/64/2 | 2048/128/2 | 2048/256/4 |
| 5018 5 nM | 512/32/1 | 1024/64/2 | 1024/128/2 |
| 5018 25 nM | 256/16/1 | 512/32/1 | 1024/64/1 |
| 5457 1 nM | 1024/128/2 | 2048/256/4 | 2048/512/8 |
| 5457 5 nM | 512/32/1 | 1024/64/2 | 1024/128/4 |
| 5457 25 nM | 512/32/1 | 1024/64/2 | 1024/128/4 |
| 7736 1 nM | 1024/64/2 | 1024/128/4 | 2048/256/8 |
| 7736 5 nM | 512/64/1 | 1024/128/2 | 2048/256/4 |
| 7736 25 nM | 512/64/1 | 512/64/1 | 2048/256/4 |
| NT | 512/128/4 | 1024/256/8 | 1024/256/32 |
| Sicontrol 25 nM | 512/128/4 | 1024/256/8 | 1024/256/32 |
| 1121 1 nM | 256/128/4 | 512/256/8 | 512/256/32 |
| 1121 5 nM | 256/64/1 | 512/128/4 | 512/128/8 |
| 1121 25 nM | 128/32/1 | 256/64/1 | 512/64/4 |
| 5773 1 nM | 256/64/2 | 512/256/8 | 512/256/8 |
| 5773 5 nM | 256/64/1 | 512/128/2 | 512/128/4 |
| 5773 25 nM | 128/32/1 | 256/64/1 | 256/64/4 |
| PB1-2257 1 nM | 512/64/1 | 1024/128/4 | 1024/128/8 |
| PB1-2257 5 nM | 256/64/1 | 512/128/4 | 512/128/8 |
| PB1-2257 25 nM | 256/64/1 | 512/128/4 | 512/128/8 |
| NT | 1024/256/4 | 2048/512/16 | 2048/1024/32 |
| sicontrol 25 nM | 1024/256/4 | 2048/512/16 | 2048/1024/32 |
| 1499 1 nM | 1024/256/4 | 2048/256/8 | 2048/512/16 |
| 1499 5 nM | 512/64/2 | 2048/128/4 | 2048/256/8 |
| 1499 25 nM | 512/32/1 | 2048/64/2 | 2048/64/4 |
| 8286 1 nM | 1024/128/2 | 2048/256/4 | 2048/256/8 |
| 8286 5 nM | 512/64/2 | 2048/128/4 | 2048/256/8 |
| 8286 25 nM | 512/64/2 | 2048/128/4 | 2048/256/8 |
| 1499 + 8286 2.5 + 2.5 | 1024/256/2 | 2048/256/4 | 2048/256/8 |

### Example 22

### Effective Inhibition of Influenza Virus Using siRNA Combinations

Certain siRNAs selected from the third HTS and some siRNAs from the second HTS were tested for their anti-influenza effect in combination. The transfection, infection, and viral titer tests were performed as described in Example 21. The transfection of 1499 and 4276 together, with each siRNA at 12.5 nM, suppresses virus production slightly more effectively than either 1499 or 4276 alone at 25 nM.

TABLE 25
Effect of siRNAs in Combination

| siRNA | 24 h | 36 h | 48 h |
|---|---|---|---|
| NT | 1024/128/4 | 2048/256/8 | 2048/512/16 |
| sicontrol 50 nM | 1024/128/4 | 2048/256/8 | 2048/512/16 |
| 1499 + 8282 12.5 + 12.5 | 512/32/1 | 1024/64/2 | 1024/128/8 |
| 1499 + 4276 12.5 + 12.5 | 256/16/1 | 512/32/1 | 512/64/4 |
| 8282 + 4276 12.5 + 12.5 | 512/32/1 | 1024/64/2 | 1024/128/8 |
| 1499 + 8282 25 + 25 | 512/32/1 | 1024/64/2 | 1024/128/8 |
| 1499 + 4276 25 + 25 | 256/16/1 | 512/32/1 | 512/64/2 |
| 8282 + 4276 25 + 25 | 256/32/1 | 1024/64/2 | 1024/128/4 |
| 1499 25 nM | 512/32/1 | 1024/64/2 | 1024/128/8 |
| 1499 50 nM | 512/16/1 | 1024/64/2 | 1024/128/8 |
| 8282 25 nM | 512/64/1 | 1024/128/4 | 1024/128/8 |
| 8282 50 nM | 512/32/1 | 1024/64/2 | 1024/128/8 |
| 4276 25 nM | 256/32/1 | 512/64/2 | 1024/128/8 |
| 4276 50 nM | 256/16/1 | 512/32/1 | 512/64/2 |

### Example 23

### Inhibition of Influenza Virus by Direct Delivery of Naked siRNA to the Respiratory System

Materials and Methods

siRNA preparation, viral infection, lung harvests, and influenza virus titer assays were performed as described in Example 12. Mice were anesthetized using isofluorane (administered by inhalation). siRNA was delivered in a volume of 50 μl by intranasal drip. p values were computed using Student's T test.

Results

siRNA (NP-1496) in phosphate buffered saline (PBS) was administered to groups of mice (5 mice per group). Mice were infected with influenza virus (2000 PFU) 3 hours after siRNA administration. Lungs were harvested 24 hours post-infection and virus titer measured. In a preliminary experiment mice were anesthetized with avertin and 2 mg/kg siRNA was administered by intranasal drip. A reduction in virus titer relative to controls was observed, although it did not reach statistical significance (data not shown).

In a second experiment, Black Swiss mice were anesthetized using isofluorane/O₂. Various amounts of siRNA in PBS was intranasally administered into the mice., 50 ul each mouse. Three different groups (5 mice per group) received doses of 2 mg/kg, 4 mg/kg, or 10 mg/kg siRNA in PBS by intranasal drip. A fourth group that received PBS alone served as a control. Three hours later, the mice were anesthetized again using isofluorane/O₂, 30 ul of PR8 virus (2000 pfu=4× lethal dose) was intranasally administered into the mice. 24 h after infection, the mouse lungs were harvested, homogenized and virus titer was measured by evaluation of the TCID₅₀ as described above. Serial 5-fold dilutions of the lung homogenate were performed rather than 10-fold dilutions.

A significant and dose-dependent difference in virus titer was seen between mice in each of the three treated groups and the controls. The reduction in virus titer relative to controls was 3.45-fold (p=0.0125), 4.16-fold (p=0.0063), and 4.62-fold (p=0.0057) in the groups that received doses of 2 mg/kg, 4 mg/kg, and 10 mg/kg respectively. Data for the individual mice (TCID₅₀) is presented in Table 27 and shown in FIG. 31A.

In summary, these results demonstrate the efficacy of siRNA delivered to the respiratory system in an aqueous medium in the absence of specific agents to enhance delivery.

TABLE 27
Intranasal Delivery of Naked siRNA Inhibits Influenza Virus Production

| | | | | | | | P |
|---|---|---|---|---|---|---|---|
| Treatment | log₁₀TCID50 | | | | | Average | value |
| PBS | 26718.37 | 45687.78 | 45687.78 | 15625 | 26718.37 | 32087.46 | |
| NP (2 mg/kg) | 15625 | 15625 | 3125 | 3125 | 9137.56 | 9327.51 | 0.008 |
| NP (4 mg/kg) | 9137.56 | 9137.56 | 5343.68 | 9137.56 | 5343.68 | 7620 | 0.004 |
| NP (10 mg/kg) | 9137.56 | 9137.56 | 9137.56 | 3125 | 3125 | 6732.53 | 0.003 |

### Example 24

### Inhibition of Influenza Virus Production in Mice by Direct Delivery of Naked siRNA to the Respiratory System

This example confirms results of Example 23 and demonstrates inhibition of influenza virus production in the lung by administration of siRNA targeted to NP to the respiratory system in an aqueous medium in the absense of delivery-enhancing agents. 6 μg, 15 ug, 30 μg, and 60 μg of NP-1496 siRNAs or 60 μg of GFP-949 siRNAs in PBS were intranasally instilled into mice essentially as described in Example 23, except that mice were intranasally infected with PR8 virus, 1000 pfu per mouse, 2 hours after siRNA delivery. Lungs were harvested 24 hours after infection. As shown in Table 28 and FIG. 31B, NP-specific siRNA was effective for the inhibition of influenza virus when administered by intranasal instillation in an aqueous medium in the absence of delivery agents. A significant and dose-dependent difference in virus titer was seen between mice in each of the three treated groups and the controls.

TABLE 28
Inhibition of Influenza Virus Production in the Lung Using Naked
siRNA

| Treatment | TCID50 | | | | | Average | P value |
|---|---|---|---|---|---|---|---|
| PBS | 125 | 365.5 | 213.7 | 365.5 | 125 | 239.95 | |
| GFP (60 μg) | 125 | 213.7 | 213.7 | 213.7 | 365.5 | 226.32 | |
| NP (6 μg) | 213.7 | 213.7 | 125 | 213.7 | 42.7 | 161.8 | 0.263 |
| NP (15 μg) | 125 | 125 | 42.7 | 25 | 73.1 | 78.17 | 0.024 |
| NP (30 μg) | 8.5 | 125 | 42.7 | 125 | 14.6 | 63.18 | 0.019 |
| NP (60 μg) | 73.1 | 14.6 | 25 | 25 | 25 | 32.54 | 0.006 |

### Example 25

### siRNAs Targeted to Influenza Virus Transcripts Tolerate Mismatches in the Target Region

This example demonstrates that siRNAs whose antisense strands are less than 100% complementary to the targeted transcript within the inhibitory region (e.g., within the 19 base pair region that is complementary to the target transcript) mediate effective silencing. The results demonstrate that the RNAi agents described herein will effectively inhibit a wide range of influenza strains whose sequences vary from that of PR8 within the target portion.

Materials and Methods

A dual luciferase assay, as described in Example 18, was used to evaluate the ability of siRNAs to inhibit expression of influenza genes that are not 100% complementary to the antisense strand of the siRNA within the 19 nucleotide inhibitory region. Mismatches derived from the alignment of human and avian influenza virus strains (using PR8 as standards) were introduced into the DNA vector (psiCHECK) using a site-directed mutagenesis kit (Stratagene), i.e., the influenza target site was modified to include either 1 or 2 differences relative to the PR8 sequence, with the specific differences corresponding to differences found in one or more of the human or avian influenza strains listed in Table 15.

Table 29 shows results of an experiment demonstrating that variations in the viral NP target (target for NP-1496) do not substantially reduce RNAi activity. (The data shown is the average of triplicates). Mismatches at positions near the 5′ or 3′ end of the antisense strand, or near the middle, were tested.

TABLE 29
Effect of mismatches between antisense strand and target region on
silencing by NP-1496

| | | A3 to | | C12 to | C15 to | A18 to |
|---|---|---|---|---|---|---|
| | Original | G3 | T9 to C9 | T12 | T15 | G18 |
| Renilla | 85.6 | 81.8 | 58.3 | 67.8 | 72.9 | 54.7 |
| luciferase | | | | | | |
| silencing (%) | | | | | | |
| Remaining | 100 | 91.3 | 65.1 | 75.7 | 81.4 | 61.1 |
| silencing | | | | | | |
| comparing | | | | | | |
| with original | | | | | | |
| (%) | | | | | | |

Table 30 shows results of an experiment demonstrating that variations in the viral PA target (target for PA-2087 or PA-8242) do not substantially reduce RNAi activity. (The data shown as the average of triplicates). However, G18 to Al 8 mutations found in 7 among 157 human influenza strains did substantially affect the RNA interference activity. (The data shown is the average of triplicates). Mismatches at positions near the 5′ or 3′ end of the antisense strand, or near the middle, were tested. The presence of two mismatches between the antisense strand inhibitory region and the target reduced the silencing by about 70-75%, but a useful degree of silencing was still observed.

TABLE 30
Effect of mismatches between antisense strand and target region
on silencing by PA-2087 or PA-8242

| | | | | | | | | T6 to |
|---|---|---|---|---|---|---|---|---|
| | | | T6 | T6 | | | | C6 and |
| | | A4 to | to | to | C15 to | G18 to | A19 to | C15 to |
| | Original | G4 | A6 | C6 | T15 | A18 | G19 | T15 |
| Renilla luciferase | 91.7 | 80.8 | 75.9 | 88.8 | 87.5 | 7.0 | 89.3 | 26.8 |
| silencing (%) | | | | | | | | |
| Remaining | 100 | 88.1 | 82.8 | 96.9 | 95.5 | 7.6 | 97.4 | 29.3 |
| silencing | | | | | | | | |
| comparing with | | | | | | | | |
| original (%) | | | | | | | | |

Table 31 shows results of an experiment demonstrating that variations in the viral PB2 target (target for PB2-3817) do not substantially reduce RNAi activity. (The data shown is the average of triplicates).

TABLE 31
Effect of mismatches between antisense strand and target region
on silencing by PB2-3817

| | | A17 to | |
|---|---|---|---|
| | Original | G17 | A18 to T18 |
| Renilla luciferase | 86.7 | 73.4 | 75.8 |
| silencing (%) | | | |
| Remaining silencing | 100 | 100 | 87.4 |
| comparing with | | | |
| original (%) | | | |

Table 32A shows results of an experiment demonstrating that variations in the viral PB1 target (target for PB1-6124) do not substantially reduce RNAi activity. (The data shown is the average of triplicates). Mismatches at positions near the 5′ or 3′ end of the antisense strand, or near the middle, were tested. The presence of two mismatches between the antisense strand inhibitory region and the target reduced the silencing by about 70-75%, but a useful degree of silencing was still observed.

TABLE 32A
Effect of mismatches between antisense strand and target region
on silencing by PB1-6124

| | | | | | | T8 to C8 |
|---|---|---|---|---|---|---|
| | | A1 | | | T12 to | and C15 |
| | Original | to T1 | A5 to G5 | T8 to C8 | C12 | to T15 |
| Renilla | 82.2 | 83.9 | 77.8 | 63.3 | 83.2 | 26.5 |
| luciferase | | | | | | |
| silencing | | | | | | |
| (%) | | | | | | |
| Remaining | 100 | 100 | 94.7 | 77 | 100 | 32.2 |
| silencing | | | | | | |
| comparing | | | | | | |
| with | | | | | | |
| original | | | | | | |
| (%) | | | | | | |

Table 32B shows results of an additional experiment demonstrating that variations in the viral PB1 target (target for PB1-6124) do not substantially reduce RNAi activity. (The data shown is the average of triplicates). Mismatches at positions near the 5′ or 3′ end of the antisense strand were tested. The presence of two mismatches between the antisense strand inhibitory region and the target did not substantially reduce RNAi activity.

TABLE 32B
Effect of mismatches between antisense strand and target region
on silencing by PB1-6124

| PB1 (Lab | | | | | | A1 to |
|---|---|---|---|---|---|---|
| ID# | | C15 to | A1 to | T2 to | G3 to | T1 and G3 |
| 6124 siRNA) | Original | T15 | G1 | C2 | A3 | to A3 |
| Renilla | 84.1 | 71.8 | 82.8 | 84.5 | 74.9 | 73.9 |
| luciferase | | | | | | |
| silencing (%) | | | | | | |
| Remaining | 100 | 85.4 | 98.5 | 100 | 89.1 | 87.9 |
| silencing | | | | | | |
| comparing | | | | | | |
| with original (%) | | | | | | |

Table 32C shows results of an additional experiment demonstrating that variations in the viral PB1 target (target for PB1-6129) do not substantially reduce RNAi activity. (The data shown is the average of triplicates). Mismatches at positions near the 5′ or 3′ end of the antisense strand, or near the middle, were tested. A mismatch (G:U wobble) at position 10 had only a relatively small effect on silencing, demonstrating that such mismatches at position 10 do not substantially reduce RNAi activity in this context. The presence of two mismatches between the antisense strand inhibitory region and the target moderately reduced the silencing, but a about 60-70% of the original silencing effect was still observed.

TABLE 32C
Effect of mismatches between antisense strand and target region
on silencing by PB1-6129

| PB1 (Lab ID# | | T3 to | T7 to | T2 to | C10 to | T3 to C3 and |
|---|---|---|---|---|---|---|
| 6129 siRNA) | Original | C3 | C7 | C2 | T10 | C10 to T10 |
| Ranilla luciferase | 86.4 | 87.3 | 84.4 | 84.5 | 81.3 | 59.0 |
| silencing (%) | | | | | | |
| Remaining | 100 | 100 | 97.8 | 100 | 94.2 | 68.3 |
| silencing | | | | | | |
| comparing with | | | | | | |
| original (%) | | | | | | |

### Example 26

### Modified SiRNAs Mediate Effective Silencing

To explore the silencing potential of siRNAs containing modified nucleotides, NP-1496 siRNA containing sense and antisense strands with 2′-O-methyl modifications at alternate ribonucleotides in each strand were synthesized and tested in comparison with unmodified NP-1496 siRNA. The 2′-O-methyl modified NP1496 siRNA sequences were as follows: (2′-O-methyl shown as “m” in front of the modified nucleotide):

(SEQ ID NO: 381)

Sense: 5′-GmGA mUCmU UmAU mUUmC UmUC mGGmA G dTd
T-3′

(SEQ ID NO: 382)

Antisense: 5′-mCUmC CmGA mAGmA AmAU mAAmG AmUC mC
dTdT-3′

The 2′-O-methyl modified NP 1496 siRNA and unmodified NP 1496 siRNA were transfected into Vero cells in 24-well plate using lipofectamine 2000 (Invitrogen) following the manufacturer's instructions. 6 hours after transfection, the culture media was aspirated. The cells were inoculated with 200 ul of PR8 virus at MOI of 0.1. The culture supernatant was collected at 24, 36 and 48 hours after infection. Virus titer was determined as described above. The 2′-O-methyl modified NP1496 showed slightly more inhibition of virus growth than unmodified NP 1496. Results are shown in Table 33.

TABLE 33
Effective Inhibition of Influenza Virus Production Using Modified siRNA

| HA units | 24 h | 36 h | 48 h |
|---|---|---|---|
| No siRNA control | 4 | 8 | 16 |
| Unmodified NP1496 (400 uM) | 1 | 2 | 8 |
| Modified NP1496 (100 uM) | 1 | 2 | 8 |
| Modified NP1496 (200 uM) | 1 | 2 | 4 |
| Modified NP1496 (400 uM) | 1 | 1 | 4 |

### Example 27

### Summary of Screens and Tests to Identify Highly Effective siRNAs

The results of the screens and in vitro and in vivo tests described above were collected and combined to generate an overall list of influenza virus sequences that are both highly conserved and are targets of highly potent siRNAs. The list of target portions is presented in Table 34. These sequences also represent complements of the inhibitory region of antisense strands of certain highly effective RNAi-inducing agents, e.g., siRNAs.

TABLE 34
Highly Conserved Influenza Virus Sequences
that are Targets for
Highly Effective RNAi-Inducing Entities

| | | Target | |
|---|---|---|---|
| Sequence | SEQ ID NO: | Gene | Lab ID NO: |
| GCCACTGAAATCAGAGCAT | 272 | NP | 109 |
| TCAGAGCATCCGTCGGAAA | 273 | NP | 119 |
| GGACGATTCTACATCCAAA | 274 | NP | 154 |
| CAGCTTAACAATAGAGAGA | 275 | NP | 222 |
| GCTTAACAATAGAGAGAAT | 276 | NP | 224 |
| AATAGAGAGAATGGTGCTC | 277 | NP | 231 |
| GGGAAAGATCCTAAGAAAA | 278 | NP | 301 |
| GGAAAGATCCTAAGAAAAC | 279 | NP | 302 |
| TGAGAGAACTCATCCTTTA | 280 | NP | 359 |
| TTATGACAAAGAAGAAATA | 281 | NP | 375 |
| ACAAGAATTGCTTATGAAA | 282 | NP | 688 |
| GAATTGCTTATGAAAGAAT | 283 | NP | 692 |
| AAGCAATGATGGATCAAGT | 284 | NP | 752 |
| GCAATGATGGATCAAGTGA | 285 | NP | 754 |
| TGATGGATCAAGTGAGAGA | 286 | NP | 758 |
| CCACTAGAGGAGTTCAAAT | 287 | NP | 1121 |
| CACTAGAGGAGTTCAAATT | 288 | NP | 1122 |
| GAGGAAACACCAATCAACA | 289 | NP | 1223 |
| GGAAACACCAATCAACAGA | 290 | NP | 1225 |
| GGGCCAAATCAGCATACAA | 291 | NP | 1254 |
| CAACCATTATGGCAGCATT | 292 | NP | 1313 |
| CCATTATGGCAGCATTCAA | 293 | NP | 1316 |
| AGGATGATGGAAAGTGCAA | 294 | NP | 1381 |
| GATGATGGAAAGTGCAAGA | 295 | NP | 1383 |
| GAGTAATGAAGGATCTTAT | 296 | NP | 1488 |
| GGATCTTATTTCTTCGGAG | 297 | NP | 1498 |
| GATCTTATTTCTTCGGAGA | 298 | NP | 1499 |
| TCTTATTTCTTCGGAGACA | 299 | NP | 1501 |
| GGAGTACGACAATTAAAGA | 300 | NP | 1527 |
| GAACTAAGAAATCTAATGT | 301 | PB2 | 1590 |
| TGAAATGGATGATGGCAAT | 302 | PB2 | 1714 |
| GGAACATGCTGGGAACAGA | 303 | PB2 | 2283 |
| GAATGATGATGTTGATCAA | 304 | PB2 | 2327 |
| AATGGAATTTGAACCATTT | 305 | PB2 | 3276 |
| ATGGAATTTGAACCATTTC | 306 | PB2 | 3277 |
| GCACTAAGCATCAATGAAC | 307 | PB2 | 3693 |
| GCATCAATGAACTGAGCAA | 308 | PB2 | 3700 |
| GGAGACGTGGTGTTGGTAA | 379 | PB2 | 3759 |
| CGGGACTCTAGCATACTTA | 309 | PB2 | 3789 |
| ACTGACAGCCAGACAGCGA | 310 | PB2 | 3807 |
| AGACAGCGACCAAAAGAAT | 311 | PB2 | 3817 |
| GAATTCGGATGGCCATCAA | 312 | PB2 | 3832 |
| GCAGGCAAACCATTTGAAT | 313 | PB1 | 3878 |
| AGGCAAACCATTTGAATGG | 314 | PB1 | 3880 |
| GCAAACCATTTGAATGGAT | 315 | PB1 | 3882 |
| CCATTTGAATGGATGTCAA | 316 | PB1 | 3887 |
| CAGGATACACCATGGATAC | 380 | PB1 | 4001 |
| GACAATGAACCAAGTGGTT | 317 | PB1 | 4120 |
| AAGCAATGGCTTTCCTTGA | 318 | PB1 | 4163 |
| ACCTATGACTGGACTCTAA | 319 | PB1 | 4276 |
| ACTGGACTCTAAATAGAAA | 320 | PB1 | 4283 |
| CTCCAATAATGTTCTCAAA | 321 | PB1 | 4913 |
| CCAATAATGTTCTCAAACA | 322 | PB1 | 4915 |
| GAAACTTAGAACTCAAATA | 323 | PB1 | 4980 |
| GCATCGATTTGAAATATTT | 324 | PB1 | 5018 |
| ACATTTGAATTCACAAGTT | 325 | PB1 | 5359 |
| CATTTGAATTCACAAGTTT | 326 | PB1 | 5360 |
| GGACATGAGTATTGGAGTT | 327 | PB1 | 5457 |
| CATGAGTATTGGAGTTACT | 328 | PB1 | 5460 |
| ATGCCATAGAGGTGACACA | 329 | PB1 | 5574 |
| CATAGAGGTGACACACAAA | 330 | PB1 | 5578 |
| TAGAGGTGACACACAAATA | 331 | PB1 | 5580 |
| GGTGACACACAAATACAAA | 332 | PB1 | 5584 |
| CCAAATTTATACAACATTA | 333 | PB1 | 5680 |
| CCACTGAACCCATTTGTCA | 334 | PB1 | 5773 |
| AGCCATAAAGAAATTGAAT | 335 | PB1 | 5791 |
| CCATAAAGAAATTGAATCA | 336 | PB1 | 5793 |
| ATAAAGAAATTGAATCAAT | 337 | PB1 | 5795 |
| AGAAATCGATCCATCTTGA | 338 | PB1 | 5902 |
| TTGAAGATGAACAAATGTA | 339 | PB1 | 5942 |
| AAGATGAACAAATGTACCA | 340 | PB1 | 5945 |
| GATGAACAAATGTACCAAA | 341 | PB1 | 5947 |
| CAGCAGTTCATACAGAAGA | 342 | PB1 | 5997 |
| TTTCGAATCTGGAAGGATA | 343 | PB1 | 6081 |
| ATCTGGAAGGATAAAGAAA | 344 | PB1 | 6087 |
| CTGGAAGGATAAAGAAAGA | 345 | PB1 | 6089 |
| ATGAAGATCTGTTCCACCA | 346 | PB1 | 6124 |
| GATCTGTTCCACCATTGAA | 347 | PB1 | 6129 |
| ACCTGAAAATCGAAACAAA | 348 | PA | 6351 |
| GCACAGATTTGAAATAATC | 349 | PA | 6436 |
| GAATAGATTCATCGAAATT | 350 | PA | 6559 |
| ACTACACTCTCGATGAAGA | 351 | PA | 6696 |
| AAACCAGACTATTCACCAT | 352 | PA | 6732 |
| TCACCATAAGACAAGAAAT | 353 | PA | 6744 |
| GGAATAAATCCAAATTATC | 354 | PA | 7202 |
| CTAGCAAGTTGGATTCAGA | 355 | PA | 7424 |
| CCAATTGAACACATTGCAA | 356 | PA | 7517 |
| GCCACAGAATACATAATGA | 357 | PA | 7580 |
| CCACAGAATACATAATGAA | 358 | PA | 7581 |
| GGATGATTTCCAATTAATT | 359 | PA | 7648 |
| GGAAGATCCCACTTAAGGA | 360 | PA | 7736 |
| ACCCAAGACTTGAACCACA | 361 | PA | 7803 |
| AAGACTTGAACCACATAAA | 362 | PA | 7807 |
| CTCCACAACTAGAAGGATT | 362 | PA | 8172 |
| GGCTATATGAAGCAATTGA | 363 | PA | 8271 |
| GCAATTGAGGAGTGCCTGA | 364 | PA | 8282 |
| ATTGAGGAGTGCCTGATTA | 365 | PA | 8285 |
| TTGAGGAGTGCCTGATTAA | 366 | PA | 8286 |
| AGGAGTGCCTGATTAATGA | 367 | PA | 8289 |
| GATTAATGATCCCTGGGTT | 368 | PA | 8299 |
| ATTAATGATCCCTGGGTTT | 369 | PA | 8300 |
| TGATCCCTGGGTTTTGCTT | 370 | PA | 8305 |
| CCGAGGTCGAAACGTACGT | 371 | M | 8447 |
| ACCAATCCTGTCACCTCTG | 372 | M | 8580 |
| CAGTGAGCGAGGACTGCAG | 373 | M | 8640 |
| GACGCTTTGTCCAAAATGC | 374 | M | 8663 |
| TGGCTGGATCGAGTGAGCA | 375 | M | 9008 |
| TGTGGATTCTTGATCGTCT | 376 | M | 9240 |
| GTCTATGAGGGAAGAATAT | 377 | M | 9331 |
| GTCAGGCTAGGCAAATGGT | 378 | M | M645 |

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the claims that follow the reference list.

### References

1. Lamb, R. A., and R. M. Krug. 2001. Orthomyxoviridae: the viruese and their replication. Fundamental Virology Ed. D. M. Knipe & P. M. Hpwley:725-770.
2. Simonsen, L., K. Fukuda, L. B. Schomberger, and N. J. Cox. 2000. The impact of influenza epidemics on hospitalizations. J. Infect. Dis. 181:831-837.
3. Webster, R. G., W. J. Bean, O. T. Gorman, T. M. Chambers, and Y. Kawaoka. 1992. Evolution and ecology of influenza A viruses. Microbiol. Rev. 56:152-179.
4. Parvin, J. D., A. Moscona, W. T. Pan, J. M. Leider, and P. Palese. 1986. Measurement of the mutation rates of animal viruses: influenza A virus and poliovirus type 1. J. Virol. 59:377-383.
5. Smith, F. L., and P. Palese. 1989. Variation in influenza virus genes:epidemiology, pathogenic, and evolutionary consequences. The influenza viruses Krug, R. M. ed.:New York:Plenum.
6. Webster, R. G., W. G. Laver, G. M. air, and S. G. C. 1982. Molecular mechanisms of variation in influenza viruses. Nature 296:115-121.
7. Webby, R. J., and R. G. Webster. 2001. Emergence of influenza A viruses. Phil. Trans. R. Soc. Lond. 356:1817-1828.
8. Patterson, K. D., and G. F. Pyle. 1991. The geography and mortality of the 1918 influenza pandemic. Bull. Hist. Med. 65:4-21.
9. Taubenberger, J. K., A. H. Reid, T. A. Janczewski, and T. G. GFanning. 2001. Integrating historical, clinical and molecular genetic data in order to explain the origin and virulence of the 1918 Spanish influenza virus. Phil. Trans. R. Soc. Lond. 356:1829-1839.
10. Claas, E. C., A. D. Osterhaus, R. van Beek, J. C. De Jong, G. F. Rimmelzwaan, D. A. Senne, S. Krauss, K. F. Shortridge, and R. G. Webster. 1998. Human influenza A H5N1 virus related to a highly pathogenic avian influenza virus. Lancet 351:472-477.
11. Yuen, K. Y., P. K. Chan, M. Peiris, D. N. Tsang, T. L. Que, K. F. Shortridge, P. T. Cheung, W. K. To, E. T. Ho, R. Sung, and A. F. Cheng. 1998. Clinical features and rapid viral diagnosis of human disease associated with avian influenza A H5N1 virus. Lancet 351:467-471.
12. Fukuda, F., C. B. Bridges, and T. L.e.a. Brammer. 1999. Prevention and control of influenza: recommendations of the advisory committee on immunization practices (ACIP). MMWR Morb. Mortal. Wkly. Rep. 48:1-28.
13. Castle, S. C. 2000. Clinical relevane of age-related immune dysfunction. Clin. Infect. Dis.

31:578-585.
14. Luscher-Mattli, M. 2000. Influenza chemotherapy: a review of the present state of art and of new drugs in development. Arch. Virol. 145:2233-2248.
15. Cox, N. J., and K. Subbarao. 1999. Influenza. Lancet 354:1277-1282.
16. Vaucheret, H., C. Beclin, and M. Fagard. 2001. Post-transcriptional gene silencing in plants. J. Cell Sci. 114:3083-3091.
17. Sharp, P. A. 2001. RNA interference-2001. Genes Dev. 15:485-490.
18. Brant, S. 2002. Antisense-RNA regulation and RNA interference. Biochem. Biophy. Acta 1575:15-25.
19. Baulcombe, D. 2002. RNA silencing. Curr. Biol. 12:R82-R84.
20. Fire, A., S. Xu, M. K. Montgomery, S. A. Kostas, S. E. Driver, and M. C. C. 1998. Potent and specific genetic interference by double-stranded RNA in Caenorhabditis elegans. Nature 391:806-811.
21. Elbashir, S., J. Harborth, W. Lendeckel, A. Yalcin, K. Weber, and T. Tuschl. 2001. Duplexes of 21-nucleotide RNAs mediate RNA interference in cultured mammalian cells. Nature 411:494-498.
22. McManus, M. T., and P. A. Sharp. 2002. Gene silencing in mammals by short interfering RNAs. Nature Rev. Gene. 3:737-747.
23. Kumar, M., and G. G. Carmichael. 1998. Antisense RNA: function and fate of duplex RNA in cells of higher eukaryotes. Microbiol. Mol. Biol. Rev. 62:1415-1434.
24. Gitlin, L., S. Karelsky, and R. Andino. 2002. Short interfering RNA confers intracellular antiviral immunity in human cells. Nature 418:430-434.
25. Pderoso de Lima, M. C., S. Simoes, P. Pires, H. Faneca, and N. Duzgunes. 2001. Cationic lipid-DNA complexes in gene delivery: from biophysics to biological applications. Adv. Drug Deliv. Rev. 47:277-294.
26. Holen, T., M. Amarzguioui, M. T. Wiiger, E. Babaie, and H. Prydz. 2002. Positional effects of short interfering RNAs targeting the human coagulation trigger tissue factor. Nucleic Acids Res. 30:1757-1766.
27. McManus, M. T., Haines, B. B., Dillon, C. P., Whitehurst, C. E., van Parijs, L., Chen, J., and Sharp, P. A. (2002). Small interfering RNA-mediated gene silencing in T lymphocytes. J Immunol 169, 5754-5760.
28. Elbashir, S. M., J. Martinez, A. Patkaniowska, W. Lendeckel, and T. Tuschl. 2001. Functional anatomy of siRNAs for mediating efficient RNAi in Drosophila melanogaster embryo lysate. EMBO J. 20:6877-6888.
29. Yang, D., H. Lu, and J. W. Erickson. 2000. Evidence that processed small dsRNAs may mediate sequence-specific mRNA degradation during RNAi in Drosophila embryos. Curr. Biol. 10:1191-1200.
30. Caplen, N. J., J. Fleenor, A. Fire, and R. A. Morgan. 2000. dsRNA-mediated gene silencing in cultured Drosophila cells: a tissue culture model for the analysis of RAN interference. Gene 252:95-105.
31. Edwards, D. A., J. Hanes, G. Caponetti, J. Hrkach, A. Ben-Jebria, M. L. Eskew, J. Mintzes, D. Deaver, N. Lotan, and R. Langer. 1997. Large porous particles for pulmonary drug delivery. Science 276:1868-1871.
32. Putnam, D., C. A. Gentry, D. W. Pack, and R. Langer. 2001. Polymer-based gene delivery with low cytotoxicity by a unique balance of side-chain termini. Proc. Natl. Acad. Sci. USA 98:1200-1205.
33. Lynn, D. M., and R. Langer. 2000. Degradable Poly(-amino esters): Synthesis, Characterization, and Self-Assembly with Plasmid DNA. J. Am. Chem. Soc. 122:10761-10768.
34. Lynn, D. M., D. G. Anderson, D. Putnam, and R. Langer. 2001. Accelerated discovery of synthetic transfection vectors: parallel synthesis and screening of a degrable polymer library. J. Am. Chem. Soc. 123:8155-8156.
35. Han, S.-O., R. I. Mahato, Y. K. Sung, and S. W. Kim. 2000. Development of Biomaterials for gene therapy. Mol. Therapy 2:302-317.
36. Soane, R. J., M. Frier, A. C. Perkins, N. S. Jones, S. S. Davis, and L. Illum. 1999. Evaluation of the clearance characteristics of bioadhesive systems in humans. Int. J. Pharm. 178:55-65.
37. Boussif, O., F. Lezoualc'h, M. A. Zanta, M. D. Mergny, D. Scherman, B. Demeneix, and J. P. Behr. 1995. A versatile vector for gene and oligonucleotide transfer into cells in culture and in vivo: polyethylenimine. Proc. Natl. Acad. Sci. USA 92:7297-7301.
38. Astafieva, I., I. Maksimova, E. Lukanidin, V. Alakhov, and A. Kabanov. 1996. Enhancement of the polycation-mediated DNA uptake and cell transfection with Pluronic P85 block copolymer. FESB Lett. 389:278-280.
39. Davis, S. S. 1999. Delivery of peptide and non-peptide drugs through the respiratory tract. Pharm. Sci. Technol. Today 2:450-457.
40. Roy, K., H.-Q. Mao, S.-K. Huang, and K. W. Leong. 1999. Oral delivery with chitosan/DNA nanoparticles generates immunologic protection in murine model of peanut allergy. Nat. Med. 5:387-391.
41. Hansen, M. B., S. E. Nielsen, and K. Berg. 1989. Re-examination and further development of a precise and rapid dye method for measuring cell growth/cell kill. J. Immunol. Methods 119:203-210.
42. Green, M., and P. M. Loewenstein. 1988. Autonomous functional domains of chemically synthesized human immunodeficiency virus tat trans-activator protein. Cell 55:1179-1188.
43. Frankel, A. D., and C. O. Pabo. 1988. Cellular uptake of the tat protein from human immunodeficiency virus. Cell 55:1189-1193.
44. Elliott, G., and P. O'Hare. 1997. Intercellular trafficking and protein delivery by a herpesvirus structural protein. Cell 88:223-233.
45. Joliot, A., C. Pernelle, H. Deagostini-Bazin, and A. Prochiantz. 1991. Antennapedia homeobox peptide regulates neural morphogenesis. Proc. Natl. Acad. Sci. USA 88:1864-1868.
46. Fawell, S., J. Seery, Y. Daikh, C. Moore, L. L. Chen, B. Pepinsky, and J. Barsoum. 1994. Tat-mediated delivery of heterologous proteins into cells. Proc. Natl. Acad. Sci. USA 91:664-668.
47. Schwarze, S. R., A. Ho, A. Vocero-Akbani, and S. F. Dowdy. 1999. In vivo protein transduction:delivery of a biologically active protein. Science 285:1569-1572.
48. Derossi, D., G. Chassaing, and A. Prochiantz. 1998. Trojan peptides: the penetratin system for intracellular delivery. Trends Cell Biol. 8:84-87.
49. Troy, C. M., D. Derossi, A. Prochiantz, L. A. Greene, and M. L. Shelanski. 1996. Downregulation of Cu/Zn superoxide dismutase leads to cell death via the nitric oxide-peroxynitrite pathway. J. Neurosci. 16:253-261.
50. Allinquant, B., P. Hantraye, P. Mailleux, K. Moya, C. Bouillot, and A. Prochiantz. 1995. Downregulation of amyloid precursor protein inhibits neurite outgrowth in vitro. J. Cell Biol. 128:919-927.
51. Futaki, S., T. Suzuki, W. Ohashi, T. Yagami, S. Tanaka, K. Ueda, and Y. Sugiura. 2001. Arginine-rich peptides. An abundant source of membrane-permeable peptides having potential as carriers for intracellular protein delivery. J. Biol. Chem. 276:5836-5840.
52. Densmore, C. L., F. M. Orson, B. Xu, B. M. Kinsey, J. C. Waldrep, P. Hua, B. Bhogal, and V. Knight. 1999. Aerosol delivery of robust polyethyleneimine-DNA complexes for gene therapy and genetic immunization. Mol. Therapy 1:180-188.
53. Arppe, J., M. Widgred, and J. C. Waldrep. 1998. Pulmonary pharmacokinetics of cyclosporin A liposomes. Intl. J. Pharm. 161:205-214.
54. Griesenbach, U., A. chonn, R. Cassady, V. Hannam, C. Ackerley, M. Post, A. K. Transwell, K. Olek, H. O'Brodovich, and L.-C. Tsui. 1998. Comparison between intratracheal and intravenous administration of liposome-DNA complexes for cystic fibrosis lung gene therapy. Gene Ther. 5:181-188.
55. Orson, F. M., L. Song, A. Gautam, C. L. DEnsmore, B. Bhogal, and B. M. Kinsey. 2002. Gene delivery to the lung using protein/polyethyleneimine/plasmid complexes. Gene Therapy 9:463-471.
56. Gautam, A., C. L. Densmore, E. Golunski, B. Xu, and J. C. Waldrep: 2001. Transgene expression in mouse airway epithelium by aerosol gene therapy with PEI-DNA complexes. Mol. Therapy 3:551-556.
57. Tabata, Y., and Y. Ikada. 1988. Effect of size and surface charge of polymer microspheres on their phagocytosis by macrophage. J. Biomed. Mater. Res. 22:837-842.
58. Vanbever, R., J. D. Mintzes, J. Wang, J. Nice, D. chen, R. Batycky, L. R., and D. A. Edwards. 1999. Formulation and physical characterization of large porous particles for inhalation. Pharmaceutical Res. 16:1735-1742.
59. McManus, M. T., C. P. Peterson, B. B. Haines, J. Chen, and P. A. Sharp. 2002. Gene silencing using micro-RNA designed hairpins. RNA 8:842-850.
60. Myslinski, E., J. C. Ame, A. Krol, and P. Carbon. 2001. An unusually compact external promoter for RNA polymerase III transcription of the human H1 RNA gene. Nucleic Acids Res. 29:2502-2509.
61. Brummelkamp, T. R., R. Bernards, and R. Agami. 2002. A system for stable expression of short interfering RNAs in mammalian cells. Science 296:550-553.
62. Paddison, P. J., A. A. Caudy, E. Bernstein, G. J. Hannon, and D. S. Conklin. 2002. Short hairpin RNAs (shRNAs) induce sequence-sepcific silencing in mammalian cells. Genes Dev. 16:948-958.
63. Gil, J., and M. Esteban. 2000. Induction of apoptosis by the dsRNA-dependent protein kinase (PKR): mechanism of action. Apoptosis 5:107-114.
64. Bitko, V., and S. Barik. 2001. Phenotypic silencing of cytoplasmic genes using sequence-specific double-stranded short interfering RNA and its application in the reverse genetic of wild type negative-strand RNA viruses. BMC Microbial. 1:34-43.
65. Garcia-Sastre, A. (2002) Microbes & Inf. 4, 647-655.
66. Katze, M. G., He, Y. & Gale Jr., M. (2002) Nature Rev. Immunol. 2, 675-687.
67. Diaz, M. O., Ziemin, S., Le Beau, M. M., Pitha, P., Smith, S. D., Chilcote, R. R. & Rowley, J. D. (1988) Proc. Natl. Acad. Sci. USA 85, 5259-5263.
68. Diaz, M. O., Pomykala, H. M., Bohlander, S. K., Maltepe, E., Malik, K., Brownstein, B. & Olopade, O. I. (1994) Genomics 22, 540-552.
69. Kim, M.-J., Latham, A. G. & Krug, R. M. (2002) Proc. Natl. Acad. Sci. USA 99, 10096-10101.
70. Medcalf, L., Poole, E., Elton, D. & Digard, P. (1999) J. Viral. 73, 7349-7356.
71. Shapiro, G. I. & Krug, R. M. (1988) J. Virol. 62, 2285-2290.
72. Beaton, A. R. & Krug, R. M. (1986) Proc. Natl. Acad. Sci. USA 83, 6282-6286
73. Lois, C., E. J. Hong, S. Pease, E. J. Brown, and D. Baltimore. (2002) Science 295:868-872.
74. Weiss, D. J., G. M. Mutlu, L. Bonneau, M. Mendez, Y. Wang, V. Dumasius, and P. Factor. (2002) Mol. Ther. 6:43-49.
75. Hansen, M. B., S. E. Nielsen, and K. Berg. (1989) J. Immunol. Methods 119:203-210.
76. Kunath, K, von Harpe A, Fischer D, Petersen H, Bickel U, Voigt K, Kissel T. (2003) J Control Release 89(1):113-25.
77. Jobe A. Surfactant treatment for respiratory distress syndrome. Respir Care 1986;31(6):467-476.
78. Berry D. Neonatology in the 1990's: surfactant replacement therapy becomes a reality. Clin Pediatr 1991;30(3):167-170.
79. Avery ME, Mead J. Surface properties in relation to atelectasis and hyaline membrane disease. Am J Dis Child 1959;97:517-523.
80. von Neergard K. Neue Auffassungen fiber einen Grundbegriff der Atemmechanik: die Retraktionskraft der Lunge, äbhangig von der Oberflachenspannung in den Alveolen. Z Ges Exp Med 1929;66:373.
81. Hallman M, Teramo K, Ylikorkala O, Merritt T A. Natural surfactant substitution in respiratory distress syndrome. J Perinat Med 1987;15:463-468.
82. Bloom B T, Kattwinkel J, Hall R T, et al. Comparison of Infasurf (calf lung surfactant extract) to Survanta (beractant) in the treatment and prevention of respiratory distress syndrome. Pediatrics. 1997;100:31-38.
83. Mizuno K, Ikegami M, Chen C-M, et al. Surfactant protein-B supplementation improves in vivo function of a modified natural surfactant. Pediatr Res. 1995;37:271-276.
84. Hall S B, Venkitaraman A R, Whitsett J A, et al. Importance of hydrophobic apoproteins as constituents of clinical exogenous surfactants. Am Rev Respir Dis. 1992;145:24-30.
85. C. H. Ahn, S. Y. Chae, Y. H. Bae and S. W. Kim, Biodegradable poly(ethylenimine) for plasmid DNA delivery. J Control Release 80, 273-82, 2002.
86. Kichler, A., Leborgne C, Coeytaux E, Danos O. Polyethylenimine-mediated gene delivery: a mechanistic study. J Gene Med. 2001 Mar-April;3(2):135-44.
87. Brissault B, Kichler A, Guis C, Leborgne C, Danos O, Cheradame H. Synthesis of linear polyethylenimine derivatives for DNA transfectionBioconjug Chem. 2003 May-June; 14(3):581-7
88. Kichler A, Leborgne C, Marz J, Danos O, Bechinger B. Histidine-rich amphipathic peptide antibiotics promote efficient delivery of DNA into mammalian cellsProc Natl Acad Sci USA. 2003 Feb. 18;100(4):1564-8
89. Brantl, S. (2002). Antisense-RNA regulation and RNA interference. Biochim Biophys Acta 1575, 15-25.
90. Semizarov, D., Frost, L., Sarthy, A., Kroeger, P., Halbert, D. N., and Fesik, S. W. (2003). Specificity of short interfering RNA determined through gene expression signatures. Proc Natl Acad Sci USA 100, 6347-6352.
91. Chi, J. T., Chang, H. Y., Wang, N. N., Chang, D. S., Dunphy, N., and Brown, P. O. (2003). Genomewide view of gene silencing by small interfering RNAs. Proc Natl Acad Sci USA 100, 6343-6346.
92. Bitko, V., and Barik, S. (2001). Phenotypic silencing of cytoplasmic genes using sequence-specific double-stranded short interfering RNA and its application in the reverse genetics of wild type negative-strand RNA viruses. BMC Microbiol 1, 34.
93. Anderson, D. G., Lynn, D. M., and Langer, R. (2003). Semi-Automated Synthesis and Screening of a Large Library of Degradable Cationic Polymers for Gene Delivery. Angew Chem Int Ed Engl 42, 3153-3158.
94. Ge, Q., McManus, M., Nguyen, T., Shen, C.-H., Sharp, P. A., Eisen, H. N., and Chen, J. (2003). RNA interference of influenza virus production by directly targeting mRNA for degradation and indirectly inhibiting all viral RNA transcription. Proc Natl Acad Sci USA 100, 2718-2723.
95. Kumar, M., and Carmichael, G. G. (1998). Antisense RNA: function and fate of duplex RNA in cells of higher eukaryotes. Microbiol Mol Biol Rev 62, 1415-1434.
96. Lèuscher-Mattli, M. (2000). Influenza chemotherapy: a review of the present state of art and of new drugs in development. Arch Virol 145, 2233-2248.
97. McCaffrey, A. P., Meuse, L., Pham, T. T., Conklin, D. S., Hannon, G. J., and Kay, M. A. (2002). RNA interference in adult mice. Nature 418, 38-39.
98. McCaffrey, A. P., Nakai, H., Pandey, K., Huang, Z., Salazar, F. H., Xu, H., Wieland, S. F., Marion, P. L., and Kay, M. A. (2003). Inhibition of hepatitis B virus in mice by RNA interference. Nat Biotechnol 21, 639-644,
99. Rubinson, D. A., Dillon, C. P., Kwiatkowski, A. V., Sievers, C., Yang, L., Kopinja, J., Rooney, D. L., Ihrig, M. M., McManus, M. T., Gertler, F. B., et al. (2003). A lentivirus-based system to functionally silence genes in primary mammalian cells, stem cells and transgenic mice by RNA interference. Nat Genet 33, 401-406.
100. Shapiro, G. I., and Krug, R. M. (1988). Influenza virus RNA replication in vitro: synthesis of viral template RNAs and virion RNAs in the absence of an added primer. J Virol 62, 2285-2290.
101. Shen, C., Buck, A. K., Liu, X., Winkler, M., and Reske, S. N. (2003). Gene silencing by adenovirus-delivered siRNA. FEBS lett 539, 111-114.
102. Simeoni, F., Morris, M. C., Heitz, F., and Divita, G. (2003). Insight into the mechanism of the peptide-based gene delivery system MPG: implications for delivery of siRNA into mammalian cells. Nucleic Acids Res 31, 2717-2724.
103. Soane, R. J., Frier, M., Perkins, A. C., Jones, N. S., Davis, S. S., and Ilium, L. (1999). Evaluation of the clearance characteristics of bioadhesive systems in humans. Int J Pharm 178, 55-65.
104. Stiver, G. (2003). The treatment of influenza with antiviral drugs. CMAJ 168, 49-56.
105. Tachibana, R., Harashima, H., Ide, N., Ukitsu, S., Ohta, Y., Suzuki, N., Kikuchi, H., Shinohara, Y., and Kiwada, H. (2002). Quantitative analysis of correlation between number of nuclear plasmids and gene expression activity after transfection with cationic liposomes, Pharm Res 19, 377-381.
106. Thomas, C. E., Ehrhardt, A., and Kay, M. A. (2003). Progress and problems with the use of viral vectors for gene therapy. Nat Rev Genet 4, 346-358.
107. Xia, H., Mao, Q., Paulson, H. L., and Davidson, B. L. (2002). siRNA-mediated gene silencing in vitro and in vivo. Nat Biotechnol 20, 1006-1010.
108. Zhang, G., Song, Y. K., and Liu, D. (2000). Long-term expression of human alphal-antitrypsin gene in mouse liver achieved by intravenous administration of plasmid DNA using a hydrodynamics-based procedure. Gene Ther 7, 1344-1349
109. Cheung, M., and Lieberman, J. M. (2002). Influenza: update on strategies for managment. Contemporay Pediatrics 19, 82-114.
110. Yang, P., Althage, A., Chung, J., and Chisari, F. (2000) Hydrodynamic injection of viral DNA: A mouse model of acute hepatitis B virus infection, Proc. Natl. Acad. Sci., 99(21): 13825-13830.
111. Zhang, G., Budker, V., Wolff, J A (1999) High levels of foreign gene expression in hepatocytes after tail vein injections of naked plasmid DNA, Hum Gene Ther, 10:1735-1737.
112. Liu, F., Song, Y. K., Liu, D. (1999) Hydrodynamics-based transfection in animals by systemic administration of plasmid DNA. Gene Therapy; 6:1258-1266.
113. Naldini, L. Lentiviruses as gene transfer agents for delivery to non-dividing cells. Curr Opin Biotechnol 9, 457-63 (1998).
114. Naldini, L. et al. In vivo gene delivery and stable transduction of nondividing cells by a lentiviral vector. Science 272, 263-7 (1996).
115. Miyoshi, H., Blomer, U., Takahashi, M., Gage, F. H. & Verma, I. M. Development of a self-inactivating lentivirus vector. J Virol 72, 8150-7 (1998).
116. Pfeifer, A., Ikawa, M., Dayn, Y. & Verma, I. M. Transgenesis by lentiviral vectors: lack of gene silencing in mammalian embryonic stem cells and preimplantation embryos. Proc Natl Acad Sci USA 99, 2140-5 (2002).
117. Lois, C., Hong, E. J., Pease, S., Brown, E. J. & Baltimore, D. Germline transmission and tissue-specific expression of transgenes delivered by lentiviral vectors. Science 295, 868-72 (2002).
118. Anderson D G, Lynn D M, Langer R., Semi-Automated Synthesis and Screening of a Large Library of Degradable Cationic Polymers for Gene Delivery. Angew Chem Int Ed Engl. 2003 Jul. 14;42(27):3153-3158.
119. McCown, M., Diamond, M. S., and Pekosz, A., Virology, 313: 514-524 (2003).
120. Gratton, J. P., Yu, J., Griffith, J. W., Babbitt, R. W., Scotland, R. S., Hickey, R., Giordano, F. J., and Sessa, W. C., Cell-permeable peptides improve cellular uptake and therapeutic gene delivery of replication-deficient viruses in cells and in vivo. Nat. Med., 9(3): 357-362 (2003).
121. McKenzie, D. L., Kwok, K. Y., and Rice, K. G., A Potent New Class of Reductively Activated Peptide Gene Delivery Agents. J. Biol. Chem., 275(14): 9970-9977 (2000).
122. Park, Y., Kwok, K. Y., Boukarim, C., and Rice, K. G., Synthesis of Sulfhydryl Cross-Linking Poly(Ethylene Glycol Peptides and Glycopeptides as Carriers for Gene Delivery. Bioconjugate Chem.,13: 232-239 (2002).
123. Zhang, X., Sawyer, G., J., Dong, X., Qiu, Y., Collins, L., and Fabre, J. W. The in vivo use of chloroquine to promote non-viral gene delivery to the liver via the portal vein and bile duct. J. Gene Med., 5:209-218, 2003.
124. Thomas, N., and Klibanov, A. M. Non-viral gene therapy: polycation-mediated DNA delivery. Appl. Microbiol. Biotechnol. 62:27-34 (2003).
125. S.-O. Han, R. I. Mahato, Y. K. Sung, S. W. Kim, “Development of biomaterials for gene therapy”, Molecular Therapy 2:302317, 2000.
126. Weiss, D., Delivery of Gene Transfer Vectors to the Lung: Obstacles and the Role of Adjunct Techniques for Airway Administration. Mol. Therapy 6(2) (2002).
127. Ferrari S, Geddes D M, Alton E W. Barriers to and new approaches for gene therapy and gene delivery in cystic fibrosis. Adv Drug Deliv Rev, 54(11):1373-93 (2002).
128. Orson F M, Song L, Gautam A, Densmore C L, Bhogal B S, Kinsey B M. Gene delivery to the lung using protein/polyethylenimine/plasmid complexes. Gene Ther. 2002 April; 9(7):463-71.
129. Tiyaboonchai, W., Woiszwillo, J., and Middauth, C. R. Formulation and characterization of DNA-polyethylenemimine-dextran sulfate nanoparticles. Eur. J. Pharm. Sci. 19:191-202 (2003).
130. Benns, J., Mahato, R., and Kim, S. W., Optimization of factors influencing the transfection efficiency of folate-PEG-folate -graft polyethyleneimine, J. Cont. Release 79:255-269 (2002).
131. Putnam, D., Zelikin, A. N., Izumrudov, V. A., and Langer, R., Polyhistidine-PEG:DNA nanocomposites for gene delivery, Biomaterials 24: 4425-4433 (2003).
132. Benns, J., Choi, J-S., Mahato, R. I., Park, J-H., and Kim, S. W., pH-Sensitive Cationic Polymer Gene Delivery Vehicle: N-Ac-poly(L-histidine)-graft-poly(L-lysine) Comb Shaped Polymer, Bioconj. Chem. 11:637-645 (2000).
133. Panyam, J., Zhou, W-Z., Prabha, S., Sahoo, S. K., and Labhasetwar, V., Rapid endo-lysosomal escape of poly(DL-lactide-co-glycolide) nanoparticles: implications for drug and gene delivery, FASEB J., 16: 1217-1226 (2002).
134. Lindgren, M., Hallbrink, M., Prochiantz, A., and Langel, U. Cell-penetrating peptides. Trends Pharmacol. Sci. 21:99-103 (2000).
135. Morris, M. C., Depollier, J., Mery, J., Heitz, F., and Divita, G., A peptide carrier for the biologically active proteins into mammalian cells. Nat. Biotechnol. 19:1174-1176 (2001).
136. Schwarze, S. R. and Dowdy, S. F. In vivo protein transduction: intracellular delivery of biologically active proteins, compounds, and DNA. Trends Pharinacol. Sci. 21:99-103 (2000).
137. Amarzguioui, A., Holen, T., Babaie, E., and Prydz, H. Tolerance for mutations and chemical modifications in a siRNA. Nuc. Acids. Res. 31(2): 589-595 (2003).
138. Braasch, D. A., Jensen, S., Liu, Y., Kaur, K., Arar, K., White, M. A., and Corey, D. R>RNA Interference in Mammalian Cells by Chemically Modified RNA. Biochemistry 42: 7967-7975 (2003).
139. Chiu, Y-L. and Rana, T. M. siRNA function in RNAi: A chemical modification analysis. RNA 9(9): 1034-1048 (2003).
140. Satishchandran C. Characterization of a new class of DNA delivery complexe formed by the local anesthetic bupivacaine. Biochimica et Biophysica Acta 1468: 20-30 (2000).
141. Jeong J H, Park T G., Poly(L-lysine)-g-poly(D,L-lactic-co-glycolic acid) micelles for low cytotoxic biodegradable gene delivery carriers. J Control Release, 82(1):159-66 (2002)
142. Loakes, D. and Brown, D. M., Nucl. Acids Res. 22:4039-4043 (1994).
143. Ohtsuka, E. et al., J. Biol. Chem. 260(5):2605-2608 (1985).
144. Lin, P. K. T. and Brown, D. M., Nucleic Acids Res. 20(19):5149-5152 (1992).
145. Nichols, R. et al., Nature 369(6480): 492-493 (1994).
146. Rahmon, M. S. and Humayun, N. Z., Mutation Research 377 (2): 263-8 (1997).
147. Amosova, O., et al., Nucleic Acids Res. 25 (10): 1930-1934 (1997).
148. Loakes D. & Brown, D. M., Nucleic Acids Res. 22 (20): 4039-4043 (1994).
149. Loakes, D., Nucleic Acids Res. 29(12):2437-47 (2001).
150. Das, A. and Niven, R. J. Pharm. Sci., 90(9), 1366 (2001).
151. Okuda, K, et al, Vaccine 19:3681-3691, (2001).
152. Han, S.-O, et al, Molecular Therapy 2:302317, (2000.)
153. Lewis, D L, et al., Nat Genet. 32(1):107-8 (2002).

1. An RNAi-inducing agent targeted to an influenza virus transcript, wherein the RNAi-inducing agent comprises: a nucleic acid portion whose sequence comprises a sequence selected from the group consisting of: SEQ ID NOs: 272-380, its complement, or a fragment of either having a length of at least 15 nucleotides.

2-3. (canceled)

4. The RNAi-inducing agent of claim 1, which is an siRNA comprising individual nucleic acid strands, one or both of which comprises a single-stranded overhang located at its 3′.

5. The RNAi-inducing agent of claim 1, which is an shRNA comprising first and second nucleic acid portions of a single nucleic acid that form a duplex, wherein the first and second nucleic acid portions are linked together by a single-stranded nucleic acid portion, and wherein the duplex further comprises a single-stranded overhang.

6. An RNAi-inducing agent identical to that of claim 1 except that the sequence of the nucleic acid portions differs at 1 or 2 positions with respect to nucleic acid portion of said RNAi-inducing agent.

7. An isolated vector that comprises a template for transcription of the RNAi-inducing agent of claim 1, wherein the template is operably linked to a promoter.

8. A method of treating or preventing an influenza virus infection comprising administering the vector of claim 7 to a subject in need thereof.

9. A composition comprising the RNAi-inducing agent of claim 1 and a delivery agent.

10. The composition of claim 9, wherein the delivery agent is a cationic polymer.

11. The composition of claim 10, wherein the cationic polymer is selected from the group consisting of: PEI, PLL, PLA, and poly(beta) amino esters.

12. A method of treating or preventing an influenza virus infection comprising administering the RNAi-inducing agent of claim 1 to a subject in need thereof

13-25. (canceled)

26. An isolated nucleic acid, or its complement, whose sequence comprises a sequence selected from the group consisting of SEQ ID NOs: 272-380, or comprises a fragment of a sequence selected from the group consisting of SEQ ID NOs: 272-380 having a length of at least 16 nucleotides, wherein the nucleic acid has a length of 100 nucleotides or less.

27-29. (canceled)

30. The isolated nucleic acid of claim 26, which nucleic acid comprises an attached detectable moiety.

31. An RNAi-inducing agent comprising the nucleic acid of claim 26.

32. A vector that comprises a template for transcription of the RNAi-inducing agent of claim 31.

33. An isolated nucleic acid having the sequence n1-n2-n3-n4-n5-n6-n7-n8-n9-n10-n11-n12-n13-n14-n15-n16-n17-n18-n19 (N19) which is identical to a portion of an influenza virus gene selected from the group consisting of the polymerase protein PB1 gene, the polymerase protein PB2 gene, the polymerase protein PA gene, and the nucleoprotein NP gene or differs in that:
an A to G or C to U difference is allowed at any position;
a G to A or C to A difference is allowed only at positions n1, n18, and/or n19;
there are between 0, 1, 2, or 3 differences between N19 and the influenza virus sequence between positions n1 and n9;
there are no more than 2 consecutive differences; and
there is at most one difference between N19 and the influenza virus sequence between positions n11 and n17.

34. An RNAi-inducing agent targeted to the nucleic acid of claim 33.

35. An RNAi-inducing agent comprising the nucleic acid of claim 33.

36. A vector that comprises a template for transcription of the RNAi-inducing agent of claim 33.

37-105. (canceled)


SEQUENCE LISTING (272 sequences)

SEQ ID NO: 1 - DNA, 21 bp, Artificial Sequence
  Description of Artificial Sequence Synthetic oligonucleotide
aatcaagaagtacacatcagg

SEQ ID NO: 2 - DNA, 21 bp, Artificial Sequence
  Description of Artificial Sequence Synthetic oligonucleotide
aagtacacatcaggaagacag

SEQ ID NO: 3 - DNA, 21 bp, Artificial Sequence
  Description of Artificial Sequence Synthetic oligonucleotide
aatggatgatggcaatgaaat

SEQ ID NO: 4 - DNA, 21 bp, Artificial Sequence
  Description of Artificial Sequence Synthetic oligonucleotide
aattacagcagacaagaggat

SEQ ID NO: 5 - DNA, 21 bp, Artificial Sequence
  Description of Artificial Sequence Synthetic oligonucleotide
aacttactcatcgtcaatgat

SEQ ID NO: 6 - DNA, 21 bp, Artificial Sequence
  Description of Artificial Sequence Synthetic oligonucleotide
aatgtgaggggatcaggaatg

SEQ ID NO: 7 - DNA, 21 bp, Artificial Sequence
  Description of Artificial Sequence Synthetic oligonucleotide
aagcatcaatgaactgagcaa

SEQ ID NO: 8 - DNA, 21 bp, Artificial Sequence
  Description of Artificial Sequence Synthetic oligonucleotide
aaggagacgtggtgttggtaa

SEQ ID NO: 9 - DNA, 21 bp, Artificial Sequence
  Description of Artificial Sequence Synthetic oligonucleotide
aacgggactctagcatactta

SEQ ID NO: 10 - DNA, 21 bp, Artificial Sequence
  Description of Artificial Sequence Synthetic oligonucleotide
aagaattcggatggccatcaa

SEQ ID NO: 11 - DNA, 21 bp, Artificial Sequence
  Description of Artificial Sequence Synthetic oligonucleotide
aagcaggcaaaccatttgaat

SEQ ID NO: 12 - DNA, 21 bp, Artificial Sequence
  Description of Artificial Sequence Synthetic oligonucleotide
aaccatttgaatggatgtcaa

SEQ ID NO: 13 - DNA, 21 bp, Artificial Sequence
  Description of Artificial Sequence Synthetic oligonucleotide
aatccgaccttacttttctta

SEQ ID NO: 14 - DNA, 21 bp, Artificial Sequence
  Description of Artificial Sequence Synthetic oligonucleotide
aagtgccagcacaaaatgcta

SEQ ID NO: 15 - DNA, 21 bp, Artificial Sequence
  Description of Artificial Sequence Synthetic oligonucleotide
aacaggatacaccatggatac

SEQ ID NO: 16 - DNA, 21 bp, Artificial Sequence
  Description of Artificial Sequence Synthetic oligonucleotide
aatgttctcaaacaaaatggc

SEQ ID NO: 17 - DNA, 21 bp, Artificial Sequence
  Description of Artificial Sequence Synthetic oligonucleotide
aatgatgatgggcatgttcaa

SEQ ID NO: 18 - DNA, 21 bp, Artificial Sequence
  Description of Artificial Sequence Synthetic oligonucleotide
aagatctgttccaccattgaa

SEQ ID NO: 19 - DNA, 21 bp, Artificial Sequence
  Description of Artificial Sequence Synthetic oligonucleotide
aagcaggtactgatccaaaat

SEQ ID NO: 20 - DNA, 21 bp, Artificial Sequence
  Description of Artificial Sequence Synthetic oligonucleotide
aatggaagattttgtgcgaca

SEQ ID NO: 21 - DNA, 21 bp, Artificial Sequence
  Description of Artificial Sequence Synthetic oligonucleotide
ttgtgcgacaatgcttcaatc

SEQ ID NO: 22 - DNA, 21 bp, Artificial Sequence
  Description of Artificial Sequence Synthetic oligonucleotide
aatgcttcaatccgatgattg

SEQ ID NO: 23 - DNA, 21 bp, Artificial Sequence
  Description of Artificial Sequence Synthetic oligonucleotide
aatccgatgattgtcgagctt

SEQ ID NO: 24 - DNA, 21 bp, Artificial Sequence
  Description of Artificial Sequence Synthetic oligonucleotide
aacaaatttgcagcaatatgc

SEQ ID NO: 25 - DNA, 21 bp, Artificial Sequence
  Description of Artificial Sequence Synthetic oligonucleotide
aagagacaattgaagaaaggt

SEQ ID NO: 26 - DNA, 21 bp, Artificial Sequence
  Description of Artificial Sequence Synthetic oligonucleotide
tagagcctatgtggatggatt

SEQ ID NO: 27 - DNA, 21 bp, Artificial Sequence
  Description of Artificial Sequence Synthetic oligonucleotide
aacggctacattgagggcaag

SEQ ID NO: 28 - DNA, 21 bp, Artificial Sequence
  Description of Artificial Sequence Synthetic oligonucleotide
aaccacacgaaaagggaataa

SEQ ID NO: 29 - DNA, 21 bp, Artificial Sequence
  Description of Artificial Sequence Synthetic oligonucleotide
aacctgggacctttgatcttg

SEQ ID NO: 30 - DNA, 21 bp, Artificial Sequence
  Description of Artificial Sequence Synthetic oligonucleotide
aagcaattgaggagtgcctga

SEQ ID NO: 31 - DNA, 21 bp, Artificial Sequence
  Description of Artificial Sequence Synthetic oligonucleotide
aatgatccctgggttttgctt

SEQ ID NO: 32 - DNA, 21 bp, Artificial Sequence
  Description of Artificial Sequence Synthetic oligonucleotide
aatgcttcttggttcaactcc

SEQ ID NO: 33 - DNA, 21 bp, Artificial Sequence
  Description of Artificial Sequence Synthetic oligonucleotide
ttggagccattgccggtttta

SEQ ID NO: 34 - DNA, 21 bp, Artificial Sequence
  Description of Artificial Sequence Synthetic oligonucleotide
ggagccattgccggttttatt

SEQ ID NO: 35 - DNA, 21 bp, Artificial Sequence
  Description of Artificial Sequence Synthetic oligonucleotide
aatgggacttatgattatccc

SEQ ID NO: 36 - DNA, 21 bp, Artificial Sequence
  Description of Artificial Sequence Synthetic oligonucleotide
aatcactcactgagtgacatc

SEQ ID NO: 37 - DNA, 21 bp, Artificial Sequence
  Description of Artificial Sequence Synthetic oligonucleotide
aatcatggcgtcccaaggcac

SEQ ID NO: 38 - DNA, 21 bp, Artificial Sequence
  Description of Artificial Sequence Synthetic oligonucleotide
aatagagagaatggtgctctc

SEQ ID NO: 39 - DNA, 21 bp, Artificial Sequence
  Description of Artificial Sequence Synthetic oligonucleotide
aataaggcgaatctggcgcca

SEQ ID NO: 40 - DNA, 21 bp, Artificial Sequence
  Description of Artificial Sequence Synthetic oligonucleotide
aaggcgaatctggcgccaagc

SEQ ID NO: 41 - DNA, 21 bp, Artificial Sequence
  Description of Artificial Sequence Synthetic oligonucleotide
aatgtgcaacattctcaaagg

SEQ ID NO: 42 - DNA, 21 bp, Artificial Sequence
  Description of Artificial Sequence Synthetic oligonucleotide
aatgaaggatcttatttcttc

SEQ ID NO: 43 - DNA, 21 bp, Artificial Sequence
  Description of Artificial Sequence Synthetic oligonucleotide
aaggatcttatttcttcggag

SEQ ID NO: 44 - DNA, 21 bp, Artificial Sequence
  Description of Artificial Sequence Synthetic oligonucleotide
aatgcagaggagtacgacaat

SEQ ID NO: 45 - DNA, 21 bp, Artificial Sequence
  Description of Artificial Sequence Synthetic oligonucleotide
aatgaatccaaatcagaaaat

SEQ ID NO: 46 - DNA, 21 bp, Artificial Sequence
  Description of Artificial Sequence Synthetic oligonucleotide
gaggacacaagagtctgaatg

SEQ ID NO: 47 - DNA, 21 bp, Artificial Sequence
  Description of Artificial Sequence Synthetic oligonucleotide
gaggaatgttcctgttaccct

SEQ ID NO: 48 - DNA, 21 bp, Artificial Sequence
  Description of Artificial Sequence Synthetic oligonucleotide
gtgtgtgcagagacaattggc

SEQ ID NO: 49 - DNA, 21 bp, Artificial Sequence
  Description of Artificial Sequence Synthetic oligonucleotide
aatggctaaagacaagaccaa

SEQ ID NO: 50 - DNA, 21 bp, Artificial Sequence
  Description of Artificial Sequence Synthetic oligonucleotide
aatcctgtcacctctgactaa

SEQ ID NO: 51 - DNA, 21 bp, Artificial Sequence
  Description of Artificial Sequence Synthetic oligonucleotide
acgctcaccgtgcccagtgag

SEQ ID NO: 52 - DNA, 21 bp, Artificial Sequence
  Description of Artificial Sequence Synthetic oligonucleotide
actgcagcgtagacgctttgt

SEQ ID NO: 53 - DNA, 21 bp, Artificial Sequence
  Description of Artificial Sequence Synthetic oligonucleotide
actcagttattctgctggtgc

SEQ ID NO: 54 - DNA, 21 bp, Artificial Sequence
  Description of Artificial Sequence Synthetic oligonucleotide
agttattctgctggtgcactt

SEQ ID NO: 55 - DNA, 21 bp, Artificial Sequence
  Description of Artificial Sequence Synthetic oligonucleotide
aacagattgctgactcccagc

SEQ ID NO: 56 - DNA, 21 bp, Artificial Sequence
  Description of Artificial Sequence Synthetic oligonucleotide
aaggctatggagcaaatggct

SEQ ID NO: 57 - DNA, 21 bp, Artificial Sequence
  Description of Artificial Sequence Synthetic oligonucleotide
aatggctggatcgagtgagca

SEQ ID NO: 58 - DNA, 21 bp, Artificial Sequence
  Description of Artificial Sequence Synthetic oligonucleotide
actcatcctagctccagtgct

SEQ ID NO: 59 - DNA, 21 bp, Artificial Sequence
  Description of Artificial Sequence Synthetic oligonucleotide
aatttgcaggcctatcagaaa

SEQ ID NO: 60 - DNA, 21 bp, Artificial Sequence
  Description of Artificial Sequence Synthetic oligonucleotide
attgtggattcttgatcgtct

SEQ ID NO: 61 - DNA, 21 bp, Artificial Sequence
  Description of Artificial Sequence Synthetic oligonucleotide
aagaatatcgaaaggaacagc

SEQ ID NO: 62 - DNA, 21 bp, Artificial Sequence
  Description of Artificial Sequence Synthetic oligonucleotide
attttgtcagcatagagctgg

SEQ ID NO: 63 - DNA, 21 bp, Artificial Sequence
  Description of Artificial Sequence Synthetic oligonucleotide
aagaactaggtgatgccccat

SEQ ID NO: 64 - DNA, 21 bp, Artificial Sequence
  Description of Artificial Sequence Synthetic oligonucleotide
aactaggtgatgccccattcc

SEQ ID NO: 65 - DNA, 21 bp, Artificial Sequence
  Description of Artificial Sequence Synthetic oligonucleotide
atcggcttcgccgagatcaga

SEQ ID NO: 66 - DNA, 21 bp, Artificial Sequence
  Description of Artificial Sequence Synthetic oligonucleotide
gccgagatcagaaatccctaa

SEQ ID NO: 67 - DNA, 21 bp, Artificial Sequence
  Description of Artificial Sequence Synthetic oligonucleotide
ggagtcctcatcggaggactt

SEQ ID NO: 68 - DNA, 21 bp, Artificial Sequence
  Description of Artificial Sequence Synthetic oligonucleotide
aatgataacacagttcgagtc

SEQ ID NO: 69 - DNA, 21 bp, Artificial Sequence
  Description of Combined DNA/RNA Molecule Synthetic oligonucleotide
ggagacgugguguugguaatt

SEQ ID NO: 70 - DNA, 21 bp, Artificial Sequence
  Description of Combined DNA/RNA Molecule Synthetic oligonucleotide
uuaccaacaccacgucucctt

SEQ ID NO: 71 - DNA, 21 bp, Artificial Sequence
  Description of Combined DNA/RNA Molecule Synthetic oligonucleotide
cgggacucuagcauacuuatt

SEQ ID NO: 72 - DNA, 21 bp, Artificial Sequence
  Description of Combined DNA/RNA Molecule Synthetic oligonucleotide
uaaguaugcuagagucccgtt

SEQ ID NO: 73 - DNA, 21 bp, Artificial Sequence
  Description of Combined DNA/RNA Molecule Synthetic oligonucleotide
gcaggcaaaccauuugaautt

SEQ ID NO: 74 - DNA, 21 bp, Artificial Sequence
  Description of Combined DNA/RNA Molecule Synthetic oligonucleotide
auucaaaugguuugccugctt

SEQ ID NO: 75 - DNA, 21 bp, Artificial Sequence
  Description of Combined DNA/RNA Molecule Synthetic oligonucleotide
caggauacaccauggauactt

SEQ ID NO: 76 - DNA, 21 bp, Artificial Sequence
  Description of Combined DNA/RNA Molecule Synthetic oligonucleotide
guauccaugguguauccugtt

SEQ ID NO: 77 - DNA, 21 bp, Artificial Sequence
  Description of Combined DNA/RNA Molecule Synthetic oligonucleotide
gaucuguuccaccauugaatt

SEQ ID NO: 78 - DNA, 21 bp, Artificial Sequence
  Description of Combined DNA/RNA Molecule Synthetic oligonucleotide
uucaaugguggaacagauctt

SEQ ID NO: 79 - DNA, 21 bp, Artificial Sequence
  Description of Combined DNA/RNA Molecule Synthetic oligonucleotide
ugcuucaauccgaugauugtt

SEQ ID NO: 80 - DNA, 21 bp, Artificial Sequence
  Description of Combined DNA/RNA Molecule Synthetic oligonucleotide
caaucaucggauugaagcatt

SEQ ID NO: 81 - DNA, 21 bp, Artificial Sequence
  Description of Combined DNA/RNA Molecule Synthetic oligonucleotide
cggcuacauugagggcaagtt

SEQ ID NO: 82 - DNA, 21 bp, Artificial Sequence
  Description of Combined DNA/RNA Molecule Synthetic oligonucleotide
cuugcccucaauguagccgtt

SEQ ID NO: 83 - DNA, 21 bp, Artificial Sequence
  Description of Combined DNA/RNA Molecule Synthetic oligonucleotide
gcaauugaggagugccugatt

SEQ ID NO: 84 - DNA, 21 bp, Artificial Sequence
  Description of Combined DNA/RNA Molecule Synthetic oligonucleotide
ucaggcacuccucaauugctt

SEQ ID NO: 85 - DNA, 21 bp, Artificial Sequence
  Description of Combined DNA/RNA Molecule Synthetic oligonucleotide
ugaucccuggguuuugcuutt

SEQ ID NO: 86 - DNA, 21 bp, Artificial Sequence
  Description of Combined DNA/RNA Molecule Synthetic oligonucleotide
aagcaaaacccagggaucatt

SEQ ID NO: 87 - DNA, 21 bp, Artificial Sequence
  Description of Combined DNA/RNA Molecule Synthetic oligonucleotide
ugcuucuugguucaacucctt

SEQ ID NO: 88 - DNA, 21 bp, Artificial Sequence
  Description of Combined DNA/RNA Molecule Synthetic oligonucleotide
ggaguugaaccaagaagcatt

SEQ ID NO: 89 - DNA, 21 bp, Artificial Sequence
  Description of Combined DNA/RNA Molecule Synthetic oligonucleotide
uagagagaauggugcucuctt

SEQ ID NO: 90 - DNA, 21 bp, Artificial Sequence
  Description of Combined DNA/RNA Molecule Synthetic oligonucleotide
gagagcaccauucucucuatt

SEQ ID NO: 91 - DNA, 21 bp, Artificial Sequence
  Description of Combined DNA/RNA Molecule Synthetic oligonucleotide
uaaggcgaaucuggcgccatt

SEQ ID NO: 92 - DNA, 21 bp, Artificial Sequence
  Description of Combined DNA/RNA Molecule Synthetic oligonucleotide
uggcgccagauucgccuuatt

SEQ ID NO: 93 - DNA, 21 bp, Artificial Sequence
  Description of Combined DNA/RNA Molecule Synthetic oligonucleotide
ggaucuuauuucuucggagtt

SEQ ID NO: 94 - DNA, 21 bp, Artificial Sequence
  Description of Combined DNA/RNA Molecule Synthetic oligonucleotide
cuccgaagaaauaagaucctt

SEQ ID NO: 95 - DNA, 21 bp, Artificial Sequence
  Description of Combined DNA/RNA Molecule Synthetic oligonucleotide
ccgaggucgaaacguacgutt

SEQ ID NO: 96 - DNA, 21 bp, Artificial Sequence
  Description of Combined DNA/RNA Molecule Synthetic oligonucleotide
acguacguuucgaccucggtt

SEQ ID NO: 97 - DNA, 21 bp, Artificial Sequence
  Description of Combined DNA/RNA Molecule Synthetic oligonucleotide
cagauugcugacucccagctt

SEQ ID NO: 98 - DNA, 21 bp, Artificial Sequence
  Description of Combined DNA/RNA Molecule Synthetic oligonucleotide
gcugggagucagcaaucugtt

SEQ ID NO: 99 - DNA, 21 bp, Artificial Sequence
  Description of Combined DNA/RNA Molecule Synthetic oligonucleotide
uggcuggaucgagugagcatt

SEQ ID NO: 100 - DNA, 21 bp, Artificial Sequence
  Description of Combined DNA/RNA Molecule Synthetic oligonucleotide
ugcucacucgauccagccatt

SEQ ID NO: 101 - DNA, 21 bp, Artificial Sequence
  Description of Combined DNA/RNA Molecule Synthetic oligonucleotide
gaauaucgaaaggaacagctt

SEQ ID NO: 102 - DNA, 21 bp, Artificial Sequence
  Description of Combined DNA/RNA Molecule Synthetic oligonucleotide
gcuguuccuuucgauauuctt

SEQ ID NO: 103 - DNA, 21 bp, Artificial Sequence
  Description of Combined DNA/RNA Molecule Synthetic oligonucleotide
cggcuucgccgagaucagaat

SEQ ID NO: 104 - DNA, 21 bp, Artificial Sequence
  Description of Combined DNA/RNA Molecule Synthetic oligonucleotide
ucugaucucggcgaagccgat

SEQ ID NO: 105 - DNA, 21 bp, Artificial Sequence
  Description of Combined DNA/RNA Molecule Synthetic oligonucleotide
guccuccgaugaggacucctt

SEQ ID NO: 106 - DNA, 21 bp, Artificial Sequence
  Description of Combined DNA/RNA Molecule Synthetic oligonucleotide
ggaguccucaucggaggactt

SEQ ID NO: 107 - DNA, 21 bp, Artificial Sequence
  Description of Combined DNA/RNA Molecule Synthetic oligonucleotide
ugauaacacaguucgaguctt

SEQ ID NO: 108 - DNA, 21 bp, Artificial Sequence
  Description of Combined DNA/RNA Molecule Synthetic oligonucleotide
gacucgaacuguguuaucatt

SEQ ID NO: 109 - DNA, 19 bp, Artificial Sequence
  Description of Artificial Sequence Synthetic oligonucleotide
tgcttcaatccgatgattg

SEQ ID NO: 110 - RNA, 21 nt, Artificial Sequence
  Description of Artificial Sequence Synthetic oligonucleotide
ggcuacguccaggagcgcauu

SEQ ID NO: 111 - RNA, 21 nt, Artificial Sequence
  Description of Artificial Sequence Synthetic oligonucleotide
ugcgcuccuggacguagccuu

SEQ ID NO: 112 - DNA, 18 bp, Artificial Sequence
  Description of Artificial Sequence Synthetic primer
tttttttttttttttttt

SEQ ID NO: 113 - DNA, 22 bp, Artificial Sequence
  Description of Artificial Sequence Synthetic primer
ctcgtcgcttatgacaaagaag

SEQ ID NO: 114 - DNA, 36 bp, Artificial Sequence
  Description of Artificial Sequence Synthetic primer
atatcgtctcgtattagtagaaacaagggtattttt

SEQ ID NO: 115 - DNA, 21 bp, Artificial Sequence
  Description of Artificial Sequence Synthetic primer
caggacatactgatgaggatg

SEQ ID NO: 116 - DNA, 35 bp, Artificial Sequence
  Description of Artificial Sequence Synthetic primer
atatcgtctcgtattagtagaaacaagggtgtttt

SEQ ID NO: 117 - DNA, 22 bp, Artificial Sequence
  Description of Artificial Sequence Synthetic primer
ctcgtcgcttatgacaaagaag

SEQ ID NO: 118 - DNA, 21 bp, Artificial Sequence
  Description of Artificial Sequence Synthetic primer
agatcatcatgtgagtcagac

SEQ ID NO: 119 - DNA, 21 bp, Artificial Sequence
  Description of Artificial Sequence Synthetic primer
caggacatactgatgaggatg

SEQ ID NO: 120 - DNA, 21 bp, Artificial Sequence
  Description of Artificial Sequence Synthetic primer
gtttcagagactcgaactgtg

SEQ ID NO: 121 - DNA, 2233 bp, Influenza A virus
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 122 - DNA, 2233 bp, Influenza A virus
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaattttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 123 - DNA, 2233 bp, Influenza A virus
agcaaaagcaggtactgatccgaaatggaagaatttgtgcgacaatgcttcaatccgatgattgtcgagcttgctgaaaaagcaatgaaagagtatggagaggatcggaaaatcgaaacaaacaaatttgcagcaatatgcactcacttggaagtatgcttcatgtattcagattttcatttcatcaatgagcaaggcgagtcaataatagtagagcttgatgatccaaatgcacttttgaagcacagatttgaaataatagagggaagagatcgcacaatggcctggacagtagtaaacagtatttgcaacactacaggagctgagaaaccgaagtttctgccagatttgtatgattacaaggagaatagattcatcgagattggagtgacaaggagggaagtccacatatactatcttgaaaaggccaataaaattaaatctgagaagacacacatccacattttctcattcactggggaagaaatggccacaaaggccgactacactctcgatgaggaaagcagggctaggatcaagaccagactattcaccataagacaagaaatggctagcagaggcctctgggattcctttcgtcagtccgaaagaggcgaagaaacaattgaagaaagatttgaaatcacagggacaatgcgcaggctcgccgaccaaagtctcccgccgaacttctcctgccttgagaattttagagcctatgtggatggattcgaacccaacggctacattgagggcaagctttctcaaatgtccaaagaagtaaatgctaaaattgagccttttctgaaaacaacaccaagaccaattaaacttccggatgggcctccttgctctcagcggtccaaattcctgctgatggatgctttaaaattaagcattgaggacccaagtcacgaaggagagggaataccactatatgatgcgatcaagtgtatgagaacattctttggatggaaagaaccctatgttgttaaaccacacgataagggaataaatccaaattatctgctgtcatggaagcaattactggcagaactgcaggacattgagaatgaggagaagattccaagaaccaaaaacatgaagaaaacgagtcagctaaagtgggcacttggtgagaacatggcaccagagaaggtagactttgacgactgtagagatataagcgatttgaagcaatatgatagtgatgaacctgaattaaggtcactttcaagctggatccagaatgagttcaacaaggcatgcgagctgaccgattcaatctggatagagctcgatgagattggagaagatgtggctccaattgaacacattgcaagcatgagaaggaattacttcacagcagaggtgtctcagtgcagagccacagaatatataatgaagggggtatacattaatactgccttgcttaatgcatcctgtgcagcaatggacgatttccaactaattcccatgataagcaaatgtagaactaaagagggaaggcgaaagaccaatttatatggtttcatcataaaaggaagatctcacttaaggaatgacaccgacgtggtaaactttgtgagcatggagttttctctcactgacccaagacttgagccacacaaatgggagaagtactgtgttcttgagataggagatatgctactaagaagtgccataggccaggtgtcaaggcccatgttcttgtatgtgaggacaaatggaacatcaaagattaaaatgaaatggggaatggagatgaggcgttgcctccttcagtcactccaacaaatcgagagtatgattgaagccgagtcctctgtcaaggagaaagacatgaccaaagagtttttcgagaataaatcagaaacatggcccattggagagtcccccaaaggagtggaagaaggttccattgggaaggtctgcaggactttattagccaagtcggtattcaatagcctgtatgcatctccacaattagaaggattttcagctgaatcaagaaaactgcttcttgtcgttcaggctcttagggacaatcttgaacctgggacctttgatcttggggggctatatgaagcaattgaggagtgcctgattaatgatccctgggttttgcttaatgcgtcttggttcaactccttcctaacacatgcattaagatagttgtggcaatgctactatttgctatccatactgtccaaaaaagtaccttgtttctact

SEQ ID NO: 124 - DNA, 2209 bp, Influenza A virus
atggaagattttgtacgacaatgctttaatccgatgattgtcgaacttgcggaaaaggcaatgaaagagtatggagaggatcttaaaatcgaaacaaacaaatttgcagcaatatgcactcacttggaagtatgcttcatgtattcagattttcatttcatcaatgagcaaggcgagtcaatagtggtagaacttgatgatccaaatgcacttttgaagcacagatttgaaataatagagggaagagaccgcacaatggcctggacagtagtaaacagtatttgcaacaccacaggagctgagaaaccgaagtttctgccagatttgtatgattacaaggagaatagattcatcgagattggagtgacaaggagagaagtccacatatactaccttgaaaaggccaataaaattaaatctgagaatacacacatccacattttctcattcactggggaagaaatggccacaaaggccgactacactctcgatgaggaaagcagggctaggatcaaaaccagactattcaccataagacaagagatggccaacagaggcctctgggattcctttcgtcagtccgaaagaggcgaagaaacaattgaagaaagatttgaaatcacagggacaatgcgcaggcttgccgaccaaagtctcccgccgaacttctcctgccttgagaattttagagcctatgtggatggattcgaaccgaacggctacattgagggcaagctttctcaaatgtccaaagaagtgaatgcaaaaattgaaccttttctgaaaacaacaccaagaccaattagacttccggatgggcctccttgttttcagcggtccaaattccttctgatggatgctttaaagttaagcattgaggatccaagtcacgagggggagggaataccactatatgatgcgatcaaatgcatgagaacattttttggatggaaagaaccctatattgttaaaccacacgaaaaggggataaatccaaattatctgctgtcatggaagcaagtactggcagaactgcaggacattgaaaatgaggagaaaattccaagaactaaaaacatgaagaaaacgagtcagctaaagtgggcacttggtgagaacatggcaccagagaaggtagactttgacaactgtagagacgtaagcgatttgaagcaatatgatagtgacgaacctgaattaaggtcactttcaagctggatccagaatgagttcaacaaggcatgcgagctgaccgattcaacttggatagagctcgatgagattggagaagacgtggctccaattgaatacattgcaagcatgagaaggaattacttcacagcagaggtgtcccattgcagagccacagaatatataatgaagggggtatacattaatactgccttgcttaatgcatcctgtgcagcaatggacgatttccaactaattcccatgataagcaagtgtagaactaaagaaggaaggcgaaagaccaatttatatggcttcatcataaaaggaagatctcacttaaggaatgacaccgacgtggtaaactttgtgagcatggagttttctctcactgacccgagacttgagccacacaaatgggagaaatactgtgtccttgagataggagatatgctactaagaagtgctataggccagatgtcaaggcctatgttcttgtatgtgagaacaaatggaacatcaaagattaaaatgaaatggggaatggagatgaggcgttgcctccttcagtcactccaacaaatcgagagtatgattgaagcagagtcatctgtcaaagagaaagacatgaccaaagagttttttgagaataaatcagaaacatggcccattggggagtcccccaagggagtggaagatggttccattgggaaggtctgcaggactttattggccaagtcggtattcaatagcctgtatgcatccccgcaattggaagggttttcagctgagtcaagaaaactgcttcttgtcgttcaggctcttaaggacaatcttgaacctggaacctttgatcttgaggggctatatgaagcaattgaggagtgcctgattaatgatccctgggttttgcttaatgcgtcgtggttcaactccttcctaacacatgcattaagatagttgtggcaatgctactatttgctatccatactgtccaaaaaagtaccttgtttctact

SEQ ID NO: 125 - DNA, 2233 bp, Influenza A virus
agcaaaagcaggtactgatccaaaatggaagactttgtgcgacaatgcttcaatccaatgattgtcgagcttgcggaaaagacaatgaaggagtatggggaagatccgaagattgaaacaaacaagttcgctgcaatatgcacacacttagaagtctgcttcatgtattcagacttccatttcattgacgaacgaggcgaatcaataattgtggaatctggtgatccgaatgcattgttgaaacaccggtttgaaataattgaaggaagagaccgagcaatggcctggacagtggtgaatagcatctgcaacaccacaggagtcgataaacccaaatttcttccggatctatacgactacaaggaaaaccgattcactgaaattggtgtgacacggagggaagttcatatatattacttagagaaagctaacaagataaaatccgagaaaacacatatccacatcttctcattcactggagaagaaatggccactaaagctgactacacccttgatgaagagagcagggcaagaatcaaaaccagactattcaccataagacaggaaatggcaagcaggggtctatgggactcctttcgtcagtccgagagaggcgaagagacaattgaagaaagatttgaaatcacagggaccatgcgtaggcttgccgaccaaagtctcccacctaacttctccagccttgaaaactttagagcctatgtggatggatttaaaccgaacggctgcattgagggcaagctttctcaaatgtcgaaagaagtgaacgccagaattgagccatttctgaagacaacaccacgtcctctcagattgcctgatggacctccctgctcccagcggtcgaaattcttgctgatggatgctctgaaattaagcattgaggacccgagccatgagggggaggggataccgctatatgatgcgatcaaatgcatgaaaacattctttggctggagagagcccaacatcatcaaaccacacgaaaaaggcataaatccaaattatctcctggcttggaagcaggtgctggcagaactccaggatattgaaaatgaggataaaatcccaaaaacaaagaacatgaagaaaacaagccaattaatgtgggcactcggagagaatatggcaccggaaaaagtggactttgaggattgcaaagacattgatgatctgaaacagtaccacagtgatgagccagagcttagatcgctagcaagctggatccagaatgagttcaacaaggcatgtgaattgaccgattcgagctggatagaacttgatgagataggggaagatgttgccccaattgagcacattgcaagtatgagaaggaactacttcacagcggaggtgtcccattgcagggctactgagtacataatgaagggggtttacataaatacagctttgctcaatgcatcttgtgcagccatggatgacttccaactgattccaatgataagcaaatgcagaacaaaagaaggaaggaggaggacaaacctgtatgggttcattgtaaaaggaaggtcccatttgagaaatgatactgacgtggtgaactttgtgagtatggaattctcccttactgacccaaggctggagccacacaaatgggaaaagtactgtgttcttgaaataggggaaatgctcttgcggactgcaataggtcaagtgtcaaggcccatgttcctgtatgtgagaaccaacggaacctcaaaaattaagatgaaatgggggatggaaatgaggcgctgccttcttcaatctcttcaacagattgagagcatgatcgaggctgagtcttctatcaaagagaaagacatgaccaaagaattctttgaaaacagatcggagacatggccaattggagagtcacctaagggagtggaggaaggctccatcgggaaggtgtgcagaaccttactagcaaaatctgtgttcaacagcctatattcatctccacaactcgaaggattttcagctgaatcgagaaaactactactcattgttcaagcacttagggacaacctggaacctggaaccttcgatcttgaagggctatatggagcaattgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 126 - DNA, 2233 bp, Influenza A virus
agcaaaagcaggtactgatccgaaatggaagactttgtgcgacaatgcttcaatccaatgattgtcgagcttgcggaaaagacaatgaaggaatatggggaagacccgaaaattgaaacaaataagttcgctgcaatatgcacacacttagaagtctgcttcatgtattcagacttccatttcattgacgaacgaggcgaatcaataattgtggaatctggtgatccaaatgcattgttgaagcacaggtttgaaataattgaaggaagagaccgagcaatggcctggacagtggtgaatagcatctgcaacacaacaggagtcgataaacccaaatttcttccggatctatacgactacaaggaaaaccgattcactgaaattggtgtgacacggagggaagttcacatatattacttagaaaaagctaacaagataaaatccgagaaaacacatatccacatcttttcattcactggagaagaaatggccactaaagctgactacacccttgatgaagagagcagggcaagaataaaaaccagactattcaccataagacaggaaatggcaagcaggggtctatgggattcctttcgtcagtccgagagaggcgaagagacaattgaagaaagatttgaaatcacagggaccatgcgtaggcttgccgaccaaagtctcccacctaacttctccagccttgaaaactttagagcctatgtggatggattcaaaccgaacggctgcattgagggcaagctttctcaaatgtcgaaagaagtgaacgccagaattgagccatttctgaagacaacaccacgtcccctcagattgcctgatggacctccctgctcccagcggtcgaaattcttgctgatggatgctctgaaattaagcattgaggacccgagccatgagggggaggggataccgctatatgatgcgataaaatgcatgaaaacattcttcggctggagagagcccaacatcatcaagccacacgagaagggcataaatcccaattatcttctggcttggaagcaggtgctggcagaactccaggatattgaaaatgaggataaaatcccaaaaacaaagaacatgaagaaaacaagccaattaatgtgggcactcggggagaatatggcaccggaaaaattggactttgaggactgcaaagatattggcgatctgaaacagtatcaaagtgatgagccagagctcagatcgatagcaagctggatccagagtgagttcaacaaggcatgtgaattgaccgattcgagctggatagaactcgatgagataggggaagatgttgccccaattgagcacattgcaagcatgagaaggaactacttcacagcggaagtgtctcattgcagggccactgagtacataatgaagggggtttacataaatacagctttgctcaatgcatcttgtgcagccatggatgacttccaactgattccaatgataagcaaatgcagaacaaaagaaggaagaaggaagacaaacctgtatgggttcattataaaaggaaggtcccatttgagaaatgatactgacgtggtgaactttgtgagtatggaattctcccttactgacccaaggctggagccacacaaatgggaaaagtactgtgttcttgaagtaggggaaatgctcttgcggactgcaataggccaggtgtcaaggcccatgttcctgtatgtgagaactaacggaacctccaaaattaagatgaaatgggggatggaaatgagacgctgccttcttcaatctcttcaacagattgagagcatgatcgaggctgagtcttctatcaaagagaaagacatgaccaaagaattctttgaaaacagatcggagacatggccaattggagagtcacctaagggagtggaggaaggctcaatcgggaaggtgtgcagaaccttactagcaaaatctgtgttcaacagcctatattcatctccacaactcgaaggattttcagctgaatcgagaaaactactactcattgttcaagcacttagggacaacctggaacctggaacctttgatcttgaagggctatatggagcaattgaggagtgcctgattaatgatccctgggttttgcttaatgcatcttggttcaactccttcctcacacatgcactaaaatagttgtggcaatgctactatttgctatccatactgtccaaaaaagtaccttgtttctact

SEQ ID NO: 127 - DNA, 2233 bp, Influenza A virus
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcattcactggggaggaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcactatgatagtgatgaaccagaattgaggtcgcttgcaagttggattcagaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 128 - DNA, 2233 bp, Influenza A virus
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacatcttctcattcactggggaagaaatggccactaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaattttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 129 - DNA, 2233 bp, Influenza A virus
agcgaaagcaggtactgatccaaaatggaagaatttgtgcgacaatgcttcaatccaatgatcgtcgagcttgcggaaaagacaatgaaagaatatggagaggacccgaagattgaaacaaacaaattcgcagcaatatgcacacatttggaagtgtgtttcatgtattcagatttccactttattgatgaacggggagagtcaaagattgtagaatctggtgacccaaatgcactcttgaagcaccgatttgagataattgaaggaagagatcgcacgatggcctggacggtggtgaatagcatttgcaatactacaggggttgagaaacccaagttccttccggatttgtatgattataaggagaaccgattcattgaaattggagtgacaagaagggaggtccacatatactacttagaaaaagccaataagataaaatctgagaagacacacatccacattttctcgttcactggggaagaaatggccacaaaggcggactacactcttgatgaagagagcagagcaaggatcaaaaccaggctattcaccataagacaagaaatggccagtagaggcctctgggattcctttcgtcagtccgagagaggcgaagagacaattgaagaaagatttgaaatcacaggaaccatgcgcaggcttgccgaacaaagtctcccaccgaacttctccagccttgaaaactttagagcctatgtggatggattcaaaccgaacggctgcattgagggcaagctttctcaaatgtccaaagaagtaaatgcaagaatcgagccattcttgaagacaacaccacgccctctcagattacctgatgggcctccctgttctcagcggtcaaaattcctactgatggatgctctgaaattaagcattgaagacccaagtcatgagggggaggggataccactacatgatgcaatcaaatgcatgaagacattctttggctggaaagagcccaatattgtcaaaccgcatgagaagggcataaatcccaactatctcctggcttggaaccaggtgctagcagaactgaaggatattgagaatgaggagaaaattccaaaaacaaaaaatatgaagaaaacaagccagttaaagtgggcacttggtgagaacatggcaccagagaaagtagactttgaggattgcaaggacattagcgatctgaagcagtatgacagtgatgagccagaacagagatcactagcgagttggatccagagtgaattcaacaaagcatgtgagctgaccgattcaggttggatagaacttgatgaaataggagaagatgtagccccaatcgagcacattgcaagtatgaggagaaactatttcacagcggaagtgtctcactgcagggcaacggagtacataatgaaaggggtatacataaacacggccttgctcaatgcatcttgtgcagctatggatgacttccagctgatcccaatgataagcaaatgcaggaccaaagaaggaagacggaagacgaatctgtatggattcattataaaaggaagatctcacttgaggaatgatactgatgtggtgaattttgtgagcatggagttctctctcactgacccgaggcttgagccacacaagtgggagaagtattgtgttcttgaaataggagacatgctcctgcggactgcaataggccaagtatcaaggcccatgttcctgtatgtgagaaccaatggaacctccaaaatcaagatgaaatggggtatggagatgagacgttgccttcttcagtcccttcaacaggttgaaagcatggttgaggctgagtcctctgtcaaggagaaagacatgactaaggaattctttgaaaacaagtcaaagacgtggcccattggagaatcacctaaaggagtggaagaaggttccatcgggaaagtgtgcaggaccttactggcgaagtctgtatttaacagcttatatgcatccccacaacttgagggattttcagcggaatctagaaaactgctcctcattgttcaggctcttagagacaacctggaacctggaaccttcgatcttggagggctatatggagcaattgaggagtgcctgattaatgatccctgggttttgcttaatgcatcttggttcaactccttcctcacacatgcactgaaatagttgtggcaatgctactatttgctatccatactgtccaaaaaagtaccttgtttctact

SEQ ID NO: 130 - DNA, 2140 bp, Influenza A virus
atggaagacttcgtgcgacaatgcttcaatccaatgattgtcgagcttgcggaaaaggcaatgaaagaatatggagagggcccgaaaatcgaaacaaacaaatttgcagcaatatgcactcatttggaagtgtgtttcatgtattcagactttcacttcatcgatgagcgaggcgaatcaataattgtagaatccggagatccgaatgccctcttgaagcacagatttgaaataattgagggaagagatcgcacaatggcctggacagtggtgaacagcatctgtaacactacaggggttgagaagccaaggtttctcccagatctatatgactacaaggagaacaggttcattgagattggagtgacaaggagagaagtccacatatactacctggaaaaggccaataaaataaagtctgagaagacacatatccacatcttctcgttcacaggagaagagatggccacaaaggctgactacactcttgatgaagaaagtagggccagaatcaaaactagactgttcaccataaggcaggaaatggccagtagaggtctctgggattcctttcgtcagtccgagagaggcgaagagacaattgaagaaagatttgaaatcacaggaacaatgcgcaggcttgccgaccaaagtctcccaccgaacttctccagccttgaaaactttagagcctatgtggatggattcgaaccgaacggctgcattgagggcaagctttctcaaatgtccaaagaagtgaatgcaagaattgaaccctttttgaagacaacaccacgcccactcaagctaccagatgggcctccctgctcccagcggtccaaattcctgctaatggacgctttgaaattaagcattgaggacccaagccatgaaggagaagggataccgctatatgatgcaatcaaatgcatgaaaacattctttgggtggaaagaacccaatattgttaaaccacatgaaaaaggaataaatccgaattacctcttggcatggaaacaagtactagcggaactacaggatcttgaaaatgaagagaaaattccaaagactaaaaacatgaagaaaacaagccaattaaagtgggcacttggtgagaatatggcaccagaaaaagtggattttgaggactgcaaggatgtcagcgatctgaagcaatatgacagtgatgaaccggaaccgagatcgcttgcaagttggattcagagtgagttcaataaggcgtgtgaactgactgattcaagctggatagagcttgacgagattggggaagatgttgccccaattgagcacattgcaagcatgaggaggaattatttcacagcggaggtgtctcattgtagagccacagaatacataatgaaaggggtatacatcaatactgccttgctcaatgcatcctgtgcggctatggatgactttcaactgattccaatgatcagcaagtgtagaactaaagagggaagaagaaagacaaatttgtatgggttcattataaaagggagatcccacctgaggaacgacaccgatgtggtaaactttgtgagcatggagttttccctcactgacccgaggcttgaaccgcacaaatgggagaagtactgtgttcttgaaataggggacatgcttctaagaactgccataggccaagtttcgaggcccatgttcctgtatgtgagaacgaatgggacctccaaaatcaaaatgaaatgggggatggaaatgagacgctgtcttctccagtcccttcaacaaattgagagtatgattgaagccgagtcctctgtcaaagagaaggacatgaccaaagagttctttgaaaacaaatcagaaacatggcctattggggaatctcccaagggggtggaggaaggatccattggaaaggtctgcaggactctgttggcaaagtctgtattcaacagcttgtacgcatctccacagctggaaggtttctcagctgaatcaaggaaactgcttcttatcgttcaggctcttagggacaacctggaacctggaacctttgatcttggaggattgcatgaagcaattgaggagtgcctgattaatgacccctgggttttgcttaatgcatcttggtttaactccttcctcacacatg

SEQ ID NO: 131 - DNA, 2233 bp, Influenza A virus
agcaaaagcaggtactgatccaaaatggaagactttgtgcgacaatgcttcaatccaatgatcgtcgagcttgcggaaaaggcgatgaaagaatatggagaggacccgaaaattgaaacaaacaaatttgcagcaatatgcactcacttggaagtctgcttcatgtactcggatttccactttattaatgaactgggcgagtcagtgatcatagagtctggtgatccaaatgctcttttgaagcacagatttgaaatcattgaagggagagatcgaacaatggcatggacagtagtgaacagtatctgcaacaccacaagagctgaaaaacccaagttcctcccagatttgtacgactataaagagaacaggtttgttgaaattggtgtgacaaggagagaagttcacatatactacttggagaaagccaacaaaataaagtctgagaaaacacatattcacattttctcatttacaggagaggaaatggctacaaaagcggattatacccttgatgaagaaagtagagccaggatcaaaaccagactattcaccataagacaagaaatggccagcagaggcctttgggactcctttcgtcagtccgagagaggcgaagagacaattgaagaaagatttgaaatcacagggacaatgcgcaggcttgccgattacagtctcccaccgaacttctccagccttgaaaattttagagcctatgtggatggattcgaaccgaacggctgcattgagagcaagctttctcaaatgtcaaaagaagtaaataccagaatcgagccattttcaaagacaacaccccggccactcaggacaccgggtggtccaccctgttatcagcgatccaaattcttgctgatggatgctctgaaatttagcattgaggatccaagtcacgagggagagggaataccgctgtatgatgccatcaaatgcatgaaaaccttctttggatggaaagagcccaatattgttaaaccacatgaaaagggtataaacccaaactatctccaggcttggaagcaagtgttagcagaactacaggacctcgaaaacgaagaaaaaatccctaagaccaagaatatgaaaaaaacaagtcaattgaaatgggcacttggtgagaatatggcgccagagaaagtggattttgaggattgtaaagacatcagtgatttgaaacagtatgacagtgatgagccagaaacaaggtcccttgcaagttggattcaaagtgagttcaacaaagcttgtgagctgacagattcaagctggatagagctcgatgaaattggggaggatgttgccccaatagaacacattgcgagcatgaggaggaattattttactgctgaggtttcccattgtagagcaactgaatatataatgaagggagtatacatcaacactgctctactcaatgcatcctgcgctgcgatggatgacttccaattaatcccgatgataagcaaatgcaggaccaaggaagggagaaggaagacaaatttgtatggattcatcataaagggaaggtcccatttaagaaatgacactgacgtggtaaactttgtaagcatggagttttctctcaccgatccaagacttgagccacacaattgggagaagtactgtgttctagaaatcggagacatgctcctaagaactgctgtaggccaagtgtcaagacccatgtttttgtatgtaaggaccaatgggacctctaaaattaaaatgaaatggggaatggaaatgaggcgctgcctccttcagtctctacagcagattgaaagcatgactgaagctgagtcctcagtcaaagaaaaggacatgaccaaagaattctttgagaacaaatcggagacatggcctataggagagtcccccaaaggagtggaagaaggctcaatcgggaaagtttgcaggaccttgttagcaaaatctgtgtttaacagtttatatgcatctccacaactcgaagggttttcagctgaatctaggaaaatacttctcattgttcaggcccttagggacaacctggaacctggaacctttgatattggggggctatatgaatcaattgaggagtgtctgattaatgatccctgggttttgctcaatgcatcttggttcaactccttccttacacatgcactaaagtagttgtggcaatgctactatttgctatccatactgtccaaaaaagtaccttgtttctact

SEQ ID NO: 132 - DNA, 2233 bp, Influenza A virus
agcaaaagcaggtactgattcaaaatggaagactttgtgcgacaatgcttcaatccaatgatcgtcgagcttgcggaaaaggcaatgaaagaatatggagaggatccaaaaatcgagacaaacaaattcgctgcaatatgcacacacctggaagtgtgtttcatgtattcagacttccatttcattgatgaacggggtgagtcgataattgttgagtctggtgatccaaatgcactcttaaaacatcgatttgaaataatcgaaggaagagaccgtactatggcctggacagtggtgaatagcatttgcaacaccacaggagttgagaagccaaagtttcttccggacttatatgattataaagaaaatcgtttcattgaaattggagtgacaaggagggaggtccatatatactatctagaaaaggccaataagataaagtctgagaagacacacatccatatcttttcattcactggagaagaaatggccacaaaagcagactacactcttgatgaagagagtagagcaaggatcaaaaccagactattcactataagacaagaaatggccagtagaggtctctgggattcctttcgtcagtccgagagaggcgaagagacaattgaagaaagatttgaaattacaggaaccatgcgcaggctcgccgaccaaagtctcccaccgaacttctccagccttgaaaactttagagcctatgtggatggattcgaaccgaacggctgcattgagggcaagctttctcaaatgtccaaagaagtaaatgcaagaattgaaccatttttgaagacaacaccacgccctctcagattaccagaagggcctccctgctctcagcggtcaaaatttctgttgatggatgctctgaagcttagcattgaagacccgagtcatgagggtgagggaataccactgtatgatgctatcaaatgtatgaagaccttttttggctggaaagagcccaacattgtcaagccacatgagaggggcataaaccctaattatctcctggcttggaagcaagtgcaagcagaactgcaggatattgaaaatgaagacaagattccaaagacaaaaaacatgaagaaaacaagccaattaaagtgggcacttggtgagaacatggcaccagagaaagtggactttgaagattgcaaggatgtcagcgatttgaaacagtatgacagcgatgagccagaacaaaggtcgctagcaagttgggtccaaagtgaattcaacaaagcttgtgaattgactgattcaagctggatagaactcgatgaaataggggaaaatgtcgccccaatcgagcatattgcaagcatgaggaggaattattttacagctgaagtgtctcactgcagggcaacagagtacataatgaagggagtgtacataaactcagctttactcaacgcctcttgtgcagccatggatgattttcagttgatcccaatgataagcaaatgcagaaccaaagaaggacgacggaaaacaaatttgtatggattcatcataaagggaaggtctcatttgaggaatgatactgatgtggtgaattttgtgagcatggaattttctcttactgaccctagattagaaccacacaagtgggagaagtattgtgtccttgaaataggggatatgctcctacgaactgcaataggccaagtttcaagacccatgtttctgtatgtgacaaccaatggaacttccaagatcaaaatgaaatggggtatggagatgaggcgttgtcttcttcaatccctccagcaaatcgaaagcatgattgaggccgagtcctctgtcaaggaaaaagacatgactaaagaattctttgaaaacaagtcggagacatggcccattggagaatcacccaaaggagtagaagaaggttccattgggaaagtatgcaggactctgctagcaaagtctgtattcaacagcttgtatgcatctccacaacttgaaggtttttcagctgagtcaagaaagctgcttctcattgttcaggcacttagggacaacctggaacctggcaccttcgatcttggggggctatatgaagcaattgaggagtgcctgattaatgatccctgggttttgcttaatgcatcttggttcaactccttcctcacacatgcactgaaatagttgtggcaatgctactatttgctatacatcctgtccaaaaaagtaccttgtttctact

SEQ ID NO: 133 - DNA, 1635 bp, Influenza A virus
agcaaaagcagggcaaggatcaaaactaggctgttcaccataagacaggaactggctagcaggggtctatgggattcctttcgtcagtccgagagaggcgaagagacaattgaagaaagatttgaaatcacaggaacaatgcgcaggcttgccgaccaaagtctcccaccgaatttctccagccttgaaaattttagagcctatgtggatggattcgaaccgaacggctgcattgagggcaagctttctcagatgtcaaaagaagtaacggccagaattgagccctttcttaaaacaacaccacgtcctctaagactgccgggtggacctccctgttcccaaaggtcaaaattcttactgatggatgctctgaaattaagcattgaggacccgagtcatgagggagaggggataccgctgtatgatgcgatcaaatgcatgaaaacatttttcggctggaaagagcccaaaattatcaagtcacatgagaagggtataaacccaaattatctcctagcttggaagcaggtgctggcagagctccaggacattgaaaatgatgaaaagatcccaaaaacaaagaacatgaagaaaacaagccaattaaagtgggcattaggtgagaacatggcaccagagaaagtggactttgaggattgcaaagacgttagtgacctgaaacaatatgatagtgatgaaccagagcccaaatcgctagcaagttggatccagagtgaatttaacaaggcatgtgagttgaccgattcaagctgggtagaacttgatgaaataggagaagatgttgctccaatcgagcacattgcgagtatgagaaggaattacttcacagcagaagtgtcacactgccgggctactgagtatataatgaagggagtgtatattaacacagcgttgctcaatgcatcttgtgcagccatggatgacttccaattgattccaatgataagcaaatgcagaacaaaagaagggagacggaaaacaaacctgtatgggttcattatcaagggaaggtcccatttgaggaatgatactgatgtggtaaactttgtgagcatggaattttctcttacagacccgaaactggaaccacacaagtgggagaagtactgtgttcttgaagtaggggacatgctcctgagaacttcaataggccaggtgtcaaggcccatgttcctatacgtgagaaccaatggaacctccaaaattaaaatgaaatggggaatggagatgaggcgttgcctccttcaatcccttcaacaaattgagagcatgattgaggcagagtcttctatcaaagagaaggacatgaccaaagaattttttgaaaacaagtcggagacgtggccgattggagagtcacctaagggagtggaggaaggctccatcgggaaggtgtgcaggaccttactagcaaagtctgtgttcaacagcttgtatgcatctccacaactcgaggggttttcagctgaatcaagaaaactgttactcattgttcaggcacttagggacaacctggaacctggaaccttcgacattgaaggactgtatgaagcaattgaggagtgcctgattaatgatccctgggttttgcttaatgcatcttggttcaactcc

SEQ ID NO: 134 - DNA, 100 bp, Influenza A virus
tctcggacgaaaaggcagcgagcccgatcgtgccttcctttgacatgagtaatgaaggatcttatttcttcggagacaatgcagaggagtacgacaatta

SEQ ID NO: 135 - DNA, 100 bp, Influenza A virus
tctcggacgaaaaggcagcgagcccgatcgtgccctcctttgacatgagtaatgaaggatcttatttcttcggagacaatgcagaggagtacgacaatta

SEQ ID NO: 136 - DNA, 100 bp, Influenza A virus
tctcggactaaaaggcaacgaaccccatcgtgccctcttttgacatgagtaatgaaggatcttatttcttcggagacaatgcagaggagtacgacaatta

SEQ ID NO: 137 - DNA, 100 bp, Influenza A virus
tctcggacgaaaaggcagcgaacccgatcgtgccctcttttgacatgagtaatgaaggatcttatttcttcggagacaatgcagaggagtacgacaatta

SEQ ID NO: 138 - DNA, 100 bp, Influenza A virus
tctcagacgaaaaggcaacgaacccgatcgtgccctcttttgacatgagtaatgaaggatcttatttcttcggagacaatgcagaggagtacgacaatta

SEQ ID NO: 139 - DNA, 100 bp, Influenza A virus
tctcagacgaaaaggcaacgaacccgatcgtgccttcctttgacatgagtaatgaaggatcttatttcttcggagacaatgcagaggaatatgacaattg

SEQ ID NO: 140 - DNA, 100 bp, Influenza A virus
tctcggacgaaaaggcgacgaacccgatcgtgccttcctttgacatgagtaacgagggatcttatttcttcggagacaatgcagaggaatatgacaatta

SEQ ID NO: 141 - DNA, 100 bp, Influenza A virus
tctcggacgaaaaggcaacgaacccgatcgtgccttcctttgacatgagcaatgaagggtcttatttcttcggagacaatgctgaggagtttgacagtta

SEQ ID NO: 142 - DNA, 100 bp, Influenza A virus
tctcagacgaaaaggcaacgaacccgatcgtgccttcctttgacatgagtaatgagggatcttatttcttcggagacaatgctgaggagtatgacaattg

SEQ ID NO: 143 - DNA, 100 bp, Influenza A virus
tctcggacgaaaaggcaacgaacccgatcgtgccttcctttgacatgagtaatgaaggatcttatttcttcggagacaatgcataggagtatgacaatta

SEQ ID NO: 144 - DNA, 100 bp, Influenza A virus
tctcggacgaaaaggcaacgaacccgatcgtgccttcctttgacatgagtaacgaagggtcttatttcttcggagacaatgcagaggaatatgacaatta

SEQ ID NO: 145 - RNA, 13 nt, Artificial Sequence
  Description of Artificial Sequence Synthetic oligonucleotide
aguagaaacaagg

SEQ ID NO: 146 - RNA, 13 nt, Artificial Sequence
  Description of Artificial Sequence Synthetic oligonucleotide
ccugcuuuucgcu

SEQ ID NO: 147 - DNA, 14 bp, Artificial Sequence
  Description of Artificial Sequence Synthetic oligonucleotide
gagacgaaagcagg

SEQ ID NO: 148 - DNA, 13 bp, Artificial Sequence
  Description of Artificial Sequence Synthetic oligonucleotide
agcgaaaagcagg

SEQ ID NO: 149 - RNA, 13 nt, Artificial Sequence
  Description of Artificial Sequence Synthetic oligonucleotide
ccuuguuucuacu

SEQ ID NO: 150 - DNA, 21 bp, Artificial Sequence
  Description of Combined DNA/RNA Molecule Synthetic oligonucleotide
gcuacaacuacuacaugactt

SEQ ID NO: 151 - DNA, 21 bp, Artificial Sequence
  Description of Combined DNA/RNA Molecule Synthetic oligonucleotide
gucauguaguaguuguagctt

SEQ ID NO: 152 - RNA, 62 nt, Artificial Sequence
  Description of Artificial Sequence Synthetic oligonucleotide
cgggggugcuacaacuacuacaugacgcagguccagucauguaguaguuguagcuuccccug

SEQ ID NO: 153 - DNA, 21 bp, Artificial Sequence
  Description of Combined DNA/RNA Molecule Synthetic oligonucleotide
ggaucuuauuucuucggagtt

SEQ ID NO: 154 - DNA, 21 bp, Artificial Sequence
  Description of Combined DNA/RNA Molecule Synthetic oligonucleotide
cuccgaagaaauaagaucctt

SEQ ID NO: 155 - RNA, 60 nt, Artificial Sequence
  Description of Artificial Sequence Synthetic oligonucleotide
cgggguggaucuuauuucuucggaggcagguccacuccgaagaaauaagauccuucccug

SEQ ID NO: 156 - DNA, 21 bp, Artificial Sequence
  Description of Combined DNA/RNA Molecule Synthetic oligonucleotide
ugcgcuccuggacguagcctt

SEQ ID NO: 157 - DNA, 21 bp, Artificial Sequence
  Description of Combined DNA/RNA Molecule Synthetic oligonucleotide
ggcuacguccaggagcgcatt

SEQ ID NO: 158 - RNA, 60 nt, Artificial Sequence
  Description of Artificial Sequence Synthetic oligonucleotide
cgggguugcgcuccuggacguagccgcagguccaggcuacguccaggagcgcauucccug

SEQ ID NO: 159 - RNA, 119 nt, Artificial Sequence
  Description of Artificial Sequence Synthetic polynucleotide
cgggguggaucuuauuucuucggaggcagcgggguugcgcuccuggacguagccgcagguccaggcuacguccaggagcgcauucccuguccacuccgaagaaauaagauccuucccug

SEQ ID NO: 160 - RNA, 119 nt, Artificial Sequence
  Description of Artificial Sequence Synthetic polynucleotide
cgggguugcgcuccuggacguagccgcagcgggguggaucuuauuucuucggaggcagguccacuccgaagaaauaagauccuucccuguccaggcuacguccaggagcgcauucccug

SEQ ID NO: 161 - DNA, 24 bp, Artificial Sequence
  Description of Artificial Sequence Synthetic primer
cgctcagacatgagaacagaatgg

SEQ ID NO: 162 - DNA, 36 bp, Artificial Sequence
  Description of Artificial Sequence Synthetic primer
atatcgtctcgtattagtagaaacaaggtagttttt

SEQ ID NO: 163 - DNA, 24 bp, Artificial Sequence
  Description of Artificial Sequence Synthetic primer
cgctcagacatgagaacagaatgg

SEQ ID NO: 164 - DNA, 23 bp, Artificial Sequence
  Description of Artificial Sequence Synthetic primer
taactagcctgactagcaacctc

SEQ ID NO: 165 - DNA, 25 bp, Artificial Sequence
  Description of Artificial Sequence Synthetic primer
gtgcagaaatcagcccgaatggttc

SEQ ID NO: 166 - DNA, 33 bp, Artificial Sequence
  Description of Artificial Sequence Synthetic primer
atatcgtctcgtattagtagaaacaaggcattt

SEQ ID NO: 167 - DNA, 24 bp, Artificial Sequence
  Description of Artificial Sequence Synthetic primer
gcgaaaggagagaaggctaatgtg

SEQ ID NO: 168 - DNA, 34 bp, Artificial Sequence
  Description of Artificial Sequence Synthetic primer
atatggtctcgtattagtagaaacaaggtcgttt

SEQ ID NO: 169 - DNA, 25 bp, Artificial Sequence
  Description of Artificial Sequence Synthetic primer
gcttcttatcgttcaggctcttagg

SEQ ID NO: 170 - DNA, 33 bp, Artificial Sequence
  Description of Artificial Sequence Synthetic primer
atatcgtctcgtattagtagaaacaaggtactt

SEQ ID NO: 171 - DNA, 24 bp, Artificial Sequence
  Description of Artificial Sequence Synthetic primer
cggattgatgcacggattgatttc

SEQ ID NO: 172 - DNA, 27 bp, Artificial Sequence
  Description of Artificial Sequence Synthetic primer
gacgtctgagctcttcaatggtggaac

SEQ ID NO: 173 - DNA, 24 bp, Artificial Sequence
  Description of Artificial Sequence Synthetic primer
gcgaaaggagagaaggctaatgtg

SEQ ID NO: 174 - DNA, 24 bp, Artificial Sequence
  Description of Artificial Sequence Synthetic primer
aatcgctgtctggctgtcagtaag

SEQ ID NO: 175 - DNA, 25 bp, Artificial Sequence
  Description of Artificial Sequence Synthetic primer
gcttcttatcgttcaggctcttagg

SEQ ID NO: 176 - DNA, 25 bp, Artificial Sequence
  Description of Artificial Sequence Synthetic primer
ccgagaagcattaagcaaaacccag

SEQ ID NO: 177 - DNA, 20 bp, Artificial Sequence
  Description of Artificial Sequence Synthetic primer
tctgtcagggttggaaagtc

SEQ ID NO: 178 - DNA, 20 bp, Artificial Sequence
  Description of Artificial Sequence Synthetic primer
aaatgcaaaccgcttccaac

SEQ ID NO: 179 - DNA, 57 bp, Artificial Sequence
  Description of Artificial Sequence Synthetic oligonucleotide
tggatcttatttcttcggagattcaagagatctccgaagaaataagatccttttttc

SEQ ID NO: 180 - DNA, 61 bp, Artificial Sequence
  Description of Artificial Sequence Synthetic oligonucleotide
tcgagaaaaaaggatcttatttcttcggagatctcttgaatctccgaagaaataagatcca

SEQ ID NO: 181 - DNA, 55 bp, Artificial Sequence
  Description of Artificial Sequence Synthetic oligonucleotide
tgatctgttccaccattgaattcaagagattcaatggtggaacagatcttttttc

SEQ ID NO: 182 - DNA, 59 bp, Artificial Sequence
  Description of Artificial Sequence Synthetic oligonucleotide
tcgagaaaaaagatctgttccaccattgaatctcttgaattcaatggtggaacagatca

SEQ ID NO: 183 - DNA, 56 bp, Artificial Sequence
  Description of Artificial Sequence Synthetic oligonucleotide
tgcgataatataactgcaagattcaagagatcttgcagttatattatcgttttttc

SEQ ID NO: 184 - DNA, 60 bp, Artificial Sequence
  Description of Artificial Sequence Synthetic oligonucleotide
tcgagaaaaaacgataatataactgcaagatctcttgaatcttgcagttatattatcgca

SEQ ID NO: 185 - DNA, 19 bp, Artificial Sequence
  Description of Artificial Sequence Synthetic oligonucleotide
cgataatataactgcaaga

SEQ ID NO: 186 - DNA, 19 bp, Artificial Sequence
  Description of Artificial Sequence Synthetic oligonucleotide
tcttgcagttatattatcg

SEQ ID NO: 187 - DNA, 55 bp, Artificial Sequence
  Description of Artificial Sequence Synthetic oligonucleotide
tgcaattgaggagtgcctgattcaagagatcaggcactcctcaattgcttttttc

SEQ ID NO: 188 - DNA, 22 bp, Artificial Sequence
  Description of Combined DNA/RNA Molecule Synthetic oligonucleotide
ggaucuuauuucuucggagatt

SEQ ID NO: 189 - DNA, 22 bp, Artificial Sequence
  Description of Combined DNA/RNA Molecule Synthetic oligonucleotide
ucuccgaagaaauaagaucctt

SEQ ID NO: 190 - DNA, 21 bp, Artificial Sequence
  Description of Combined DNA/RNA Molecule Synthetic oligonucleotide
gaaagcaggucaauuauautt

SEQ ID NO: 191 - DNA, 21 bp, Artificial Sequence
  Description of Combined DNA/RNA Molecule Synthetic oligonucleotide
auauaauugaccugcuuuctt

SEQ ID NO: 192 - DNA, 21 bp, Artificial Sequence
  Description of Combined DNA/RNA Molecule Synthetic oligonucleotide
gucaauuauauucaauaugtt

SEQ ID NO: 193 - DNA, 21 bp, Artificial Sequence
  Description of Combined DNA/RNA Molecule Synthetic oligonucleotide
cauauugaauauaauugactt

SEQ ID NO: 194 - DNA, 21 bp, Artificial Sequence
  Description of Combined DNA/RNA Molecule Synthetic oligonucleotide
cucgcacccgcgagauacutt

SEQ ID NO: 195 - DNA, 21 bp, Artificial Sequence
  Description of Combined DNA/RNA Molecule Synthetic oligonucleotide
aguaucucgcgggugcgagtt

SEQ ID NO: 196 - DNA, 21 bp, Artificial Sequence
  Description of Combined DNA/RNA Molecule Synthetic oligonucleotide
auaaucaagaaguacacautt

SEQ ID NO: 197 - DNA, 21 bp, Artificial Sequence
  Description of Combined DNA/RNA Molecule Synthetic oligonucleotide
auguguacuucuugauuautt

SEQ ID NO: 198 - DNA, 21 bp, Artificial Sequence
  Description of Combined DNA/RNA Molecule Synthetic oligonucleotide
ugaaauggaugauggcaautt

SEQ ID NO: 199 - DNA, 21 bp, Artificial Sequence
  Description of Combined DNA/RNA Molecule Synthetic oligonucleotide
auugccaucauccauuucatt

SEQ ID NO: 200 - DNA, 21 bp, Artificial Sequence
  Description of Combined DNA/RNA Molecule Synthetic oligonucleotide
cuggucaugcagaucucagtt

SEQ ID NO: 201 - DNA, 21 bp, Artificial Sequence
  Description of Combined DNA/RNA Molecule Synthetic oligonucleotide
cugagaucugcaugaccagtt

SEQ ID NO: 202 - DNA, 21 bp, Artificial Sequence
  Description of Combined DNA/RNA Molecule Synthetic oligonucleotide
uaugcaaggcugcaaugggtt

SEQ ID NO: 203 - DNA, 21 bp, Artificial Sequence
  Description of Combined DNA/RNA Molecule Synthetic oligonucleotide
cccauugcagccuugcauatt

SEQ ID NO: 204 - DNA, 21 bp, Artificial Sequence
  Description of Combined DNA/RNA Molecule Synthetic oligonucleotide
caucgucaaugaugugggatt

SEQ ID NO: 205 - DNA, 21 bp, Artificial Sequence
  Description of Combined DNA/RNA Molecule Synthetic oligonucleotide
ucccacaucauugacgaugtt

SEQ ID NO: 206 - DNA, 21 bp, Artificial Sequence
  Description of Combined DNA/RNA Molecule Synthetic oligonucleotide
aaauaccugcagaaaugcutt

SEQ ID NO: 207 - DNA, 21 bp, Artificial Sequence
  Description of Combined DNA/RNA Molecule Synthetic oligonucleotide
agcauuucugcagguauuutt

SEQ ID NO: 208 - DNA, 21 bp, Artificial Sequence
  Description of Combined DNA/RNA Molecule Synthetic oligonucleotide
aacaauaugauaaacaaugtt

SEQ ID NO: 209 - DNA, 21 bp, Artificial Sequence
  Description of Combined DNA/RNA Molecule Synthetic oligonucleotide
cauuguuuaucauauuguutt

SEQ ID NO: 210 - DNA, 21 bp, Artificial Sequence
  Description of Combined DNA/RNA Molecule Synthetic oligonucleotide
cgaaagcagguacugaucctt

SEQ ID NO: 211 - DNA, 21 bp, Artificial Sequence
  Description of Combined DNA/RNA Molecule Synthetic oligonucleotide
ggaucaguaccugcuuucgtt

SEQ ID NO: 212 - DNA, 21 bp, Artificial Sequence
  Description of Combined DNA/RNA Molecule Synthetic oligonucleotide
aggcuauucaccauaagactt

SEQ ID NO: 213 - DNA, 21 bp, Artificial Sequence
  Description of Combined DNA/RNA Molecule Synthetic oligonucleotide
gucuuauggugaauagccutt

SEQ ID NO: 214 - DNA, 21 bp, Artificial Sequence
  Description of Combined DNA/RNA Molecule Synthetic oligonucleotide
gggauuccuuucgucaguctt

SEQ ID NO: 215 - DNA, 21 bp, Artificial Sequence
  Description of Combined DNA/RNA Molecule Synthetic oligonucleotide
gacugacgaaaggaauccctt

SEQ ID NO: 216 - DNA, 21 bp, Artificial Sequence
  Description of Combined DNA/RNA Molecule Synthetic oligonucleotide
gcaucuugugcagcaauggtt

SEQ ID NO: 217 - DNA, 21 bp, Artificial Sequence
  Description of Combined DNA/RNA Molecule Synthetic oligonucleotide
ccauugcugcacaagaugctt

SEQ ID NO: 218 - DNA, 21 bp, Artificial Sequence
  Description of Combined DNA/RNA Molecule Synthetic oligonucleotide
guuguggcagugcuacuautt

SEQ ID NO: 219 - DNA, 21 bp, Artificial Sequence
  Description of Combined DNA/RNA Molecule Synthetic oligonucleotide
auaguagcacugccacaactt

SEQ ID NO: 220 - DNA, 21 bp, Artificial Sequence
  Description of Combined DNA/RNA Molecule Synthetic oligonucleotide
uacuauuugcuauccauactt

SEQ ID NO: 221 - DNA, 21 bp, Artificial Sequence
  Description of Combined DNA/RNA Molecule Synthetic oligonucleotide
guauggauagcaaauaguatt

SEQ ID NO: 222 - DNA, 21 bp, Artificial Sequence
  Description of Combined DNA/RNA Molecule Synthetic oligonucleotide
uagauaaucacucacugagtt

SEQ ID NO: 223 - DNA, 21 bp, Artificial Sequence
  Description of Combined DNA/RNA Molecule Synthetic oligonucleotide
cucagugagugauuaucuatt

SEQ ID NO: 224 - DNA, 21 bp, Artificial Sequence
  Description of Combined DNA/RNA Molecule Synthetic oligonucleotide
cgucccaaggcaccaaacgtt

SEQ ID NO: 225 - DNA, 21 bp, Artificial Sequence
  Description of Combined DNA/RNA Molecule Synthetic oligonucleotide
cguuuggugccuugggacgtt

SEQ ID NO: 226 - DNA, 21 bp, Artificial Sequence
  Description of Combined DNA/RNA Molecule Synthetic oligonucleotide
auuucuucggagacaaugctt

SEQ ID NO: 227 - DNA, 21 bp, Artificial Sequence
  Description of Combined DNA/RNA Molecule Synthetic oligonucleotide
gcauugucuccgaagaaautt

SEQ ID NO: 228 - DNA, 21 bp, Artificial Sequence
  Description of Combined DNA/RNA Molecule Synthetic oligonucleotide
ugcagaggaguacgacaautt

SEQ ID NO: 229 - DNA, 21 bp, Artificial Sequence
  Description of Combined DNA/RNA Molecule Synthetic oligonucleotide
auugucguacuccucugcatt

SEQ ID NO: 230 - DNA, 21 bp, Artificial Sequence
  Description of Combined DNA/RNA Molecule Synthetic oligonucleotide
gagtaatgaaggatcttattt

SEQ ID NO: 231 - DNA, 21 bp, Artificial Sequence
  Description of Combined DNA/RNA Molecule Synthetic oligonucleotide
ataagatccttcattactctt

SEQ ID NO: 232 - DNA, 21 bp, Artificial Sequence
  Description of Combined DNA/RNA Molecule Synthetic oligonucleotide
cgaaagcagguagauauugtt

SEQ ID NO: 233 - DNA, 21 bp, Artificial Sequence
  Description of Combined DNA/RNA Molecule Synthetic oligonucleotide
caauaucuaccugcuuucgtt

SEQ ID NO: 234 - DNA, 21 bp, Artificial Sequence
  Description of Combined DNA/RNA Molecule Synthetic oligonucleotide
uagauauugaaagaugagutt

SEQ ID NO: 235 - DNA, 21 bp, Artificial Sequence
  Description of Combined DNA/RNA Molecule Synthetic oligonucleotide
acucaucuuucaauaucuatt

SEQ ID NO: 236 - DNA, 21 bp, Artificial Sequence
  Description of Combined DNA/RNA Molecule Synthetic oligonucleotide
ucauggaauggcuaaagactt

SEQ ID NO: 237 - DNA, 21 bp, Artificial Sequence
  Description of Combined DNA/RNA Molecule Synthetic oligonucleotide
gucuuuagccauuccaugatt

SEQ ID NO: 238 - DNA, 21 bp, Artificial Sequence
  Description of Combined DNA/RNA Molecule Synthetic oligonucleotide
accaauccugucaccucugtt

SEQ ID NO: 239 - DNA, 21 bp, Artificial Sequence
  Description of Combined DNA/RNA Molecule Synthetic oligonucleotide
cagaggugacaggauuggutt

SEQ ID NO: 240 - DNA, 21 bp, Artificial Sequence
  Description of Combined DNA/RNA Molecule Synthetic oligonucleotide
uguguucacgcucaccgugtt

SEQ ID NO: 241 - DNA, 21 bp, Artificial Sequence
  Description of Combined DNA/RNA Molecule Synthetic oligonucleotide
cacggugagcgugaacacatt

SEQ ID NO: 242 - DNA, 21 bp, Artificial Sequence
  Description of Combined DNA/RNA Molecule Synthetic oligonucleotide
cagugagcgaggacugcagtt

SEQ ID NO: 243 - DNA, 21 bp, Artificial Sequence
  Description of Combined DNA/RNA Molecule Synthetic oligonucleotide
cugcaguccucgcucacugtt

SEQ ID NO: 244 - DNA, 21 bp, Artificial Sequence
  Description of Combined DNA/RNA Molecule Synthetic oligonucleotide
gacgcuuuguccaaaaugctt

SEQ ID NO: 245 - DNA, 21 bp, Artificial Sequence
  Description of Combined DNA/RNA Molecule Synthetic oligonucleotide
gcauuuuggacaaagcguctt

SEQ ID NO: 246 - DNA, 21 bp, Artificial Sequence
  Description of Combined DNA/RNA Molecule Synthetic oligonucleotide
gucaggcuaggcaaauggutt

SEQ ID NO: 247 - DNA, 21 bp, Artificial Sequence
  Description of Combined DNA/RNA Molecule Synthetic oligonucleotide
accauuugccuagccugactt

SEQ ID NO: 248 - DNA, 21 bp, Artificial Sequence
  Description of Combined DNA/RNA Molecule Synthetic oligonucleotide
uucuugaaaauuugcaggctt

SEQ ID NO: 249 - DNA, 21 bp, Artificial Sequence
  Description of Combined DNA/RNA Molecule Synthetic oligonucleotide
gccugcaaauuuucaagaatt

SEQ ID NO: 250 - DNA, 21 bp, Artificial Sequence
  Description of Combined DNA/RNA Molecule Synthetic oligonucleotide
ucauugggaucuugcacuutt

SEQ ID NO: 251 - DNA, 21 bp, Artificial Sequence
  Description of Combined DNA/RNA Molecule Synthetic oligonucleotide
aagugcaagaucccaaugatt

SEQ ID NO: 252 - DNA, 21 bp, Artificial Sequence
  Description of Combined DNA/RNA Molecule Synthetic oligonucleotide
uguggauucuugaucgucutt

SEQ ID NO: 253 - DNA, 21 bp, Artificial Sequence
  Description of Combined DNA/RNA Molecule Synthetic oligonucleotide
agacgaucaagaauccacatt

SEQ ID NO: 254 - DNA, 21 bp, Artificial Sequence
  Description of Combined DNA/RNA Molecule Synthetic oligonucleotide
ugucagcauagagcuggagtt

SEQ ID NO: 255 - DNA, 21 bp, Artificial Sequence
  Description of Combined DNA/RNA Molecule Synthetic oligonucleotide
cuccagcucuaugcugacatt

SEQ ID NO: 256 - DNA, 21 bp, Artificial Sequence
  Description of Combined DNA/RNA Molecule Synthetic oligonucleotide
gtcgaaacgcctatcagaatt

SEQ ID NO: 257 - DNA, 21 bp, Artificial Sequence
  Description of Combined DNA/RNA Molecule Synthetic oligonucleotide
uucugauaggcguuucgactt

SEQ ID NO: 258 - DNA, 21 bp, Artificial Sequence
  Description of Combined DNA/RNA Molecule Synthetic oligonucleotide
aaaagcagggugacaaagatt

SEQ ID NO: 259 - DNA, 21 bp, Artificial Sequence
  Description of Combined DNA/RNA Molecule Synthetic oligonucleotide
ucuuugucacccugcuuuutt

SEQ ID NO: 260 - DNA, 21 bp, Artificial Sequence
  Description of Combined DNA/RNA Molecule Synthetic oligonucleotide
gcagggugacaaagacauatt

SEQ ID NO: 261 - DNA, 21 bp, Artificial Sequence
  Description of Combined DNA/RNA Molecule Synthetic oligonucleotide
uaugucuuugucacccugctt

SEQ ID NO: 262 - DNA, 21 bp, Artificial Sequence
  Description of Combined DNA/RNA Molecule Synthetic oligonucleotide
ggaugucaaaaaugcaguutt

SEQ ID NO: 263 - DNA, 21 bp, Artificial Sequence
  Description of Combined DNA/RNA Molecule Synthetic oligonucleotide
aacugcauuuuugacaucctt

SEQ ID NO: 264 - DNA, 21 bp, Artificial Sequence
  Description of Combined DNA/RNA Molecule Synthetic oligonucleotide
agagauucgcuuggagaagtt

SEQ ID NO: 265 - DNA, 21 bp, Artificial Sequence
  Description of Combined DNA/RNA Molecule Synthetic oligonucleotide
cuucuccaagcgaaucucutt

SEQ ID NO: 266 - DNA, 21 bp, Artificial Sequence
  Description of Combined DNA/RNA Molecule Synthetic oligonucleotide
caguaaugagaaugggagatt

SEQ ID NO: 267 - DNA, 21 bp, Artificial Sequence
  Description of Combined DNA/RNA Molecule Synthetic oligonucleotide
ucucccauucucauuacugtt

SEQ ID NO: 268 - DNA, 21 bp, Artificial Sequence
  Description of Combined DNA/RNA Molecule Synthetic oligonucleotide
uuguggauucuugaucguctt

SEQ ID NO: 269 - DNA, 21 bp, Artificial Sequence
  Description of Combined DNA/RNA Molecule Synthetic oligonucleotide
gacgaucaagaauccacaatt

SEQ ID NO: 270 - DNA, 59 bp, Artificial Sequence
  Description of Artificial Sequence Synthetic oligonucleotide
tcgagaaaaaagcaattgaggagtgcctgatctcttgaatcaggcactcctcaattgca

SEQ ID NO: 271 - RNA, 51 nt, Artificial Sequence
  Description of Artificial Sequence Synthetic oligonucleotide
ggaucuuauuucuucggagauucaagagaucuccgaagaaauaagauccuu

SEQ ID NO: 272 - DNA, 22 bp, Artificial Sequence
  Description of Artificial Sequence Synthetic oligonucleotide
aaaaaaaaaaaaaaaaaaaaaa